SEQUENCE LISTING (174 sequences)

SEQ ID NO: 1 - DNA, 117 bp, Artificial Sequence
  RAG1 exon 1
agaaacaagagggcaaggagagagcagagaacacactttgccttctctttggtattgagtaatatcaaccaaattgcagacatctcaacactttggccaggcagcctgctgagcaag

SEQ ID NO: 2 - DNA, 5168 bp, Artificial Sequence
  RAG1 intron 1
gtaacactcatacttttcatgccttgagccaaaatatttattacatttttatgtttctaactagaagtgcttgagctttttttccttccaggtgatgaggggatggaatgagcaaagctacatcaatttttttttaatgtatgaaaataaaaaaggtacaagaggccaagtttagggccactgaaggttcatagaaagatgcaaaatatctgaattactataaatgaatgctattgtcagaggaaaggtttaaggagtgcttcttgaatgaatgtgtacaaatcagcagaaggtaaggtgtgagactcttggaaatgaatactggtagttcaggtgagaaaaataatcaggaacataatagggtgggaggaaatgtatggtttcccaggtattaacaagtattgccaggcatttcctgaactagattggcctaagtaggagaccaatgtttctcaaaatattcactcattttagaatcactgaatgtttaaaaatgcaatttctggattccttcccaaacagccagactctttgggacctgatgatctgcatttctttttaaaaacaaactcgctcatgattctgatttgtattaattttgagaattgccatggtagagaccctgctttgaggttatgttcttgagtcaggattcctggccagggattgtgatgatatatttctctttctgaagtggttcatgcaagaggttgtctgaaggaagagcaagaattgtagtgttattttgtggatacttgagacttataaaaaggctttttattttgtcacatttttgatacatgatgtttggcaaaaaacagacgatagtatttgcagagtgaatgaataagtggaacaggtgtgataatgagaggtcacacttgagcacacagttattacttggaaattgtgtacagactaagttgaagatgttaggagggaagattgtgggccaagtaacggggtgtatgtgtgtgggtatagggtgggcagctgggatggaaatggggggctgctgctgctgctgcaccctggcctcctgaactaatgatatcactcaccagaaactactgttcctgcactgtccaagccaccccaaactagtttgtcaaaatgaatctgtgctgtgtggagggaggcacgcctgtagctctgatgtcagatggcaatgtcgagatggcagtggccggtggggacagggctgagccagcaccaaccactcagcctttgagatcccgaggctggtctactgctgagaccttttgttagaagagaggagatcaagcatttgcaaggtttctgagtgtcaaaatatgaatccaagataactctttcacaatcctaacttcatgctgtctacaggtccatattttagcctgctttctccatgttcatccgaaaagaaagaaaagctaagggtggtggtcatatttgaaattagccagatcttaagtttttctgggggaaatttagaagaaaatatggaaaagtgactatgagcacatatacagctagtctttaaaacagttttatccaaaataaatgtatcacaaaattaataaaaatagttacttgcttgttttgaataattcaaatgatacaaaaattaataaaataaaaagtgcaaaaggccctcttatcaatgccaattctatttttttcagaaattaaacactgttaagattttagtgtgtatcctttcagaattcctgtgatttcatatatgtacaaatacaaacgtatctacataaagggaatcctactatacttgctattgtcattctattctctgctttttcatgtgagcatctttccatgtcactgatgcatacagaaattgcacatatgcatcagtgcatacagaaaattaaattttctgcatggttttccactgtatgtctggaccatagtttatttaataataatgccctttgggtaattatttatattgtttcctgctttttcaaagtaacagcttttgaaacaaatctctctctgtctttatataaatattgttgcattcctgtggaaatgtttctattggataacttcccaaaaggagatttattgcatcaaagataatatattcaaaaattttaaagatattgctaaattgtctagtaggtattttataccaatttatactcctcccaagaatgtatggagatatcttaatttctccatgccttcattaatgctgaaccatataagtagttttaatctttgctaattgaatagataaaaaatatctaatctaagtctagttcttaaaagttctatcttctaccaaaagtaatacacgtctattttagggagtaaaaatcacaagtaaggataaaaaatagtgcagcaataaacacaggagtgtagatgtctctgaacatactgatttaacttcctttggataaatacccagtagtaggactgctggatcatataataattctatctttagtttttttgaggacctccatactattcttcatagtggctgtactaatttacattcctaccaactgtgtatgaaggttcccttttctctacatccttgccagcattcattattgcttgtcatttggatacaatctattttaactggggtgagatgacatctcattgtagttttgatatgcatttctctgatgatcagtggtgttgagcaccttttcatatacctgtttgccatttgtatgtcttcctttgagaaatgtctattcagatattttacctattttaaaatcggattattagattgtttcctgtagagttgtttgagctccttgtatattctggttattaatctcttgtcagatgcatagcttacaaatattttctcccatcatgtggattgtgtcttcactttgtggattgtttactttgctgtgcagaagcttttaacttgatgcaatcccatttgtccacttttgctttggttgccttccacaggagtatttaaataaatgtagtttggtagattttggtatagtaatgcaggccagtgggagtcaggggagaaatgtgtagggaagtgagatagttctaaggatcctacaaacatgccttatgattgacttactcaatgtgaaagtcaatattaaacttgatgagctctagagatggtcatgcattttaaaaagaattactcaaaatattgtcttggaataccagagagcaagtgctttaagtataggctgggaagtaaaatgctaaaggaatgagaaggcatttggggttgagttcaacctaagaggcaggggagccacagggaaagacctagcacctgccacagaagagaattaggaagcagaattgaactataagcaattttgaggtgttcgttgggctgcagttgaaatattttttgaggttaatgagacatttgaaatggccgtgtattgtttaactcttgcatagtcctgcatagggaacaatctaataggatttctctgtgaatcaagtcttagaaatttgcttttaatttttatgaaaaacgcccatttctttgtttttgagacagagtcctgctctgtcatccaggctgggttgcagtggcgtgatcttggcccactgcaatctctgcctcctgggttcaggcaattttcctgtctcagcctcccgagtagctgggatttcaagtgcctgccaccatgcccggctaaatttttttgtatttttggtacagatggagtatcaccatgttggccaggctggtctcgaactcctgacctcaagtgattcaccagccttgacctcccaaagtgttgggatcacaggcatgagccactgtgcctgtgccccaaaacaccaatttctgatgtgtgatgcatgtaagatagaacaaacttcagtaaagcggggacttgaaaagaggctttggtaacagctgtcagcattaacccttgcccctccgtacctcctaatcccacccctgctcaaagtatgttcatctgagaatttgtctccataactatgtgactataaaaattctcatcgattttgttagttgatcaattgagggaaaaacatatgttacttgatataactggtgggtcaaaagaattaacccaggcaaatttgagataggtggatgggatgatggattgaaaatacagctgctctctttccaatcatgtactaagtaatttgggaaagattgatctaattgggtctagagagtacacttcacatggcattgtttgactttttttctgcatcgctagcgatctgtgcattacaactcaaatcagtcgggtttcctggcatatgtaattgccaatgttttttaccagaagagaaacattactcccacctcttcttattatgttacaaactatagtgctaatgaccatcgaccaacagtgactttcaggatgacctgtgtgagttttatctgaaaccatgtgaatttttcatcttaaaagtcccttagaatctcagtctatgtacactcaggtttgttgcaggtttagagttccgtgttttttgtttctaatgtagacacagccttataatttacaacagcattcactaattaaaattgtaagcataattactatccacgatacttattattagtttgcattcataaagctcaaaattcacttcatcctttcaagtagtgaataattagtttctttgggtttgcagctttatcatccttttatgacccatttggaagaaataaacaaccaaccccctggaagactgctttaaaaagctggaaatacattgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaaggtttttgtggaaggaaataaaactttacatgtatatatttaagcacttatatgtgtgtaacaggtataagtaaccataaacactgtcagaagaggaaataactctatgatcagcacctaacatgatatattaaggtagaagatttaatacatatcttttggaatacatgaataaataattgaatgtatttatttttattatttataagatacatcagtgggatattgatattggtcttaatatgacttgttttcattgttctcag

SEQ ID NO: 3 - DNA, 6472 bp, Artificial Sequence
  RAG1 exon 2
gtacctcagccagcatggcagcctctttcccacccaccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcacaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtcagtttggaaaaatataatcaccacatctcaagtcacaaggaatcaaaagagatttttgtgcacattaataaagggggccggccccgccaacatcttctgtcgctgactcggagagctcagaagcaccggctgagggagctcaagctgcaagtcaaagcctttgctgacaaagaagaaggtggagatgtgaagtccgtgtgcatgaccttgttcctgctggctctgagggcgaggaatgagcacaggcaagctgatgagctggaggccatcatgcagggaaagggctctggcctgcagccagctgtttgcttggccatccgtgtcaacaccttcctcagctgcagtcagtaccacaagatgtacaggactgtgaaagccatcacagggagacagatttttcagcctttgcatgcccttcggaatgctgagaaggtacttctgccaggctaccaccactttgagtggcagccacctctgaagaatgtgtcttccagcactgatgttggcattattgatgggctgtctggactatcatcctctgtggatgattacccagtggacaccattgcaaagaggttccgctatgattcagctttggtgtctgctttgatggacatggaagaagacatcttggaaggcatgagatcccaagaccttgatgattacctgaatggccccttcactgtggtggtgaaggagtcttgtgatggaatgggagacgtgagtgagaagcatgggagtgggcctgtagttccagaaaaggcagtccgtttttcattcacaatcatgaaaattactattgcccacagctctcagaatgtgaaagtatttgaagaagccaaacctaactctgaactgtgttgcaagccattgtgccttatgctggcagatgagtctgaccacgagacgctgactgccatcctgagtcctctcattgctgagagggaggccatgaagagcagtgaattaatgcttgagctgggaggcattctccggactttcaagttcatcttcaggggcaccggctatgatgaaaaacttgtgcgggaagtggaaggcctcgaggcttctggctcagtctacatttgtactctttgtgatgccacccgtctggaagcctctcaaaatcttgtcttccactctataaccagaagccatgctgagaacctggaacgttatgaggtctggcgttccaacccttaccatgagtctgtggaagaactgcgggatcgggtgaaaggggtctcagctaaacctttcattgagacagtcccttccatagatgcactccactgtgacattggcaatgcagctgagttctacaagatcttccagctagagataggggaagtgtataagaatcccaatgcttccaaagaggaaaggaaaaggtggcaggccacactggacaagcatctccggaagaagatgaacctcaaaccaatcatgaggatgaatggcaactttgccaggaagctcatgaccaaagagactgtggatgcagtttgtgagttaattccttccgaggagaggcacgaggctctgagggagctgatggatctttacctgaagatgaaaccagtatggcgatcatcatgccctgctaaagagtgcccagaatccctctgccagtacagtttcaattcacagcgttttgctgagctcctttctacgaagttcaagtataggtatgagggaaaaatcaccaattattttcacaaaaccctggcccatgttcctgaaattattgagagggatggctccattggggcatgggcaagtgagggaaatgagtctggtaacaaactgtttaggcgcttccggaaaatgaatgccaggcagtccaaatgctatgagatggaagatgtcctgaaacaccactggttgtacacctccaaatacctccagaagtttatgaatgctcataatgcattaaaaacctctgggtttaccatgaaccctcaggcaagcttaggggacccattaggcatagaggactctctggaaagccaagattcaatggaattttaagtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgctggcaatgtagagcaggcatggaattttatatgctagtgagtcataatgatatgttagtgttaattagttttttcttcctttgattttattggccataattgctactcttcatacacagtatatcaaagagcttgataatttagttgtcaaaagtgcatcggcgacattatctttaattgtatgtatttggtgcttcttcagggattgaactcagtatctttcattaaaaaacacagcagttttccttgctttttatatgcagaatatcaaagtcatttctaatttagttgtcaaaaacatatacatattttaacattagtttttttgaaaactcttggttttgtttttttggaaatgagtgggccactaagccacactttcccttcatcctgcttaatccttccagcatgtctctgcactaataaacagctaaattcacataatcatcctatttactgaagcatggtcatgctggtttatagattttttacccatttctactctttttctctattggtggcactgtaaatactttccagtattaaattatccttttctaacactgtaggaactattttgaatgcatgtgactaagagcatgatttatagcacaacctttccaataatcccttaatcagatcacattttgataaaccctgggaacatctggctgcaggaatttcaatatgtagaaacgctgcctatggttttttgcccttactgttgagactgcaatatcctagaccctagttttatactagagttttatttttagcaatgcctattgcaagtgcaattatatactccagggaaattcaccacactgaatcgagcatttgtgtgtgtatgtgtgaagtatatactgggacttcagaagtgcaatgtatttttctcctgtgaaacctgaatctacaagttttcctgccaagccactcaggtgcattgcagggaccagtgataatggctgatgaaaattgatgattggtcagtgaggtcaaaaggagccttgggattaataaacatgcactgagaagcaagaggaggagaaaaagatgtctttttcttccaggtgaactggaatttagttttgcctcagatttttttcccacaagatacagaagaagataaagatttttttggttgagagtgtgggtcttgcattacatcaaacagagttcaaattccacacagataagaggcaggatatataagcgccagtggtagttgggaggaataaaccattatttggatgcaggtggtttttgattgcaaatatgtgtgtgtcttcagtgattgtatgacagatgatgtattcttttgatgttaaaagattttaagtaagagtagatacattgtacccattttacattttcttattttaactacagtaatctacataaatatacctcagaaatcatttttggtgattattttttgttttgtagaattgcacttcagtttattttcttacaaataaccttacattttgtttaatggcttccaagagccttttttttttttgtatttcagagaaaattcaggtaccaggatgcaatggatttatttgattcaggggacctgtgtttccatgtcaaatgttttcaaataaaatgaaatatgagtttcaatactttttatattttaatatttccattcattaatattatggttattgtcagcaattttatgtttgaatatttgaaataaaagtttaagatttgaaaa

SEQ ID NO: 4 - protein, 1043 aa, Artificial Sequence
  RAG1 isoform 1
MAASFPPTLGLSSAPDEIQHPHIKFSEWKFKLFRVRSFEKTPEEAQKEKKDSFEGKPSLEQSPAVLDKADGQKPVPTQPLLKAHPKFSKKFHDNEKARGKAIHQANLRHLCRICGNSFRADEHNRRYPVHGPVDGKTLGLLRKKEKRATSWPDLIAKVFRIDVKADVDSIHPTEFCHNCWSIMHRKFSSAPCEVYFPRNVTMEWHPHTPSCDICNTARRGLKRKSLQPNLQLSKKLKTVLDQARQARQHKRRAQARISSKDVMKKIANCSKIHLSTKLLAVDFPEHFVKSISCQICEHILADPVETNCKHVFCRVCILRCLKVMGSYCPSCRYPCFPTDLESPVKSFLSVLNSLMVKCPAKECNEEVSLEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKCYEMEDVLKHHWLYTSKYLQKFMNAHNALKTSGFTMNPQASLGDPLGIEDSLESQDSMEF

SEQ ID NO: 5 - protein, 931 aa, Artificial Sequence
  RAG1 isoform 2
MAASFPPTLGLSSAPDEIQHPHIKFSEWKFKLFRVRSFEKTPEEAQKEKKDSFEGKPSLEQSPAVLDKADGQKPVPTQPLLKAHPKFSKKFHDNEKARGKAIHQANLRHLCRICGNSFRADEHNRRYPVHGPVDGKTLGLLRKKEKRATSWPDLIAKVFRIDVKADVDSIHPTEFCHNCWSIMHRKFSSAPCEVYFPRNVTMEWHPHTPSCDICNTARRGLKRKSLQPNLQLSKKLKTVLDQARQARQHKRRAQARISSKDVMKKIANCSKIHLSTKLLAVDFPEHFVKSISCQICEHILADPVETNCKHVFCRVCILRCLKVMGSYCPSCRYPCFPTDLESPVKSFLSVLNSLMVKCPAKECNEEVSLEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRN

SEQ ID NO: 6 - DNA, 3131 bp, Artificial Sequence
  RAG1 polynucleotide
atggccgcctccttcccacctacccttggattgtcctccgcccctgacgaaattcaacatccccacatcaaattctcggagtggaagttcaagctctttcgcgtgcgctcgttcgaaaagacccccgaggaagcccaaaaggagaagaaagactcattcgaaggaaaacccagcctcgaacagtccccggccgtcctggacaaggccgacgggcagaagcctgtgccgacccagccgctgctgaaagcgcacccgaaattctccaagaagtttcacgataacgagaaggcccggggaaaggccatccaccaagcaaaccttagacacctgtgccgcatctgtgggaactcattcagagccgacgaacataaccggagataccctgtgcatggccctgtcgacggaaagaccctggggctcctgagaaagaaggagaagagggcgacatcctggccggacctgatcgcaaaggtgttcagaatcgacgtgaaggcagatgtggacagcatccacccaaccgagttctgccacaactgctggagcattatgcaccggaagttcagctcagcgccctgtgaagtgtacttcccgcgcaacgtgactatggagtggcatccacacactccgtcctgcgacatctgtaacactgctcggcgcggactcaagaggaagtccctgcagccgaatctgcagctgagcaagaagcttaagaccgtgctggaccaggctcggcaggcccgccagcacaagcgacgcgcccaggcccggatctcatctaaggatgtgatgaagaagatcgccaattgcagcaaaatccacctgtctaccaagctgctggcggtggacttcccggagcacttcgtgaagtccatcagctgtcagatctgcgagcatattctcgccgaccccgtggagactaattgcaagcacgtgttctgccgcgtgtgcatcctgcgctgcctgaaggtcatgggctcctattgcccttcctgccggtacccctgtttccctactgatctggagtccccggtcaagtccttcttgtccgtgctgaactccctgatggtcaaatgtcccgcaaaggagtgcaatgaggaagtgtccctggaaaagtacaaccaccacatcagcagccacaaggagtccaaagaaatctttgtgcacattaacaagggcggtcggccccggcagcatctgctctcgctgactcgccgggcccagaagcacaggctccgggagctgaagctgcaagtcaaggccttcgccgacaaggaagagggaggagatgtgaagtccgtgtgcatgaccctgtttttgctggcgctgcgggctcggaacgaacacagacaagctgatgaactggaggccatcatgcagggcaaaggatcgggactccagccggctgtgtgtctcgccatccgcgtcaacacattcctctcatgctcccaataccacaagatgtacaggactgtgaaggccatcaccggacggcagatctttcagccactccacgcccttcggaacgcagaaaaggtcttgctgccgggataccatcatttcgaatggcagccgcccttgaaaaacgtgtcctcgtccaccgacgtgggcattattgatgggctgagcggcctgtcctcctctgtggatgactaccctgtggataccatcgccaaacggttcagatacgattccgcgctggtgtcggccctgatggacatggaggaggacatcctggagggaatgagatcacaagatctggacgactacctcaacgggcccttcacggtggtggtcaaggaatcgtgcgatggaatgggcgacgtgtcggagaagcacggttccggacctgtggtgccggaaaaggccgtgcgcttctccttcaccatcatgaagatcaccattgcgcatagctcccagaacgtcaaagtgttcgaagaggccaagccgaactcagagctctgctgcaagccgctgtgcctgatgttggcggacgagagcgatcacgaaaccctgaccgccattctgtcgcctctgatcgcggagagggaggccatgaagtcctccgaactgatgctggagctgggcggtattttgcggacttttaagttcatcttccggggaaccggttatgacgaaaagctcgtgcgcgaagtggagggcctggaagcctcaggctccgtctacatctgcactctctgcgacgccacccggctggaggcgtcacagaatcttgtgttccactcgatcactaggtcccacgcggagaacctggaacgctatgaggtctggcgctctaacccataccacgaatccgtggaagaacttcgggacagagtgaagggagtgtcagcaaagcctttcattgaaaccgtgcctagcatcgacgccctccattgcgacatcggcaacgccgccgagttctacaagatcttccagcttgagatcggggaagtgtacaagaacccgaacgcctccaaggaagaaagaaagcggtggcaggctacccttgacaaacacctccgcaagaagatgaacctgaagcccattatgcggatgaacggaaacttcgctaggaagctgatgactaaggaaacggtcgacgcggtctgtgaactgatccccagcgaagaacgacatgaagcgctgcgcgaactcatggacctgtacctgaagatgaagcctgtctggcggagctcgtgccctgccaaggagtgcccggagtcgctgtgtcagtacagctttaacagccaaaggttcgcagagctgctgtcgaccaagttcaagtacagatacgaaggaaagattaccaactacttccacaagactctcgctcacgtgcccgagattatcgaacgcgatggttccatcggggcctgggcctccgagggcaacgagtcgggcaacaagttgttccgccggtttagaaagatgaacgcccgccagtccaagtgctacgaaatggaagatgtgctgaagcatcactggctgtatacctccaagtacctccagaagttcatgaacgcacataacgccctcaagacctccgggttcaccatgaacccccaggcctccctcggtgaccctctgggaattgaagatagcttggagagccaggactcgatggaattcta

SEQ ID NO: 7 - DNA, 50 bp, Artificial Sequence
  Synthetic polynucleotide
tgctgtgtggagggaggcacgcctgtagctctgatgtcagatggcaatgt

SEQ ID NO: 8 - DNA, 50 bp, Artificial Sequence
  Synthetic polynucleotide
aagagagctacttcctggccggacctcattgccaaggttttccggatcga

SEQ ID NO: 9 - DNA, 50 bp, Artificial Sequence
  Synthetic polynucleotide
aagcaagaggcaaagcgatccatcaagccaaccttcgacatctctgccgc

SEQ ID NO: 10 - DNA, 50 bp, Artificial Sequence
  Synthetic polynucleotide
cagcatggcagcctctttcccacccaccttgggactcagttctgccccag

SEQ ID NO: 11 - DNA, 50 bp, Artificial Sequence
  Synthetic polynucleotide
ttgtgtacagactaagttgaagatgttaggagggaagattgtgggccaag

SEQ ID NO: 12 - DNA, 50 bp, Artificial Sequence
  Synthetic polynucleotide
ttactcccacctcttcttattatgttacaaactatagtgctaatgaccat

SEQ ID NO: 13 - DNA, 50 bp, Artificial Sequence
  Synthetic polynucleotide
acagaagagaattaggaagcagaattgaactataagcaattttgaggtgt

SEQ ID NO: 14 - DNA, 50 bp, Artificial Sequence
  Synthetic polynucleotide
gggaagtaaaatgctaaaggaatgagaaggcatttggggttgagttcaac

SEQ ID NO: 15 - DNA, 50 bp, Artificial Sequence
  Synthetic polynucleotide
aaccaaccccctggaagactgctttaaaaagctggaaatacattgtccag

SEQ ID NO: 16 - DNA, 50 bp, Artificial Sequence
  Synthetic polynucleotide
cacagaagagaattaggaagcagaattgaactataagcaattttgaggtg

SEQ ID NO: 17 - DNA, 50 bp, Artificial Sequence
  Synthetic polynucleotide
ggcagtggccggtggggacagggctgagccagcaccaaccactcagcctt

SEQ ID NO: 18 - DNA, 50 bp, Artificial Sequence
  Synthetic polynucleotide
ttttttctgcatcgctagcgatctgtgcattacaactcaaatcagtcggg

SEQ ID NO: 19 - DNA, 50 bp, Artificial Sequence
  Synthetic polynucleotide
atggcagtggccggtggggacagggctgagccagcaccaaccactcagcc

SEQ ID NO: 20 - DNA, 50 bp, Artificial Sequence
  Synthetic polynucleotide
aaggcagatgttgactcgatccaccccactgagttctgccataactgctg

SEQ ID NO: 21 - DNA, 50 bp, Artificial Sequence
  Synthetic polynucleotide
gtgggaattcttttagagctgatgagcacaacaggagatatccagtccat

SEQ ID NO: 22 - DNA, 50 bp, Artificial Sequence
  Synthetic polynucleotide
aattcagcacccacatattaaattttcagaatggaaatttaagctgttcc

SEQ ID NO: 23 - DNA, 50 bp, Artificial Sequence
  Synthetic polynucleotide
ggggtgtatgtgtgtgggtatagggtgggcagctgggatggaaatggggg

SEQ ID NO: 24 - DNA, 50 bp, Artificial Sequence
  Synthetic polynucleotide
caacagtgactttcaggatgacctgtgtgagttttatctgaaaccatgtg

SEQ ID NO: 25 - DNA, 50 bp, Artificial Sequence
  Synthetic polynucleotide
tgggctgcagttgaaatattttttgaggttaatgagacatttgaaatggc

SEQ ID NO: 26 - DNA, 50 bp, Artificial Sequence
  Synthetic polynucleotide
gaggcaggggagccacagggaaagacctagcacctgccacagaagagaat

SEQ ID NO: 27 - DNA, 50 bp, Artificial Sequence
  Synthetic polynucleotide
tacaatgaggctaatacaatgtggaaaatattacttttctttgattttag

SEQ ID NO: 28 - DNA, 50 bp, Artificial Sequence
  Synthetic polynucleotide
ttgggctgcagttgaaatattttttgaggttaatgagacatttgaaatgg

SEQ ID NO: 29 - DNA, 50 bp, Artificial Sequence
  Synthetic polynucleotide
atcccgaggctggtctactgctgagaccttttgttagaagagaggagatc

SEQ ID NO: 30 - DNA, 50 bp, Artificial Sequence
  Synthetic polynucleotide
ctggcatatgtaattgccaatgttttttaccagaagagaaacattactcc

SEQ ID NO: 31 - DNA, 290 bp, Artificial Sequence
  Homology region
tgagcacacagttattacttggaaattgtgtacagactaagttgaagatgttaggagggaagattgtgggccaagtaacggggtgtatgtgtgtgggtatagggtgggcagctgggatggaaatggggggctgctgctgctgctgcaccctggcctcctgaactaatgatatcactcaccagaaactactgttcctgcactgtccaagccaccccaaactagtttgtcaaaatgaatctgtgctgtgtggagggaggcacgcctgtagctctgatgtcagatggcaatgt

SEQ ID NO: 32 - DNA, 328 bp, Artificial Sequence
  Homology region
atggcagtggccggtggggacagggctgagccagcaccaaccactcagcctttgagatcccgaggctggtctactgctgagaccttttgttagaagagaggagatcaagcatttgcaaggtttctgagtgtcaaaatatgaatccaagataactctttcacaatcctaacttcatgctgtctacaggtccatattttagcctgctttctccatgttcatccgaaaagaaagaaaagctaagggtggtggtcatatttgaaattagccagatcttaagtttttctgggggaaatttagaagaaaatatggaaaagtgactatgagcaca

SEQ ID NO: 33 - DNA, 25 bp, Artificial Sequence
  splice acceptor
ctgacctcttctcttcctcccacag

SEQ ID NO: 34 - DNA, 8 bp, Artificial Sequence
  Splice donor
aggtaagt

SEQ ID NO: 35 - DNA, 226 bp, Artificial Sequence
  BGH polyA seq
gctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 36 - DNA, 12 bp, Artificial Sequence
  Kozak
gccgccaccatg

SEQ ID NO: 37 - DNA, 592 bp, Artificial Sequence
  WPRE
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 38 - DNA, 3381 bp, Artificial Sequence
  RAG1 3UTR
gtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgctggcaatgtagagcaggcatggaattttatatgctagtgagtcataatgatatgttagtgttaattagttttttcttcctttgattttattggccataattgctactcttcatacacagtatatcaaagagcttgataatttagttgtcaaaagtgcatcggcgacattatctttaattgtatgtatttggtgcttcttcagggattgaactcagtatctttcattaaaaaacacagcagttttccttgctttttatatgcagaatatcaaagtcatttctaatttagttgtcaaaaacatatacatattttaacattagtttttttgaaaactcttggttttgtttttttggaaatgagtgggccactaagccacactttcccttcatcctgcttaatccttccagcatgtctctgcactaataaacagctaaattcacataatcatcctatttactgaagcatggtcatgctggtttatagattttttacccatttctactctttttctctattggtggcactgtaaatactttccagtattaaattatccttttctaacactgtaggaactattttgaatgcatgtgactaagagcatgatttatagcacaacctttccaataatcccttaatcagatcacattttgataaaccctgggaacatctggctgcaggaatttcaatatgtagaaacgctgcctatggttttttgcccttactgttgagactgcaatatcctagaccctagttttatactagagttttatttttagcaatgcctattgcaagtgcaattatatactccagggaaattcaccacactgaatcgagcatttgtgtgtgtatgtgtgaagtatatactgggacttcagaagtgcaatgtatttttctcctgtgaaacctgaatctacaagttttcctgccaagccactcaggtgcattgcagggaccagtgataatggctgatgaaaattgatgattggtcagtgaggtcaaaaggagccttgggattaataaacatgcactgagaagcaagaggaggagaaaaagatgtctttttcttccaggtgaactggaatttagttttgcctcagatttttttcccacaagatacagaagaagataaagatttttttggttgagagtgtgggtcttgcattacatcaaacagagttcaaattccacacagataagaggcaggatatataagcgccagtggtagttgggaggaataaaccattatttggatgcaggtggtttttgattgcaaatatgtgtgtgtcttcagtgattgtatgacagatgatgtattcttttgatgttaaaagattttaagtaagagtagatacattgtacccattttacattttcttattttaactacagtaatctacataaatatacctcagaaatcatttttggtgattattttttgttttgtagaattgcacttcagtttattttcttacaaataaccttacattttgtttaatggcttccaagagccttttttttttttgtatttcagagaaaattcaggtaccaggatgcaatggatttatttgattcaggggacctgtgtttccatgtcaaatgttttcaaataaaatgaaatatgagtttcaatactttttatattttaatatttccattcattaatattatggttattgtcagcaattttatgtttgaatatttgaaataaaagtttaagatttgaaaatggtatgtattataatttctattcaaatattaataataatattgagtgcagcatt

SEQ ID NO: 39 - DNA, 4021 bp, Artificial Sequence
  Polynucleotide
tgagcacacagttattacttggaaattgtgtacagactaagttgaagatgttaggagggaagattgtgggccaagtaacggggtgtatgtgtgtgggtatagggtgggcagctgggatggaaatggggggctgctgctgctgctgcaccctggcctcctgaactaatgatatcactcaccagaaactactgttcctgcactgtccaagccaccccaaactagtttgtcaaaatgaatctgtgctgtgtggagggaggcacgcctgtagctctgatgtcagatggcaatgtgaattcctgacctcttctcttcctcccacaggccgccaccatggccgcctccttcccacctacccttggattgtcctccgcccctgacgaaattcaacatccccacatcaaattctcggagtggaagttcaagctctttcgcgtgcgctcgttcgaaaagacccccgaggaagcccaaaaggagaagaaagactcattcgaaggaaaacccagcctcgaacagtccccggccgtcctggacaaggccgacgggcagaagcctgtgccgacccagccgctgctgaaagcgcacccgaaattctccaagaagtttcacgataacgagaaggcccggggaaaggccatccaccaagcaaaccttagacacctgtgccgcatctgtgggaactcattcagagccgacgaacataaccggagataccctgtgcatggccctgtcgacggaaagaccctggggctcctgagaaagaaggagaagagggcgacatcctggccggacctgatcgcaaaggtgttcagaatcgacgtgaaggcagatgtggacagcatccacccaaccgagttctgccacaactgctggagcattatgcaccggaagttcagctcagcgccctgtgaagtgtacttcccgcgcaacgtgactatggagtggcatccacacactccgtcctgcgacatctgtaacactgctcggcgcggactcaagaggaagtccctgcagccgaatctgcagctgagcaagaagcttaagaccgtgctggaccaggctcggcaggcccgccagcacaagcgacgcgcccaggcccggatctcatctaaggatgtgatgaagaagatcgccaattgcagcaaaatccacctgtctaccaagctgctggcggtggacttcccggagcacttcgtgaagtccatcagctgtcagatctgcgagcatattctcgccgaccccgtggagactaattgcaagcacgtgttctgccgcgtgtgcatcctgcgctgcctgaaggtcatgggctcctattgcccttcctgccggtacccctgtttccctactgatctggagtccccggtcaagtccttcttgtccgtgctgaactccctgatggtcaaatgtcccgcaaaggagtgcaatgaggaagtgtccctggaaaagtacaaccaccacatcagcagccacaaggagtccaaagaaatctttgtgcacattaacaagggcggtcggccccggcagcatctgctctcgctgactcgccgggcccagaagcacaggctccgggagctgaagctgcaagtcaaggccttcgccgacaaggaagagggaggagatgtgaagtccgtgtgcatgaccctgtttttgctggcgctgcgggctcggaacgaacacagacaagctgatgaactggaggccatcatgcagggcaaaggatcgggactccagccggctgtgtgtctcgccatccgcgtcaacacattcctctcatgctcccaataccacaagatgtacaggactgtgaaggccatcaccggacggcagatctttcagccactccacgcccttcggaacgcagaaaaggtcttgctgccgggataccatcatttcgaatggcagccgcccttgaaaaacgtgtcctcgtccaccgacgtgggcattattgatgggctgagcggcctgtcctcctctgtggatgactaccctgtggataccatcgccaaacggttcagatacgattccgcgctggtgtcggccctgatggacatggaggaggacatcctggagggaatgagatcacaagatctggacgactacctcaacgggcccttcacggtggtggtcaaggaatcgtgcgatggaatgggcgacgtgtcggagaagcacggttccggacctgtggtgccggaaaaggccgtgcgcttctccttcaccatcatgaagatcaccattgcgcatagctcccagaacgtcaaagtgttcgaagaggccaagccgaactcagagctctgctgcaagccgctgtgcctgatgttggcggacgagagcgatcacgaaaccctgaccgccattctgtcgcctctgatcgcggagagggaggccatgaagtcctccgaactgatgctggagctgggcggtattttgcggacttttaagttcatcttccggggaaccggttatgacgaaaagctcgtgcgcgaagtggagggcctggaagcctcaggctccgtctacatctgcactctctgcgacgccacccggctggaggcgtcacagaatcttgtgttccactcgatcactaggtcccacgcggagaacctggaacgctatgaggtctggcgctctaacccataccacgaatccgtggaagaacttcgggacagagtgaagggagtgtcagcaaagcctttcattgaaaccgtgcctagcatcgacgccctccattgcgacatcggcaacgccgccgagttctacaagatcttccagcttgagatcggggaagtgtacaagaacccgaacgcctccaaggaagaaagaaagcggtggcaggctacccttgacaaacacctccgcaagaagatgaacctgaagcccattatgcggatgaacggaaacttcgctaggaagctgatgactaaggaaacggtcgacgcggtctgtgaactgatccccagcgaagaacgacatgaagcgctgcgcgaactcatggacctgtacctgaagatgaagcctgtctggcggagctcgtgccctgccaaggagtgcccggagtcgctgtgtcagtacagctttaacagccaaaggttcgcagagctgctgtcgaccaagttcaagtacagatacgaaggaaagattaccaactacttccacaagactctcgctcacgtgcccgagattatcgaacgcgatggttccatcggggcctgggcctccgagggcaacgagtcgggcaacaagttgttccgccggtttagaaagatgaacgcccgccagtccaagtgctacgaaatggaagatgtgctgaagcatcactggctgtatacctccaagtacctccagaagttcatgaacgcacataacgccctcaagacctccgggttcaccatgaacccccaggcctccctcggtgaccctctgggaattgaagatagcttggagagccaggactcgatggaattctagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtctagaatggcagtggccggtggggacagggctgagccagcaccaaccactcagcctttgagatcccgaggctggtctactgctgagaccttttgttagaagagaggagatcaagcatttgcaaggtttctgagtgtcaaaatatgaatccaagataactctttcacaatcctaacttcatgctgtctacaggtccatattttagcctgctttctccatgttcatccgaaaagaaagaaaagctaagggtggtggtcatatttgaaattagccagatcttaagtttttctgggggaaatttagaagaaaatatggaaaagtgactatgagcaca

SEQ ID NO: 40 - DNA, 3403 bp, Artificial Sequence
  Polynucleotide
gaattcctgacctcttctcttcctcccacaggccgccaccatggccgcctccttcccacctacccttggattgtcctccgcccctgacgaaattcaacatccccacatcaaattctcggagtggaagttcaagctctttcgcgtgcgctcgttcgaaaagacccccgaggaagcccaaaaggagaagaaagactcattcgaaggaaaacccagcctcgaacagtccccggccgtcctggacaaggccgacgggcagaagcctgtgccgacccagccgctgctgaaagcgcacccgaaattctccaagaagtttcacgataacgagaaggcccggggaaaggccatccaccaagcaaaccttagacacctgtgccgcatctgtgggaactcattcagagccgacgaacataaccggagataccctgtgcatggccctgtcgacggaaagaccctggggctcctgagaaagaaggagaagagggcgacatcctggccggacctgatcgcaaaggtgttcagaatcgacgtgaaggcagatgtggacagcatccacccaaccgagttctgccacaactgctggagcattatgcaccggaagttcagctcagcgccctgtgaagtgtacttcccgcgcaacgtgactatggagtggcatccacacactccgtcctgcgacatctgtaacactgctcggcgcggactcaagaggaagtccctgcagccgaatctgcagctgagcaagaagcttaagaccgtgctggaccaggctcggcaggcccgccagcacaagcgacgcgcccaggcccggatctcatctaaggatgtgatgaagaagatcgccaattgcagcaaaatccacctgtctaccaagctgctggcggtggacttcccggagcacttcgtgaagtccatcagctgtcagatctgcgagcatattctcgccgaccccgtggagactaattgcaagcacgtgttctgccgcgtgtgcatcctgcgctgcctgaaggtcatgggctcctattgcccttcctgccggtacccctgtttccctactgatctggagtccccggtcaagtccttcttgtccgtgctgaactccctgatggtcaaatgtcccgcaaaggagtgcaatgaggaagtgtccctggaaaagtacaaccaccacatcagcagccacaaggagtccaaagaaatctttgtgcacattaacaagggcggtcggccccggcagcatctgctctcgctgactcgccgggcccagaagcacaggctccgggagctgaagctgcaagtcaaggccttcgccgacaaggaagagggaggagatgtgaagtccgtgtgcatgaccctgtttttgctggcgctgcgggctcggaacgaacacagacaagctgatgaactggaggccatcatgcagggcaaaggatcgggactccagccggctgtgtgtctcgccatccgcgtcaacacattcctctcatgctcccaataccacaagatgtacaggactgtgaaggccatcaccggacggcagatctttcagccactccacgcccttcggaacgcagaaaaggtcttgctgccgggataccatcatttcgaatggcagccgcccttgaaaaacgtgtcctcgtccaccgacgtgggcattattgatgggctgagcggcctgtcctcctctgtggatgactaccctgtggataccatcgccaaacggttcagatacgattccgcgctggtgtcggccctgatggacatggaggaggacatcctggagggaatgagatcacaagatctggacgactacctcaacgggcccttcacggtggtggtcaaggaatcgtgcgatggaatgggcgacgtgtcggagaagcacggttccggacctgtggtgccggaaaaggccgtgcgcttctccttcaccatcatgaagatcaccattgcgcatagctcccagaacgtcaaagtgttcgaagaggccaagccgaactcagagctctgctgcaagccgctgtgcctgatgttggcggacgagagcgatcacgaaaccctgaccgccattctgtcgcctctgatcgcggagagggaggccatgaagtcctccgaactgatgctggagctgggcggtattttgcggacttttaagttcatcttccggggaaccggttatgacgaaaagctcgtgcgcgaagtggagggcctggaagcctcaggctccgtctacatctgcactctctgcgacgccacccggctggaggcgtcacagaatcttgtgttccactcgatcactaggtcccacgcggagaacctggaacgctatgaggtctggcgctctaacccataccacgaatccgtggaagaacttcgggacagagtgaagggagtgtcagcaaagcctttcattgaaaccgtgcctagcatcgacgccctccattgcgacatcggcaacgccgccgagttctacaagatcttccagcttgagatcggggaagtgtacaagaacccgaacgcctccaaggaagaaagaaagcggtggcaggctacccttgacaaacacctccgcaagaagatgaacctgaagcccattatgcggatgaacggaaacttcgctaggaagctgatgactaaggaaacggtcgacgcggtctgtgaactgatccccagcgaagaacgacatgaagcgctgcgcgaactcatggacctgtacctgaagatgaagcctgtctggcggagctcgtgccctgccaaggagtgcccggagtcgctgtgtcagtacagctttaacagccaaaggttcgcagagctgctgtcgaccaagttcaagtacagatacgaaggaaagattaccaactacttccacaagactctcgctcacgtgcccgagattatcgaacgcgatggttccatcggggcctgggcctccgagggcaacgagtcgggcaacaagttgttccgccggtttagaaagatgaacgcccgccagtccaagtgctacgaaatggaagatgtgctgaagcatcactggctgtatacctccaagtacctccagaagttcatgaacgcacataacgccctcaagacctccgggttcaccatgaacccccaggcctccctcggtgaccctctgggaattgaagatagcttggagagccaggactcgatggaattctagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtctaga

SEQ ID NO: 41 - DNA, 19 bp, Artificial Sequence
  Synthetic polynucleotide
tcagatggcaatgtcgaga

SEQ ID NO: 42 - DNA, 18 bp, Artificial Sequence
  Synthetic polynucleotide
ttttccggatcgatgtga

SEQ ID NO: 43 - DNA, 19 bp, Artificial Sequence
  Synthetic polynucleotide
gacatctctgccgcatctg

SEQ ID NO: 44 - DNA, 19 bp, Artificial Sequence
  Synthetic polynucleotide
gtgggtgctgaatttcatc

SEQ ID NO: 45 - DNA, 19 bp, Artificial Sequence
  Synthetic polynucleotide
gattgtgggccaagtaacg

SEQ ID NO: 46 - DNA, 19 bp, Artificial Sequence
  Synthetic polynucleotide
gaaagtcactgttggtcga

SEQ ID NO: 47 - DNA, 19 bp, Artificial Sequence
  Synthetic polynucleotide
caattttgaggtgttcgtt

SEQ ID NO: 48 - DNA, 19 bp, Artificial Sequence
  Synthetic polynucleotide
gggttgagttcaacctaag

SEQ ID NO: 49 - DNA, 19 bp, Artificial Sequence
  Synthetic polynucleotide
ttagcctcattgtactagc

SEQ ID NO: 50 - DNA, 19 bp, Artificial Sequence
  Synthetic polynucleotide
gcaattttgaggtgttcgt

SEQ ID NO: 51 - DNA, 19 bp, Artificial Sequence
  Synthetic polynucleotide
accagcctcgggatctcaa

SEQ ID NO: 52 - DNA, 19 bp, Artificial Sequence
  Synthetic polynucleotide
tcaaatcagtcgggtttcc

SEQ ID NO: 53 - DNA, 20 bp, Artificial Sequence
  Synthetic polynucleotide
gtcagatggcaatgtcgaga

SEQ ID NO: 54 - DNA, 20 bp, Artificial Sequence
  Synthetic polynucleotide
tgtgggtgctgaatttcatc

SEQ ID NO: 55 - DNA, 20 bp, Artificial Sequence
  Synthetic polynucleotide
ggggttgagttcaacctaag

SEQ ID NO: 56 - DNA, 19 bp, Artificial Sequence
  Synthetic polynucleotide
gccaatgtcaatgtcgaga

SEQ ID NO: 57 - DNA, 19 bp, Artificial Sequence
  Synthetic polynucleotide
gtcaaagtcaatgtcgaga

SEQ ID NO: 58 - DNA, 19 bp, Artificial Sequence
  Synthetic polynucleotide
atcagatggcaagtcgaga

SEQ ID NO: 59 - DNA, 20 bp, Artificial Sequence
  Synthetic polynucleotide
ttcagatggaaatgtcaaga

SEQ ID NO: 60 - DNA, 19 bp, Artificial Sequence
  Synthetic polynucleotide
gtaagatgcaatgtagaga

SEQ ID NO: 61 - DNA, 19 bp, Artificial Sequence
  Synthetic polynucleotide
gtcagatggcaatgtcaga

SEQ ID NO: 62 - DNA, 226 bp, Artificial Sequence
  BGH polyA seq
actgtgccttctagttgccagccatctgttgtttgcccctcccccgtgccttccttgaccctggaaggtgccactcccactgccctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 63 - DNA, 607 bp, Artificial Sequence
  IRES
gaattaactcgaggaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccaacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 64 - DNA, 843 bp, Artificial Sequence
  NGFR polynucleotide
atgggagctggtgctaccggcagagctatggatggacctagactgctgctcctgctgctgctcggagtttctcttggcggagccaaagaggcctgtcctaccggcctgtatacacactctggcgagtgctgcaaggcctgcaatcttggagaaggcgtggcacagccttgcggcgctaatcagacagtgtgcgagccttgcctggacagcgtgacctttagcgacgtggtgtctgccaccgagccatgcaagccttgtaccgagtgtgtgggcctgcagagcatgtctgccccttgtgtggaagccgacgatgccgtgtgtagatgcgcctacggctactaccaggacgagacaacaggcagatgcgaggcctgtagagtgtgtgaagccggctctggactggtgttcagctgccaagacaagcagaacaccgtgtgcgaggaatgccccgatggcacctatagcgacgaggccaaccatgtagatccctgcctgccttgtactgtgtgcgaagataccgagcggcagctgcgcgagtgtacaagatgggctgatgccgagtgcgaagagatccccggcagatggatcaccagaagcacacctccagagggcagcgatagcacagccccttctacacaagagcccgaggctcctcctgagcaggatctgattgcctctacagtggccggcgtggtcacaacagtgatgggatcttctcagcccgtggtcaccagaggcaccaccgacaatctgatccccgtgtactgtagcatcctggccgccgtggttgtgggactcgtggcctatatcgccttcaagcggtggaaccggggcatcctgtaa

SEQ ID NO: 65 - DNA, 225 bp, Artificial Sequence
  BgH polyA seq
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 66 - DNA, 78 bp, Artificial Sequence
  PEST polynucleotide
atgaggaccgaggcccccgagggcaccgagagcgagatggagacccccagcgccatcaacggcaaccccagctggcac

SEQ ID NO: 67 - protein, 94 aa, Artificial Sequence
  GSE56
CPGRDRRTEEENFRKKEEHCPELPPGSAKRALPTSTSSSPQQKKKPLDGEYFTLKIRGRERFEMFRELNEALELKDARAAEESGDSRAHSSYPK

SEQ ID NO: 68 - DNA, 17 bp, Artificial Sequence
  Synthetic polynucleotide
ccttctcagcattccga

SEQ ID NO: 69 - DNA, 20 bp, Artificial Sequence
  Synthetic polynucleotide
aacatcttctgtcgctgact

SEQ ID NO: 70 - DNA, 20 bp, Artificial Sequence
  Synthetic polynucleotide
gtaccttctcagcattccga

SEQ ID NO: 71 - DNA, 22 bp, Artificial Sequence
  Synthetic polynucleotide
ccataaacactgtcagaagagg

SEQ ID NO: 72 - DNA, 19 bp, Artificial Sequence
  Synthetic polynucleotide
gtgttgcagatgtcacagg

SEQ ID NO: 73 - DNA, 20 bp, Artificial Sequence
  Synthetic polynucleotide
gaagtggttcatgcaagagg

SEQ ID NO: 74 - DNA, 21 bp, Artificial Sequence
  Synthetic polynucleotide
ggatgaacatggagaaagcag

SEQ ID NO: 75 - DNA, 21 bp, Artificial Sequence
  Synthetic polynucleotide
ggggagaaatgtgtagggaag

SEQ ID NO: 76 - DNA, 22 bp, Artificial Sequence
  Synthetic polynucleotide
ctcaaaaacaaagaaatgggcg

SEQ ID NO: 77 - DNA, 20 bp, Artificial Sequence
  Synthetic polynucleotide
ataggtggatgggatgatgg

SEQ ID NO: 78 - DNA, 22 bp, Artificial Sequence
  Synthetic polynucleotide
cctcttctgacagtgtttatgg

SEQ ID NO: 79 - DNA, 20 bp, Artificial Sequence
  Synthetic polynucleotide
ggaaaatgaatgccaggcag

SEQ ID NO: 80 - DNA, 22 bp, Artificial Sequence
  Synthetic polynucleotide
aggtcatcatgctgtacaaatg

SEQ ID NO: 81 - DNA, 19 bp, Artificial Sequence
  Synthetic polynucleotide
tccatgcttccctactgac

SEQ ID NO: 82 - DNA, 20 bp, Artificial Sequence
  Synthetic polynucleotide
ctcccattccatcacaagac

SEQ ID NO: 83 - DNA, 1667 bp, Artificial Sequence
  Synthetic polynucleotide
tgagcacacagttattacttggaaattgtgtacagactaagttgaagatgttaggagggaagattgtgggccaagtaacggggtgtatgtgtgtgggtatagggtgggcagctgggatggaaatggggggctgctgctgctgctgcaccctggcctcctgaactaatgatatcactcaccagaaactactgttcctgcactgtccaagccaccccaaactagtttgtcaaaatgaatctgtgctgtgtggagggaggcacgcctgtagctctgatgtcagatggcaatgtgaattcctgacctcttctcttcctcccacaggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagtcgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcctgacaaccactacctgagcacccagtccgccctgagcaaagaccccaacgagaagcgcgatcacatggtcctgctggagttcgtgaccgccgccgggatcactctcggcatggacgagctgtacaagtaaactgtgccttctagttgccagccatctgttgtttgcccctcccccgtgccttccttgaccctggaaggtgccactcccactgccctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatccggaacaggtgtgataatgagagatcttgcgttccaacgagaaactaatgtttctagaatggcagtggccggtggggacagggctgagccagcaccaaccactcagcctttgagatcccgaggctggtctactgctgagaccttttgttagaagagaggagatcaagcatttgcaaggtttctgagtgtcaaaatatgaatccaagataactctttcacaatcctaacttcatgctgtctacaggtccatattttagcctgctttctccatgttcatccgaaaagaaagaaaagctaagggtggtggtcatatttgaaattagccagatcttaagtttttctgggggaaatttagaagaaaatatggaaaagtgactatgagcaca

SEQ ID NO: 84 - DNA, 290 bp, Artificial Sequence; identical to SEQ ID NO: 31
  Synthetic polynucleotide

SEQ ID NO: 85 - DNA, 25 bp, Artificial Sequence
  Synthetic polynucleotide
ctgacctcttctcttcctcccacag

SEQ ID NO: 86 - DNA, 12 bp, Artificial Sequence
  Synthetic polynucleotide
gccgccaccatg

SEQ ID NO: 87 - DNA, 720 bp, Artificial Sequence
  Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagtcgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcctgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 88 - DNA, 226 bp, Artificial Sequence; identical to SEQ ID NO: 62
  Synthetic polynucleotide

SEQ ID NO: 89 - DNA, 328 bp, Artificial Sequence; identical to SEQ ID NO: 32
  Synthetic polynucleotide

SEQ ID NO: 90 - DNA, 2228 bp, Artificial Sequence
  Synthetic polynucleotide
tgagcacacagttattacttggaaattgtgtacagactaagttgaagatgttaggagggaagattgtgggccaagtaacggggtgtatgtgtgtgggtatagggtgggcagctgggatggaaatggggggctgctgctgctgctgcaccctggcctcctgaactaatgatatcactcaccagaaactactgttcctgcactgtccaagccaccccaaactagtttgtcaaaatgaatctgtgctgtgtggagggaggcacgcctgtagctctgatgtcagatggcaatgtgaattcctgacctcttctcttcctcccacaggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagtcgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcctgacaaccactacctgagcacccagtccgccctgagcaaagaccccaacgagaagcgcgatcacatggtcctgctggagttcgtgaccgccgccgggatcactctcggcatggacgagctgtacaagtaaactagtactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaatggatcctaaactgtgccttctagttgccagccatctgttgtttgcccctcccccgtgccttccttgaccctggaaggtgccactcccactgccctttcctaataaaatgaggaaattgcatcgcattgtctgagtaggtgtcattctattctggggggtggggtggggcaggacagcaagggggaggattgggaagacaatagcaggcatgctggggatgcggtgggctctatggtctagaatggcagtggccggtggggacagggctgagccagcaccaaccactcagcctttgagatcccgaggctggtctactgctgagaccttttgttagaagagaggagatcaagcatttgcaaggtttctgagtgtcaaaatatgaatccaagataactctttcacaatcctaacttcatgctgtctacaggtccatattttagcctgctttctccatgttcatccgaaaagaaagaaaagctaagggtggtggtcatatttgaaattagccagatcttaagtttttctgggggaaatttagaagaaaatatggaaaagtgactatgagcaca

SEQ ID NO: 91 - DNA, 290 bp, Artificial Sequence; identical to SEQ ID NO: 31
  Synthetic polynucleotide

SEQ ID NO: 92 - DNA, 25 bp, Artificial Sequence
  Synthetic polynucleotide
ctgacctcttctcttcctcccacag

SEQ ID NO: 93 - DNA, 12 bp, Artificial Sequence
  Synthetic polynucleotide
gccgccaccatg

SEQ ID NO: 94 - DNA, 720 bp, Artificial Sequence; identical to SEQ ID NO: 87
  Synthetic polynucleotide

SEQ ID NO: 95 - DNA, 592 bp, Artificial Sequence; identical to SEQ ID NO: 37
  Synthetic polynucleotide

SEQ ID NO: 96 - DNA, 226 bp, Artificial Sequence; identical to SEQ ID NO: 62
  Synthetic polynucleotide

SEQ ID NO: 97 - DNA, 328 bp, Artificial Sequence; identical to SEQ ID NO: 32
  Synthetic polynucleotide

SEQ ID NO: 98 - DNA, 1401 bp, Artificial Sequence
  Synthetic polynucleotide
tgagcacacagttattacttggaaattgtgtacagactaagttgaagatgttaggagggaagattgtgggccaagtaacggggtgtatgtgtgtgggtatagggtgggcagctgggatggaaatggggggctgctgctgctgctgcaccctggcctcctgaactaatgatatcactcaccagaaactactgttcctgcactgtccaagccaccccaaactagtttgtcaaaatgaatctgtgctgtgtggagggaggcacgcctgtagctctgatgtcagatggcaatgtgaattcctgacctcttctcttcctcccacaggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagtcgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcctgacaaccactacctgagcacccagtccgccctgagcaaagaccccaacgagaagcgcgatcacatggtcctgctggagttcgtgaccgccgccgggatcactctcggcatggacgagctgtacaagtaaactggatccaggtaagttctagaatggcagtggccggtggggacagggctgagccagcaccaaccactcagcctttgagatcccgaggctggtctactgctgagaccttttgttagaagagaggagatcaagcatttgcaaggtttctgagtgtcaaaatatgaatccaagataactctttcacaatcctaacttcatgctgtctacaggtccatattttagcctgctttctccatgttcatccgaaaagaaagaaaagctaagggtggtggtcatatttgaaattagccagatcttaagtttttctgggggaaatttagaagaaaatatggaaaagtgactatgagcaca

SEQ ID NO: 99 - DNA, 290 bp, Artificial Sequence; identical to SEQ ID NO: 31
  Synthetic polynucleotide

SEQ ID NO: 100 - DNA, 25 bp, Artificial Sequence
  Synthetic polynucleotide
ctgacctcttctcttcctcccacag

SEQ ID NO: 101 - DNA, 12 bp, Artificial Sequence
  Synthetic polynucleotide
gccgccaccatg

SEQ ID NO: 102 - DNA, 720 bp, Artificial Sequence; identical to SEQ ID NO: 87
  Synthetic polynucleotide

SEQ ID NO: 103 - DNA, 8 bp, Artificial Sequence
  Synthetic polynucleotide
aggtaagt

SEQ ID NO: 104 - DNA, 328 bp, Artificial Sequence; identical to SEQ ID NO: 32
  Synthetic polynucleotide

SEQ ID NO: 105 - DNA, 2164 bp, Artificial Sequence
  Synthetic polynucleotide
tgagcacacagttattacttggaaattgtgtacagactaagttgaagatgttaggagggaagattgtgggccaagtaacggggtgtatgtgtgtgggtatagggtgggcagctgggatggaaatggggggctgctgctgctgctgcaccctggcctcctgaactaatgatatcactcaccagaaactactgttcctgcactgtccaagccaccccaaactagtttgtcaaaatgaatctgtgctgtgtggagggaggcacgcctgtagctctgatgtcagatggcaatgtgaattcccatggccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagtcgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcctgacaaccactacctgagcacccagtccgccctgagcaaagaccccaacgagaagcgcgatcacatggtcctgctggagttcgtgaccgccgccgggatcactctcggcatggacgagctgtacaagtaaactgtgccttctagttgccagccatctgttgtttgcccctcccccgtgccttccttgaccctggaaggtgccactcccactgccctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatccggaacaggtgtgataatgagagatcttgcgttccaacgagaaactaatgtttctagaatggcagtggccggtggggacagggctgagccagcaccaaccactcagcctttgagatcccgaggctggtctactgctgagaccttttgttagaagagaggagatcaagcatttgcaaggtttctgagtgtcaaaatatgaatccaagataactctttcacaatcctaacttcatgctgtctacaggtccatattttagcctgctttctccatgttcatccgaaaagaaagaaaagctaagggtggtggtcatatttgaaattagccagatcttaagtttttctgggggaaatttagaagaaaatatggaaaagtgactatgagcaca

SEQ ID NO: 106 - DNA, 290 bp, Artificial Sequence; identical to SEQ ID NO: 31
  Synthetic polynucleotide

SEQ ID NO: 107 - DNA, 516 bp, Artificial Sequence
  Synthetic polynucleotide
ccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagg

SEQ ID NO: 108 - DNA, 5 bp, Artificial Sequence
  Synthetic polynucleotide
ccatg

SEQ ID NO: 109 - DNA, 720 bp, Artificial Sequence; identical to SEQ ID NO: 87
  Synthetic polynucleotide

SEQ ID NO: 110 - DNA, 226 bp, Artificial Sequence; identical to SEQ ID NO: 62
  Synthetic polynucleotide

SEQ ID NO: 111 - DNA, 328 bp, Artificial Sequence; identical to SEQ ID NO: 32
  Synthetic polynucleotide

SEQ ID NO: 112 - DNA, 4774 bp, Artificial Sequence
  Synthetic polynucleotide
tgagcacacagttattacttggaaattgtgtacagactaagttgaagatgttaggagggaagattgtgggccaagtaacggggtgtatgtgtgtgggtatagggtgggcagctgggatggaaatggggggctgctgctgctgctgcaccctggcctcctgaactaatgatatcactcaccagaaactactgttcctgcactgtccaagccaccccaaactagtttgtcaaaatgaatctgtgctgtgtggagggaggcacgcctgtagctctgatgtcagatggcaatgtgaattcctgacctcttctcttcctcccacaggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagtcgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcctgacaaccactacctgagcacccagtccgccctgagcaaagaccccaacgagaagcgcgatcacatggtcctgctggagttcgtgaccgccgccgggatcactctcggcatggacgagctgtacaagtaaactggatccgtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgctggcaatgtagagcaggcatggaattttatatgctagtgagtcataatgatatgttagtgttaattagttttttcttcctttgattttattggccataattgctactcttcatacacagtatatcaaagagcttgataatttagttgtcaaaagtgcatcggcgacattatctttaattgtatgtatttggtgcttcttcagggattgaactcagtatctttcattaaaaaacacagcagttttccttgctttttatatgcagaatatcaaagtcatttctaatttagttgtcaaaaacatatacatattttaacattagtttttttgaaaactcttggttttgtttttttggaaatgagtgggccactaagccacactttcccttcatcctgcttaatccttccagcatgtctctgcactaataaacagctaaattcacataatcatcctatttactgaagcatggtcatgctggtttatagattttttacccatttctactctttttctctattggtggcactgtaaatactttccagtattaaattatccttttctaacactgtaggaactattttgaatgcatgtgactaagagcatgatttatagcacaacctttccaataatcccttaatcagatcacattttgataaaccctgggaacatctggctgcaggaatttcaatatgtagaaacgctgcctatggttttttgcccttactgttgagactgcaatatcctagaccctagttttatactagagttttatttttagcaatgcctattgcaagtgcaattatatactccagggaaattcaccacactgaatcgagcatttgtgtgtgtatgtgtgaagtatatactgggacttcagaagtgcaatgtatttttctcctgtgaaacctgaatctacaagttttcctgccaagccactcaggtgcattgcagggaccagtgataatggctgatgaaaattgatgattggtcagtgaggtcaaaaggagccttgggattaataaacatgcactgagaagcaagaggaggagaaaaagatgtctttttcttccaggtgaactggaatttagttttgcctcagatttttttcccacaagatacagaagaagataaagatttttttggttgagagtgtgggtcttgcattacatcaaacagagttcaaattccacacagataagaggcaggatatataagcgccagtggtagttgggaggaataaaccattatttggatgcaggtggtttttgattgcaaatatgtgtgtgtcttcagtgattgtatgacagatgatgtattcttttgatgttaaaagattttaagtaagagtagatacattgtacccattttacattttcttattttaactacagtaatctacataaatatacctcagaaatcatttttggtgattattttttgttttgtagaattgcacttcagtttattttcttacaaataaccttacattttgtttaatggcttccaagagccttttttttttttgtatttcagagaaaattcaggtaccaggatgcaatggatttatttgattcaggggacctgtgtttccatgtcaaatgttttcaaataaaatgaaatatgagtttcaatactttttatattttaatatttccattcattaatattatggttattgtcagcaattttatgtttgaatatttgaaataaaagtttaagatttgaaaatggtatgtattataatttctattcaaatattaataataatattgagtgcagcatttctagaatggcagtggccggtggggacagggctgagccagcaccaaccactcagcctttgagatcccgaggctggtctactgctgagaccttttgttagaagagaggagatcaagcatttgcaaggtttctgagtgtcaaaatatgaatccaagataactctttcacaatcctaacttcatgctgtctacaggtccatattttagcctgctttctccatgttcatccgaaaagaaagaaaagctaagggtggtggtcatatttgaaattagccagatcttaagtttttctgggggaaatttagaagaaaatatggaaaagtgactatgagcaca

SEQ ID NO: 113 - DNA, 290 bp, Artificial Sequence; identical to SEQ ID NO: 31
  Synthetic polynucleotide

SEQ ID NO: 114 - DNA, 25 bp, Artificial Sequence
  Synthetic polynucleotide
ctgacctcttctcttcctcccacag

SEQ ID NO: 115 - DNA, 12 bp, Artificial Sequence
  Synthetic polynucleotide
gccgccaccatg

SEQ ID NO: 116 - DNA, 720 bp, Artificial Sequence; identical to SEQ ID NO: 87
  Synthetic polynucleotide

SEQ ID NO: 117 - DNA, 3381 bp, Artificial Sequence; identical to SEQ ID NO: 38
  Synthetic polynucleotide

SEQ ID NO: 118 - DNA, 328 bp, Artificial Sequence; identical to SEQ ID NO: 32
  Synthetic polynucleotide

SEQ ID NO: 119 - DNA, 4021 bp, Artificial Sequence; identical to SEQ ID NO: 39
  Synthetic polynucleotide

SEQ ID NO: 120 - DNA, 290 bp, Artificial Sequence; identical to SEQ ID NO: 31
  Synthetic polynucleotide

SEQ ID NO: 121 - DNA, 25 bp, Artificial Sequence
  Synthetic polynucleotide
ctgacctcttctcttcctcccacag

SEQ ID NO: 122 - DNA, 12 bp, Artificial Sequence
  Synthetic polynucleotide
gccgccaccatg

SEQ ID NO: 123 - DNA, 3451 bp, Artificial Sequence
  Synthetic polynucleotide
ctgacctcttctcttcctcccacaggtacctcagccagcatggccgcctccttcccacctacccttggattgtcctccgcccctgacgaaattcaacatccccacatcaaattctcggagtggaagttcaagctctttcgcgtgcgctcgttcgaaaagacccccgaggaagcccaaaaggagaagaaagactcattcgaaggaaaacccagcctcgaacagtccccggccgtcctggacaaggccgacgggcagaagcctgtgccgacccagccgctgctgaaagcgcacccgaaattctccaagaagtttcacgataacgagaaggcccggggaaaggccatccaccaagcaaaccttagacacctgtgccgcatctgtgggaactcattcagagccgacgaacataaccggagataccctgtgcatggccctgtcgacggaaagaccctggggctcctgagaaagaaggagaagagggcgacatcctggccggacctgatcgcaaaggtgttcagaatcgacgtgaaggcagatgtggacagcatccacccaaccgagttctgccacaactgctggagcattatgcaccggaagttcagctcagcgccctgtgaagtgtacttcccgcgcaacgtgactatggagtggcatccacacactccgtcctgcgacatctgtaacactgctcggcgcggactcaagaggaagtccctgcagccgaatctgcagctgagcaagaagcttaagaccgtgctggaccaggctcggcaggcccgccagcacaagcgacgcgcccaggcccggatctcatctaaggatgtgatgaagaagatcgccaattgcagcaaaatccacctgtctaccaagctgctggcggtggacttcccggagcacttcgtgaagtccatcagctgtcagatctgcgagcatattctcgccgaccccgtggagactaattgcaagcacgtgttctgccgcgtgtgcatcctgcgctgcctgaaggtcatgggctcctattgcccttcctgccggtacccctgtttccctactgatctggagtccccggtcaagtccttcttgtccgtgctgaactccctgatggtcaaatgtcccgcaaaggagtgcaatgaggaagtgtccctggaaaagtacaaccaccacatcagcagccacaaggagtccaaagaaatctttgtgcacattaacaagggcggtcggccccggcagcatctgctctcgctgactcgccgggcccagaagcacaggctccgggagctgaagctgcaagtcaaggccttcgccgacaaggaagagggaggagatgtgaagtccgtgtgcatgaccctgtttttgctggcgctgcgggctcggaacgaacacagacaagctgatgaactggaggccatcatgcagggcaaaggatcgggactccagccggctgtgtgtctcgccatccgcgtcaacacattcctctcatgctcccaataccacaagatgtacaggactgtgaaggccatcaccggacggcagatctttcagccactccacgcccttcggaacgcagaaaaggtcttgctgccgggataccatcatttcgaatggcagccgcccttgaaaaacgtgtcctcgtccaccgacgtgggcattattgatgggctgagcggcctgtcctcctctgtggatgactaccctgtggataccatcgccaaacggttcagatacgattccgcgctggtgtcggccctgatggacatggaggaggacatcctggagggaatgagatcacaagatctggacgactacctcaacgggcccttcacggtggtggtcaaggaatcgtgcgatggaatgggcgacgtgtcggagaagcacggttccggacctgtggtgccggaaaaggccgtgcgcttctccttcaccatcatgaagatcaccattgcgcatagctcccagaacgtcaaagtgttcgaagaggccaagccgaactcagagctctgctgcaagccgctgtgcctgatgttggcggacgagagcgatcacgaaaccctgaccgccattctgtcgcctctgatcgcggagagggaggccatgaagtcctccgaactgatgctggagctgggcggtattttgcggacttttaagttcatcttccggggaaccggttatgacgaaaagctcgtgcgcgaagtggagggcctggaagcctcaggctccgtctacatctgcactctctgcgacgccacccggctggaggcgtcacagaatcttgtgttccactcgatcactaggtcccacgcggagaacctggaacgctatgaggtctggcgctctaacccataccacgaatccgtggaagaacttcgggacagagtgaagggagtgtcagcaaagcctttcattgaaaccgtgcctagcatcgacgccctccattgcgacatcggcaacgccgccgagttctacaagatcttccagcttgagatcggggaagtgtacaagaacccgaacgcctccaaggaagaaagaaagcggtggcaggctacccttgacaaacacctccgcaagaagatgaacctgaagcccattatgcggatgaacggaaacttcgctaggaagctgatgactaaggaaacggtcgacgcggtctgtgaactgatccccagcgaagaacgacatgaagcgctgcgcgaactcatggacctgtacctgaagatgaagcctgtctggcggagctcgtgccctgccaaggagtgcccggagtcgctgtgtcagtacagctttaacagccaaaggttcgcagagctgctgtcgaccaagttcaagtacagatacgaaggaaagattaccaactacttccacaagactctcgctcacgtgcccgagattatcgaacgcgatggttccatcggggcctgggcctccgagggcaacgagtcgggcaacaagttgttccgccggtttagaaagatgaacgcccgccagtccaagtgctacgaaatggaagatgtgctgaagcatcactggctgtatacctccaagtacctccagaagttcatgaacgcacataacgccctcaagacctccgggttcaccatgaacccccaggcctccctcggtgaccctctgggaattgaagatagcttggagagccaggactcgatggaattctagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatccggaacaggtgtgataatgagagatcttgcgttccaacgagaaactaatgtt

SEQ ID NO: 124 - DNA, 226 bp, Artificial Sequence; identical to SEQ ID NO: 35
  Synthetic polynucleotide

SEQ ID NO: 125 - DNA, 328 bp, Artificial Sequence; identical to SEQ ID NO: 32
  Synthetic polynucleotide

SEQ ID NO: 126 - DNA, 2304 bp, Artificial Sequence
  Synthetic polynucleotide
gcaaagatgaatcaaagattctgtccttaaagaccttaaggtttttgtggaaggaaataaaactttacatgtatatatttaagcacttatatgtgtgtaacaggtataagtaaccataaacactgtcagaagaggaaataactctatgatcagcacctaacatgatatattaaggtagaagatttaatacatatcttttggaatacatgaataaataattgaatgtatttatttttattatttataagatacatcagtgggatattgatattggtcttaatatgacttgttttcattgttctcaggtacctcagccagcatggcagcctctttcccacccaccttgggactcagttctgccccagataccggtatggccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagtcgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcctgacaaccactacctgagcacccagtccgccctgagcaaagaccccaacgagaagcgcgatcacatggtcctgctggagttcgtgaccgccgccgggatcactctcggcatggacgagctgtacaagtaaactgtgccttctagttgccagccatctgttgtttgcccctcccccgtgccttccttgaccctggaaggtgccactcccactgccctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatccggaacaggtgtgataatgagagatcttgcgttccaacgagaaactaatgtttctagaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagc

SEQ ID NO: 127 - DNA, 367 bp, Artificial Sequence
  Synthetic polynucleotide
gcaaagatgaatcaaagattctgtccttaaagaccttaaggtttttgtggaaggaaataaaactttacatgtatatatttaagcacttatatgtgtgtaacaggtataagtaaccataaacactgtcagaagaggaaataactctatgatcagcacctaacatgatatattaaggtagaagatttaatacatatcttttggaatacatgaataaataattgaatgtatttatttttattatttataagatacatcagtgggatattgatattggtcttaatatgacttgttttcattgttctcaggtacctcagccagcatggcagcctctttcccacccaccttgggactcagttctgccccagat

SEQ ID NO: 128 - DNA, 516 bp, Artificial Sequence; identical to SEQ ID NO: 107
  Synthetic polynucleotide

SEQ ID NO: 129 - DNA, 5 bp, Artificial Sequence
  Synthetic polynucleotide
ccatg

SEQ ID NO: 130 - DNA, 720 bp, Artificial Sequence; identical to SEQ ID NO: 87
  Synthetic polynucleotide

SEQ ID NO: 131 - DNA, 226 bp, Artificial Sequence; identical to SEQ ID NO: 62
  Synthetic polynucleotide

SEQ ID NO: 132 - DNA, 393 bp, Artificial Sequence
  Synthetic polynucleotide
aattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagc

SEQ ID NO: 133 - DNA, 6793 bp, Artificial Sequence
  Synthetic polynucleotide
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaaggatccggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagcaattcaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccgaaggtagaggttctctcctcacttgtggtgatgttgaagaaaaccctggtccaatgtctagactggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaaccgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 134 - DNA, 4269 bp, Artificial Sequence
  Synthetic polynucleotide
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaag

SEQ ID NO: 135 - DNA, 512 bp, Artificial Sequence
  Synthetic polynucleotide
cggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 136 - DNA, 588 bp, Artificial Sequence
  Synthetic polynucleotide
atgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaag

SEQ ID NO: 137 - DNA, 747 bp, Artificial Sequence
  Synthetic polynucleotide
atgtctagactggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaa

SEQ ID NO: 138 - DNA, 592 bp, Artificial Sequence; identical to SEQ ID NO: 37
  Synthetic polynucleotide

SEQ ID NO: 139 - DNA, 21 bp, Artificial Sequence
  Synthetic polynucleotide
caagaggttgtctgaaggaag

SEQ ID NO: 140 - DNA, 19 bp, Artificial Sequence
  Synthetic polynucleotide
gacgtgaagaatgtgcgag

SEQ ID NO: 141 - DNA, 27 bp, Artificial Sequence
  Synthetic polynucleotide
ctgctgcaccctggcctcctgaactaa

SEQ ID NO: 142 - DNA, 22 bp, Artificial Sequence
  Synthetic polynucleotide
gtggaacaggtgtgataatgag

SEQ ID NO: 143 - DNA, 20 bp, Artificial Sequence
  Synthetic polynucleotide
ggaggacaatccaagggtag

SEQ ID NO: 144 - DNA, 25 bp, Artificial Sequence
  Synthetic polynucleotide
tgctgctgcaccctggcctcctgaa

SEQ ID NO: 145 - DNA, 5012 bp, Artificial Sequence
  Synthetic polynucleotide
tgagcacacagttattacttggaaattgtgtacagactaagttgaagatgttaggagggaagattgtgggccaagtaacggggtgtatgtgtgtgggtatagggtgggcagctgggatggaaatggggggctgctgctgctgctgcaccctggcctcctgaactaatgatatcactcaccagaaactactgttcctgcactgtccaagccaccccaaactagtttgtcaaaatgaatctgtgctgtgtggagggaggcacgcctgtagctctgatgtcagatggcaatgtgaattcctgacctcttctcttcctcccacaggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagtcgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcctgacaaccactacctgagcacccagtccgccctgagcaaagaccccaacgagaagcgcgatcacatggtcctgctggagttcgtgaccgccgccgggatcactctcggcatggacgagctgtacaagtaaactggatccgtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgctggcaatgtagagcaggcatggaattttatatgctagtgagtcataatgatatgttagtgttaattagttttttcttcctttgattttattggccataattgctactcttcatacacagtatatcaaagagcttgataatttagttgtcaaaagtgcatcggcgacattatctttaattgtatgtatttggtgcttcttcagggattgaactcagtatctttcattaaaaaacacagcagttttccttgctttttatatgcagaatatcaaagtcatttctaatttagttgtcaaaaacatatacatattttaacattagtttttttgaaaactcttggttttgtttttttggaaatgagtgggccactaagccacactttcccttcatcctgcttaatccttccagcatgtctctgcactaataaacagctaaattcacataatcatcctatttactgaagcatggtcatgctggtttatagattttttacccatttctactctttttctctattggtggcactgtaaatactttccagtattaaattatccttttctaacactgtaggaactattttgaatgcatgtgactaagagcatgatttatagcacaacctttccaataatcccttaatcagatcacattttgataaaccctgggaacatctggctgcaggaatttcaatatgtagaaacgctgcctatggttttttgcccttactgttgagactgcaatatcctagaccctagttttatactagagttttatttttagcaatgcctattgcaagtgcaattatatactccagggaaattcaccacactgaatcgagcatttgtgtgtgtatgtgtgaagtatatactgggacttcagaagtgcaatgtatttttctcctgtgaaacctgaatctacaagttttcctgccaagccactcaggtgcattgcagggaccagtgataatggctgatgaaaattgatgattggtcagtgaggtcaaaaggagccttgggattaataaacatgcactgagaagcaagaggaggagaaaaagatgtctttttcttccaggtgaactggaatttagttttgcctcagatttttttcccacaagatacagaagaagataaagatttttttggttgagagtgtgggtcttgcattacatcaaacagagttcaaattccacacagataagaggcaggatatataagcgccagtggtagttgggaggaataaaccattatttggatgcaggtggtttttgattgcaaatatgtgtgtgtcttcagtgattgtatgacagatgatgtattcttttgatgttaaaagattttaagtaagagtagatacattgtacccattttacattttcttattttaactacagtaatctacataaatatacctcagaaatcatttttggtgattattttttgttttgtagaattgcacttcagtttattttcttacaaataaccttacattttgtttaatggcttccaagagccttttttttttttgtatttcagagaaaattcaggtaccaggatgcaatggatttatttgattcaggggacctgtgtttccatgtcaaatgttttcaaataaaatgaaatatgagtttcaatactttttatattttaatatttccattcattaatattatggttattgtcagcaattttatgtttgaatatttgaaataaaagtttaagatttgaaaatggtatgtattataatttctattcaaatattaataataatattgagtgcagcatttctaggatcctaaactgtgccttctagttgccagccatctgttgtttgcccctcccccgtgccttccttgaccctggaaggtgccactcccactgccctttcctaataaaatgaggaaattgcatcgcattgtctgagtaggtgtcattctattctggggggtggggtggggcaggacagcaagggggaggattgggaagacaatagcaggcatgctggggatgcggtgggctctatggtctagatggcagtggccggtggggacagggctgagccagcaccaaccactcagcctttgagatcccgaggctggtctactgctgagaccttttgttagaagagaggagatcaagcatttgcaaggtttctgagtgtcaaaatatgaatccaagataactctttcacaatcctaacttcatgctgtctacaggtccatattttagcctgctttctccatgttcatccgaaaagaaagaaaagctaagggtggtggtcatatttgaaattagccagatcttaagtttttctgggggaaatttagaagaaaatatggaaaagtgactatgagcaca

SEQ ID NO: 146 - DNA, 290 bp, Artificial Sequence; identical to SEQ ID NO: 31
  Synthetic polynucleotide

SEQ ID NO: 147 - DNA, 25 bp, Artificial Sequence
  Synthetic polynucleotide
ctgacctcttctcttcctcccacag

SEQ ID NO: 148 - DNA, 12 bp, Artificial Sequence
  Synthetic polynucleotide
gccgccaccatg

SEQ ID NO: 149 - DNA, 720 bp, Artificial Sequence; identical to SEQ ID NO: 87
  Synthetic polynucleotide

SEQ ID NO: 150 - DNA, 3381 bp, Artificial Sequence; identical to SEQ ID NO: 38
  Synthetic polynucleotide

SEQ ID NO: 151 - DNA, 226 bp, Artificial Sequence; identical to SEQ ID NO: 62
  Synthetic polynucleotide

SEQ ID NO: 152 - DNA, 328 bp, Artificial Sequence; identical to SEQ ID NO: 32
  Synthetic polynucleotide

SEQ ID NO: 153 - DNA, 3097 bp, Artificial Sequence
  Synthetic polynucleotide
tgagcacacagttattacttggaaattgtgtacagactaagttgaagatgttaggagggaagattgtgggccaagtaacggggtgtatgtgtgtgggtatagggtgggcagctgggatggaaatggggggctgctgctgctgctgcaccctggcctcctgaactaatgatatcactcaccagaaactactgttcctgcactgtccaagccaccccaaactagtttgtcaaaatgaatctgtgctgtgtggagggaggcacgcctgtagctctgatgtcagatggcaatgtgaattcctgacctcttctcttcctcccacaggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagtcgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcctgacaaccactacctgagcacccagtccgccctgagcaaagaccccaacgagaagcgcgatcacatggtcctgctggagttcgtgaccgccgccgggatcactctcggcatggacgagctgtacaagtaaactggatccgaattaactcgaggaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccaacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgggagctggtgctaccggcagagctatggatggacctagactgctgctcctgctgctgctcggagtttctcttggcggagccaaagaggcctgtcctaccggcctgtatacacactctggcgagtgctgcaaggcctgcaatcttggagaaggcgtggcacagccttgcggcgctaatcagacagtgtgcgagccttgcctggacagcgtgacctttagcgacgtggtgtctgccaccgagccatgcaagccttgtaccgagtgtgtgggcctgcagagcatgtctgccccttgtgtggaagccgacgatgccgtgtgtagatgcgcctacggctactaccaggacgagacaacaggcagatgcgaggcctgtagagtgtgtgaagccggctctggactggtgttcagctgccaagacaagcagaacaccgtgtgcgaggaatgccccgatggcacctatagcgacgaggccaaccatgtagatccctgcctgccttgtactgtgtgcgaagataccgagcggcagctgcgcgagtgtacaagatgggctgatgccgagtgcgaagagatccccggcagatggatcaccagaagcacacctccagagggcagcgatagcacagccccttctacacaagagcccgaggctcctcctgagcaggatctgattgcctctacagtggccggcgtggtcacaacagtgatgggatcttctcagcccgtggtcaccagaggcaccaccgacaatctgatccccgtgtactgtagcatcctggccgccgtggttgtgggactcgtggcctatatcgccttcaagcggtggaaccggggcatcctgtaatgatctagcaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtctagaatggcagtggccggtggggacagggctgagccagcaccaaccactcagcctttgagatcccgaggctggtctactgctgagaccttttgttagaagagaggagatcaagcatttgcaaggtttctgagtgtcaaaatatgaatccaagataactctttcacaatcctaacttcatgctgtctacaggtccatattttagcctgctttctccatgttcatccgaaaagaaagaaaagctaagggtggtggtcatatttgaaattagccagatcttaagtttttctgggggaaatttagaagaaaatatggaaaagtgactatgagcaca

SEQ ID NO: 154 - DNA, 290 bp, Artificial Sequence; identical to SEQ ID NO: 31
  Synthetic polynucleotide

SEQ ID NO: 155 - DNA, 25 bp, Artificial Sequence
  Synthetic polynucleotide
ctgacctcttctcttcctcccacag

SEQ ID NO: 156 - DNA, 12 bp, Artificial Sequence
  Synthetic polynucleotide
gccgccaccatg

SEQ ID NO: 157 - DNA, 720 bp, Artificial Sequence; identical to SEQ ID NO: 87
  Synthetic polynucleotide

SEQ ID NO: 158 - DNA, 607 bp, Artificial Sequence; identical to SEQ ID NO: 63
  Synthetic polynucleotide

SEQ ID NO: 159 - DNA, 843 bp, Artificial Sequence; identical to SEQ ID NO: 64
  Synthetic polynucleotide

SEQ ID NO: 160 - DNA, 225 bp, Artificial Sequence; identical to SEQ ID NO: 65
  Synthetic polynucleotide

SEQ ID NO: 161 - DNA, 328 bp, Artificial Sequence; identical to SEQ ID NO: 32
  Synthetic polynucleotide

SEQ ID NO: 162 - DNA, 2094 bp, Artificial Sequence
  Synthetic polynucleotide
tgagcacacagttattacttggaaattgtgtacagactaagttgaagatgttaggagggaagattgtgggccaagtaacggggtgtatgtgtgtgggtatagggtgggcagctgggatggaaatggggggctgctgctgctgctgcaccctggcctcctgaactaatgatatcactcaccagaaactactgttcctgcactgtccaagccaccccaaactagtttgtcaaaatgaatctgtgctgtgtggagggaggcacgcctgtagctctgatgtcagatggcaatgtgaattcctgacctcttctcttcctcccacaggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagtcgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcctgacaaccactacctgagcacccagtccgccctgagcaaagaccccaacgagaagcgcgatcacatggtcctgctggagttcgtgaccgccgccgggatcactctcggcatggacgagctgtacaagtaaactggatccgaattaactcgaggaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccaacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgaggaccgaggcccccgagggcaccgagagcgagatggagacccccagcgccatcaacggcaaccccagctggcacccggatccaggtaagttctagaatggcagtggccggtggggacagggctgagccagcaccaaccactcagcctttgagatcccgaggctggtctactgctgagaccttttgttagaagagaggagatcaagcatttgcaaggtttctgagtgtcaaaatatgaatccaagataactctttcacaatcctaacttcatgctgtctacaggtccatattttagcctgctttctccatgttcatccgaaaagaaagaaaagctaagggtggtggtcatatttgaaattagccagatcttaagtttttctgggggaaatttagaagaaaatatggaaaagtgactatgagcaca

SEQ ID NO: 163 - DNA, 290 bp, Artificial Sequence; identical to SEQ ID NO: 31
  Synthetic polynucleotide

SEQ ID NO: 164 - DNA, 25 bp, Artificial Sequence
  Synthetic polynucleotide
ctgacctcttctcttcctcccacag

SEQ ID NO: 165 - DNA, 12 bp, Artificial Sequence
  Synthetic polynucleotide
gccgccaccatg

SEQ ID NO: 166 - DNA, 720 bp, Artificial Sequence; identical to SEQ ID NO: 87
  Synthetic polynucleotide

SEQ ID NO: 167 - DNA, 607 bp, Artificial Sequence; identical to SEQ ID NO: 63
  Synthetic polynucleotide

SEQ ID NO: 168 - DNA, 78 bp, Artificial Sequence; identical to SEQ ID NO: 66
  Synthetic polynucleotide

SEQ ID NO: 169 - DNA, 8 bp, Artificial Sequence
  Synthetic polynucleotide
aggtaagt

SEQ ID NO: 170 - DNA, 328 bp, Artificial Sequence; identical to SEQ ID NO: 32
  Synthetic polynucleotide

SEQ ID NO: 171 - DNA, 19 bp, Artificial Sequence
  Synthetic polynucleotide
atcagatcgcaagtcgaga

SEQ ID NO: 172 - DNA, 19 bp, Artificial Sequence
  Synthetic polynucleotide
gccagagccaatgtcgaga

SEQ ID NO: 173 - DNA, 20 bp, Artificial Sequence
  Synthetic polynucleotide
gacagctggcaatgttaaga

SEQ ID NO: 174 - DNA, 20 bp, Artificial Sequence
  Synthetic polynucleotide
ttcacatggcaatgtcaagt

(19) United States
(12) Patent Application Publication
(10) Pub. No.: US 2023/0365996 A1
(43) Pub. Date: Nov. 16, 2023

(54) REPLACEMENT OF RAG1 FOR USE IN THERAPY

(71) Applicants: Ospedale San Raffaele S.R.L., Milan (IT); Fondazione Telethon, Rome (IT)

(72) Inventors: Anna Villa, Milan (IT); Pietro Genovese, Milan (IT); Pietro Genovese, Milan (IT); Nicolo Sacchetti, Milan (IT); Maria Carmina Castiello, Milan (IT); Samuele Ferrari, Milan (IT)

(21) Appl. No.: 18/030,711

(22) PCT Filed: Oct. 12, 2021
(86) PCT No.: PCT/EP2021/078222

§ 371 Date: Apr. 6, 2023

(30) Foreign Application Priority Data
Oct. 12, 2020 (GB) 2016139.4
Apr. 28, 2021 (AU) 2021202657

(51) Int. Cl. C12N 15/90; C12N 9/10; C12N 9/22; C12N 5/0789

(52) U.S. Cl. CPC C12N 15/907; C12N 5/0647; C12N 9/104; C12N 9/22; A61K 48/00; C12N 2750/14143

(57) ABSTRACT

The present invention relates to an isolated polynucleotide comprising from 5′ to 3′: a first homology region, a splice acceptor sequence, a nucleotide sequence encoding a RAG1 polypeptide, and a second homology region for use in treating a RAG-deficient immunodeficiency.

### FIELD OF THE INVENTION

The present invention relates to methods for gene-editing cells to introduce a RAG1 polypeptide, for example as a treatment for severe combined immunodeficiency. The present invention also relates to polynucleotides, vectors, guide RNAs, kits, compositions, and gene editing systems for use in said methods. The present invention also relates to genomes and cells obtained or obtainable by said methods.

### BACKGROUND TO THE INVENTION

The RAG1 and RAG2 proteins initiate V(D)J recombination, allowing generation of a diverse repertoire of T and B cells (Teng G, Schatz DG. Advances in Immunology. 2015;128:1-39). RAG mutations in humans cause a broad spectrum of phenotypes, including T⁻ B⁻ SCID, Omenn syndrome (OS), atypical SCID (AS) and combined immunodeficiency with granuloma/autoimmunity (CID-G/AI) (Notarangelo LD, et al. Nat Rev Immunol. 2016; 16(4):234-246).

Hematopoietic stem cell transplantation (HSCT) is the mainstay for severe forms of RAG1 deficiency, including T⁻ B⁻ SCID, OS and AS with an overall survival of ~80% after transplantation from donors other than matched siblings (Haddad E, et al. Blood. 2018;132(17):1737-49). However, overall survival rate is lower in non-matched-sibling donors and a high rate of graft failure and poor T and B cell immune reconstitution are observed in the absence of myeloablative or reduced intensity conditioning. Besides donor type and conditioning, other factors associated with worse outcomes after HSCT include age (>3.5 months of life) and infections at the time of transplantation.

An alternative approach to overcome the obstacles with HSCT is represented by gene therapy. Selective advantage of gene-corrected hematopoietic stem cells (HSCs) to overcome the block of T and B cells that occur in the absence of RAG activity represents the rationale for developing such a strategy. In recent years, lentiviral vectors have become the strategy of choice to deliver the transgene of interest, and allow its expression under the control of suitable promoters (Naldini L, Nature. 2015;526:351-360). In the case of RAG1 deficiency, the observation that endogenous RAG1 gene expression is tightly regulated during cell cycle and during lymphoid development, may expose to the risk that ectopic or dysregulated gene expression could lead to immune dysregulation or leukemia (Lagresle-Peyrou C, et al. Blood. 2006;107(1):63-72; Pike-Overzet K, et al. Leukemia. 2011;25(9):1471-83; and Pike-Overzet K, et al. Journal of Allergy and Clinical Immunology . 2014;134:242-243). Several groups have examined the safety and efficacy of lentivirus-mediated gene therapy for RAG deficiency in preclinical models showing poor immune reconstitution or severe signs of inflammation, with cellular infiltrates in the skin, lung, liver, kidney, and presence of circulating anti-double strand DNA (van Til NP, et al. J Allergy Clin Immunol. 2014;133(4):1116-23).

Overall, these data raise significant concerns on the clinical use of conventional RAG1 gene therapy vectors that allow suboptimal levels and deregulated pattern of gene expression.

Thus, there is a demand for improved treatments for RAG1 deficiency.

### SUMMARY OF THE INVENTION

The present inventors have developed a gene editing strategy to correct mutations in the RAG1 gene by targeting the genomic region located at the 5′ of the second exon, which contains the entire coding sequence of the gene.

The present inventors have designed and selected a panel of CRISPR-Cas9 nucleases and identified specific sites in non-repeated regions of the first intron of the human RAG1 gene. The present inventors have identified guide RNAs and optimal conditions for the delivery of the CRISPR-Cas9 nuclease ribonucleoprotein complexes. In parallel, the present inventors have developed a donor DNA carrying the human RAG1 cDNA.

The gene editing strategy allows a high level of activity (measured as frequency of NHEJ-mutagenesis) and targeting efficiency (measured as GFP expression), both in a surrogate cell line deficient in RAG1 expression and expressing a recombination cassette, and in humans CD34+ HSCs obtained from mobilized peripheral blood (mPB). High editing efficiencies were reached in mobilized peripheral blood (mPB) CD34⁺ cells using the gene editing strategy.

In one aspect, the present invention provides a polynucleotide comprising from 5′ to 3′: a first homology region, a splice acceptor sequence, a nucleotide sequence encoding a RAG1 polypeptide, and a second homology region.

In another aspect, the present invention provides a polynucleotide comprising from 5′ to 3′: a first homology region, a nucleotide sequence encoding a RAG1 polypeptide, and a second homology region.

In some embodiments:
(i) the first homology region is homologous to a first region of the RAG1 intron 1 and the second homology region is homologous to a second region of the RAG1 intron 1; or
(ii) the first homology region is homologous to a first region of the RAG1 intron 1 or the RAG1 exon 2 and the second homology region is homologous to a second region of the RAG1 exon 2.

In some embodiments, the first homology region is homologous to a first region of the RAG1 intron 1 and the second homology region is homologous to a second region of the RAG1 intron 1.

In some embodiments, the first homology region is homologous to a first region of the RAG1 intron 1 and the second homology region is homologous to a second region of the RAG1 exon 2.

In some embodiments, the first homology region is homologous to a first region of the RAG1 exon 2 and the second homology region is homologous to a second region of the RAG1 exon 2.

In some embodiments:
(i) the first homology region is homologous to a region upstream of chr 11: 36569295 and the second homology region is homologous to a region downstream of chr 11: 36569298;
(ii) the first homology region is homologous to a region upstream of chr 11: 36573790 and the second homology region is homologous to a region downstream of chr 11: 36573793;
(iii) the first homology region is homologous to a region upstream of chr 11: 36573641 and the second homology region is homologous to a region downstream of chr 11: 36573644;
(iv) the first homology region is homologous to a region upstream of chr 11: 36573351 and the second homology region is homologous to a region downstream of chr 11: 36573354;
(v) the first homology region is homologous to a region upstream of chr 11: 36569080 and the second homology region is homologous to a region downstream of chr 11: 36569083;
(vi) the first homology region is homologous to a region upstream of chr 11: 36572472 and the second homology region is homologous to a region downstream of chr 11: 36572475;
(vii) the first homology region is homologous to a region upstream of chr 11: 36571458 and the second homology region is homologous to a region downstream of chr 11: 36571461;
(viii) the first homology region is homologous to a region upstream of chr 11: 36571366 and the second homology region is homologous to a region downstream of chr 11: 36571369;
(ix) the first homology region is homologous to a region upstream of chr 11: 36572859 and the second homology region is homologous to a region downstream of chr 11: 36572862;
(x) the first homology region is homologous to a region upstream of chr 11: 36571457 and the second homology region is homologous to a region downstream of chr 11: 36571460;
(xi) the first homology region is homologous to a region upstream of chr 11: 36569351 and the second homology region is homologous to a region downstream of chr 11: 36569354; or
(xii) the first homology region is homologous to a region upstream of chr 11: 36572375 and the second homology region is homologous to a region downstream of chr 11: 36572378.

In some embodiments:
(i) the first homology region is homologous to a region upstream of chr 11: 36569295 and the second homology region is homologous to a region downstream of chr 11: 36569298;
(ii) the first homology region is homologous to a region upstream of chr 11: 36573351 and the second homology region is homologous to a region downstream of chr 11: 36573354; or
(iii) the first homology region is homologous to a region upstream of chr 11: 36571366 and the second homology region is homologous to a region downstream of chr 11: 36571369.

In preferred embodiments, the first homology region is homologous to a region upstream of chr 11: 36569295 and the second homology region is homologous to a region downstream of chr 11: 36569298.

In some embodiments, the first homology region is homologous to a region upstream of chr 11: 36573790 and the second homology region is homologous to a region downstream of chr 11: 36573793.

In some embodiments, the first homology region is homologous to a region upstream of chr 11: 36573641 and the second homology region is homologous to a region downstream of chr 11: 36573644.

In some embodiments, the first homology region is homologous to a region upstream of chr 11: 36573351 and the second homology region is homologous to a region downstream of chr 11: 36573354.

In some embodiments, the first homology region is homologous to a region upstream of chr 11: 36569080 and the second homology region is homologous to a region downstream of chr 11: 36569083.

In some embodiments, the first homology region is homologous to a region upstream of chr 11: 36572472 and the second homology region is homologous to a region downstream of chr 11: 36572475.

In some embodiments, the first homology region is homologous to a region upstream of chr 11: 36571458 and the second homology region is homologous to a region downstream of chr 11: 36571461.

In some embodiments, the first homology region is homologous to a region upstream of chr 11: 36571366 and the second homology region is homologous to a region downstream of chr 11: 36571369.

In some embodiments, the first homology region is homologous to a region upstream of chr 11: 36572859 and the second homology region is homologous to a region downstream of chr 11: 36572862.

In some embodiments, the first homology region is homologous to a region upstream of chr 11: 36571457 and the second homology region is homologous to a region downstream of chr 11: 36571460.

In some embodiments, the first homology region is homologous to a region upstream of chr 11: 36569351 and the second homology region is homologous to a region downstream of chr 11: 36569354.

In some embodiments, the first homology region is homologous to a region upstream of chr 11: 36572375 and the second homology region is homologous to a region downstream of chr 11: 36572378.

In preferred embodiments, the first homology region is homologous to a region comprising chr 11: 36569245-chr 11: 36569294 and/or the second homology region is homologous to a region comprising chr 11: 36569299-chr 11: 36569348.

In some embodiments, the 3′ terminal sequence of the first homology region comprises or consists of a nucleotide sequence that has at least 70% identity to SEQ ID NO: 7 and/or the 5′ terminal sequence of the second homology region comprises or consists of a nucleotide sequence that has at least 70% identity to SEQ ID NO: 19.

In some embodiments, the first homology region comprises or consists of a nucleotide sequence that has at least 70% identity to SEQ ID NO: 31, or a fragment thereof and/or the second homology region comprises or consists of a nucleotide sequence that has at least 70% identity to SEQ ID NO: 32, or a fragment thereof.

In some embodiments, the first and second homology regions are each 50-1000 bp in length, 100-500 bp in length, or 200-400 bp in length.

In some embodiments, the nucleotide sequence encoding a RAG1 polypeptide comprises or consists of a nucleotide sequence encoding an amino acid sequence that has at least 70% identity to SEQ ID NO: 4 or SEQ ID NO: 5.

In some embodiments, the nucleotide sequence encoding a RAG1 polypeptide comprises or consists of a nucleotide sequence that has at least 70% identity to SEQ ID NO: 6.

In some embodiments, the splice acceptor site comprises or consists of a nucleotide sequence that has at least 70% identity to SEQ ID NO: 33.

In preferred embodiments, the nucleotide sequence encoding a RAG1 polypeptide is operably linked to a polyadenylation sequence, optionally wherein the polyadenylation sequence is a bGH polyadenylation sequence.

In some embodiments, the nucleotide sequence encoding a RAG1 polypeptide is operably linked to a polyadenylation sequence comprising or consisting of a nucleotide sequence that has at least 70% identity to SEQ ID NO: 35.

In some embodiments, the nucleotide sequence encoding a RAG1 polypeptide is operably linked a Kozak sequence, optionally wherein the Kozak sequence comprises or consists of a nucleotide sequence that has at least 70% identity to SEQ ID NO: 36.

In some embodiments, the polynucleotide comprises or consists of a nucleotide sequence that has at least 70% identity to SEQ ID NO: 39.

In another aspect, the present invention provides a vector comprising the polynucleotide of the invention.

In some embodiments, the vector is a viral vector, optionally an adeno-associated viral (AAV) vector such as an AAV6 vector. In some embodiments, the vector is a lentiviral vector, such as an integration-defective lentiviral vector (IDLV).

In another aspect, the present invention provides a guide RNA comprising or consisting of a nucleotide sequence that has at least 90% identity to any of SEQ ID NOs: 41-52.

In another aspect, the present invention provides a guide RNA comprising or consisting of a nucleotide sequence that has at least 90% identity to any of SEQ ID NOs: 53-55.

In preferred embodiments, the guide RNA comprises or consists of a nucleotide sequence that has at least 90% identity to SEQ ID NO: 41. In preferred embodiments, the guide RNA comprises or consists of a nucleotide sequence that has at least 90% identity to SEQ ID NO: 53. In some embodiments, the guide RNA comprises or consists of a nucleotide sequence that has at least 90% identity to SEQ ID NO: 42. In some embodiments, the guide RNA comprises or consists of a nucleotide sequence that has at least 90% identity to SEQ ID NO: 43. In some embodiments, the guide RNA comprises or consists of a nucleotide sequence that has at least 90% identity to SEQ ID NO: 44. In some embodiments, the guide RNA comprises or consists of a nucleotide sequence that has at least 90% identity to SEQ ID NO: 45. In some embodiments, the guide RNA comprises or consists of a nucleotide sequence that has at least 90% identity to SEQ ID NO: 46. In some embodiments, the guide RNA comprises or consists of a nucleotide sequence that has at least 90% identity to SEQ ID NO: 47. In some embodiments, the guide RNA comprises or consists of a nucleotide sequence that has at least 90% identity to SEQ ID NO: 48. In some embodiments, the guide RNA comprises or consists of a nucleotide sequence that has at least 90% identity to SEQ ID NO: 49. In some embodiments, the guide RNA comprises or consists of a nucleotide sequence that has at least 90% identity to SEQ ID NO: 50. In some embodiments, the guide RNA comprises or consists of a nucleotide sequence that has at least 90% identity to SEQ ID NO: 51. In some embodiments, the guide RNA comprises or consists of a nucleotide sequence that has at least 90% identity to SEQ ID NO: 52. In some embodiments, the guide RNA comprises or consists of a nucleotide sequence that has at least 90% identity to SEQ ID NO: 54. In some embodiments, the guide RNA comprises or consists of a nucleotide sequence that has at least 90% identity to SEQ ID NO: 55.

In some embodiments, from one to five of the terminal nucleotides at 5′ end and/or 3′ end of the guide RNA are chemically modified to enhance stability, optionally wherein three terminal nucleotides at 5′ end and/or 3′ end if the guide RNA are chemically modified to enhance stability, optionally wherein the chemical modification is modification with 2′-O-methyl 3′phosphorothioate.

In another aspect, the present invention provides a kit comprising the polynucleotide or the vector of the invention.

In another aspect, the present invention provides a composition comprising the polynucleotide or the vector of the invention.

In another aspect, the present invention provides a gene-editing system comprising the polynucleotide or the vector of the invention.

In some embodiments, the kit, composition, or gene-editing system further comprises a guide RNA of the invention. In some embodiments, the kit, composition, or gene-editing system further comprises a RNA-guided nuclease, optionally wherein the RNA-guided nuclease is a Cas9 endonuclease

In another aspect, the present invention provides for use of the polynucleotide, the vector, the kit, the composition, or the gene-editing system, for gene editing a cell or a population of cells. In some embodiments, the use is ex vivo or in vitro use.

In another aspect, the present invention provides a genome comprising the polynucleotide of the invention.

In another aspect, the present invention provides a genome comprising a splice acceptor sequence and a nucleotide sequence encoding a RAG1 polypeptide located in the RAG1 intron 1 or RAG1 exon 2. In some embodiments, the splice acceptor sequence and the nucleotide sequence encoding RAG1 are located in the RAG1 intron 1.

In some embodiments:
(i) the splice acceptor sequence and the nucleotide sequence encoding RAG1 replace chr 11: 36569295 to chr 11: 36569298;
(ii) the splice acceptor sequence and the nucleotide sequence encoding RAG1 replace chr 11: 36573790 to chr 11: 36573793;
(iii) the splice acceptor sequence and the nucleotide sequence encoding RAG1 replace chr 11: 36573641 to chr 11: 36573644;
(iv) the splice acceptor sequence and the nucleotide sequence encoding RAG1 replace chr 11: 36573351 to chr 11: 36573354;
(v) the splice acceptor sequence and the nucleotide sequence encoding RAG1 replace chr 11: 36569080 to chr 11: 36569083;
(vi) the splice acceptor sequence and the nucleotide sequence encoding RAG1 replace chr 11: 36572472 to chr 11: 36572475;
(vii) the splice acceptor sequence and the nucleotide sequence encoding RAG1 replace chr 11: 36571458 to chr 11: 36571461;
(viii) the splice acceptor sequence and the nucleotide sequence encoding RAG1 replace chr 11: 36571366 to chr 11: 36571369;
(ix) the splice acceptor sequence and the nucleotide sequence encoding RAG1 replace chr 11: 36572859 to chr 11: 36572862;
(x) the splice acceptor sequence and the nucleotide sequence encoding RAG1 replace chr 11: 36571457 to chr 11: 36571460;
(xi) the splice acceptor sequence and the nucleotide sequence encoding RAG1 replace chr 11: 36569351 to chr 11: 36569354; or
(xii) the splice acceptor sequence and the nucleotide sequence encoding RAG1 replace chr 11: 36572375 to chr 11: 36572378.

In some embodiments:
(i) the splice acceptor sequence and the nucleotide sequence encoding RAG1 replace chr 11: 36569295 to chr 11: 36569298;
(ii) the splice acceptor sequence and the nucleotide sequence encoding RAG1 replace chr 11: 36573351 to chr 11: 36573354; or
(iii) the splice acceptor sequence and the nucleotide sequence encoding RAG1 replace chr 11: 36571366 to chr 11: 36571369.

In some embodiments, the splice acceptor sequence and the nucleotide sequence encoding RAG1 replace chr 11: 36569295 to chr 11: 36569298.

In another aspect, the present invention provides a cell comprising the polynucleotide, the vector, or the genome of the invention.

In another aspect, the present invention provides a population of cells comprising one or more cells of the present invention.

In another aspect, the present invention provides a method of gene editing a population of cells comprising delivering the polynucleotide or the vector of the invention to a population of cells to obtain a population of gene-edited cells. In some embodiments, the method is an ex vivo or in vitro method.

In another aspect, the present invention provides a method of treating immunodeficiency in a subject in need thereof, comprising delivering the polynucleotide or the vector of the invention to a population of cells to obtain a population of gene-edited cells and administering the population of gene-edited cells to the subject.

In another aspect, the present invention provides a population of gene-edited cells obtainable by the method of the invention.

In another aspect, the present invention provides the polynucleotide, the vector, the guide RNA, the kit, the composition, or the gene-editing system, for use in treating immunodeficiency in a subject.

In another aspect, the present invention provides a method of treating a subject comprising administering a cell, a population of cells, or a population of gene edited cells of the present invention to the subject.

In another aspect, the present invention provides a method of treating immunodeficiency in a subject in need thereof comprising administering a cell, a population of cells, or a population of gene edited cells of the present invention to the subject.

In another aspect, the present invention provides a cell, a population of cells, or a population of gene edited cells of the present invention for use as a medicament.

In another aspect, the present invention provides a cell, a population of cells, or a population of gene edited cells of the present invention for use in treating immunodeficiency in a subject.

### DESCRIPTION OF DRAWINGS

FIG. 1. Generation of NALM6 Cas9 and K562 Cas9 cell lines A) Schematic representation of the gene correction approach; B) Schematic representation of the protocol for generation of K562 Cas9 and NALM6 Cas9 cell lines; C) Vector Copy Number (VCN) of the integrated Cas9 containing cassette measured by ddPCR, telomerase was used as normalizer; D) Cas9 expression for scaling doses of doxycycline measured by qPCR in NALM6 Cas9 (left panel) and K562 Cas9 (right panel) cell lines, represented as fold change Vs actin.

FIG. 2. Selection of the best performing gRNA A) Schematic representation of the intronic and exonic loci targeted by the different gRNA tested; B) Schematic representation of the experimental protocol; C) Percentages of NHEJ induced indels in K562 Cas9 treated with different doses of plasmids encoding for different guides, 7 days after transfection, n=1; D) Percentages of NHEJ induced indels in NALM6 Cas9 treated with different doses of plasmids encoding for guides 3, 7 and 9, 7 days after transfection, n=1; E) Percentages of NHEJ induced indels in NALM6 Cas9 treated with different doses of guides 3 and 9 in vitro preassembled RNPs 7 days after transfection, n=1.

FIG. 3. Donor DNA optimization A) RAG1 gene expression measured by RT-qPCR, represented as fold change vs RAG1 expression in 293T cell line, actin was used as normalizer; B) Schematic representation of different SA_GFP DNA donor tested; C) Schematic representation of the splicing mechanism with SA_GFP_SD donor; D) Percentage of targeted cells measured by flow cytometry as GFP+ cells, 7 days after transfection; E) GFP expression levels measured as Mean Fluorescence Intensity (MFI) gating on GFP+ events; F) Representative FlowJo plots; One-way ANOVA, Geisser-Greenhouse correction for multiple comparison, n=3. P values: *<0.05; **<0.005; ***<0.0005; ****<0.0001. Mean±SD are shown.

FIG. 4. Off-target analysis A) Table shows the top 10 off-target sites predicted by in silico COSMID tool for guide 9. The off-target sequence, type of PAM, score, number of mismatches and chromosomal position are shown. B-C) Cutting efficiency measured as percentage of NHEJ (D) and dsDNA tag integration (ODN) on target site are evaluated by RFLP in K562 cells. D-E) Plots show the coverage of on-target reads (chromosome 11) of guide 9 (D) and guide 7 (E) and off-target reads identified for guide 7 by relaxed constraints (chromosome 20 and 9).

FIG. 5. Optimization of the gene editing protocol, guide 3 efficiency A) Schematic representation of the gene editing protocol; B) Schematic representation of the gating strategy; C) Percentages of NHEJ induced indels in hCB-CD34⁺ cells treated with different doses of guides 3 and 9 as in vitro preassembled RNPs, n=2; D) Percentage of targeted cells using guide 3, measured by flow cytometry as GFP⁺ cells in the hCD34⁺ gate, n=1; E) Percentage of targeted cells using guide 3, measured by flow cytometry as GFP⁺ cells in the three main hCD34+ cell subpopulations for hCD133 hCD90 expression, n=1.

FIG. 6. Optimization of the gene editing protocol, guide 9 efficiency A) Percentages of viable cells measured by flow cytometry as 7AAD⁻/AnnexinV⁻ at day 4; B) Total number of cells at day 7 expressed as fold increase compared day 3; C) Frequency of hCD34⁺ cells at day 7 measured by flow cytometry; D) Distribution of the 3 hCD34⁺ cell subpopulations measured by flow cytometry based on the expression of hCD133 and hCD90 at day 7; E) Frequency of targeted cells measured by flow cytometry as GFP⁺ cells in the 3 hCD34⁺ cell subpopulations based on the expression of hCD133 and hCD90 at day 7; F) Percentages of targeted cells measured by ddPCR at day 7, telomerase genomic site was used as normalizer; G) Total number of edited cells at day 7 calculated on frequency of targeted cells by ddPCR. One-way ANOVA, Geisser-Greenhouse correction for multiple comparison, n=3. P values: *<0.05; **<0.005; ***<0.0005; ****<0.0001. Mean±SD are shown.

FIG. 7. In vivo transplantation of gene edited hCB-CD34⁺ cells A) Percentages of targeted cells measured by ddPCR at day 4, telomerase genomic site was used as normalizer; B) Treated cell engraftment measured by flow cytometry as frequency of hCD45⁺ cells in peripheral blood (PB); C) Targeted cell engraftment measured in PB by flow cytometry as frequency of GFP⁺ cells in hCD45⁺ gate; D, F, H) B cell, T cell and Myeloid cell frequency in PB measured as percentage of hCD19⁺ cells (D), hCD3⁺ cells (F), hCD13⁺ cells (F) in hCD45⁺ gate, respectively. E, G, I) Targeted cells among the B-cell, T-cell and Myeloid-cell compartment in PB measured as GFP⁺ cells in the hCD19⁺ gate (E), hCD3⁺ gate (G) and hCD13⁺ gate (I), respectively; L) Frequency of hCD34⁺ cells measured by flow cytometry among hCD45⁺ cells in the bone marrow; M) Frequency of targeted cells measured by flow cytometry as GFP⁺ cells among hCD34⁺ cells in the bone marrow; N) Frequency of GFP⁺ expressing cells measured by flow cytometry, among different T-cell development stages in the thymus (according to the expression of hCD4 and hCD8), in the peripheral blood and in the spleen (according to the expression of hCD3, hCD4 and hCD8), 17 weeks after transplant. Mann-Whitney test at 17 weeks after transplant. Group size: SA_GFP n=5; PGK_GFP n=4. P values: *<0.05; **<0.005; ***<0.0005; ****<0.0001. Mean±SD are shown.

FIG. 8. Test corrective donor on hMPB-CD34⁺ cells A) Schematic representation of the corrective donor; B) Schematic representation of the experimental protocol; C) Percentages of targeted cells measured by ddPCR on sorted hCD34⁺ cell subpopulation according to the expression of hCD133 and hCD90 at day 4, telomerase genomic region was used as normalizer; D) Total number of cells at day 4 represented as fold increase compared day 0. N=3.

FIG. 9. In vivo transplantation of edited hMPB-CD34⁺ cells from HD and RAG1-patient A) Schematic representation of the experimental groups; B) Percentage of targeted cells measured by ddPCR at day 4, telomerase genomic region was used as normalizer; C) Cell engraftment measured by flow cytometry in PB as frequency of hCD45⁺ cells; D) Frequency of targeted cells among human cells measured by ddPCR in PB 8 weeks after transplant, telomerase genomic region was used as normalizer; E) Immune cell distribution in PB of mice transplanted with MPB-CD34⁺ of HD treated and untreated cells measured by flow cytometry according to the expression of hCD19, hCD3 and hCD13 in the hCD45⁺ gate; F) Immune cell distribution in PB of mice transplanted with MPB-CD34⁺ cells derived from a RAG1-patient treated and untreated cells measured by flow cytometry according to the expression of hCD19, hCD3 and hCD13 in the hCD45+ gate; G, H) Analyses in bone marrow (G) and spleen (H) of the proportion of human engraftment measured as frequency of hCD45⁺ cells by flow cytometry (left panels) and of targeting efficiency measured as HDR by ddPCR (right panels). Mean±SD are shown.

FIG. 10. Multiparametric analysis of hMPB-CD34⁺ cells from HD and RAG1-patient before and after gene editing manipulation. A, B) Analysis of HSPC composition was performed in MPB-CD34⁺ cells derived from healthy donor (HD, A) and a RAG1-Patient (Pt, B) by flow-cytometry. The analysis was performed before the expansion phase (day-3) and 1 day after the gene editing procedure (GE). Untreated cells (UT) were also analyzed the same day of edited cells. Graphs show 20 subtypes analyzed in the Lineage negative (Lin⁻) CD34⁺ gate including: Hematopoietic Stem cells (HSC), Multipotent Progenitors (MPP), Multi-Lymphoid Progenitors (MLP), Early T Progenitors (ETP), B and NK cell precursors (Pre-B/NK), common myeloid progenitors (CMP), granulocyte-monocyte progenitors (GMP), megakaryoerythroid progenitors (MEP), megakaryocyte progenitors (MKp) and erythroid progenitors (EP).

FIG. 11. Donor Screening for RAG1 editing. A) Schematic representations of donor constructs. HA_L, left homology arm; HA_R, right homology arm; SA, splice acceptor; SD, splice donor; BGHpA, bovine growth hormone poly A; WPRE, Woodchuck hepatitis virus post-transcriptional regulatory element; IRES, the internal ribosome entry site sequence; PEST, proline (P), glutamic acid (E), serine (S), and threonine (T). B) schematic representation of the experimental protocol. C) GFP expression levels shown as Mean Fluorescence Intensity (MFI) gating on GFP+ events measured by flow cytometry over time (d, days after editing). D) Modulation of GFP expression in serum starved cells is shown as ratio of GFP MFI of starved cells (- FBS) and GFP MFI of not starved cells (+ FBS) (1 experiment representative of 3).

FIG. 12. Editing enhancer effects on HDR efficiency of RAG1 locus. A) Schematic representation of the gene editing protocol (upper panel) and artificial thymic organoid protocol (ATO) (lower panel). B) HDR efficiency is shown as percentages of edited alleles measured by ddPCR 7 days after editing; C) Frequency of targeted cells measured by flow cytometry as GFP⁺ cells among hCD34⁺ subsets 7 days after editing; D) Analysis of HSPC composition was performed in MPB or BM CD34⁺ cells derived from healthy donor by flow-cytometry. The analysis was performed before the expansion phase (day 0) and 1 day after the gene editing procedure (GE, day 4). Untreated cells (UT) were also analyzed the same day of edited cells. Graphs show 20 subtypes analyzed in the Lineage negative (Lin⁻) CD34⁺ gate including: Hematopoietic Stem cells (HSC), Multipotent Progenitors (MPP), Multi-Lymphoid Progenitors (MLP), Early T Progenitors (ETP), B and NK cell precursors (Pre-B/NK), common myeloid progenitors (CMP), granulocyte-monocyte progenitors (GMP), megakaryoerythroid progenitors (MEP), megakaryocyte progenitors (MKp) and erythroid progenitors (EP).

FIG. 13. Editing enhancer effects on T cell differentiation potential. Representative images of artificial thymic organoid (ATO) 4 weeks after ATO seeding with Untreated cells (UT) or edited cells with or without HDR enhancers. B) total number of cells harvested from ATOs 4 weeks after ATO seeding. C) HDR efficiency is shown as percentages of edited alleles measured by ddPCR in bulk differentiated T cells 4 weeks after ATO seeding. D) HDR efficiency is measured as percentage of GFP+ cells within distinct T cell subpopulation by flow cytometry 4 weeks after ATO seeding.

FIG. 14. Donor constructs for the intronic correction strategy. Schematic representation of the SA_coRAG1 CDS_BGHpA (A) and SA_coRAG1 CDS_SD (B) donor templates used for the intronic correction strategy. HA, homology arm; SA, splice acceptor; SD, splice donor; coRAG1 CDS, codon optimized RAG1 coding sequence; BGHpA, bovine growth hormone poly A; Ex., exon; gRNA, guide RNA; 3′UTR, 3′ untranslated region; HDR, homology directed repair.

FIG. 15. Corrective donor comparison in NALM6.Rag1KO cells. (A) Schematic representation of the experiment performed to compare the correction efficacy of the two donors: the SA_coRAG1 CDS_BGHpA vs the SA_coRAG1 CDS_SD donor. (B) RAG1 CDS expression was evaluated in various NALM6.Rag1KO edited clones by RT-qPCR and measured as relative expression to the housekeeping beta-actin. (C) Recombination activity was evaluated 7 days after serum-starvation as proportion of GFP+ cells gated on transduced cells by flow cytometry.

FIG. 16. Corrective donor comparison in HD-HSPC. (A) Hematopoietic stem and progenitor cells were edited by guide 9 and Cas9 as RNP in combination with SA_coRAG1 CDS_BGHpA or SA_coRAG1 CDS_SD donor. The proportion of edited alleles was evaluated by ddPCR in bulk HSPC 4 days after the editing. (B) The proportion of edited alleles was evaluated by ddPCR in HSPC subsets isolated by cell sorting. (C) Kinetics of cell growth in untreated (UT) or edited HSPC according to the indicated donors, doses and days after gene editing (GE). (D) Colony forming unit (CFU) assay was performed on untreated or edited HSPC by counting the number of red (erythroid), white (myeloid) and mixed colonies at microscope 14 days after the plating. (E) Distribution of the CD34+ cell subpopulations and CD34- cells measured by flow cytometry based on the expression of hCD133 and hCD90 analysed 4 days after the editing. (F) Representative plots of the T cell differentiation stages analysed by flow cytometry 7 weeks after ATO seeding. (G) HDR efficiency is measured as proportion of edited alleles in bulk, CD4+ CD8+ double positive (DP) cells and CD4- CD8- double negative (DN) cells by flow cytometry 6 weeks after ATO seeding.

### DETAILED DESCRIPTION

It must be noted that as used herein and in the appended claims, the singular forms “a”, “an”, and “the” include plural referents unless the context clearly dictates otherwise.

The terms “comprising”, “comprises” and “comprised of′ as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

All recited genomic locations are based on human genome assembly GRCh38.p13 (GCF_000001405.39). One of skill in the art will be able to identify the corresponding genome locations in alternative genome assemblies and convert the recited genomic location accordingly. For example, RAG1 is located at chr 11: 36510353 to 36579762 in assembly GRCh38.p13 and at chr 11: 36532053 to 36601312 in assembly GRCh37.p13.

The publications discussed herein are provided solely for their disclosure prior to the filing date of the present application. Nothing herein is to be construed as an admission that such publications constitute prior art to the claims appended hereto.

### Recombination Activating Gene 1 (RAG1)

The present invention relates to methods for gene-editing cells to introduce a RAG1 polypeptide, for example as a treatment for severe combined immunodeficiency. The present invention also relates to polynucleotides, vectors, guide RNAs, kits, compositions, and gene editing systems for use in said methods, and genomes and cells obtained or obtainable by said methods.

“RAG1” is the abbreviated name of the polypeptide encoded by recombination activating gene 1 and is also known as RAG-1, RNF74, and recombination activating 1.

RAG1 is the catalytic component of the RAG complex, a multiprotein complex that mediates the DNA cleavage phase during V(D)J recombination. V(D)J recombination assembles a diverse repertoire of immunoglobulin and T-cell receptor genes in developing B and T-lymphocytes through rearrangement of different V (variable), in some cases D (diversity), and J (joining) gene segments. In the RAG complex, RAG1 mediates the DNA-binding to the conserved recombination signal sequences (RSS) and catalyses the DNA cleavage activities by introducing a double-strand break between the RSS and the adjacent coding segment. RAG2 is not a catalytic component but is required for all known catalytic activities.

A “RAG1 polypeptide” is a polypeptide having RAG1 activity, for example a polypeptide which is able to form a RAG complex, mediate DNA-binding to the RSS, and introduce a double-strand break between the RSS and the adjacent coding segment. Suitably, a RAG1 polypeptide may have the same or similar activity to a wild-type RAG1, e.g. may have at least 40%, at least 50%, at least 60%, at least 70%, at least 80%, at least 90%, at least 95%, at least 100%, at least 110%, at least 120%, at least 130%, at least 140%, or at least 150% of the activity of a wild-type RAG1 polypeptide.

The RAG1 polypeptide may be a fragment of RAG1 and/or a RAG1 variant.

A “fragment of RAG1” may refer to a portion or region of a full-length RAG1 polypeptide that has the same of similar activity as a full-length RAG1 polypeptide, i.e. the fragment may be a functional fragment. The fragment may have at least 40%, at least 50%, at least 60%, at least 70%, at least 80%, at least 90%, at least 95%, or 100% of the activity of a full-length RAG1 polypeptide. A person skilled in the art would be able to generate fragments based on the known structural and functional features of RAG1. These are described, for instance, in Arbuckle, J.L., et al., 2011. BMC biochemistry, 12(1), p.23; Ru, H., et al., 2015. Cell, 163(5), pp.1138-1152; and Kim, M.S., et al., 2015. Nature, 518(7540), pp.507-511.

The minimal regions of RAG1 required for catalysis have been identified. These regions are referred to as the core proteins. Core RAG1 consists of multiple structural domains, termed the nonamer binding domain (NBD; residues 389-464), the central domain (residues 528-760), and the C-terminal domain (residues 761-980) domains. Besides the ability to recognize the RSS nonamer and heptamer through the NBD and the central domain, respectively, core RAG1 contains the essential acidic active site residues (Arbuckle, J.L., et al., 2011. BMC biochemistry, 12(1), p.23). Suitably, a fragment of RAG1 comprises the nonamer binding domain, the central domain, and/or the C-terminal domain.

A “RAG1 variant” may include an amino acid sequence or a nucleotide sequence which may be at least 50%, at least 55%, at least 65%, at least 70%, at least 75%, at least 80%, at least 85% or at least 90% identical, optionally at least 95% or at least 97% or at least 99% identical to a wild-type RAG1 polypeptide. RAG1 variants may have the same or similar activity to a wild-type RAG1 polypeptide, e.g. may have at least at least 40%, at least 50%, at least 60%, at least 70%, at least 80%, at least 90%, at least 95%, at least 100%, at least 110%, at least 120%, at least 130%, at least 140%, or at least 150% of the activity of a wild-type RAG1 polypeptide. A person skilled in the art would be able to generate RAG1 variants based on the known structural and functional features of RAG1 and/or using conservative substitutions.

The gene encoding RAG1 (NCBI gene ID: 5896) is located in the human genome at chr 11: 36510353 to 36579762.

Several alternative mRNAs are transcribed from the RAG1 gene. Transcript variant 1 (NM_000448) has two exons and one intron. As used herein, the region of the RAG1 gene corresponding to the first exon of transcript variant 1 is called the “RAG1 exon 1”, the region of the RAG1 gene corresponding to the intron of transcript variant 1 is called the “RAG1 intron 1”, and the region of the RAG1 gene corresponding to the second exon (which encodes a RAG1 polypeptide) is called the “RAG1 exon 2”.

Suitably, the RAG1 exon 1 is from chr 11: 36568006 to chr 11: 36568122; the RAG1 intron 1 is from chr 11: 36568123 to chr 11: 36573290; and/or the RAG1 exon 2 is from chr 11: 36573291 to chr 11: 36579762.

Suitably, the RAG1 exon 1 consists of the nucleotide sequence of SEQ ID NO: 1, or variants thereof; the RAG1 intron 1 consists of the nucleotide sequence of SEQ ID NO: 2, or variants thereof; and/or the RAG1 exon 2 consists of the nucleotide sequence of SEQ ID NO: 3, or variants thereof.

Illustrative RAG1 exon 1 (SEQ ID NO: 1)

agaaacaagagggcaaggagagagcagagaacacactttgccttctcttt
ggtattgagtaatatcaaccaaattgcagacatctcaacactttggccag
gcagcctgctgagcaag

Illustrative RAG1 intron 1 (SEQ ID NO: 2)

gtaacactcatacttttcatgccttgagccaaaatatttattacattttt
atgtttctaactagaagtgcttgagctttttttccttccaggtgatgagg
ggatggaatgagcaaagctacatcaatttttttttaatgtatgaaaataa
aaaaggtacaagaggccaagtttagggccactgaaggttcatagaaagat
gcaaaatatctgaattactataaatgaatgctattgtcagaggaaaggtt
taaggagtgcttcttgaatgaatgtgtacaaatcagcagaaggtaaggtg
tgagactcttggaaatgaatactggtagttcaggtgagaaaaataatcag
gaacataatagggtgggaggaaatgtatggtttcccaggtattaacaagt
attgccaggcatttcctgaactagattggcctaagtaggagaccaatgtt
tctcaaaatattcactcattttagaatcactgaatgtttaaaaatgcaat
ttctggattccttcccaaacagccagactctttgggacctgatgatctgc
atttctttttaaaaacaaactcgctcatgattctgatttgtattaatttt
gagaattgccatggtagagaccctgctttgaggttatgttcttgagtcag
gattcctggccagggattgtgatgatatatttctctttctgaagtggttc
atgcaagaggttgtctgaaggaagagcaagaattgtagtgttattttgtg
gatacttgagacttataaaaaggctttttattttgtcacatttttgatac
atgatgtttggcaaaaaacagacgatagtatttgcagagtgaatgaataa
gtggaacaggtgtgataatgagaggtcacacttgagcacacagttattac
ttggaaattgtgtacagactaagttgaagatgttaggagggaagattgtg
ggccaagtaacggggtgtatgtgtgtgggtatagggtgggcagctgggat
ggaaatggggggctgctgctgctgctgcaccctggcctcctgaactaatg
atatcactcaccagaaactactgttcctgcactgtccaagccaccccaaa
ctagtttgtcaaaatgaatctgtgctgtgtggagggaggcacgcctgtag
ctctgatgtcagatggcaatgtcgagatggcagtggccggtggggacagg
gctgagccagcaccaaccactcagcctttgagatcccgaggctggtctac
tgctgagaccttttgttagaagagaggagatcaagcatttgcaaggtttc
tgagtgtcaaaatatgaatccaagataactctttcacaatcctaacttca
tgctgtctacaggtccatattttagcctgctttctccatgttcatccgaa
aagaaagaaaagctaagggtggtggtcatatttgaaattagccagatctt
aagtttttctgggggaaatttagaagaaaatatggaaaagtgactatgag
cacatatacagctagtctttaaaacagttttatccaaaataaatgtatca
caaaattaataaaaatagttacttgcttgttttgaataattcaaatgata
caaaaattaataaaataaaaagtgcaaaaggccctcttatcaatgccaat
tctatttttttcagaaattaaacactgttaagattttagtgtgtatcctt
tcagaattcctgtgatttcatatatgtacaaatacaaacgtatctacata
aagggaatcctactatacttgctattgtcattctattctctgctttttca
tgtgagcatctttccatgtcactgatgcatacagaaattgcacatatgca
tcagtgcatacagaaaattaaattttctgcatggttttccactgtatgtc
tggaccatagtttatttaataataatgccctttgggtaattatttatatt
gtttcctgctttttcaaagtaacagcttttgaaacaaatctctctctgtc
tttatataaatattgttgcattcctgtggaaatgtttctattggataact
tcccaaaaggagatttattgcatcaaagataatatattcaaaaattttaa
agatattgctaaattgtctagtaggtattttataccaatttatactcctc
ccaagaatgtatggagatatcttaatttctccatgccttcattaatgctg
aaccatataagtagttttaatctttgctaattgaatagataaaaaatatc
taatctaagtctagttcttaaaagttctatcttctaccaaaagtaataca
cgtctattttagggagtaaaaatcacaagtaaggataaaaaatagtgcag
caataaacacaggagtgtagatgtctctgaacatactgatttaacttcct
ttggataaatacccagtagtaggactgctggatcatataataattctatc
tttagtttttttgaggacctccatactattcttcatagtggctgtactaa
tttacattcctaccaactgtgtatgaaggttcccttttctctacatcctt
gccagcattcattattgcttgtcatttggatacaatctattttaactggg
gtgagatgacatctcattgtagttttgatatgcatttctctgatgatcag
tggtgttgagcaccttttcatatacctgtttgccatttgtatgtcttcct
ttgagaaatgtctattcagatattttacctattttaaaatcggattatta
gattgtttcctgtagagttgtttgagctccttgtatattctggttattaa
tctcttgtcagatgcatagcttacaaatattttctcccatcatgtggatt
gtgtcttcactttgtggattgtttactttgctgtgcagaagcttttaact
tgatgcaatcccatttgtccacttttgctttggttgccttccacaggagt
atttaaataaatgtagtttggtagattttggtatagtaatgcaggccagt
gggagtcaggggagaaatgtgtagggaagtgagatagttctaaggatcct
acaaacatgccttatgattgacttactcaatgtgaaagtcaatattaaac
ttgatgagctctagagatggtcatgcattttaaaaagaattactcaaaat
attgtcttggaataccagagagcaagtgctttaagtataggctgggaagt
aaaatgctaaaggaatgagaaggcatttggggttgagttcaacctaagag
gcaggggagccacagggaaagacctagcacctgccacagaagagaattag
gaagcagaattgaactataagcaattttgaggtgttcgttgggctgcagt
tgaaatattttttgaggttaatgagacatttgaaatggccgtgtattgtt
taactcttgcatagtcctgcatagggaacaatctaataggatttctctgt
gaatcaagtcttagaaatttgcttttaatttttatgaaaaacgcccattt
ctttgtttttgagacagagtcctgctctgtcatccaggctgggttgcagt
ggcgtgatcttggcccactgcaatctctgcctcctgggttcaggcaattt
tcctgtctcagcctcccgagtagctgggatttcaagtgcctgccaccatg
cccggctaaatttttttgtatttttggtacagatggagtatcaccatgtt
ggccaggctggtctcgaactcctgacctcaagtgattcaccagccttgac
ctcccaaagtgttgggatcacaggcatgagccactgtgcctgtgccccaa
aacaccaatttctgatgtgtgatgcatgtaagatagaacaaacttcagta
aagcggggacttgaaaagaggctttggtaacagctgtcagcattaaccct
tgcccctccgtacctcctaatcccacccctgctcaaagtatgttcatctg
agaatttgtctccataactatgtgactataaaaattctcatcgattttgt
tagttgatcaattgagggaaaaacatatgttacttgatataactggtggg
tcaaaagaattaacccaggcaaatttgagataggtggatgggatgatgga
ttgaaaatacagctgctctctttccaatcatgtactaagtaatttgggaa
agattgatctaattgggtctagagagtacacttcacatggcattgtttga
ctttttttctgcatcgctagcgatctgtgcattacaactcaaatcagtcg
ggtttcctggcatatgtaattgccaatgttttttaccagaagagaaacat
tactcccacctcttcttattatgttacaaactatagtgctaatgaccatc
gaccaacagtgactttcaggatgacctgtgtgagttttatctgaaaccat
gtgaatttttcatcttaaaagtcccttagaatctcagtctatgtacactc
aggtttgttgcaggtttagagttccgtgttttttgtttctaatgtagaca
cagccttataatttacaacagcattcactaattaaaattgtaagcataat
tactatccacgatacttattattagtttgcattcataaagctcaaaattc
acttcatcctttcaagtagtgaataattagtttctttgggtttgcagctt
tatcatccttttatgacccatttggaagaaataaacaaccaaccccctgg
aagactgctttaaaaagctggaaatacattgtccagctagtacaatgagg
ctaatacaatgtggaaaatattacttttctttgattttagtagcctgttt
atctttacatttactgaacaaataactattgagcacctaatgtatactgg
gacccttggggaggcaaagatgaatcaaagattctgtccttaaagacctt
aaggtttttgtggaaggaaataaaactttacatgtatatatttaagcact
tatatgtgtgtaacaggtataagtaaccataaacactgtcagaagaggaa
ataactctatgatcagcacctaacatgatatattaaggtagaagatttaa
tacatatcttttggaatacatgaataaataattgaatgtatttattttta
ttatttataagatacatcagtgggatattgatattggtcttaatatgact
tgttttcattgttctcag

Illustrative RAG1 exon 2 (SEQ ID NO: 3)

gtacctcagccagcATGGCAGCCTCTTTCCCACCCACCTTGGGACTCAGT
TCTGCCCCAGATGAAATTCAGCACCCACATATTAAATTTTCAGAATGGAA
ATTTAAGCTGTTCCGGGTGAGATCCTTTGAAAAGACACCTGAAGAAGCTC
AAAAGGAAAAGAAGGATTCCTTTGAGGGGAAACCCTCTCTGGAGCAATCT
CCAGCAGTCCTGGACAAGGCTGATGGTCAGAAGCCAGTCCCAACTCAGCC
ATTGTTAAAAGCCCACCCTAAGTTTTCAAAGAAATTTCACGACAACGAGA
AAGCAAGAGGCAAAGCGATCCATCAAGCCAACCTTCGACATCTCTGCCGC
ATCTGTGGGAATTCTTTTAGAGCTGATGAGCACAACAGGAGATATCCAGT
CCATGGTCCTGTGGATGGTAAAACCCTAGGCCTTTTACGAAAGAAGGAAA
AGAGAGCTACTTCCTGGCCGGACCTCATTGCCAAGGTTTTCCGGATCGAT
GTGAAGGCAGATGTTGACTCGATCCACCCCACTGAGTTCTGCCATAACTG
CTGGAGCATCATGCACAGGAAGTTTAGCAGTGCCCCATGTGAGGTTTACT
TCCCGAGGAACGTGACCATGGAGTGGCACCCCCACACACCATCCTGTGAC
ATCTGCAACACTGCCCGTCGGGGACTCAAGAGGAAGAGTCTTCAGCCAAA
CTTGCAGCTCAGCAAAAAACTCAAAACTGTGCTTGACCAAGCAAGACAAG
CCCGTCAGCACAAGAGAAGAGCTCAGGCAAGGATCAGCAGCAAGGATGTC
ATGAAGAAGATCGCCAACTGCAGTAAGATACATCTTAGTACCAAGCTCCT
TGCAGTGGACTTCCCAGAGCACTTTGTGAAATCCATCTCCTGCCAGATCT
GTGAACACATTCTGGCTGACCCTGTGGAGACCAACTGTAAGCATGTCTTT
TGCCGGGTCTGCATTCTCAGATGCCTCAAAGTCATGGGCAGCTATTGTCC
CTCTTGCCGATATCCATGCTTCCCTACTGACCTGGAGAGTCCAGTGAAGT
CCTTTCTGAGCGTCTTGAATTCCCTGATGGTGAAATGTCCAGCAAAAGAG
TGCAATGAGGAGGTCAGTTTGGAAAAATATAATCACCACATCTCAAGTCA
CAAGGAATCAAAAGAGATTTTTGTGCACATTAATAAAGGGGGCCGGCCCC
GCCAACATCTTCTGTCGCTGACTCGGAGAGCTCAGAAGCACCGGCTGAGG
GAGCTCAAGCTGCAAGTCAAAGCCTTTGCTGACAAAGAAGAAGGTGGAGA
TGTGAAGTCCGTGTGCATGACCTTGTTCCTGCTGGCTCTGAGGGCGAGGA
ATGAGCACAGGCAAGCTGATGAGCTGGAGGCCATCATGCAGGGAAAGGGC
TCTGGCCTGCAGCCAGCTGTTTGCTTGGCCATCCGTGTCAACACCTTCCT
CAGCTGCAGTCAGTACCACAAGATGTACAGGACTGTGAAAGCCATCACAG
GGAGACAGATTTTTCAGCCTTTGCATGCCCTTCGGAATGCTGAGAAGGTA
CTTCTGCCAGGCTACCACCACTTTGAGTGGCAGCCACCTCTGAAGAATGT
GTCTTCCAGCACTGATGTTGGCATTATTGATGGGCTGTCTGGACTATCAT
CCTCTGTGGATGATTACCCAGTGGACACCATTGCAAAGAGGTTCCGCTAT
GATTCAGCTTTGGTGTCTGCTTTGATGGACATGGAAGAAGACATCTTGGA
AGGCATGAGATCCCAAGACCTTGATGATTACCTGAATGGCCCCTTCACTG
TGGTGGTGAAGGAGTCTTGTGATGGAATGGGAGACGTGAGTGAGAAGCAT
GGGAGTGGGCCTGTAGTTCCAGAAAAGGCAGTCCGTTTTTCATTCACAAT
CATGAAAATTACTATTGCCCACAGCTCTCAGAATGTGAAAGTATTTGAAG
AAGCCAAACCTAACTCTGAACTGTGTTGCAAGCCATTGTGCCTTATGCTG
GCAGATGAGTCTGACCACGAGACGCTGACTGCCATCCTGAGTCCTCTCAT
TGCTGAGAGGGAGGCCATGAAGAGCAGTGAATTAATGCTTGAGCTGGGAG
GCATTCTCCGGACTTTCAAGTTCATCTTCAGGGGCACCGGCTATGATGAA
AAACTTGTGCGGGAAGTGGAAGGCCTCGAGGCTTCTGGCTCAGTCTACAT
TTGTACTCTTTGTGATGCCACCCGTCTGGAAGCCTCTCAAAATCTTGTCT
TCCACTCTATAACCAGAAGCCATGCTGAGAACCTGGAACGTTATGAGGTC
TGGCGTTCCAACCCTTACCATGAGTCTGTGGAAGAACTGCGGGATCGGGT
GAAAGGGGTCTCAGCTAAACCTTTCATTGAGACAGTCCCTTCCATAGATG
CACTCCACTGTGACATTGGCAATGCAGCTGAGTTCTACAAGATCTTCCAG
CTAGAGATAGGGGAAGTGTATAAGAATCCCAATGCTTCCAAAGAGGAAAG
GAAAAGGTGGCAGGCCACACTGGACAAGCATCTCCGGAAGAAGATGAACC
TCAAACCAATCATGAGGATGAATGGCAACTTTGCCAGGAAGCTCATGACC
AAAGAGACTGTGGATGCAGTTTGTGAGTTAATTCCTTCCGAGGAGAGGCA
CGAGGCTCTGAGGGAGCTGATGGATCTTTACCTGAAGATGAAACCAGTAT
GGCGATCATCATGCCCTGCTAAAGAGTGCCCAGAATCCCTCTGCCAGTAC
AGTTTCAATTCACAGCGTTTTGCTGAGCTCCTTTCTACGAAGTTCAAGTA
TAGGTATGAGGGAAAAATCACCAATTATTTTCACAAAACCCTGGCCCATG
TTCCTGAAATTATTGAGAGGGATGGCTCCATTGGGGCATGGGCAAGTGAG
GGAAATGAGTCTGGTAACAAACTGTTTAGGCGCTTCCGGAAAATGAATGC
CAGGCAGTCCAAATGCTATGAGATGGAAGATGTCCTGAAACACCACTGGT
TGTACACCTCCAAATACCTCCAGAAGTTTATGAATGCTCATAATGCATTA
AAAACCTCTGGGTTTACCATGAACCCTCAGGCAAGCTTAGGGGACCCATT
AGGCATAGAGGACTCTCTGGAAAGCCAAGATTCAATGGAATTTTAAgtag
ggcaaccacttatgagttggtttttgcaattgagtttccctctgggttgc
attgagggcttctcctagcaccctttactgctgtgtatggggcttcacca
tccaagaggtggtaggttggagtaagatgctacagatgctctcaagtcag
gaatagaaactgatgagctgattgcttgaggcttttagtgagttccgaaa
agcaacaggaaaaatcagttatctgaaagctcagtaactcagaacaggag
taactgcaggggaccagagatgagcaaagatctgtgtgtgttggggagct
gtcatgtaaatcaaagccaaggttgtcaaagaacagccagtgaggccagg
aaagaaattggtcttgtggttttcatttttttcccccttgattgattata
ttttgtattgagatatgataagtgccttctatttcatttttgaataattc
ttcatttttataattttacatatcttggcttgctatataagattcaaaag
agctttttaaatttttctaataatatcttacatttgtacagcatgatgac
ctttacaaagtgctctcaatgcatttacccattcgttatataaatatgtt
acatcaggacaactttgagaaaatcagtccttttttatgtttaaattatg
tatctattgtaaccttcagagtttaggaggtcatctgctgtcatggattt
ttcaataatgaatttagaatacacctgttagctacagttagttattaaat
cttctgataatatatgtttacttagctatcagaagccaagtatgattctt
tatttttactttttcatttcaagaaatttagagtttccaaatttagagct
tctgcatacagtcttaaagccacagaggcttgtaaaaatataggttagct
tgatgtctaaaaatatatttcatgtcttactgaaacattttgccagactt
tctccaaatgaaacctgaatcaatttttctaaatctaggtttcatagagt
cctctcctctgcaatgtgttattctttctataatgatcagtttactttca
gtggattcagaattgtgtagcaggataaccttgtatttttccatccgcta
agtttagatggagtccaaacgcagtacagcagaagagttaacatttacac
agtgctttttaccactgtggaatgttttcacactcatttttccttacaac
aattctgaggagtaggtgttgttattatctccatttgatgggggtttaaa
tgatttgctcaaagtcatttaggggtaataaatacttggcttggaaattt
aacacagtccttttgtctccaaagcccttcttctttccaccacaaattaa
tcactatgtttataaggtagtatcagaatttttttaggattcacaactaa
tcactatagcacatgaccttgggattacatttttatggggcaggggtaag
caagtttttaaatcatttgtgtgctctggctcttttgatagaagaaagca
acacaaaagctccaaagggccccctaaccctcttgtggctccagttattt
ggaaactatgatctgcatccttaggaatctgggatttgccagttgctggc
aatgtagagcaggcatggaattttatatgctagtgagtcataatgatatg
ttagtgttaattagttttttcttcctttgattttattggccataattgct
actcttcatacacagtatatcaaagagcttgataatttagttgtcaaaag
tgcatcggcgacattatctttaattgtatgtatttggtgcttcttcaggg
attgaactcagtatctttcattaaaaaacacagcagttttccttgctttt
tatatgcagaatatcaaagtcatttctaatttagttgtcaaaaacatata
catattttaacattagtttttttgaaaactcttggttttgtttttttgga
aatgagtgggccactaagccacactttcccttcatcctgcttaatccttc
cagcatgtctctgcactaataaacagctaaattcacataatcatcctatt
tactgaagcatggtcatgctggtttatagattttttacccatttctactc
tttttctctattggtggcactgtaaatactttccagtattaaattatcct
tttctaacactgtaggaactattttgaatgcatgtgactaagagcatgat
ttatagcacaacctttccaataatcccttaatcagatcacattttgataa
accctgggaacatctggctgcaggaatttcaatatgtagaaacgctgcct
atggttttttgcccttactgttgagactgcaatatcctagaccctagttt
tatactagagttttatttttagcaatgcctattgcaagtgcaattatata
ctccagggaaattcaccacactgaatcgagcatttgtgtgtgtatgtgtg
aagtatatactgggacttcagaagtgcaatgtatttttctcctgtgaaac
ctgaatctacaagttttcctgccaagccactcaggtgcattgcagggacc
agtgataatggctgatgaaaattgatgattggtcagtgaggtcaaaagga
gccttgggattaataaacatgcactgagaagcaagaggaggagaaaaaga
tgtctttttcttccaggtgaactggaatttagttttgcctcagatttttt
tcccacaagatacagaagaagataaagatttttttggttgagagtgtggg
tcttgcattacatcaaacagagttcaaattccacacagataagaggcagg
atatataagcgccagtggtagttgggaggaataaaccattatttggatgc
aggtggtttttgattgcaaatatgtgtgtgtcttcagtgattgtatgaca
gatgatgtattcttttgatgttaaaagattttaagtaagagtagatacat
tgtacccattttacattttcttattttaactacagtaatctacataaata
tacctcagaaatcatttttggtgattattttttgttttgtagaattgcac
ttcagtttattttcttacaaataaccttacattttgtttaatggcttcca
agagccttttttttttttgtatttcagagaaaattcaggtaccaggatgc
aatggatttatttgattcaggggacctgtgtttccatgtcaaatgttttc
aaataaaatgaaatatgagtttcaatactttttatattttaatatttcca
ttcattaatattatggttattgtcagcaattttatgtttgaatatttgaa
ataaaagtttaagatttgaaaa

In the illustrative RAG1 exon 2 (SEQ ID NO: 3), upper case letters indicate a nucleotide sequence which encodes a RAG1 polypeptide.

### RAG1 Polypeptides

The RAG1 polypeptide may be a human RAG1 polypeptide. Suitably, the RAG1 polypeptide may comprise or consist of a polypeptide sequence of UniProtKB accession P15918, or a fragment or variant thereof.

In some embodiments of the invention, the RAG1 polypeptide comprises or consists of an amino acid sequence which is at least 70% identical to SEQ ID NO: 4 or a fragment thereof. Suitably, the RAG1 polypeptide comprises or consists of an amino acid sequence which is at least 80%, at least 85%, at least 90%, at least 95%, at least 98% or at least 99% identical to SEQ ID NO: 4 or a fragment thereof.

In some embodiments, the RAG1 polypeptide comprises or consists of SEQ ID NO: 4 or a fragment thereof.

RAG1 polypeptide isoform 1, UniProtKB accession P15918 (SEQ ID NO: 4)

MAASFPPTLGLSSAPDEIQHPHIKFSEWKFKLFRVRSFEKTPEEAQKEKK
DSFEGKPSLEQSPAVLDKADGQKPVPTQPLLKAHPKFSKKFHDNEKARGK
AIHQANLRHLCRICGNSFRADEHNRRYPVHGPVDGKTLGLLRKKEKRATS
WPDLIAKVFRIDVKADVDSIHPTEFCHNCWSIMHRKFSSAPCEVYFPRNV
TMEWHPHTPSCDICNTARRGLKRKSLQPNLQLSKKLKTVLDQARQARQHK
RRAQARISSKDVMKKIANCSKIHLSTKLLAVDFPEHFVKSISCQICEHIL
ADPVETNCKHVFCRVCILRCLKVMGSYCPSCRYPCFPTDLESPVKSFLSV
LNSLMVKCPAKECNEEVSLEKYNHHISSHKESKEIFVHINKGGRPRQHLL
SLTRRAQKHRLRELKLQVKAFADKEEGGDVKSVCMTLFLLALRARNEHRQ
ADELEAIMQGKGSGLQPAVCLAIRVNTFLSCSQYHKMYRTVKAITGRQIF
QPLHALRNAEKVLLPGYHHFEWQPPLKNVSSSTDVGIIDGLSGLSSSVDD
YPVDTIAKRFRYDSALVSALMDMEEDILEGMRSQDLDDYLNGPFTVVVKE
SCDGMGDVSEKHGSGPVVPEKAVRFSFTIMKITIAHSSQNVKVFEEAKPN
SELCCKPLCLMLADESDHETLTAILSPLIAEREAMKSSELMLELGGILRT
FKFIFRGTGYDEKLVREVEGLEASGSVYICTLCDATRLEASQNLVFHSIT
RSHAENLERYEVWRSNPYHESVEELRDRVKGVSAKPFIETVPSIDALHCD
IGNAAEFYKIFQLEIGEVYKNPNASKEERKRWQATLDKHLRKKMNLKPIM
RMNGNFARKLMTKETVDAVCELIPSEERHEALRELMDLYLKMKPVWRSSC
PAKECPESLCQYSFNSQRFAELLSTKFKYRYEGKITNYFHKTLAHVPEII
ERDGSIGAWASEGNESGNKLFRRFRKMNARQSKCYEMEDVLKHHWLYTSK
YLQKFMNAHNALKTSGFTMNPQASLGDPLGIEDSLESQDSMEF

In some embodiments of the invention, the RAG1 polypeptide comprises or consists of an amino acid sequence which is at least 70% identical to SEQ ID NO: 5 or a fragment thereof. Suitably, the RAG1 polypeptide comprises or consists of an amino acid sequence which is at least 80%, at least 85%, at least 90%, at least 95%, at least 98% or at least 99% identical to SEQ ID NO: 5 or a fragment thereof.

In some embodiments, the RAG1 polypeptide comprises or consists of SEQ ID NO: 5 or a fragment thereof.

RAG1 polypeptide isoform 2, UniProtKB accession P15918 (SEQ ID NO: 5)

MAASFPPTLGLSSAPDEIQHPHIKFSEWKFKLFRVRSFEKTPEEAQKEKK
DSFEGKPSLEQSPAVLDKADGQKPVPTQPLLKAHPKFSKKFHDNEKARGK
AIHQANLRHLCRICGNSFRADEHNRRYPVHGPVDGKTLGLLRKKEKRATS
WPDLIAKVFRIDVKADVDSIHPTEFCHNCWSIMHRKFSSAPCEVYFPRNV
TMEWHPHTPSCDICNTARRGLKRKSLQPNLQLSKKLKTVLDQARQARQHK
RRAQARISSKDVMKKIANCSKIHLSTKLLAVDFPEHFVKSISCQICEHIL
ADPVETNCKHVFCRVCILRCLKVMGSYCPSCRYPCFPTDLESPVKSFLSV
LNSLMVKCPAKECNEEVSLEKYNHHISSHKESKEIFVHINKGGRPRQHLL
SLTRRAQKHRLRELKLQVKAFADKEEGGDVKSVCMTLFLLALRARNEHRQ
ADELEAIMQGKGSGLQPAVCLAIRVNTFLSCSQYHKMYRTVKAITGRQIF
QPLHALRNAEKVLLPGYHHFEWQPPLKNVSSSTDVGIIDGLSGLSSSVDD
YPVDTIAKRFRYDSALVSALMDMEEDILEGMRSQDLDDYLNGPFTVVVKE
SCDGMGDVSEKHGSGPVVPEKAVRFSFTIMKITIAHSSQNVKVFEEAKPN
SELCCKPLCLMLADESDHETLTAILSPLIAEREAMKSSELMLELGGILRT
FKFIFRGTGYDEKLVREVEGLEASGSVYICTLCDATRLEASQNLVFHSIT
RSHAENLERYEVWRSNPYHESVEELRDRVKGVSAKPFIETVPSIDALHCD
IGNAAEFYKIFQLEIGEVYKNPNASKEERKRWQATLDKHLRKKMNLKPIM
RMNGNFARKLMTKETVDAVCELIPSEERHEALRELMDLYLKMKPVWRSSC
PAKECPESLCQYSFNSQRFAELLSTKFKYRN

### RAG1 Polynucleotides

The nucleotide sequence encoding a RAG1 polypeptide may be codon-optimised. Suitably, the nucleotide sequence encoding a RAG1 polypeptide may be codon optimised for expression in a human cell.

Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. Codon usage tables are known in the art for mammalian cells (e.g. humans), as well as for a variety of other organisms.

In some embodiments of the invention, the nucleotide sequence encoding a RAG1 polypeptide comprises or consists of a nucleotide sequence which is at least 70% identical to SEQ ID NO: 6 or a fragment thereof. Suitably, the nucleotide sequence encoding a RAG1 polypeptide comprises or consists of a nucleotide sequence which is at least 80%, at least 85%, at least 90%, at least 95%, at least 98% or at least 99% identical to SEQ ID NO: 6 or a fragment thereof.

In some embodiments of the invention, the nucleotide sequence encoding a RAG1 polypeptide comprises or consists of the nucleotide sequence SEQ ID NO: 6 or a fragment thereof.

Exemplary nucleotide sequence encoding a RAG1 polypeptide (SEQ ID NO: 6)

atggccgcctccttcccacctacccttggattgtcctccgcccctgacga
aattcaacatccccacatcaaattctcggagtggaagttcaagctctttc
gcgtgcgctcgttcgaaaagacccccgaggaagcccaaaaggagaagaaa
gactcattcgaaggaaaacccagcctcgaacagtccccggccgtcctgga
caaggccgacgggcagaagcctgtgccgacccagccgctgctgaaagcgc
acccgaaattctccaagaagtttcacgataacgagaaggcccggggaaag
gccatccaccaagcaaaccttagacacctgtgccgcatctgtgggaactc
attcagagccgacgaacataaccggagataccctgtgcatggccctgtcg
acggaaagaccctggggctcctgagaaagaaggagaagagggcgacatcc
tggccggacctgatcgcaaaggtgttcagaatcgacgtgaaggcagatgt
ggacagcatccacccaaccgagttctgccacaactgctggagcattatgc
accggaagttcagctcagcgccctgtgaagtgtacttcccgcgcaacgtg
actatggagtggcatccacacactccgtcctgcgacatctgtaacactgc
tcggcgcggactcaagaggaagtccctgcagccgaatctgcagctgagca
agaagcttaagaccgtgctggaccaggctcggcaggcccgccagcacaag
cgacgcgcccaggcccggatctcatctaaggatgtgatgaagaagatcgc
caattgcagcaaaatccacctgtctaccaagctgctggcggtggacttcc
cggagcacttcgtgaagtccatcagctgtcagatctgcgagcatattctc
gccgaccccgtggagactaattgcaagcacgtgttctgccgcgtgtgcat
cctgcgctgcctgaaggtcatgggctcctattgcccttcctgccggtacc
cctgtttccctactgatctggagtccccggtcaagtccttcttgtccgtg
ctgaactccctgatggtcaaatgtcccgcaaaggagtgcaatgaggaagt
gtccctggaaaagtacaaccaccacatcagcagccacaaggagtccaaag
aaatctttgtgcacattaacaagggcggtcggccccggcagcatctgctc
tcgctgactcgccgggcccagaagcacaggctccgggagctgaagctgca
agtcaaggccttcgccgacaaggaagagggaggagatgtgaagtccgtgt
gcatgaccctgtttttgctggcgctgcgggctcggaacgaacacagacaa
gctgatgaactggaggccatcatgcagggcaaaggatcgggactccagcc
ggctgtgtgtctcgccatccgcgtcaacacattcctctcatgctcccaat
accacaagatgtacaggactgtgaaggccatcaccggacggcagatcttt
cagccactccacgcccttcggaacgcagaaaaggtcttgctgccgggata
ccatcatttcgaatggcagccgcccttgaaaaacgtgtcctcgtccaccg
acgtgggcattattgatgggctgagcggcctgtcctcctctgtggatgac
taccctgtggataccatcgccaaacggttcagatacgattccgcgctggt
gtcggccctgatggacatggaggaggacatcctggagggaatgagatcac
aagatctggacgactacctcaacgggcccttcacggtggtggtcaaggaa
tcgtgcgatggaatgggcgacgtgtcggagaagcacggttccggacctgt
ggtgccggaaaaggccgtgcgcttctccttcaccatcatgaagatcacca
ttgcgcatagctcccagaacgtcaaagtgttcgaagaggccaagccgaac
tcagagctctgctgcaagccgctgtgcctgatgttggcggacgagagcga
tcacgaaaccctgaccgccattctgtcgcctctgatcgcggagagggagg
ccatgaagtcctccgaactgatgctggagctgggcggtattttgcggact
tttaagttcatcttccggggaaccggttatgacgaaaagctcgtgcgcga
agtggagggcctggaagcctcaggctccgtctacatctgcactctctgcg
acgccacccggctggaggcgtcacagaatcttgtgttccactcgatcact
aggtcccacgcggagaacctggaacgctatgaggtctggcgctctaaccc
ataccacgaatccgtggaagaacttcgggacagagtgaagggagtgtcag
caaagcctttcattgaaaccgtgcctagcatcgacgccctccattgcgac
atcggcaacgccgccgagttctacaagatcttccagcttgagatcgggga
agtgtacaagaacccgaacgcctccaaggaagaaagaaagcggtggcagg
ctacccttgacaaacacctccgcaagaagatgaacctgaagcccattatg
cggatgaacggaaacttcgctaggaagctgatgactaaggaaacggtcga
cgcggtctgtgaactgatccccagcgaagaacgacatgaagcgctgcgcg
aactcatggacctgtacctgaagatgaagcctgtctggcggagctcgtgc
cctgccaaggagtgcccggagtcgctgtgtcagtacagctttaacagcca
aaggttcgcagagctgctgtcgaccaagttcaagtacagatacgaaggaa
agattaccaactacttccacaagactctcgctcacgtgcccgagattatc
gaacgcgatggttccatcggggcctgggcctccgagggcaacgagtcggg
caacaagttgttccgccggtttagaaagatgaacgcccgccagtccaagt
gctacgaaatggaagatgtgctgaagcatcactggctgtatacctccaag
tacctccagaagttcatgaacgcacataacgccctcaagacctccgggtt
caccatgaacccccaggcctccctcggtgaccctctgggaattgaagata
gcttggagagccaggactcgatggaattcta

### Polynucleotides and Genomes

In one aspect, the present invention provides a polynucleotide comprising from 5′ to 3′: a first homology region, a splice acceptor sequence, a nucleotide sequence encoding a RAG1 polypeptide, and a second homology region. The polynucleotide may be an isolated polynucleotide. The polynucleotide may be a DNA molecule, e.g. a double-stranded DNA molecule.

Suitably, the polynucleotide of the invention may be limited to a size suitable to be inserted into a vector (e.g. an adeno-associated viral (AAV) vector, such as AAV6). Suitably, the polynucleotide of the invention may be 5.0 kb or less, 4.9 kb or less, 4.8 kb or less, 4.7 kb or less, 4.6 kb or less, 4.5 kb or less, 4.4 kb or less, 4.3 kb or less, 4.2 kb or less, 4.1 kb or less, 4.0 kb or less in total size. In some embodiments, the polynucleotide of the invention is 4.1 kb or less or 4.0 kb or less in size.

In another aspect, the present invention provides a genome comprising a splice acceptor sequence and a nucleotide sequence encoding a RAG1 polypeptide. Suitably, the genome may comprise the polynucleotide of the present invention. The genome may be an isolated genome. The genome may be a mammalian genome, e.g. a human genome.

### Homology Regions

A “homology region” (also known as “homology arm”) is a nucleotide sequence which is located upstream or downstream of a nucleotide sequence to be inserted (a “nucleotide sequence insert” e.g. a splice acceptor sequence and a nucleotide sequence encoding a RAG1 polypeptide). The polynucleotide of the present invention comprises two homology regions, one upstream of the nucleotide sequence insert (the “first homology region”) and one downstream of the nucleotide insert (the “second homology region”).

Each “homology region” is designed such that the nucleotide sequence insert can be introduced into a genome at a site of a double strand break (DSB) by homology-directed repair (HDR). One of skill in the art will be able to design homology arms depending on the desired insertion site (i.e. the site of the DSB) (see e.g. Ran, F.A., et al., 2013. Nature protocols, 8(11), pp.2281-2308). Each “homology region” is homologous to a region either side of the DSB. For example, the first homology region may be homologous to a region upstream of the DSB and the second homology region may be homologous to a region downstream of the DSB.

As used herein, the term “homologous” means that the nucleotide sequences are similar or identical. For example, the nucleotide sequences may be at least 70% identical, at least 75% identical, at least 80% identical, at least 85% identical, at least 90% identical, at least 95% identical, at least 98% identical, at least 99% identical, or 100% identical.

As used herein, “upstream” and “downstream” both refer to relative positions in DNA or RNA. Each strand of DNA or RNA has a 5′ end and a 3′ end and, by convention, “upstream” and “downstream” relate to the 5′ to 3′ direction respectively in which RNA transcription takes place. For example, when considering double-stranded DNA, “upstream” is toward the 5′ end of the coding strand for the gene in question (e.g. RAG1) and downstream is toward the 3′ end of the coding strand for the gene in question (e.g. RAG1).

The homology regions may be any length suitable for HDR. The homology regions may be the same or different lengths. Suitably, the homology regions are each independently 50-1000 bp in length, 100-500 bp in length, or 200-400 bp in length. For example, the first homology may be 50-1000 bp in length and homologous to a region upstream of a DSB and the second homology region may be 50-1000 bp in length and homologous to a region downstream of the DSB.

In some embodiments:
(i) the first homology region is homologous to a first region of the RAG1 intron 1 and the second homology region is homologous to a second region of the RAG1 intron 1; or
(ii) the first homology region is homologous to a first region of the RAG1 intron 1 or the RAG1 exon 2 and the second homology region is homologous to a second region of the RAG1 exon 2.

In some embodiments, the first homology region is homologous to a first region of the RAG1 intron 1 and the second homology region is homologous to a second region of the RAG1 intron 1.

In some embodiments:
(i) the first homology region is homologous to a region upstream of chr 11: 36569295 and the second homology region is homologous to a region downstream of chr 11: 36569298;
(ii) the first homology region is homologous to a region upstream of chr 11: 36573790 and the second homology region is homologous to a region downstream of chr 11: 36573793;
(iii) the first homology region is homologous to a region upstream of chr 11: 36573641 and the second homology region is homologous to a region downstream of chr 11: 36573644;
(iv) the first homology region is homologous to a region upstream of chr 11: 36573351 and the second homology region is homologous to a region downstream of chr 11: 36573354;
(v) the first homology region is homologous to a region upstream of chr 11: 36569080 and the second homology region is homologous to a region downstream of chr 11: 36569083;
(vi) the first homology region is homologous to a region upstream of chr 11: 36572472 and the second homology region is homologous to a region downstream of chr 11: 36572475;
(vii) the first homology region is homologous to a region upstream of chr 11: 36571458 and the second homology region is homologous to a region downstream of chr 11: 36571461;
(viii) the first homology region is homologous to a region upstream of chr 11: 36571366 and the second homology region is homologous to a region downstream of chr 11: 36571369;
(ix) the first homology region is homologous to a region upstream of chr 11: 36572859 and the second homology region is homologous to a region downstream of chr 11: 36572862;
(x) the first homology region is homologous to a region upstream of chr 11: 36571457 and the second homology region is homologous to a region downstream of chr 11: 36571460;
(xi) the first homology region is homologous to a region upstream of chr 11: 36569351 and the second homology region is homologous to a region downstream of chr 11: 36569354; or
(xii) the first homology region is homologous to a region upstream of chr 11: 36572375 and the second homology region is homologous to a region downstream of chr 11: 36572378.

In some embodiments:
(i) the first homology region is homologous to a region upstream of chr 11: 36569295 and the second homology region is homologous to a region downstream of chr 11: 36569298;
(ii) the first homology region is homologous to a region upstream of chr 11: 36573351 and the second homology region is homologous to a region downstream of chr 11: 36573354; or
(iii) the first homology region is homologous to a region upstream of chr 11: 36571366 and the second homology region is homologous to a region downstream of chr 11: 36571369.

In some embodiments, the first homology region is homologous to a region upstream of chr 11: 36569295 and the second homology region is homologous to a region downstream of chr 11: 36569298.

In some embodiments:
(i) the first homology region is homologous to a region comprising chr 11: 36569245-36569294 and the second homology region is homologous to a region comprising chr 11: 36569299-36569348;
(ii) the first homology region is homologous to a region comprising chr 11: 36573740-36573789 and the second homology region is homologous to a region comprising chr 11: 36573794-36573843;
(iii) the first homology region is homologous to a region comprising chr 11: 36573591-36573640 and the second homology region is homologous to a region comprising chr 11: 36573645-36573694;
(iv) the first homology region is homologous to a region comprising chr 11: 36573301-36573350 and the second homology region is homologous to a region comprising chr 11: 36573355-36573404;
(v) the first homology region is homologous to a region comprising chr 11: 36569030-36569079 and the second homology region is homologous to a region comprising chr 11: 36569084-36569133;
(vi) the first homology region is homologous to a region comprising chr 11: 36572422-36572471 and the second homology region is homologous to a region comprising chr 11: 36572476-36572525;
(vii) the first homology region is homologous to a region comprising chr 11: 36571408-36571457 and the second homology region is homologous to a region comprising chr 11: 36571462-36571511;
(viii) the first homology region is homologous to a region comprising chr 11: 36571316-36571365 and the second homology region is homologous to a region comprising chr 11: 36571370-36571419;
(ix) the first homology region is homologous to a region comprising chr 11: 36572809-36572858 and the second homology region is homologous to a region comprising chr 11: 36572863-36572912;
(x) the first homology region is homologous to a region comprising chr 11: 36571407-36571456 and the second homology region is homologous to a region comprising chr 11: 36571461-36571510;
(xi) the first homology region is homologous to a region comprising chr 11: 36569301-36569350 and the second homology region is homologous to a region comprising chr 11: 36569355-36569404; or
(xii) the first homology region is homologous to a region comprising chr 11: 36572325-36572374 and the second homology region is homologous to a region comprising chr 11: 36572379-36572428.

In some embodiments:
(i) the first homology region is homologous to a region comprising chr 11: 36569245-36569294 and the second homology region is homologous to a region comprising chr 11: 36569299-36569348;
(ii) the first homology region is homologous to a region comprising chr 11: 36573301-36573350 and the second homology region is homologous to a region comprising chr 11: 36573355-36573404; or
(iii) the first homology region is homologous to a region comprising chr 11: 36571316-36571365 and the second homology region is homologous to a region comprising chr 11: 36571370-36571419.

In some embodiments, the first homology region is homologous to a region comprising chr 11: 36569245-36569294 and the second homology region is homologous to a region comprising chr 11: 36569299-36569348.

In some embodiments, the first homology region is homologous to a region comprising chr 11: 36573740-36573789 and the second homology region is homologous to a region comprising chr 11: 36573794-36573843.

In some embodiments, the first homology region is homologous to a region comprising chr 11: 36573591-36573640 and the second homology region is homologous to a region comprising chr 11: 36573645-36573694.

In some embodiments, the first homology region is homologous to a region comprising chr 11: 36573301-36573350 and the second homology region is homologous to a region comprising chr 11: 36573355-36573404.

In some embodiments, the first homology region is homologous to a region comprising chr 11: 36569030-36569079 and the second homology region is homologous to a region comprising chr 11: 36569084-36569133.

In some embodiments, the first homology region is homologous to a region comprising chr 11: 36572422-36572471 and the second homology region is homologous to a region comprising chr 11: 36572476-36572525.

In some embodiments, the first homology region is homologous to a region comprising chr 11: 36571408-36571457 and the second homology region is homologous to a region comprising chr 11: 36571462-36571511.

In some embodiments, the first homology region is homologous to a region comprising chr 11: 36571316-36571365 and the second homology region is homologous to a region comprising chr 11: 36571370-36571419.

In some embodiments, the first homology region is homologous to a region comprising chr 11: 36572809-36572858 and the second homology region is homologous to a region comprising chr 11: 36572863-36572912.

In some embodiments, the first homology region is homologous to a region comprising chr 11: 36571407-36571456 and the second homology region is homologous to a region comprising chr 11: 36571461-36571510.

In some embodiments, the first homology region is homologous to a region comprising chr 11: 36569301-36569350 and the second homology region is homologous to a region comprising chr 11: 36569355-36569404.

In some embodiments, the first homology region is homologous to a region comprising chr 11: 36572325-36572374 and the second homology region is homologous to a region comprising chr 11: 36572379-36572428.

Exemplary homology regions are shown below in Table 1.

In some embodiments, the first homology region comprises or consists of a nucleotide sequence that has at least 70% identity, at least 80% identity, at least 90% identity, at least 95% identity, at least 98% identity, or 100% identity to any of SEQ ID NOs: 7-18 and/or the second homology region comprises or consists of a nucleotide sequence that has at least 70% identity, at least 80% identity, at least 90% identity, at least 95% identity, at least 98% identity, or 100% identity to any of SEQ ID NOs: 19-30.

TABLE 1

Exemplary homology regions

| Guide RNA | First homology region | Second homology region |
|---|---|---|
| 9 | TGCTGTGTGGAGGGAGGCACGC CTGTAGCTCTGATGTCAGATGGC AATGT (SEQ ID NO: 7) | ATGGCAGTGGCCGGTGGGGACAG GGCTGAGCCAGCACCAACCACTCA GCC (SEQ ID NO: 19) |
| 1 | AAGAGAGCTACTTCCTGGCCGGA CCTCATTGCCAAGGTTTTCCGGA TCGA (SEQ ID NO: 8) | AAGGCAGATGTTGACTCGATCCAC CCCACTGAGTTCTGCCATAACTGCT G (SEQ ID NO: 20) |
| 2 | AAGCAAGAGGCAAAGCGATCCAT CAAGCCAACCTTCGACATCTCTG CCGC (SEQ ID NO: 9) | GTGGGAATTCTTTTAGAGCTGATGA GCACAACAGGAGATATCCAGTCCA T (SEQ ID NO: 21) |
| 3 | CAGCATGGCAGCCTCTTTCCCAC CCACCTTGGGACTCAGTTCTGCC CCAG (SEQ ID NO: 10) | AATTCAGCACCCACATATTAAATTTT CAGAATGGAAATTTAAGCTGTTCC (SEQ ID NO: 22) |
| 4 | TTGTGTACAGACTAAGTTGAAGAT GTTAGGAGGGAAGATTGTGGGCC AAG (SEQ ID NO: 11) | GGGGTGTATGTGTGTGGGTATAGG GTGGGCAGCTGGGATGGAAATGGG GG (SEQ ID NO: 23) |
| 5 | TTACTCCCACCTCTTCTTATTATG TTACAAACTATAGTGCTAATGACC AT (SEQ ID NO: 12) | CAACAGTGACTTTCAGGATGACCTG TGTGAGTTTTATCTGAAACCATGTG (SEQ ID NO: 24) |
| 6 | ACAGAAGAGAATTAGGAAGCAGA ATTGAACTATAAGCAATTTTGAGG TGT (SEQ ID NO: 13) | TGGGCTGCAGTTGAAATATTTTTTG AGGTTAATGAGACATTTGAAATGGC (SEQ ID NO: 25) |
| 7 | GGGAAGTAAAATGCTAAAGGAAT GAGAAGGCATTTGGGGTTGAGTT CAAC (SEQ ID NO: 14) | GAGGCAGGGGAGCCACAGGGAAA GACCTAGCACCTGCCACAGAAGAG AAT (SEQ ID NO: 26) |
| 8 | AACCAACCCCCTGGAAGACTGCT TTAAAAAGCTGGAAATACATTGTC CAG (SEQ ID NO: 15) | TACAATGAGGCTAATACAATGTGGA AAATATTACTTTTCTTTGATTTTAG (SEQ ID NO: 27) |
| 10 | CACAGAAGAGAATTAGGAAGCAG AATTGAACTATAAGCAATTTTGAG GTG (SEQ ID NO: 16) | TTGGGCTGCAGTTGAAATATTTTTT GAGGTTAATGAGACATTTGAAATGG (SEQ ID NO: 28) |
| 11 | GGCAGTGGCCGGTGGGGACAGG GCTGAGCCAGCACCAACCACTCA GCCTT (SEQ ID NO: 17) | ATCCCGAGGCTGGTCTACTGCTGA GACCTTTTGTTAGAAGAGAGGAGAT C (SEQ ID NO: 29) |
| 12 | TTTTTTCTGCATCGCTAGCGATCT GTGCATTACAACTCAAATCAGTC GGG (SEQ ID NO: 18) | CTGGCATATGTAATTGCCAATGTTT TTTACCAGAAGAGAAACATTACTCC (SEQ ID NO: 30) |

Preferably, the first and second homology regions comprise or consist of nucleotide sequences that have at least 70% identity, at least 80% identity, at least 90% identity, at least 95% identity, at least 98% identity, or 100% identity to first and second homology regions in the same row of Table 1. Suitably, the first homology region comprises or consists of a nucleotide sequence that has at least 70% identity, at least 80% identity, at least 90% identity, at least 95% identity, at least 98% identity, or 100% identity to any of SEQ ID NOs: 7-18 and the second homology region comprises or consists of a nucleotide sequence that has at least 70% identity, at least 80% identity, at least 90% identity, at least 95% identity, at least 98% identity, or 100% identity to the corresponding nucleotide sequence in Table 1 (i.e. SEQ ID NOs: 19-30). For example, in some embodiments:
(i) the first homology region comprises or consists of a nucleotide sequence that has at least 70% identity, at least 80% identity, at least 90% identity, at least 95% identity, at least 98% identity, or 100% identity to SEQ ID NO: 7 and the second homology region comprises or consists of a nucleotide sequence that has at least 70% identity, at least 80% identity, at least 90% identity, at least 95% identity, at least 98% identity, or 100% identity to SEQ ID NO: 19;
(ii) the first homology region comprises or consists of a nucleotide sequence that has at least 70% identity, at least 80% identity, at least 90% identity, at least 95% identity, at least 98% identity, or 100% identity to SEQ ID NO: 8 and the second homology region comprises or consists of a nucleotide sequence that has at least 70% identity, at least 80% identity, at least 90% identity, at least 95% identity, at least 98% identity, or 100% identity to SEQ ID NO: 20;
(iii) the first homology region comprises or consists of a nucleotide sequence that has at least 70% identity, at least 80% identity, at least 90% identity, at least 95% identity, at least 98% identity, or 100% identity to SEQ ID NO: 9 and the second homology region comprises or consists of a nucleotide sequence that has at least 70% identity, at least 80% identity, at least 90% identity, at least 95% identity, at least 98% identity, or 100% identity to SEQ ID NO: 21;
(iv) the first homology region comprises or consists of a nucleotide sequence that has at least 70% identity, at least 80% identity, at least 90% identity, at least 95% identity, at least 98% identity, or 100% identity to SEQ ID NO: 10 and the second homology region comprises or consists of a nucleotide sequence that has at least 70% identity, at least 80% identity, at least 90% identity, at least 95% identity, at least 98% identity, or 100% identity to SEQ ID NO: 22;
(v) the first homology region comprises or consists of a nucleotide sequence that has at least 70% identity, at least 80% identity, at least 90% identity, at least 95% identity, at least 98% identity, or 100% identity to SEQ ID NO: 11 and the second homology region comprises or consists of a nucleotide sequence that has at least 70% identity, at least 80% identity, at least 90% identity, at least 95% identity, at least 98% identity, or 100% identity to SEQ ID NO: 23;
(vi) the first homology region comprises or consists of a nucleotide sequence that has at least 70% identity, at least 80% identity, at least 90% identity, at least 95% identity, at least 98% identity, or 100% identity to SEQ ID NO: 12 and the second homology region comprises or consists of a nucleotide sequence that has at least 70% identity, at least 80% identity, at least 90% identity, at least 95% identity, at least 98% identity, or 100% identity to SEQ ID NO: 24;
(vii) the first homology region comprises or consists of a nucleotide sequence that has at least 70% identity, at least 80% identity, at least 90% identity, at least 95% identity, at least 98% identity, or 100% identity to SEQ ID NO: 13 and the second homology region comprises or consists of a nucleotide sequence that has at least 70% identity, at least 80% identity, at least 90% identity, at least 95% identity, at least 98% identity, or 100% identity to SEQ ID NO: 25;
(viii) the first homology region comprises or consists of a nucleotide sequence that has at least 70% identity, at least 80% identity, at least 90% identity, at least 95% identity, at least 98% identity, or 100% identity to SEQ ID NO: 14 and the second homology region comprises or consists of a nucleotide sequence that has at least 70% identity, at least 80% identity, at least 90% identity, at least 95% identity, at least 98% identity, or 100% identity to SEQ ID NO: 26;
(ix) the first homology region comprises or consists of a nucleotide sequence that has at least 70% identity, at least 80% identity, at least 90% identity, at least 95% identity, at least 98% identity, or 100% identity to SEQ ID NO: 15 and the second homology region comprises or consists of a nucleotide sequence that has at least 70% identity, at least 80% identity, at least 90% identity, at least 95% identity, at least 98% identity, or 100% identity to SEQ ID NO: 27;
(x) the first homology region comprises or consists of a nucleotide sequence that has at least 70% identity, at least 80% identity, at least 90% identity, at least 95% identity, at least 98% identity, or 100% identity to SEQ ID NO: 16 and the second homology region comprises or consists of a nucleotide sequence that has at least 70% identity, at least 80% identity, at least 90% identity, at least 95% identity, at least 98% identity, or 100% identity to SEQ ID NO: 28;
(xi) the first homology region comprises or consists of a nucleotide sequence that has at least 70% identity, at least 80% identity, at least 90% identity, at least 95% identity, at least 98% identity, or 100% identity to SEQ ID NO: 17 and the second homology region comprises or consists of a nucleotide sequence that has at least 70% identity, at least 80% identity, at least 90% identity, at least 95% identity, at least 98% identity, or 100% identity to SEQ ID NO: 29; or
(xii) the first homology region comprises or consists of a nucleotide sequence that has at least 70% identity, at least 80% identity, at least 90% identity, at least 95% identity, at least 98% identity, or 100% identity to SEQ ID NO: 18 and the second homology region comprises or consists of a nucleotide sequence that has at least 70% identity, at least 80% identity, at least 90% identity, at least 95% identity, at least 98% identity, or 100% identity to SEQ ID NO: 30.

In some embodiments:
(i) the first homology region comprises or consists of a nucleotide sequence that has at least 70% identity, at least 80% identity, at least 90% identity, at least 95% identity, at least 98% identity, or 100% identity to SEQ ID NO: 7 and the second homology region comprises or consists of a nucleotide sequence that has at least 70% identity, at least 80% identity, at least 90% identity, at least 95% identity, at least 98% identity, or 100% identity to SEQ ID NO: 19;
(ii) the first homology region comprises or consists of a nucleotide sequence that has at least 70% identity, at least 80% identity, at least 90% identity, at least 95% identity, at least 98% identity, or 100% identity to SEQ ID NO: 10 and the second homology region comprises or consists of a nucleotide sequence that has at least 70% identity, at least 80% identity, at least 90% identity, at least 95% identity, at least 98% identity, or 100% identity to SEQ ID NO: 22; or
(iii) the first homology region comprises or consists of a nucleotide sequence that has at least 70% identity, at least 80% identity, at least 90% identity, at least 95% identity, at least 98% identity, or 100% identity to SEQ ID NO: 14 and the second homology region comprises or consists of a nucleotide sequence that has at least 70% identity, at least 80% identity, at least 90% identity, at least 95% identity, at least 98% identity, or 100% identity to SEQ ID NO: 26.

In some embodiments, the first homology region comprises or consists of a nucleotide sequence that has at least 70% identity, at least 80% identity, at least 90% identity, at least 95% identity, at least 98% identity, or 100% identity to SEQ ID NO: 7 and the second homology region comprises or consists of a nucleotide sequence that has at least 70% identity, at least 80% identity, at least 90% identity, at least 95% identity, at least 98% identity, or 100% identity to SEQ ID NO: 19.

In some embodiments, the first homology region comprises or consists of a nucleotide sequence that has at least 70% identity, at least 80% identity, at least 90% identity, at least 95% identity, at least 98% identity, or 100% identity to SEQ ID NO: 8 and the second homology region comprises or consists of a nucleotide sequence that has at least 70% identity, at least 80% identity, at least 90% identity, at least 95% identity, at least 98% identity, or 100% identity to SEQ ID NO: 20.

In some embodiments, the first homology region comprises or consists of a nucleotide sequence that has at least 70% identity, at least 80% identity, at least 90% identity, at least 95% identity, at least 98% identity, or 100% identity to SEQ ID NO: 9 and the second homology region comprises or consists of a nucleotide sequence that has at least 70% identity, at least 80% identity, at least 90% identity, at least 95% identity, at least 98% identity, or 100% identity to SEQ ID NO: 21.

In some embodiments, the first homology region comprises or consists of a nucleotide sequence that has at least 70% identity, at least 80% identity, at least 90% identity, at least 95% identity, at least 98% identity, or 100% identity to SEQ ID NO: 10 and the second homology region comprises or consists of a nucleotide sequence that has at least 70% identity, at least 80% identity, at least 90% identity, at least 95% identity, at least 98% identity, or 100% identity to SEQ ID NO: 22.

In some embodiments, the first homology region comprises or consists of a nucleotide sequence that has at least 70% identity, at least 80% identity, at least 90% identity, at least 95% identity, at least 98% identity, or 100% identity to SEQ ID NO: 11 and the second homology region comprises or consists of a nucleotide sequence that has at least 70% identity, at least 80% identity, at least 90% identity, at least 95% identity, at least 98% identity, or 100% identity to SEQ ID NO: 23.

In some embodiments, the first homology region comprises or consists of a nucleotide sequence that has at least 70% identity, at least 80% identity, at least 90% identity, at least 95% identity, at least 98% identity, or 100% identity to SEQ ID NO: 12 and the second homology region comprises or consists of a nucleotide sequence that has at least 70% identity, at least 80% identity, at least 90% identity, at least 95% identity, at least 98% identity, or 100% identity to SEQ ID NO: 24.

In some embodiments, the first homology region comprises or consists of a nucleotide sequence that has at least 70% identity, at least 80% identity, at least 90% identity, at least 95% identity, at least 98% identity, or 100% identity to SEQ ID NO: 13 and the second homology region comprises or consists of a nucleotide sequence that has at least 70% identity, at least 80% identity, at least 90% identity, at least 95% identity, at least 98% identity, or 100% identity to SEQ ID NO: 25.

In some embodiments, the first homology region comprises or consists of a nucleotide sequence that has at least 70% identity, at least 80% identity, at least 90% identity, at least 95% identity, at least 98% identity, or 100% identity to SEQ ID NO: 14 and the second homology region comprises or consists of a nucleotide sequence that has at least 70% identity, at least 80% identity, at least 90% identity, at least 95% identity, at least 98% identity, or 100% identity to SEQ ID NO: 26.

In some embodiments, the first homology region comprises or consists of a nucleotide sequence that has at least 70% identity, at least 80% identity, at least 90% identity, at least 95% identity, at least 98% identity, or 100% identity to SEQ ID NO: 15 and the second homology region comprises or consists of a nucleotide sequence that has at least 70% identity, at least 80% identity, at least 90% identity, at least 95% identity, at least 98% identity, or 100% identity to SEQ ID NO: 27.

In some embodiments, the first homology region comprises or consists of a nucleotide sequence that has at least 70% identity, at least 80% identity, at least 90% identity, at least 95% identity, at least 98% identity, or 100% identity to SEQ ID NO: 16 and the second homology region comprises or consists of a nucleotide sequence that has at least 70% identity, at least 80% identity, at least 90% identity, at least 95% identity, at least 98% identity, or 100% identity to SEQ ID NO: 28.

In some embodiments, the first homology region comprises or consists of a nucleotide sequence that has at least 70% identity, at least 80% identity, at least 90% identity, at least 95% identity, at least 98% identity, or 100% identity to SEQ ID NO: 17 and the second homology region comprises or consists of a nucleotide sequence that has at least 70% identity, at least 80% identity, at least 90% identity, at least 95% identity, at least 98% identity, or 100% identity to SEQ ID NO: 29.

In some embodiments, the first homology region comprises or consists of a nucleotide sequence that has at least 70% identity, at least 80% identity, at least 90% identity, at least 95% identity, at least 98% identity, or 100% identity to SEQ ID NO: 18 and the second homology region comprises or consists of a nucleotide sequence that has at least 70% identity, at least 80% identity, at least 90% identity, at least 95% identity, at least 98% identity, or 100% identity to SEQ ID NO: 30.

In some embodiments, the first homology region comprises or consists of a nucleotide sequence that has at least 98% identity to SEQ ID NO: 7 and the second homology region comprises or consists of a nucleotide sequence that has at least 98% identity to SEQ ID NO: 19.

In some embodiments, the first homology region comprises or consists of the nucleotide sequence of SEQ ID NO: 7 and the second homology region comprises or consists of the nucleotide sequence of SEQ ID NO: 19.

In some embodiments, the 3′ terminal sequence of the first homology region consists of a nucleotide sequence that has at least 70% identity, at least 80% identity, at least 90% identity, at least 95% identity, at least 98% identity, or 100% identity to any of SEQ ID NOs: 7-18 and/or the 5′ terminal sequence of the second homology region comprises or consists of a nucleotide sequence that has at least 70% identity, at least 80% identity, at least 90% identity, at least 95% identity, at least 98% identity, or 100% identity to any of SEQ ID NOs: 19-30.

Suitably, the 3′ terminal sequence of the first homology region comprises or consists of a nucleotide sequence that has at least 70% identity, at least 80% identity, at least 90% identity, at least 95% identity, at least 98% identity, or 100% identity to any of SEQ ID NOs: 7-18 and the 5′ terminal sequence of the second homology region comprises or consists of a nucleotide sequence that has at least 70% identity, at least 80% identity, at least 90% identity, at least 95% identity, at least 98% identity, or 100% identity to the corresponding nucleotide sequence in Table 1 (i.e. SEQ ID NOs: 19-30).

For example, in some embodiments:
(i) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 7 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 19;
(ii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 8 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 20;
(iii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 9 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 21;
(iv) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 10 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 22;
(v) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 11 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 23;
(vi) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 12 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 24;
(vii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 13 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 25;
(viii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 14 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 26;
(ix) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 15 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 27;
(x) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 16 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 28;
(xi) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 17 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 29; or
(xii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 18 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 30.

In some embodiments:
(i) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 7 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 19;
(ii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 10 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 22; or
(iii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 14 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 26.

In some embodiments, the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 7 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 19.

In some embodiments, the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 8 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 20.

In some embodiments, the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 9 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 21.

In some embodiments, the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 10 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 22.

In some embodiments, the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 11 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 23.

In some embodiments, the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 12 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 24.

In some embodiments, the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 13 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 25.

In some embodiments, the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 14 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 26.

In some embodiments, the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 15 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 27.

In some embodiments, the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 16 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 28.

In some embodiments, the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 17 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 29.

In some embodiments, the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 18 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 30.

In some embodiments, the 3′ terminal sequence of the first homology region comprises or consists of a nucleotide sequence that has at least 98% identity to SEQ ID NO: 7 and the 5′ terminal sequence of the second homology region comprises or consists of a nucleotide sequence that has at least 98% identity to SEQ ID NO: 19.

In some embodiments, the 3′ terminal sequence of the first homology region comprises or consists of the nucleotide sequence of SEQ ID NO: 7 and the 5′ terminal sequence of the second homology region comprises or consists of the nucleotide sequence of SEQ ID NO: 19.

In some embodiments, the first homology region comprises or consists of a nucleotide sequence that has at least 70% identity, at least 80% identity, at least 90% identity, at least 95% identity, at least 98% identity, or 100% identity to SEQ ID NO: 31, or a fragment thereof; and the second homology region comprises or consists of a nucleotide sequence that has at least 70% identity, at least 80% identity, at least 90% identity, at least 95% identity, at least 98% identity, or 100% identity to SEQ ID NO: 32, or a fragment thereof. Suitably, the fragments are at least 50 bp in length, for example 50-250 bp or 100-200 bp in length.

In some embodiments, the first homology region comprises or consists of a nucleotide sequence that has at least 98% identity to SEQ ID NO: 31, or a fragment thereof; and the second homology region comprises or consists of a nucleotide sequence that has at least 98% identity to SEQ ID NO: 32, or a fragment thereof.

In some embodiments, the first homology region comprises or consists of the nucleotide of SEQ ID NO: 31, or a fragment thereof, and the second homology region comprises or consists of the nucleotide sequence of SEQ ID NO: 32, or a fragment thereof.

Illustrative first homology region for guide RNA 9 (SEQ ID NO: 31)

tgagcacacagttattacttggaaattgtgtacagactaagttgaagatg
ttaggagggaagattgtgggccaagtaacggggtgtatgtgtgtgggtat
agggtgggcagctgggatggaaatggggggctgctgctgctgctgcaccc
tggcctcctgaactaatgatatcactcaccagaaactactgttcctgcac
tgtccaagccaccccaaactagtttgtcaaaatgaatctgtgctgtgtgg
agggaggcacgcctgtagctctgatgtcagatggcaatgt

Illustrative second homology region for guide RNA 9 (SEQ ID NO: 32)

atggcagtggccggtggggacagggctgagccagcaccaaccactcagcc
tttgagatcccgaggctggtctactgctgagaccttttgttagaagagag
gagatcaagcatttgcaaggtttctgagtgtcaaaatatgaatccaagat
aactctttcacaatcctaacttcatgctgtctacaggtccatattttagc
ctgctttctccatgttcatccgaaaagaaagaaaagctaagggtggtggt
catatttgaaattagccagatcttaagtttttctgggggaaatttagaag
aaaatatggaaaagtgactatgagcaca

### Genome Insertion Sites

The site of the double-strand break (DSB) can be introduced specifically by any suitable technique, for example using a CRISPR/Cas9 system and the guide RNAs disclosed herein. In the present invention, the DSB is introduced into the RAG1 intron 1 or RAG1 exon 2. For example, a DSB may be introduced at any of the sites recited in Table 2 below. Optionally, a DSB is introduced into the RAG1 intron 1.

TABLE 2

Exemplary DSB sites in RAG1 intron 1 or RAG1 exon 2

| Guide | Exemplary DSB site |
|---|---|
| 9 | between chr 11: 36569296 and 36569297 |
| 1 | between chr 11: 36573791 and 36573792 |
| 2 | between chr 11: 36573642 and 36573643 |
| 3 | between chr 11: 36573352 and 36573353 |
| 4 | between chr 11: 36569081 and 36569082 |
| 5 | between chr 11: 36572473 and 36572474 |
| 6 | between chr 11: 36571459 and 36571460 |
| 7 | between chr 11: 36571367 and 36571368 |
| 8 | between chr 11: 36572860 and 36572861 |
| 10 | between chr 11: 36571458 and 36571459 |
| 11 | between chr 11: 36569352 and 36569353 |
| 12 | between chr 11: 36572376 and 36572377 |

Suitably, each homology region is homologous to a fragment of the RAG1 intron 1 and/or RAG1 exon 2 either side of the DSB. For example, the first homology region may be homologous to a region in the RAG1 intron 1 and/or RAG1 exon 2 upstream of the DSB and the second homology region may be homologous to a region downstream of the DSB.

In the present invention, the nucleotide sequence insert (e.g. a splice acceptor sequence and a nucleotide sequence encoding a RAG1 polypeptide) may be introduced at the DSB site by homology-directed repair (HDR). Thus, the nucleotide insert (e.g. a splice acceptor sequence and a nucleotide sequence encoding a RAG1 polypeptide) may replace the region of the genome flanked by the homology regions and comprising the DSB.

As used herein, the “nucleotide sequence insert” may consist of the region of the polynucleotide flanked by the first homology region and the second homology region. For example, the nucleotide sequence insert may comprise a splice acceptor sequence and a nucleotide sequence encoding a RAG1 polypeptide.

The nucleotide sequence insert may be introduced into a genome at any of the sites recited in Table 2 above. In other words, the genome of the present invention may comprise the nucleotide sequence insert at any of the sites recited in Table 2 above.

In some embodiments, the nucleotide sequence insert is introduced:
(i) between chr 11: 36569296 and 36569297;
(ii) between chr 11: 36573352 and 36573353; or
(iii) between chr 11: 36571367 and 36571368.

In some embodiments, the nucleotide sequence insert is introduced between chr 11: 36569296 and 36569297.

In some embodiments, the genome of the present invention comprises a nucleotide sequence comprising a splice acceptor sequence and a nucleotide sequence encoding a RAG1 polypeptide, which is introduced:
(i) between chr 11: 36569296 and 36569297;
(ii) between chr 11: 36573352 and 36573353; or
(iii) between chr 11: 36571367 and 36571368.

In some embodiments, the genome of the present invention comprises a nucleotide sequence comprising a splice acceptor sequence and a nucleotide sequence encoding a RAG1 polypeptide, which is introduced between chr 11: 36569296 and 36569297.

The nucleotide sequence insert may replace any of the regions recited in Table 3 below. In other words, the genome of the present invention may comprise the nucleotide sequence insert replacing any of the regions recited in Table 3.

TABLE 3

Exemplary insertion sites in RAG1 intron 1 or RAG1 exon 2

| Guide | Exemplary region to replace |
|---|---|
| 9 | chr 11: 36569295 to 36569298 |
| 1 | chr 11: 36573790 to 36573793 |
| 2 | chr 11: 36573641 to 36573644 |
| 3 | chr 11: 36573351 to 36573354 |
| 4 | chr 11: 36569080 to 36569083 |
| 5 | chr 11: 36572472 to 36572475 |
| 6 | chr 11: 36571458 to 36571461 |
| 7 | chr 11: 36571366 to 36571369 |
| 8 | chr 11: 36572859 to 36572862 |
| 10 | chr 11: 36571457 to 36571460 |
| 11 | chr 11: 36569351 to 36569354 |
| 12 | chr 11: 36572375 to 36572378 |

In some embodiments, the nucleotide sequence insert replaces:
(i) chr 11: 36569295 to 36569298;
(ii) chr 11: 36573351 to 36573354; or
(iii) chr 11: 36571366 to 36571369.

In some embodiments, the nucleotide sequence insert replaces chr 11: 36569295 to 36569298.

In some embodiments, the genome of the present invention comprises a nucleotide sequence comprising a splice acceptor sequence and a nucleotide sequence encoding a RAG1 polypeptide, which replaces:
(i) chr 11: 36569295 to 36569298;
(ii) chr 11: 36573351 to 36573354; or
(iii) chr 11: 36571366 to 36571369.

In some embodiments, the genome of the present invention comprises a nucleotide sequence comprising a splice acceptor sequence and a nucleotide sequence encoding a RAG1 polypeptide, which replaces chr 11: 36569295 to 36569298.

### Splice Acceptor and Donor Sequences

RNA splicing is a form of RNA processing in which a newly made precursor messenger RNA (pre-mRNA) transcript is transformed into a mature messenger RNA (mRNA). During splicing, introns (non-coding regions) are removed and exons (coding regions) are joined together.

Within introns, a donor site (5′ end of the intron), a branch site (near the 3′ end of the intron) and an acceptor site (3′ end of the intron) are required for splicing. The splice donor site includes an almost invariant sequence GU at the 5′ end of the intron, within a larger, less highly conserved region. The splice acceptor site at the 3′ end of the intron terminates the intron with an almost invariant AG sequence. Upstream (5′-ward) from the AG there is a region high in pyrimidines (C and U), or polypyrimidine tract. Further upstream from the polypyrimidine tract is the branchpoint.

A “splice acceptor sequence” is a nucleotide sequence which can function as an acceptor site at the 3′ end of the intron. Consensus sequences and frequencies of human splice site regions are described in Ma, S.L., et al., 2015. PLoS One, 10(6), p.e0130729.

Suitably, the splice acceptor sequence may comprise the nucleotide sequence (Y)ₙNYAG, where n is 10-20, or a variant with at least 90% or at least 95% sequence identity. Suitably, the splice acceptor sequence may comprise the sequence (Y)ₙNCAG, where n is 10-20, or a variant with at least 90% or at least 95% sequence identity.

In some embodiments of the invention, the splice acceptor sequence comprises or consists of a nucleotide sequence which is at least 70% identical to SEQ ID NO: 33 or a fragment thereof. Suitably, the splice acceptor sequence comprises or consists of a nucleotide sequence which is at least 80%, at least 85%, at least 90%, at least 95%, at least 98% or at least 99% identical to SEQ ID NO: 33 or a fragment thereof.

In some embodiments of the invention, the splice acceptor sequence comprises or consists of the nucleotide sequence SEQ ID NO: 33 or a fragment thereof.

Exemplary splice acceptor sequence (SEQ ID NO: 33)

ctgacctcttctcttcctcccacag

The polynucleotide of the invention may comprise a splice donor sequence. The genome may comprise a splice donor sequence in the RAG1 intron 1. Suitably, the splice donor sequence nucleotide sequence is 3′ of the nucleotide sequence encoding a RAG1 polypeptide. The splice donor sequence may be used to provide an mRNA comprising the RAG1 polypeptide and RAG1 exon 2.

A “splice donor sequence” is a nucleotide sequence which can function as a donor site at the 5′ end of the intron. Consensus sequences and frequencies of human splice site regions are describe in Ma, S.L., et al., 2015. PLoS One, 10(6), p.e0130729.

In some embodiments of the invention, the splice donor sequence comprises or consists of a nucleotide sequence which is at least 85% identical to SEQ ID NO: 34 or a fragment thereof. In some embodiments of the invention, the splice donor sequence comprises or consists of the nucleotide sequence SEQ ID NO: 34 or a fragment thereof.

Exemplary splice donor sequence (SEQ ID NO: 34)

aggtaagt

In some embodiments of the invention, the polynucleotide of the invention does not comprise a splice donor sequence.

### Regulatory Elements

The polynucleotide of the invention may comprise one or more regulatory elements which may act pre- or post-transcriptionally. Suitably, the nucleotide sequence encoding a RAG1 polypeptide is operably linked to one or more regulatory elements which may act pre- or post-transcriptionally. The one or more regulatory elements may facilitate expression of the RAG1 polypeptide in the cells of the invention.

A “regulatory element” is any nucleotide sequence which facilitates expression of a polypeptide, e.g. acts to increase expression of a transcript or to enhance mRNA stability. Suitable regulatory elements include for example promoters, enhancer elements, post-transcriptional regulatory elements and polyadenylation sites.

### Polyadenylation Sequence

The polynucleotide of the invention may comprise a polyadenylation sequence. Suitably, the nucleotide sequence encoding a RAG1 polypeptide is operably linked to a polyadenylation sequence. The polyadenylation sequence may improve gene expression.

Suitable polyadenylation sequences will be well known to those of skill in the art. Suitable polyadenylation sequences include a bovine growth hormone (BGH) polyadenylation sequence or an early SV40 polyadenylation signal. In some embodiments of the invention, the polyadenylation sequence is a BGH polyadenylation sequence.

In some embodiments of the invention, the polyadenylation sequence comprises or consists of a nucleotide sequence which is at least 70% identical to SEQ ID NO: 35, 62 or 65 or a fragment thereof. Suitably, the polyadenylation sequence comprises or consists of a nucleotide sequence which is at least 80%, at least 85%, at least 90%, at least 95%, at least 98% or at least 99% identical to SEQ ID NO: 35, 62 or 65 or a fragment thereof.

In some embodiments of the invention, the polyadenylation sequence comprises or consists of the nucleotide sequence SEQ ID NO: 35, 62 or 65 or a fragment thereof.

Exemplary BGH polyadenylation sequence (SEQ ID NO: 35)

Gctgtgccttctagttgccagccatctgttgtttgcccctcccccgtgcc
ttccttgaccctggaaggtgccactcccactgtcctttcctaataaaatg
aggaaattgcatcgcattgtctgagtaggtgtcattctattctggggggt
ggggtggggcaggacagcaagggggaggattgggaagacaatagcaggca
tgctggggatgcggtgggctctatgg

Exemplary BGH polyadenylation sequence (SEQ ID NO: 62)

Actgtgccttctagttgccagccatctgttgtttgcccctcccccgtgcc
ttccttgaccctggaaggtgccactcccactgccctttcctaataaaatg
aggaaattgcatcgcattgtctgagtaggtgtcattctattctggggggt
ggggtggggcaggacagcaagggggaggattgggaagacaatagcaggca
tgctggggatgcggtgggctctatgg

Exemplary BGH polyadenylation sequence (SEQ ID NO: 65)

ctgtgccttctagttgccagccatctgttgtttgcccctcccccgtgcct
tccttgaccctggaaggtgccactcccactgtcctttcctaataaaatga
ggaaattgcatcgcattgtctgagtaggtgtcattctattctggggggtg
gggtggggcaggacagcaagggggaggattgggaagacaatagcaggcat
gctggggatgcggtgggctctatgg

### Kozak Sequence

The polynucleotide of the invention may comprise a Kozak sequence. Suitably, the nucleotide sequence encoding a RAG1 polypeptide is operably linked to a Kozak sequence. A Kozak sequence may be inserted before the start codon of the RAG1 polypeptide to improve the initiation of translation.

Suitable Kozak sequences will be well known to those of skill in the art.

In some embodiments of the invention, the Kozak sequence comprises or consists of a nucleotide sequence which is at least 70% identical to SEQ ID NO: 36 or a fragment thereof. Suitably, the Kozak sequence comprises or consists of a nucleotide sequence which is at least 80%, or at least 90% identical to SEQ ID NO: 36 or a fragment thereof.

In some embodiments of the invention, the Kozak sequence comprises or consists of the nucleotide sequence SEQ ID NO: 36 or a fragment thereof.

Exemplary Kozak sequence (SEQ ID NO: 36)

gccgccaccatg

### Post-Transcriptional Regulatory Elements

The polynucleotide of the invention may comprise a post-transcriptional regulatory element. Suitably, the nucleotide sequence encoding a RAG1 polypeptide is operably linked to a post-transcriptional regulatory element. The post-transcriptional regulatory element may improve gene expression.

Suitable post-transcriptional regulatory elements will be well known to those of skill in the art.

The polynucleotide of the invention may comprise a Woodchuck Hepatitis Virus Post-transcriptional Regulatory Element (WPRE). Suitably, the nucleotide sequence encoding a RAG1 polypeptide is operably linked to a WPRE.

In some embodiments of the invention, the WPRE comprises or consists of a nucleotide sequence which is at least 70% identical to SEQ ID NO: 37 or a fragment thereof. Suitably, the WPRE comprises or consists of a nucleotide sequence which is at least 80%, or at least 90% identical to SEQ ID NO: 37 or a fragment thereof.

In some embodiments of the invention, the WPRE comprises or consists of the nucleotide sequence SEQ ID NO: 37 or a fragment thereof.

Exemplary WPRE (SEQ ID NO: 37)

aatcaacctctggattacaaaatttgtgaaagattgactggtattcttaa
ctatgttgctccttttacgctatgtggatacgctgctttaatgcctttgt
atcatgctattgcttcccgtatggctttcattttctcctccttgtataaa
tcctggttgctgtctctttatgaggagttgtggcccgttgtcaggcaacg
tggcgtggtgtgcactgtgtttgctgacgcaacccccactggttggggca
ttgccaccacctgtcagctcctttccgggactttcgctttccccctccct
attgccacggcggaactcatcgccgcctgccttgcccgctgctggacagg
ggctcggctgttgggcactgacaattccgtggtgttgtcggggaaatcat
cgtcctttccttggctgctcgcctgtgttgccacctggattctgcgcggg
acgtccttctgctacgtcccttcggccctcaatccagcggaccttccttc
ccgcggcctgctgccggctctgcggcctcttccgcgtcttcgccttcgcc
ctcagacgagtcggatctccctttgggccgcctccccgcctg

In some embodiments of the invention, the RAG1 polypeptide is not operably linked to a post-transcriptional regulatory element. In some embodiments of the invention, the RAG1 polypeptide is not operably linked to a WPRE.

### Endogenous 3′UTR

The polynucleotide of the invention may comprise an endogenous RAG1 3′UTR. Suitably, the nucleotide sequence encoding a RAG1 polypeptide is operably linked to an endogenous RAG1 3′UTR.

In some embodiments of the invention, the RAG1 3′UTR comprises or consists of a nucleotide sequence which is at least 70% identical to SEQ ID NO: 38 or a fragment thereof. Suitably, the RAG1 3′UTR comprises or consists of a nucleotide sequence which is at least 80%, or at least 90% identical to SEQ ID NO: 38 or a fragment thereof.

In some embodiments of the invention, the RAG1 3′UTR comprises or consists of the nucleotide sequence SEQ ID NO: 38 or a fragment thereof.

Exemplary RAG1 3′UTR (SEQ ID NO: 38)

gtagggcaaccacttatgagttggtttttgcaattgagtttccctctggg
ttgcattgagggcttctcctagcaccctttactgctgtgtatggggcttc
accatccaagaggtggtaggttggagtaagatgctacagatgctctcaag
tcaggaatagaaactgatgagctgattgcttgaggcttttagtgagttcc
gaaaagcaacaggaaaaatcagttatctgaaagctcagtaactcagaaca
ggagtaactgcaggggaccagagatgagcaaagatctgtgtgtgttgggg
agctgtcatgtaaatcaaagccaaggttgtcaaagaacagccagtgaggc
caggaaagaaattggtcttgtggttttcatttttttcccccttgattgat
tatattttgtattgagatatgataagtgccttctatttcatttttgaata
attcttcatttttataattttacatatcttggcttgctatataagattca
aaagagctttttaaatttttctaataatatcttacatttgtacagcatga
tgacctttacaaagtgctctcaatgcatttacccattcgttatataaata
tgttacatcaggacaactttgagaaaatcagtccttttttatgtttaaat
tatgtatctattgtaaccttcagagtttaggaggtcatctgctgtcatgg
atttttcaataatgaatttagaatacacctgttagctacagttagttatt
aaatcttctgataatatatgtttacttagctatcagaagccaagtatgat
tctttatttttactttttcatttcaagaaatttagagtttccaaatttag
agcttctgcatacagtcttaaagccacagaggcttgtaaaaatataggtt
agcttgatgtctaaaaatatatttcatgtcttactgaaacattttgccag
actttctccaaatgaaacctgaatcaatttttctaaatctaggtttcata
gagtcctctcctctgcaatgtgttattctttctataatgatcagtttact
ttcagtggattcagaattgtgtagcaggataaccttgtatttttccatcc
gctaagtttagatggagtccaaacgcagtacagcagaagagttaacattt
acacagtgctttttaccactgtggaatgttttcacactcatttttcctta
caacaattctgaggagtaggtgttgttattatctccatttgatgggggtt
taaatgatttgctcaaagtcatttaggggtaataaatacttggcttggaa
atttaacacagtccttttgtctccaaagcccttcttctttccaccacaaa
ttaatcactatgtttataaggtagtatcagaatttttttaggattcacaa
ctaatcactatagcacatgaccttgggattacatttttatggggcagggg
taagcaagtttttaaatcatttgtgtgctctggctcttttgatagaagaa
agcaacacaaaagctccaaagggccccctaaccctcttgtggctccagtt
atttggaaactatgatctgcatccttaggaatctgggatttgccagttgc
tggcaatgtagagcaggcatggaattttatatgctagtgagtcataatga
tatgttagtgttaattagttttttcttcctttgattttattggccataat
tgctactcttcatacacagtatatcaaagagcttgataatttagttgtca
aaagtgcatcggcgacattatctttaattgtatgtatttggtgcttcttc
agggattgaactcagtatctttcattaaaaaacacagcagttttccttgc
tttttatatgcagaatatcaaagtcatttctaatttagttgtcaaaaaca
tatacatattttaacattagtttttttgaaaactcttggttttgtttttt
tggaaatgagtgggccactaagccacactttcccttcatcctgcttaatc
cttccagcatgtctctgcactaataaacagctaaattcacataatcatcc
tatttactgaagcatggtcatgctggtttatagattttttacccatttct
actctttttctctattggtggcactgtaaatactttccagtattaaatta
tccttttctaacactgtaggaactattttgaatgcatgtgactaagagca
tgatttatagcacaacctttccaataatcccttaatcagatcacattttg
ataaaccctgggaacatctggctgcaggaatttcaatatgtagaaacgct
gcctatggttttttgcccttactgttgagactgcaatatcctagacccta
gttttatactagagttttatttttagcaatgcctattgcaagtgcaatta
tatactccagggaaattcaccacactgaatcgagcatttgtgtgtgtatg
tgtgaagtatatactgggacttcagaagtgcaatgtatttttctcctgtg
aaacctgaatctacaagttttcctgccaagccactcaggtgcattgcagg
gaccagtgataatggctgatgaaaattgatgattggtcagtgaggtcaaa
aggagccttgggattaataaacatgcactgagaagcaagaggaggagaaa
aagatgtctttttcttccaggtgaactggaatttagttttgcctcagatt
tttttcccacaagatacagaagaagataaagatttttttggttgagagtg
tgggtcttgcattacatcaaacagagttcaaattccacacagataagagg
caggatatataagcgccagtggtagttgggaggaataaaccattatttgg
atgcaggtggtttttgattgcaaatatgtgtgtgtcttcagtgattgtat
gacagatgatgtattcttttgatgttaaaagattttaagtaagagtagat
acattgtacccattttacattttcttattttaactacagtaatctacata
aatatacctcagaaatcatttttggtgattattttttgttttgtagaatt
gcacttcagtttattttcttacaaataaccttacattttgtttaatggct
tccaagagccttttttttttttgtatttcagagaaaattcaggtaccagg
atgcaatggatttatttgattcaggggacctgtgtttccatgtcaaatgt
tttcaaataaaatgaaatatgagtttcaatactttttatattttaatatt
tccattcattaatattatggttattgtcagcaattttatgtttgaatatt
tgaaataaaagtttaagatttgaaaatggtatgtattataatttctattc
aaatattaataataatattgagtgcagcatt

In some embodiments of the invention, the RAG1 polypeptide is not operably linked to a RAG1 3′UTR.

### Further Coding Sequences

The polynucleotide of the invention may comprise a further coding sequence. The polynucleotide of the invention may comprise an internal ribosome entry site sequence (IRES). The IRES may increase or allow expression of the further coding sequence. The IRES may be operably linked to the further coding sequence.

In some embodiments of the invention, the IRES comprises or consists of a nucleotide sequence which is at least 70% identical to SEQ ID NO: 63 or a fragment thereof. Suitably, the IRES comprises or consists of a nucleotide sequence which is at least 80%, or at least 90% identical to SEQ ID NO: 63 or a fragment thereof.

In some embodiments of the invention, the IRES comprises or consists of the nucleotide sequence SEQ ID NO: 63 or a fragment thereof.

Exemplary IRES (SEQ ID NO: 63)

gaattaactcgaggaattccgCccctctccctcccccccccctaacgtta
ctggccgaagccgcttggaataaggccggtgtgcgtttgtctatatgtta
ttttccaccatattgccgtcttttggcaatgtgagggcccggaaacctgg
ccctgtcttcttgacgagcattctaggggtctttcccctctcgccaaagg
aatgcaaggtctgttgaatgtcgtgaaggaagcagttcctctggaagctt
cttgaagacaaacaacgtctgtagcgaccctttgcaggcagcggaacccc
ccacctggcgacaggtgcctctgcggccaaaagccaacgtgtataagata
cacctgcaaaggcggcacaaccccagtgccacgttgtgagttggatagtt
gtggaaagagtcaaatggctctcctcaagcgtattcaacaaggggctgaa
ggatgcccagaaggtaccccattgtatgggatctgatctggggcctcggt
gcacatgctttacatgtgtttagtcgaggttaaaaaacgtctaggccccc
cgaaccacggggacgtggttttcctttgaaaaacacgatgataatatggc
cacaacc

The further coding sequence may encode a selector, for example a NGFR receptor, e.g. a low affinity NGFR, such as a C-terminal truncated low affinity NGFR. The selector may be used for enrichment of cells.

In some embodiments of the invention, the NGFR-encoding sequence comprises or consists of a nucleotide sequence which is at least 70% identical to SEQ ID NO: 64 or a fragment thereof. Suitably, the NGFR-encoding sequence comprises or consists of a nucleotide sequence which is at least 80%, or at least 90% identical to SEQ ID NO: 64 or a fragment thereof.

In some embodiments of the invention, the NGFR-encoding sequence comprises or consists of the nucleotide sequence SEQ ID NO: 64 or a fragment thereof.

Exemplary NGFR-encoding sequence (SEQ ID NO: 64)

atgggagctggtgctaccggcagagctatggatggacctagactgctgct
cctgctgctgctcggagtttctcttggcggagccaaagaggcctgtccta
ccggcctgtatacacactctggcgagtgctgcaaggcctgcaatcttgga
gaaggcgtggcacagccttgcggcgctaatcagacagtgtgcgagccttg
cctggacagcgtgacctttagcgacgtggtgtctgccaccgagccatgca
agccttgtaccgagtgtgtgggcctgcagagcatgtctgccccttgtgtg
gaagccgacgatgccgtgtgtagatgcgcctacggctactaccaggacga
gacaacaggcagatgcgaggcctgtagagtgtgtgaagccggctctggac
tggtgttcagctgccaagacaagcagaacaccgtgtgcgaggaatgcccc
gatggcacctatagcgacgaggccaaccatgtagatccctgcctgccttg
tactgtgtgcgaagataccgagcggcagctgcgcgagtgtacaagatggg
ctgatgccgagtgcgaagagatccccggcagatggatcaccagaagcaca
cctccagagggcagcgatagcacagccccttctacacaagagcccgaggc
tcctcctgagcaggatctgattgcctctacagtggccggcgtggtcacaa
cagtgatgggatcttctcagcccgtggtcaccagaggcaccaccgacaat
ctgatccccgtgtactgtagcatcctggccgccgtggttgtgggactcgt
ggcctatatcgccttcaagcggtggaaccggggcatcctgtaa

The further coding sequence may encode a destabilisation domain, for example a peptide sequence rich in proline (P), glutamic acid (E), serine (S), and threonine (T) (PEST). Endogenous RAG1 protein may be destabilized by the destabilisation domain, e.g. PEST signal peptide via proteasome degradation.

In some embodiments of the invention, the PEST-encoding sequence comprises or consists of a nucleotide sequence which is at least 70% identical to SEQ ID NO: 66 or a fragment thereof. Suitably, the PEST-encoding sequence comprises or consists of a nucleotide sequence which is at least 80%, or at least 90% identical to SEQ ID NO: 66 or a fragment thereof.

In some embodiments of the invention, the PEST-encoding sequence comprises or consists of the nucleotide sequence SEQ ID NO: 66 or a fragment thereof.

Exemplary PEST-encoding sequence (SEQ ID NO: 66)

atgaggaccgaggcccccgagggcaccgagagcgagatggagacccccag
cgccatcaacggcaaccccagctggcac

### Promoters and Enhancers

Suitably, the nucleotide sequence encoding a RAG1 polypeptide is operably linked to a promoter and/or enhancer element.

A “promoter” is a region of DNA that leads to initiation of transcription of a gene. Promoters are located near the transcription start sites of genes, upstream on the DNA (towards the 5′ region of the sense strand). Any suitable promoter may be used, the selection of which may be readily made by the skilled person.

An “enhancer” is a region of DNA that can be bound by proteins (activators) to increase the likelihood that transcription of a particular gene will occur. Enhancers are cis-acting. They can be located up to 1 Mbp (1,000,000 bp) away from the gene, upstream or downstream from the start site. Any suitable enhancer may be used, the selection of which may be readily made by the skilled person.

Transcription of the nucleotide sequence encoding a RAG1 polypeptide may be driven by an endogenous promoter. For example, if the polynucleotide of the present invention is inserted into the RAG1 intron 1, transcription of the nucleotide sequence encoding a RAG1 polypeptide may be driven by the endogenous RAG1 promoter.

In some embodiments of the invention, the polynucleotide of the invention does not comprise a promoter and/or enhancer element. In some embodiments of the invention, the genome of the invention does not comprise a promoter and/or enhancer element (e.g. an exogenous promoter and/or enhancer element) in the RAG1 intron 1.

### Exemplary Polynucleotides and Genomes

In some embodiments, the polynucleotide of the invention comprises, essentially consists of, or consists of from 5′ to 3′: a first homology region, a splice acceptor sequence, a nucleotide sequence encoding a RAG1 polypeptide, a polyadenylation sequence and a second homology region.

In some embodiments, the polynucleotide of the invention comprises, essentially consists of, or consists of from 5′ to 3′: a first homology region, a splice acceptor sequence, a kozak sequence, a nucleotide sequence encoding a RAG1 polypeptide, a polyadenylation sequence and a second homology region.

In some embodiments, the polynucleotide of the invention comprises, essentially consists of, or consists of from 5′ to 3′: a first homology region, a splice acceptor sequence, a nucleotide sequence encoding a RAG1 polypeptide, a WPRE, a polyadenylation sequence and a second homology region.

In some embodiments, the polynucleotide of the invention comprises, essentially consists of, or consists of from 5′ to 3′: a first homology region, a splice acceptor sequence, a kozak sequence, a nucleotide sequence encoding a RAG1 polypeptide, a WPRE, a polyadenylation sequence and a second homology region.

In some embodiments, the polynucleotide of the invention comprises, essentially consists of, or consists of from 5′ to 3′: a first homology region, a splice acceptor sequence, a kozak sequence, a nucleotide sequence encoding a RAG1 polypeptide, a 3′ UTR, a polyadenylation sequence and a second homology region.

In some embodiments, the polynucleotide of the invention comprises, essentially consists of, or consists of from 5′ to 3′: a first homology region, a splice acceptor sequence, a kozak sequence, a nucleotide sequence encoding a RAG1 polypeptide, an IRES, a nucleotide sequence encoding a selector (e.g. NGFR), a polyadenylation sequence and a second homology region.

In some embodiments, the polynucleotide of the invention comprises, essentially consists of, or consists of from 5′ to 3′: a first homology region, a splice acceptor sequence, a kozak sequence, a nucleotide sequence encoding a RAG1 polypeptide, an IRES, a nucleotide sequence encoding a destabilisation domain (e.g. a PEST sequence), a splice donor sequence, and a second homology region.

In some embodiments, the polynucleotide of the invention comprises, essentially consists of, or consists of from 5′ to 3′: a first homology region, a splice acceptor sequence, a nucleotide sequence encoding a RAG1 polypeptide, a splice donor sequence and a second homology region.

In some embodiments, the polynucleotide of the invention comprises or consists of a nucleotide sequence that has at least 70%, at least 75%, at least 80%, at least 85%, at least 90%, at least 95%, at least 98%, or at least 99% identity to SEQ ID NO: 39.

In some embodiments, the polynucleotide of the invention comprises or consists of the nucleotide sequence of SEQ ID NO: 39.

In some embodiments, the genome of the invention comprises a nucleotide sequence that has at least 70%, at least 75%, at least 80%, at least 85%, at least 90%, at least 95%, at least 98%, or at least 99% identity to SEQ ID NO: 39.

In some embodiments, the genome of the invention comprises the nucleotide sequence of SEQ ID NO: 39.

In some embodiments, the genome of the invention comprises a nucleotide sequence that has at least 70%, at least 75%, at least 80%, at least 85%, at least 90%, at least 95%, at least 98%, or at least 99% identity to nucleotides 297-3687 of SEQ ID NO: 39 or nucleotides 291-3693 of SEQ ID NO: 39.

In some embodiments, the genome of the invention comprises the nucleotide sequence of nucleotides 297-3687 of SEQ ID NO: 39 or nucleotides 291-3693 of SEQ ID NO: 39.

Exemplary polynucleotide (SEQ ID NO: 39)

tgagcacacagttattacttggaaattgtgtacagactaagttgaagatg
ttaggagggaagattgtgggccaagtaacggggtgtatgtgtgtgggtat
agggtgggcagctgggatggaaatggggggctgctgctgctgctgcaccc
tggcctcctgaactaatgatatcactcaccagaaactactgttcctgcac
tgtccaagccaccccaaactagtttgtcaaaatgaatctgtgctgtgtgg
agggaggcacgcctgtagctctgatgtcagatggcaatgtgaattcctga
cctcttctcttcctcccacaggccgccaccatggccgcctccttcccacc
tacccttggattgtcctccgcccctgacgaaattcaacatccccacatca
aattctcggagtggaagttcaagctctttcgcgtgcgctcgttcgaaaag
acccccgaggaagcccaaaaggagaagaaagactcattcgaaggaaaacc
cagcctcgaacagtccccggccgtcctggacaaggccgacgggcagaagc
ctgtgccgacccagccgctgctgaaagcgcacccgaaattctccaagaag
tttcacgataacgagaaggcccggggaaaggccatccaccaagcaaacct
tagacacctgtgccgcatctgtgggaactcattcagagccgacgaacata
accggagataccctgtgcatggccctgtcgacggaaagaccctggggctc
ctgagaaagaaggagaagagggcgacatcctggccggacctgatcgcaaa
ggtgttcagaatcgacgtgaaggcagatgtggacagcatccacccaaccg
agttctgccacaactgctggagcattatgcaccggaagttcagctcagcg
ccctgtgaagtgtacttcccgcgcaacgtgactatggagtggcatccaca
cactccgtcctgcgacatctgtaacactgctcggcgcggactcaagagga
agtccctgcagccgaatctgcagctgagcaagaagcttaagaccgtgctg
gaccaggctcggcaggcccgccagcacaagcgacgcgcccaggcccggat
ctcatctaaggatgtgatgaagaagatcgccaattgcagcaaaatccacc
tgtctaccaagctgctggcggtggacttcccggagcacttcgtgaagtcc
atcagctgtcagatctgcgagcatattctcgccgaccccgtggagactaa
ttgcaagcacgtgttctgccgcgtgtgcatcctgcgctgcctgaaggtca
tgggctcctattgcccttcctgccggtacccctgtttccctactgatctg
gagtccccggtcaagtccttcttgtccgtgctgaactccctgatggtcaa
atgtcccgcaaaggagtgcaatgaggaagtgtccctggaaaagtacaacc
accacatcagcagccacaaggagtccaaagaaatctttgtgcacattaac
aagggcggtcggccccggcagcatctgctctcgctgactcgccgggccca
gaagcacaggctccgggagctgaagctgcaagtcaaggccttcgccgaca
aggaagagggaggagatgtgaagtccgtgtgcatgaccctgtttttgctg
gcgctgcgggctcggaacgaacacagacaagctgatgaactggaggccat
catgcagggcaaaggatcgggactccagccggctgtgtgtctcgccatcc
gcgtcaacacattcctctcatgctcccaataccacaagatgtacaggact
gtgaaggccatcaccggacggcagatctttcagccactccacgcccttcg
gaacgcagaaaaggtcttgctgccgggataccatcatttcgaatggcagc
cgcccttgaaaaacgtgtcctcgtccaccgacgtgggcattattgatggg
ctgagcggcctgtcctcctctgtggatgactaccctgtggataccatcgc
caaacggttcagatacgattccgcgctggtgtcggccctgatggacatgg
aggaggacatcctggagggaatgagatcacaagatctggacgactacctc
aacgggcccttcacggtggtggtcaaggaatcgtgcgatggaatgggcga
cgtgtcggagaagcacggttccggacctgtggtgccggaaaaggccgtgc
gcttctccttcaccatcatgaagatcaccattgcgcatagctcccagaac
gtcaaagtgttcgaagaggccaagccgaactcagagctctgctgcaagcc
gctgtgcctgatgttggcggacgagagcgatcacgaaaccctgaccgcca
ttctgtcgcctctgatcgcggagagggaggccatgaagtcctccgaactg
atgctggagctgggcggtattttgcggacttttaagttcatcttccgggg
aaccggttatgacgaaaagctcgtgcgcgaagtggagggcctggaagcct
caggctccgtctacatctgcactctctgcgacgccacccggctggaggcg
tcacagaatcttgtgttccactcgatcactaggtcccacgcggagaacct
ggaacgctatgaggtctggcgctctaacccataccacgaatccgtggaag
aacttcgggacagagtgaagggagtgtcagcaaagcctttcattgaaacc
gtgcctagcatcgacgccctccattgcgacatcggcaacgccgccgagtt
ctacaagatcttccagcttgagatcggggaagtgtacaagaacccgaacg
cctccaaggaagaaagaaagcggtggcaggctacccttgacaaacacctc
cgcaagaagatgaacctgaagcccattatgcggatgaacggaaacttcgc
taggaagctgatgactaaggaaacggtcgacgcggtctgtgaactgatcc
ccagcgaagaacgacatgaagcgctgcgcgaactcatggacctgtacctg
aagatgaagcctgtctggcggagctcgtgccctgccaaggagtgcccgga
gtcgctgtgtcagtacagctttaacagccaaaggttcgcagagctgctgt
cgaccaagttcaagtacagatacgaaggaaagattaccaactacttccac
aagactctcgctcacgtgcccgagattatcgaacgcgatggttccatcgg
ggcctgggcctccgagggcaacgagtcgggcaacaagttgttccgccggt
ttagaaagatgaacgcccgccagtccaagtgctacgaaatggaagatgtg
ctgaagcatcactggctgtatacctccaagtacctccagaagttcatgaa
cgcacataacgccctcaagacctccgggttcaccatgaacccccaggcct
ccctcggtgaccctctgggaattgaagatagcttggagagccaggactcg
atggaattctagctgtgccttctagttgccagccatctgttgtttgcccc
tcccccgtgccttccttgaccctggaaggtgccactcccactgtcctttc
ctaataaaatgaggaaattgcatcgcattgtctgagtaggtgtcattcta
ttctggggggtggggtggggcaggacagcaagggggaggattgggaagac
aatagcaggcatgctggggatgcggtgggctctatggtctagaatggcag
tggccggtggggacagggctgagccagcaccaaccactcagcctttgaga
tcccgaggctggtctactgctgagaccttttgttagaagagaggagatca
agcatttgcaaggtttctgagtgtcaaaatatgaatccaagataactctt
tcacaatcctaacttcatgctgtctacaggtccatattttagcctgcttt
ctccatgttcatccgaaaagaaagaaaagctaagggtggtggtcatattt
gaaattagccagatcttaagtttttctgggggaaatttagaagaaaatat
ggaaaagtgactatgagcaca

In some embodiments, the genome of the invention comprises a nucleotide sequence that has at least 70%, at least 75%, at least 80%, at least 85%, at least 90%, at least 95%, at least 98%, or at least 99% identity to SEQ ID NO: 40.

In some embodiments, the genome of the invention comprises the nucleotide sequence of SEQ ID NO: 40.

Exemplary nucleotide sequence insert (SEQ ID NO: 40)

gaattcctgacctcttctcttcctcccacaggccgccaccatggccgcct
ccttcccacctacccttggattgtcctccgcccctgacgaaattcaacat
ccccacatcaaattctcggagtggaagttcaagctctttcgcgtgcgctc
gttcgaaaagacccccgaggaagcccaaaaggagaagaaagactcattcg
aaggaaaacccagcctcgaacagtccccggccgtcctggacaaggccgac
gggcagaagcctgtgccgacccagccgctgctgaaagcgcacccgaaatt
ctccaagaagtttcacgataacgagaaggcccggggaaaggccatccacc
aagcaaaccttagacacctgtgccgcatctgtgggaactcattcagagcc
gacgaacataaccggagataccctgtgcatggccctgtcgacggaaagac
cctggggctcctgagaaagaaggagaagagggcgacatcctggccggacc
tgatcgcaaaggtgttcagaatcgacgtgaaggcagatgtggacagcatc
cacccaaccgagttctgccacaactgctggagcattatgcaccggaagtt
cagctcagcgccctgtgaagtgtacttcccgcgcaacgtgactatggagt
ggcatccacacactccgtcctgcgacatctgtaacactgctcggcgcgga
ctcaagaggaagtccctgcagccgaatctgcagctgagcaagaagcttaa
gaccgtgctggaccaggctcggcaggcccgccagcacaagcgacgcgccc
aggcccggatctcatctaaggatgtgatgaagaagatcgccaattgcagc
aaaatccacctgtctaccaagctgctggcggtggacttcccggagcactt
cgtgaagtccatcagctgtcagatctgcgagcatattctcgccgaccccg
tggagactaattgcaagcacgtgttctgccgcgtgtgcatcctgcgctgc
ctgaaggtcatgggctcctattgcccttcctgccggtacccctgtttccc
tactgatctggagtccccggtcaagtccttcttgtccgtgctgaactccc
tgatggtcaaatgtcccgcaaaggagtgcaatgaggaagtgtccctggaa
aagtacaaccaccacatcagcagccacaaggagtccaaagaaatctttgt
gcacattaacaagggcggtcggccccggcagcatctgctctcgctgactc
gccgggcccagaagcacaggctccgggagctgaagctgcaagtcaaggcc
ttcgccgacaaggaagagggaggagatgtgaagtccgtgtgcatgaccct
gtttttgctggcgctgcgggctcggaacgaacacagacaagctgatgaac
tggaggccatcatgcagggcaaaggatcgggactccagccggctgtgtgt
ctcgccatccgcgtcaacacattcctctcatgctcccaataccacaagat
gtacaggactgtgaaggccatcaccggacggcagatctttcagccactcc
acgcccttcggaacgcagaaaaggtcttgctgccgggataccatcatttc
gaatggcagccgcccttgaaaaacgtgtcctcgtccaccgacgtgggcat
tattgatgggctgagcggcctgtcctcctctgtggatgactaccctgtgg
ataccatcgccaaacggttcagatacgattccgcgctggtgtcggccctg
atggacatggaggaggacatcctggagggaatgagatcacaagatctgga
cgactacctcaacgggcccttcacggtggtggtcaaggaatcgtgcgatg
gaatgggcgacgtgtcggagaagcacggttccggacctgtggtgccggaa
aaggccgtgcgcttctccttcaccatcatgaagatcaccattgcgcatag
ctcccagaacgtcaaagtgttcgaagaggccaagccgaactcagagctct
gctgcaagccgctgtgcctgatgttggcggacgagagcgatcacgaaacc
ctgaccgccattctgtcgcctctgatcgcggagagggaggccatgaagtc
ctccgaactgatgctggagctgggcggtattttgcggacttttaagttca
tcttccggggaaccggttatgacgaaaagctcgtgcgcgaagtggagggc
ctggaagcctcaggctccgtctacatctgcactctctgcgacgccacccg
gctggaggcgtcacagaatcttgtgttccactcgatcactaggtcccacg
cggagaacctggaacgctatgaggtctggcgctctaacccataccacgaa
tccgtggaagaacttcgggacagagtgaagggagtgtcagcaaagccttt
cattgaaaccgtgcctagcatcgacgccctccattgcgacatcggcaacg
ccgccgagttctacaagatcttccagcttgagatcggggaagtgtacaag
aacccgaacgcctccaaggaagaaagaaagcggtggcaggctacccttga
caaacacctccgcaagaagatgaacctgaagcccattatgcggatgaacg
gaaacttcgctaggaagctgatgactaaggaaacggtcgacgcggtctgt
gaactgatccccagcgaagaacgacatgaagcgctgcgcgaactcatgga
cctgtacctgaagatgaagcctgtctggcggagctcgtgccctgccaagg
agtgcccggagtcgctgtgtcagtacagctttaacagccaaaggttcgca
gagctgctgtcgaccaagttcaagtacagatacgaaggaaagattaccaa
ctacttccacaagactctcgctcacgtgcccgagattatcgaacgcgatg
gttccatcggggcctgggcctccgagggcaacgagtcgggcaacaagttg
ttccgccggtttagaaagatgaacgcccgccagtccaagtgctacgaaat
ggaagatgtgctgaagcatcactggctgtatacctccaagtacctccaga
agttcatgaacgcacataacgccctcaagacctccgggttcaccatgaac
ccccaggcctccctcggtgaccctctgggaattgaagatagcttggagag
ccaggactcgatggaattctagctgtgccttctagttgccagccatctgt
tgtttgcccctcccccgtgccttccttgaccctggaaggtgccactccca
ctgtcctttcctaataaaatgaggaaattgcatcgcattgtctgagtagg
tgtcattctattctggggggtggggtggggcaggacagcaagggggagga
ttgggaagacaatagcaggcatgctggggatgcggtgggctctatggtct
aga

### Variants, Derivatives, Analogues, and Fragments

In addition to the specific proteins and nucleotides mentioned herein, the invention also encompasses variants, derivatives, and fragments thereof.

In the context of the invention, a “variant” of any given sequence is a sequence in which the specific sequence of residues (whether amino acid or nucleic acid residues) has been modified in such a manner that the polypeptide or polynucleotide in question retains at least one of its endogenous functions. For example, a variant of RAG1 may retain the ability to form a RAG complex, mediate DNA-binding to the RSS, and introduce a double-strand break between the RSS and the adjacent coding segment. A variant sequence can be obtained by addition, deletion, substitution, modification, replacement and/or variation of at least one residue present in the naturally occurring polypeptide or polynucleotide.

The term “derivative” as used herein in relation to proteins or polypeptides of the invention includes any substitution of, variation of, modification of, replacement of, deletion of and/or addition of one (or more) amino acid residues from or to the sequence, providing that the resultant protein or polypeptide retains at least one of its endogenous functions. For example, a derivative of RAG1 may retain the ability to form a RAG complex, mediate DNA-binding to the RSS, and introduce a double-strand break between the RSS and the adjacent coding segment.

Typically, amino acid substitutions may be made, for example from 1, 2 or 3, to 10 or 20 substitutions, provided that the modified sequence retains the required activity or ability. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

Typically, a variant may have a certain identity with the wild type amino acid sequence or the wild type nucleotide sequence.

In the present context, a variant sequence is taken to include an amino acid sequence which may be at least 50%, 55%, 65%, 75%, 85% or 90% identical, suitably at least 95%, 96% or 97% or 98% or 99% identical to the subject sequence. Although a variant can also be considered in terms of similarity (i.e. amino acid residues having similar chemical properties/functions), in the context of the present invention it is preferred to express in terms of sequence identity.

In the present context, a variant sequence is taken to include a nucleotide sequence which may be at least 50%, 55%, 65%, 75%, 85% or 90% identical, suitably at least 95%, 96% or 97% or 98% or 99% identical to the subject sequence. Although a variant can also be considered in terms of similarity, in the context of the present invention it is preferred to express it in terms of sequence identity.

Suitably, reference to a sequence which has a percent identity to any one of the SEQ ID NOs detailed herein refers to a sequence which has the stated percent identity over the entire length of the SEQ ID NO referred to.

Sequence identity comparisons can be conducted by eye, or more usually, with the aid of readily available sequence comparison programs. These commercially available computer programs can calculate percent identity between two or more sequences.

Percent identity may be calculated over contiguous sequences, i.e. one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid or nucleotide sequence may cause the following residues or codons to be put out of alignment, thus potentially resulting in a large reduction in percent identity when a global alignment is performed. Consequently, most sequence comparison methods are designed to produce optimal alignments that take into consideration possible insertions and deletions without penalising unduly the overall identity score. This is achieved by inserting “gaps” in the sequence alignment to try to maximise local identity.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is -12 for a gap and -4 for each extension.

Calculation of maximum percent identity therefore firstly requires the production of an optimal alignment, taking into consideration gap penalties. A suitable computer program for carrying out such an alignment is the GCG Wisconsin Bestfit package (University of Wisconsin, USA; Devereux et al. (1984) Nucleic Acids Research 12: 387). Examples of other software that can perform sequence comparisons include, but are not limited to, the BLAST package (see Ausubel et al. (1999) ibid - Ch. 18), FASTA (Atschul et al. (1990) J. Mol. Biol. 403-410), EMBOSS Needle (Madeira, F., et al., 2019. Nucleic acids research, 47(W1), pp.W636-W641) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, BLAST 2 Sequences, is also available for comparing protein and nucleotide sequences (FEMS Microbiol. Lett. (1999) 174(2):247-50; FEMS Microbiol. Lett. (1999) 177(1):187-8).

Although the final percent identity can be measured, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix (the default matrix for the BLAST suite of programs). GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent sequence identity. The software typically does this as part of the sequence comparison and generates a numerical result. The percent sequence identity may be calculated as the number of identical residues as a percentage of the total residues in the SEQ ID NO referred to.

“Fragments” are also variants and the term typically refers to a selected region of the polypeptide or polynucleotide that is of interest either functionally or, for example, in an assay.

“Fragment” thus refers to an amino acid or nucleic acid sequence that is a portion of a full-length polypeptide or polynucleotide.

Such variants, derivatives, and fragments may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

### Vector

In one aspect, the present invention provides a vector comprising the polynucleotide of the invention.

The vector may be suitable for editing a genome using the polynucleotide of the invention. The vector may be used to deliver the polynucleotide into the cell. Subsequently, the nucleotide sequence insert can be introduced into a genome at a site of a double strand break (DSB) by homology-directed repair (HDR).

The vector of the present invention may be capable of transducing mammalian cells, for example human cells. Suitably, the vector of the present invention is capable of transducing HSCs, HPCs, and/or LPCs. Suitably, the vector of the present invention is capable of transducing CD34+ cells. Suitably, the vector of the present invention is capable of transducing NALM6, K562, and/or other human cell lines (e.g. Molt4, U937, etc.). Suitably, the vector of the present invention is capable of transducing T cells.

Suitably, the vector of the present invention is a viral vector. The vector of the invention may be an adeno-associated viral (AAV) vector, although it is contemplated that other viral vectors may be used e.g. lentiviral vectors (e.g. IDLV vectors), or single or double stranded DNA.

The vector of the present invention may be in the form of a viral vector particle. Suitably, the viral vector of the present invention is in the form of an AAV vector particle. Suitably, the viral vector of the present invention is in the form of a lentiviral vector particle, for example an IDLV vector particle.

Methods of preparing and modifying viral vectors and viral vector particles, such as those derived from AAV, are well known in the art. Suitable methods are described in Ayuso, E., et al., 2010. Current gene therapy, 10(6), pp.423-436, Merten, O.W., et al., 2016. Molecular Therapy-Methods & Clinical Development, 3, p.16017; and Nadeau, I. and Kamen, A., 2003. Biotechnology advances, 20(7-8), pp.475-489.

### Adeno-Associated Viral (AAV) Vectors

The vector of the present invention may be an adeno-associated viral (AAV) vector. Optionally, the vector is an AAV6 vector. The vector of the present invention may be in the form of an AAV vector particle. Optionally, the vector is in the form of an AAV6 vector particle.

The AAV vector or AAV vector particle may comprise an AAV genome or a fragment or derivative thereof. An AAV genome is a polynucleotide sequence, which may encode functions needed for production of an AAV particle. These functions include those operating in the replication and packaging cycle of AAV in a host cell, including encapsidation of the AAV genome into an AAV particle. Naturally occurring AAVs are replication-deficient and rely on the provision of helper functions in trans for completion of a replication and packaging cycle. Accordingly, the AAV genome of the AAV vector of the invention is typically replication-deficient.

The AAV genome may be in single-stranded form, either positive or negative-sense, or alternatively in double-stranded form. The use of a double-stranded form allows bypass of the DNA replication step in the target cell and so can accelerate transgene expression.

AAVs occurring in nature may be classified according to various biological systems. The AAV genome may be from any naturally derived serotype, isolate or clade of AAV.

AAV may be referred to in terms of their serotype. A serotype corresponds to a variant subspecies of AAV which, owing to its profile of expression of capsid surface antigens, has a distinctive reactivity which can be used to distinguish it from other variant subspecies. Typically, an AAV vector particle having a particular AAV serotype does not efficiently cross-react with neutralising antibodies specific for any other AAV serotype. AAV serotypes include AAV1, AAV2, AAV3, AAV4, AAV5, AAV6, AAV7, AAV8, AAV9, AAV10 and AAV11. The AAV vector of the invention may be an AAV6 serotype.

AAV may also be referred to in terms of clades or clones. This refers to the phylogenetic relationship of naturally derived AAVs, and typically to a phylogenetic group of AAVs which can be traced back to a common ancestor, and includes all descendants thereof. Additionally, AAVs may be referred to in terms of a specific isolate, i.e. a genetic isolate of a specific AAV found in nature. The term genetic isolate describes a population of AAVs which has undergone limited genetic mixing with other naturally occurring AAVs, thereby defining a recognisably distinct population at a genetic level.

Typically, the AAV genome of a naturally derived serotype, isolate or clade of AAV comprises at least one inverted terminal repeat sequence (ITR). An ITR sequence acts in cis to provide a functional origin of replication and allows for integration and excision of the vector from the genome of a cell. ITRs may be the only sequences required in cis next to the therapeutic gene. Suitably, one or more ITR sequences flank the polynucleotide of the invention.

The AAV genome may also comprise packaging genes, such as rep and/or cap genes which encode packaging functions for an AAV particle. A promoter may be operably linked to each of the packaging genes. Specific examples of such promoters include the p5, p19 and p40 promoters. For example, the p5 and p19 promoters are generally used to express the rep gene, while the p40 promoter is generally used to express the cap gene. The rep gene encodes one or more of the proteins Rep78, Rep68, Rep52 and Rep40 or variants thereof. The cap gene encodes one or more capsid proteins such as VP1, VP2 and VP3 or variants thereof.

The AAV genome may be the full genome of a naturally occurring AAV. For example, a vector comprising a full AAV genome may be used to prepare an AAV vector or vector particle.

Suitably, the AAV genome is derivatised for the purpose of administration to patients. Such derivatisation is standard in the art and the invention encompasses the use of any known derivative of an AAV genome, and derivatives which could be generated by applying techniques known in the art. The AAV genome may be a derivative of any naturally occurring AAV. Suitably, the AAV genome is a derivative of AAV1, AAV2, AAV3, AAV4, AAV5, AAV6, AAV7, AAV8, AAV9, AAV10, or AAV11. Suitably, the AAV genome is a derivative of AAV6.

Derivatives of an AAV genome include any truncated or modified forms of an AAV genome which allow for expression of a transgene from an AAV vector of the invention in vivo. Typically, it is possible to truncate the AAV genome significantly to include minimal viral sequence yet retain the above function. This may reduce the risk of recombination of the vector with wild-type virus, and avoid triggering a cellular immune response by the presence of viral gene proteins in the target cell.

Typically, a derivative will include at least one inverted terminal repeat sequence (ITR), optionally more than one ITR, such as two ITRs or more. One or more of the ITRs may be derived from AAV genomes having different serotypes, or may be a chimeric or mutant ITR.

A suitable mutant ITR is one having a deletion of a trs (terminal resolution site). This deletion allows for continued replication of the genome to generate a single-stranded genome which contains both coding and complementary sequences, i.e. a self-complementary AAV genome. This allows for bypass of DNA replication in the target cell, and so enables accelerated transgene expression.

The AAV genome may comprise one or more ITR sequences from any naturally derived serotype, isolate or clade of AAV or a variant thereof. The AAV genome may comprise at least one, such as two, AAV1, AAV2, AAV3, AAV4, AAV5, AAV6, AAV7, AAV8, AAV9, AAV10, or AAV11 ITRs, or variants thereof.

The one or more ITRs may flank the nucleotide sequence of the invention at either end. The inclusion of one or more ITRs is can aid concatamer formation of the AAV vector in the nucleus of a host cell, for example following the conversion of single-stranded vector DNA into double-stranded DNA by the action of host cell DNA polymerases. The formation of such episomal concatamers protects the AAV vector during the life of the host cell, thereby allowing for prolonged expression of the transgene in vivo.

Suitably, ITR elements will be the only sequences retained from the native AAV genome in the derivative. Suitably, a derivative may not include the rep and/or cap genes of the native genome and any other sequences of the native genome. This may reduce the possibility of integration of the vector into the host cell genome. Additionally, reducing the size of the AAV genome allows for increased flexibility in incorporating other sequence elements (such as regulatory elements) within the vector in addition to the transgene.

The following portions could therefore be removed in a derivative of the invention: one inverted terminal repeat (ITR) sequence, the replication (rep) and capsid (cap) genes. However, derivatives may additionally include one or more rep and/or cap genes or other viral sequences of an AAV genome. Naturally occurring AAV integrates with a high frequency at a specific site on human chromosome 19, and shows a negligible frequency of random integration, such that retention of an integrative capacity in the AAV vector may be tolerated in a therapeutic setting.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

The AAV vector particle may be encapsidated by capsid proteins. Suitably, the AAV vector particles may be transcapsidated forms wherein an AAV genome or derivative having an ITR of one serotype is packaged in the capsid of a different serotype. The AAV vector particle also includes mosaic forms wherein a mixture of unmodified capsid proteins from two or more different serotypes makes up the viral capsid. The AAV vector particle also includes chemically modified forms bearing ligands adsorbed to the capsid surface. For example, such ligands may include antibodies for targeting a particular cell surface receptor.

Where a derivative comprises capsid proteins i.e. VP1, VP2 and/or VP3, the derivative may be a chimeric, shuffled or capsid-modified derivative of one or more naturally occurring AAVs. In particular, the invention encompasses the provision of capsid protein sequences from different serotypes, clades, clones, or isolates of AAV within the same vector (i.e. a pseudotyped vector). The AAV vector may be in the form of a pseudotyped AAV vector particle.

Chimeric, shuffled or capsid-modified derivatives will be typically selected to provide one or more desired functionalities for the AAV vector. Thus, these derivatives may display increased efficiency of gene delivery and/or decreased immunogenicity (humoral or cellular) compared to an AAV vector comprising a naturally occurring AAV genome. Increased efficiency of gene delivery, for example, may be effected by improved receptor or co-receptor binding at the cell surface, improved internalisation, improved trafficking within the cell and into the nucleus, improved uncoating of the viral particle and improved conversion of a single-stranded genome to double-stranded form.

Chimeric capsid proteins include those generated by recombination between two or more capsid coding sequences of naturally occurring AAV serotypes. This may be performed for example by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N-and/or C-terminus of a capsid coding sequence. The unrelated protein or peptide may advantageously be one which acts as a ligand for a particular cell type, thereby conferring improved binding to a target cell or improving the specificity of targeting of the vector to a particular cell population. The unrelated protein may also be one which assists purification of the viral particle as part of the production process, i.e. an epitope or affinity tag. The site of insertion will typically be selected so as not to interfere with other functions of the viral particle e.g. internalisation, trafficking of the viral particle.

The capsid protein may be an artificial or mutant capsid protein. The term “artificial capsid” as used herein means that the capsid particle comprises an amino acid sequence which does not occur in nature or which comprises an amino acid sequence which has been engineered (e.g. modified) from a naturally occurring capsid amino acid sequence. In other words the artificial capsid protein comprises a mutation or a variation in the amino acid sequence compared to the sequence of the parent capsid from which it is derived where the artificial capsid amino acid sequence and the parent capsid amino acid sequences are aligned. The AAV vector particle may comprise an AAV6 capsid protein.

### Retroviral and Lentiviral Vectors

The vector of the present invention may be a retroviral vector or a lentiviral vector. The vector of the present invention may be a retroviral vector particle or a lentiviral vector particle.

A retroviral vector may be derived from or may be derivable from any suitable retrovirus. A large number of different retroviruses have been identified. Examples include murine leukaemia virus (MLV), human T-cell leukaemia virus (HTLV), mouse mammary tumour virus (MMTV), Rous sarcoma virus (RSV), Fujinami sarcoma virus (FuSV), Moloney murine leukaemia virus (Mo-MLV), FBR murine osteosarcoma virus (FBR MSV), Moloney murine sarcoma virus (Mo-MSV), Abelson murine leukaemia virus (A-MLV), avian myelocytomatosis virus-29 (MC29) and avian erythroblastosis virus (AEV).

Retroviruses may be broadly divided into two categories, “simple” and “complex”. Retroviruses may be even further divided into seven groups. Five of these groups represent retroviruses with oncogenic potential. The remaining two groups are the lentiviruses and the spumaviruses.

The basic structure of retrovirus and lentivirus genomes share many common features such as a 5′ LTR and a 3′ LTR. Between or within these are located a packaging signal to enable the genome to be packaged, a primer binding site, integration sites to enable integration into a host cell genome, and gag, pol and env genes encoding the packaging components - these are polypeptides required for the assembly of viral particles. Lentiviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration and transcription. LTRs also serve as enhancer-promoter sequences and can control the expression of the viral genes.

The LTRs themselves are identical sequences that can be divided into three elements: U3, R and U5. U3 is derived from the sequence unique to the 3′ end of the RNA. R is derived from a sequence repeated at both ends of the RNA. U5 is derived from the sequence unique to the 5′ end of the RNA. The sizes of the three elements can vary considerably among different retroviruses.

In a defective retroviral vector genome gag, pol and env may be absent or not functional.

In a typical retroviral vector, at least part of one or more protein coding regions essential for replication may be removed from the virus. This makes the viral vector replication-defective. Portions of the viral genome may also be replaced by a library encoding candidate modulating moieties operably linked to a regulatory control region and a reporter moiety in the vector genome in order to generate a vector comprising candidate modulating moieties which is capable of transducing a target host cell and/or integrating its genome into a host genome.

Lentivirus vectors are part of the larger group of retroviral vectors. In brief, lentiviruses can be divided into primate and non-primate groups. Examples of primate lentiviruses include but are not limited to human immunodeficiency virus (HIV), the causative agent of human acquired immunodeficiency syndrome (AIDS); and simian immunodeficiency virus (SIV). Examples of non-primate lentiviruses include the prototype “slow virus” visna/maedi virus (VMV), as well as the related caprine arthritis-encephalitis virus (CAEV), equine infectious anaemia virus (EIAV), and the more recently described feline immunodeficiency virus (FIV) and bovine immunodeficiency virus (BIV).

The lentivirus family differs from retroviruses in that lentiviruses have the capability to infect both dividing and non-dividing cells. In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Suitably, that component part is involved in the biological mechanisms by which the vector infects cells, expresses genes or is replicated.

The lentiviral vector may be a “primate” vector. The lentiviral vector may be a “non-primate” vector (i.e. derived from a virus which does not primarily infect primates, especially humans). Examples of non-primate lentiviruses may be any member of the family of lentiviridae which does not naturally infect a primate.

As examples of lentivirus-based vectors, HIV-1- and HIV-2-based vectors are described below.

The HIV-1 vector contains cis-acting elements that are also found in simple retroviruses. It has been shown that sequences that extend into the gag open reading frame are important for packaging of HIV-1. Therefore, HIV-1 vectors often contain the relevant portion of gag in which the translational initiation codon has been mutated. In addition, most HIV-1 vectors also contain a portion of the env gene that includes the RRE. Rev binds to RRE, which permits the transport of full-length or singly spliced mRNAs from the nucleus to the cytoplasm. In the absence of Rev and/or RRE, full-length HIV-1 RNAs accumulate in the nucleus. Alternatively, a constitutive transport element from certain simple retroviruses such as Mason-Pfizer monkey virus can be used to relieve the requirement for Rev and RRE. Efficient transcription from the HIV-1 LTR promoter requires the viral protein Tat.

Most HIV-2-based vectors are structurally very similar to HIV-1 vectors. Similar to HIV-1-based vectors, HIV-2 vectors also require RRE for efficient transport of the full-length or singly spliced viral RNAs.

Optionally, the viral vector used in the present invention has a minimal viral genome.

By “minimal viral genome” it is to be understood that the viral vector has been manipulated so as to remove the non-essential elements and to retain the essential elements in order to provide the required functionality to infect, transduce and deliver a nucleotide sequence of interest to a target host cell. Further details of this strategy can be found in WO 1998/017815.

Optionally, the plasmid vector used to produce the viral genome within a host cell/packaging cell will have sufficient lentiviral genetic information to allow packaging of an RNA genome, in the presence of packaging components, into a viral particle which is capable of infecting a target cell, but is incapable of independent replication to produce infectious viral particles within the final target cell. Optionally, the vector lacks a functional gag-pol and/or env gene and/or other genes essential for replication.

However, the plasmid vector used to produce the viral genome within a host cell/packaging cell will also include transcriptional regulatory control sequences operably linked to the lentiviral genome to direct transcription of the genome in a host cell/packaging cell. These regulatory sequences may be the natural sequences associated with the transcribed viral sequence (i.e. the 5′ U3 region), or they may be a heterologous promoter, such as another viral promoter (e.g. the CMV promoter).

The vectors may be self-inactivating (SIN) vectors in which the viral enhancer and promoter sequences have been deleted. SIN vectors can be generated and transduce non-dividing cells in vivo with an efficacy similar to that of wild-type vectors. The transcriptional inactivation of the long terminal repeat (LTR) in the SIN provirus should prevent mobilisation by replication-competent virus. This should also enable the regulated expression of genes from internal promoters by eliminating any cis-acting effects of the LTR.

The vectors may be integration-defective. Integration defective lentiviral vectors (IDLVs) can be produced, for example, either by packaging the vector with catalytically inactive integrase (such as an HIV integrase bearing the D64V mutation in the catalytic site) or by modifying or deleting essential att sequences from the vector LTR, or by a combination of the above.

### Adenoviral Vectors

The vector of the present invention may be an adenoviral vector. The vector of the present invention may be an adenoviral vector particle.

The adenovirus is a double-stranded, linear DNA virus that does not go through an RNA intermediate. There are over 50 different human serotypes of adenovirus divided into 6 subgroups based on the genetic sequence homology. The natural targets of adenovirus are the respiratory and gastrointestinal epithelia, generally giving rise to only mild symptoms. Serotypes 2 and 5 (with 95% sequence homology) are most commonly used in adenoviral vector systems and are normally associated with upper respiratory tract infections in the young.

Adenoviruses have been used as vectors for gene therapy and for expression of heterologous genes. The large (36 kb) genome can accommodate up to 8 kb of foreign insert DNA and is able to replicate efficiently in complementing cell lines to produce very high titres of up to 10¹². Adenovirus is thus one of the best systems to study the expression of genes in primary non-replicative cells.

The expression of viral or foreign genes from the adenovirus genome does not require a replicating cell. Adenoviral vectors enter cells by receptor mediated endocytosis. Once inside the cell, adenovirus vectors rarely integrate into the host chromosome. Instead, they function episomally (independently from the host genome) as a linear genome in the host nucleus. Hence the use of recombinant adenovirus alleviates the problems associated with random integration into the host genome.

### Herpes Simplex Viral Vector

The vector of the present invention may be a herpes simplex viral vector. The vector of the present invention may be a herpes simplex viral vector particle.

Herpes simplex virus (HSV) is a neurotropic DNA virus with favorable properties as a gene delivery vector. HSV is highly infectious, so HSV vectors are efficient vehicles for the delivery of exogenous genetic material to cells. Viral replication is readily disrupted by null mutations in immediate early genes that in vitro can be complemented in trans, enabling straightforward production of high-titre pure preparations of non-pathogenic vector. The genome is large (152 Kb) and many of the viral genes are dispensable for replication in vitro, allowing their replacement with large or multiple transgenes. Latent infection with wild-type virus results in episomal viral persistence in sensory neuronal nuclei for the duration of the host lifetime. The vectors are non-pathogenic, unable to reactivate and persist long-term. The latency active promoter complex can be exploited in vector design to achieve long-term stable transgene expression in the nervous system. HSV vectors transduce a broad range of tissues because of the wide expression pattern of the cellular receptors recognized by the virus. Increasing understanding of the processes involved in cellular entry has allowed targeting the tropism of HSV vectors.

### Vaccinia Virus Vectors

The vector of the present invention may be a vaccinia viral vector. The vector of the present invention may be a vaccinia viral vector particle.

Vaccinia virus is large enveloped virus that has an approximately 190 kb linear, double-stranded DNA genome. Vaccinia virus can accommodate up to approximately 25 kb of foreign DNA, which also makes it useful for the delivery of large genes.

A number of attenuated vaccinia virus strains are known in the art that are suitable for gene therapy applications, for example the MVA and NYVAC strains.

### RNA-Guided Gene Editing

The vector of the present invention may be used to deliver a polynucleotide into a cell. Subsequently, a nucleotide sequence insert can be introduced into the cell’s genome at a site of a double strand break (DSB) by homology-directed repair (HDR). The site of the double-strand break (DSB) can be introduced specifically by any suitable technique, for example by using an RNA-guided gene editing system.

An “RNA-guided gene editing system” can be used to introduce a DSB and typically comprises a guide RNA and a RNA-guided nuclease. A CRISPR/Cas9 system is an example of a commonly used RNA-guided gene editing system, but other RNA-guided gene editing systems may also be used.

### Guide RNAs

A “guide RNA” (gRNA) confers target sequence specificity to a RNA-guided nuclease. Guide RNAs are non-coding short RNA sequences which bind to the complementary target DNA sequences. For example, in the CRISPR/Cas9 system, guide RNA first binds to the Cas9 enzyme and the gRNA sequence guides the resulting complex via base-pairing to a specific location on the DNA, where Cas9 performs its nuclease activity by cutting the target DNA strand.

The term “guide RNA” encompasses any suitable gRNA that can be used with any RNA-guided nuclease, and not only those gRNAs that are compatible with a particular nuclease such as Cas9.

The guide RNA may comprise a trans-activating CRISPR RNA (tracrRNA) that provides the stem loop structure and a target-specific CRISPR RNA (crRNA) designed to cleave the gene target site of interest. The tracrRNA and crRNA may be annealed, for example by heating them at 95° C. for 5 minutes and letting them slowly cool down to room temperature for 10 minutes. Alternatively, the guide RNA may be a single guide RNA (sgRNA) that consists of both the crRNA and tracrRNA as a single construct.

The guide RNA may comprise of a 3′-end, which forms a scaffold for nuclease binding, and a 5′-end which is programmable to target different DNA sites. For example, the targeting specificity of CRISPR-Cas9 may be determined by the 15-25 bp sequence at the 5′ end of the guide RNA. The desired target sequence typically precedes a protospacer adjacent motif (PAM) which is a short DNA sequence usually 2-6 bp in length that follows the DNA region targeted for cleavage by the CRISPR system, such as CRISPR-Cas9. The PAM is required for a Cas nuclease to cut and is typically found 3-4 bp downstream from the cut site. After base pairing of the guide RNA to the target, Cas9 mediates a double strand break about 3-nt upstream of PAM.

Numerous tools exist for designing guide RNAs (e.g. Cui, Y., et al., 2018. Interdisciplinary Sciences: Computational Life Sciences, 10(2), pp.455-465). For example, COSMID is a web-based tool for identifying and validating guide RNAs (Cradick TJ, et al. Mol Ther - Nucleic Acids. 2014;3(12):e214).

A list of exemplary guide RNAs for use in the present invention is provided below in Table 4.

TABLE 4

Exemplary guide RNAs

| Guide | Sequence | +/strand | DSB site |
|---|---|---|---|
| 9 | TCAGATGGCAATGTCGAGA (SEQ ID NO: 41) | + | chr 11: 36569296-36569297 |
| 1 | TTTTCCGGATCGATGTGA (SEQ ID NO: 42) | + | chr 11: 36573791-36573792 |
| 2 | GACATCTCTGCCGCATCTG (SEQ ID NO: 43) | + | chr 11: 36573642-36573643 |
| 3 | GTGGGTGCTGAATTTCATC (SEQ ID NO: 44) | - | chr 11: 36573352-36573353 |
| 4 | GATTGTGGGCCAAGTAACG (SEQ ID NO: 45) | + | chr 11: 36569081-36569082 |
| 5 | GAAAGTCACTGTTGGTCGA (SEQ ID NO: 46) | - | chr 11: 36572473-36572474 |
| 6 | CAATTTTGAGGTGTTCGTT (SEQ ID NO: 47) | + | chr 11: 36571459-36571460 |
| 7 | GGGTTGAGTTCAACCTAAG (SEQ ID NO: 48) | + | chr 11: 36571367-36571368 |
| 8 | TTAGCCTCATTGTACTAGC (SEQ ID NO: 49) | - | chr 11: 36572860-36572861 |
| 10 | GCAATTTTGAGGTGTTCGT (SEQ ID NO: 50) | + | chr 11: 36571458-36571459 |
| 11 | ACCAGCCTCGGGATCTCAA (SEQ ID NO: 51) | - | chr 11: 36569352-36569353 |
| 12 | TCAAATCAGTCGGGTTTCC (SEQ ID NO: 52) | + | chr 11: 36572376-36572377 |

In one aspect, the present invention provides a guide RNA comprising or consisting of a nucleotide sequence that has at least 90% identity or at least 95% identity to any of SEQ ID NOs: 41-52, optionally wherein the guide RNA comprises or consists of a nucleotide sequence that has at least 90% identity or at least 95% identity to SEQ ID NO: 41.

In some embodiments, the guide RNA comprises or consists of the nucleotide sequence of any of SEQ ID NOs: 41-52, optionally wherein the guide RNA comprises or consists of the nucleotide sequence of SEQ ID NO: 41.

For example, sequences for guides 9, 3 and 7 may be extended as shown below, for example when used as crRNA:

| Guide | Sequence | +/strand | DSB site |
|---|---|---|---|
| 9 | GTCAGATGGCAATGTCGAGA (SEQ ID NO: 53) | + | chr 11: 36569296-36569297 |
| 3 | TGTGGGTGCTGAATTTCATC (SEQ ID NO: 54) | - | chr 11: 36573352-36573353 |
| 7 | GGGGTTGAGTTCAACCTAAG (SEQ ID NO: 55) | + | chr 11: 36571367-36571368 |

In one aspect, the present invention provides a guide RNA comprising or consisting of a nucleotide sequence that has at least 90% identity or at least 95% identity to any of SEQ ID NOs: 53-55, optionally wherein the guide RNA comprises or consists of a nucleotide sequence that has at least 90% identity or at least 95% identity to SEQ ID NO: 53.

In some embodiments, the guide RNA comprises or consists of the nucleotide sequence of any of SEQ ID NOs: 53-55, optionally wherein the guide RNA comprises or consists of the nucleotide sequence of SEQ ID NO: 53.

Suitably, the guide RNA is chemically modified. The chemical modification may enhance the stability of the guide RNA. For example, from one to five (e.g. three) of the terminal nucleotides at 5′ end and/or 3′ end of the guide RNA may be chemically modified to enhance stability.

Any chemical modification which enhances the stability of the guide RNA may be used. For example, the chemical modification may be modification with 2′-O-methyl 3′-phosphorothioate, as described in Hendel A, et al. Nat Biotechnol. 2015;33(9):985-9.

### RNA-Guided Nuclease

A “nuclease” is an enzyme that can cleave the phosphodiester bond present within a polynucleotide chain. Suitably, the nuclease is an endonuclease. Endonucleases are capable of breaking the bond from the middle of a chain.

An “RNA-guided nuclease” is a nuclease which can be directed to a specific site by a guide RNA. The present invention can be implemented using any suitable RNA-guided nuclease, for example any RNA-guided nuclease described in Murugan, K., et al., 2017. Molecular cell, 68(1), pp.15-25. RNA-guided nucleases include, but are not limited to, Type II CRISPR nucleases such as Cas9, and Type V CRISPR nucleases such as Cas12a and Cas12b, as well as other nucleases derived therefrom. RNA-guided nucleases can be defined, in broad terms, by their PAM specificity and cleavage activity.

Suitably, the RNA-guided nuclease is a Type II CRISPR nuclease, for example a Cas9 nuclease. Cas9 is a dual RNA-guided endonuclease enzyme associated with the Clustered Regularly Interspaced Short Palindromic Repeats (CRISPR) adaptive immune system. Cas9 nucleases include the well-characterized ortholog from Streptococcus pyogenes (SpCas9). SpCas9 and other orthologs (including SaCas9, FnCa9, and AnaCas9) have been reviewed by Jiang, F. and Doudna, J.A., 2017. Annual review of biophysics, 46, pp.505-529.

The RNA-guided nuclease may be in a complex with the guide RNA, i.e. the guide RNA and the RNA-guided nuclease may together form a ribonucleoprotein (RNP). Suitably, the RNP is a Cas9 RNP. A RNP may be formed by any method known in the art, for example by incubating a RNA-guided nuclease with a guide RNA for 5-30 minutes at room temperature. Delivering Cas9 as a preassembled RNP can protect the guide RNA from intracellular degradation thus improving stability and activity of the RNA-guided nuclease (Kim S, et al. Genome Res. 2014;24(6):1012-9).

### Kit, Composition, Gene-Editing System

In one aspect, the present invention provides a kit, composition, or gene-editing system comprising the polynucleotide of the invention, the vector of the invention, and/or the guide RNA of the invention.

As used herein, a “gene-editing system” is a system which comprises all components necessary to edit a genome using the polynucleotide of the invention.

In some embodiments, the kit, composition, or gene-editing system comprises a polynucleotide and/or vector of the invention and a guide RNA. The guide RNA may correspond to the same DSB site targeted by the homology arms. For example, in some embodiments the kit, composition, or gene-editing system comprises:
(i) a polynucleotide comprising from 5′ to 3′: a first homology region, a splice acceptor sequence, a nucleotide sequence encoding a RAG1 polypeptide, and a second homology region, wherein the first homology region is homologous to a region upstream of chr 11: 36569295 and the second homology region is homologous to a region downstream of chr 11: 36569298, and/or a vector comprising said polynucleotide; and a guide RNA which comprises or consists of a nucleotide sequence that has at least 90% identity, at least 95% identity or 100% identity to SEQ ID NO: 41 or 53 (preferably SEQ ID NO: 41);
(ii) a polynucleotide comprising from 5′ to 3′: a first homology region, a splice acceptor sequence, a nucleotide sequence encoding a RAG1 polypeptide, and a second homology region, wherein the first homology region is homologous to a region upstream of chr 11: 36573790 and the second homology region is homologous to a region downstream of chr 11: 36573793 and/or a vector comprising said polynucleotide; and a guide RNA which comprises or consists of a nucleotide sequence that has at least 90% identity, at least 95% identity or 100% identity to SEQ ID NO: 42;
(iii) a polynucleotide comprising from 5′ to 3′: a first homology region, a splice acceptor sequence, a nucleotide sequence encoding a RAG1 polypeptide, and a second homology region, wherein the first homology region is homologous to a region upstream of chr 11: 36573641 and the second homology region is homologous to a region downstream of chr 11: 36573644 and/or a vector comprising said polynucleotide; and a guide RNA which comprises or consists of a nucleotide sequence that has at least 90% identity, at least 95% identity or 100% identity to SEQ ID NO: 43;
(iv) a polynucleotide comprising from 5′ to 3′: a first homology region, a splice acceptor sequence, a nucleotide sequence encoding a RAG1 polypeptide, and a second homology region, wherein the first homology region is homologous to a region upstream of chr 11: 36573351 and the second homology region is homologous to a region downstream of chr 11: 36573354 and/or a vector comprising said polynucleotide; and a guide RNA which comprises or consists of a nucleotide sequence that has at least 90% identity, at least 95% identity or 100% identity to SEQ ID NO: 44 or 54 (preferably SEQ ID NO: 44);
(v) a polynucleotide comprising from 5′ to 3′: a first homology region, a splice acceptor sequence, a nucleotide sequence encoding a RAG1 polypeptide, and a second homology region, wherein the first homology region is homologous to a region upstream of chr 11: 36569080 and the second homology region is homologous to a region downstream of chr 11: 36569083 and/or a vector comprising said polynucleotide; and a guide RNA which comprises or consists of a nucleotide sequence that has at least 90% identity, at least 95% identity or 100% identity to SEQ ID NO: 45;
(vi) a polynucleotide comprising from 5′ to 3′: a first homology region, a splice acceptor sequence, a nucleotide sequence encoding a RAG1 polypeptide, and a second homology region, wherein the first homology region is homologous to a region upstream of chr 11: 36572472 and the second homology region is homologous to a region downstream of chr 11: 36572475 and/or a vector comprising said polynucleotide; and a guide RNA which comprises or consists of a nucleotide sequence that has at least 90% identity, at least 95% identity or 100% identity to SEQ ID NO: 46;
(vii) a polynucleotide comprising from 5′ to 3′: a first homology region, a splice acceptor sequence, a nucleotide sequence encoding a RAG1 polypeptide, and a second homology region, wherein the first homology region is homologous to a region upstream of chr 11: 36571458 and the second homology region is homologous to a region downstream of chr 11: 36571461 and/or a vector comprising said polynucleotide; and a guide RNA which comprises or consists of a nucleotide sequence that has at least 90% identity, at least 95% identity or 100% identity to SEQ ID NO: 47;
(viii) a polynucleotide comprising from 5′ to 3′: a first homology region, a splice acceptor sequence, a nucleotide sequence encoding a RAG1 polypeptide, and a second homology region, wherein the first homology region is homologous to a region upstream of chr 11: 36571366 and the second homology region is homologous to a region downstream of chr 11: 36571369 and/or a vector comprising said polynucleotide; and a guide RNA which comprises or consists of a nucleotide sequence that has at least 90% identity, at least 95% identity or 100% identity to SEQ ID NO: 48 or 55 (preferably SEQ ID NO: 48);
(ix) a polynucleotide comprising from 5′ to 3′: a first homology region, a splice acceptor sequence, a nucleotide sequence encoding a RAG1 polypeptide, and a second homology region, wherein the first homology region is homologous to a region upstream of chr 11: 36572859 and the second homology region is homologous to a region downstream of chr 11: 36572862 and/or a vector comprising said polynucleotide; and a guide RNA which comprises or consists of a nucleotide sequence that has at least 90% identity, at least 95% identity or 100% identity to SEQ ID NO: 49;
(x) a polynucleotide comprising from 5′ to 3′: a first homology region, a splice acceptor sequence, a nucleotide sequence encoding a RAG1 polypeptide, and a second homology region, wherein the first homology region is homologous to a region upstream of chr 11: 36571457 and the second homology region is homologous to a region downstream of chr 11: 36571460 and/or a vector comprising said polynucleotide; and a guide RNA which comprises or consists of a nucleotide sequence that has at least 90% identity, at least 95% identity or 100% identity to SEQ ID NO: 50;
(xi) a polynucleotide comprising from 5′ to 3′: a first homology region, a splice acceptor sequence, a nucleotide sequence encoding a RAG1 polypeptide, and a second homology region, wherein the first homology region is homologous to a region upstream of chr 11: 36569351 and the second homology region is homologous to a region downstream of chr 11: 36569354 and/or a vector comprising said polynucleotide; and a guide RNA which comprises or consists of a nucleotide sequence that has at least 90% identity, at least 95% identity or 100% identity to SEQ ID NO: 51; or
(xii) a polynucleotide comprising from 5′ to 3′: a first homology region, a splice acceptor sequence, a nucleotide sequence encoding a RAG1 polypeptide, and a second homology region, wherein the first homology region is homologous to a region upstream of chr 11: 36572375 and the second homology region is homologous to a region downstream of chr 11: 36572378 and/or a vector comprising said polynucleotide; and a guide RNA which comprises or consists of a nucleotide sequence that has at least 90% identity, at least 95% identity or 100% identity to SEQ ID NO: 52.

In some embodiments, the kit, composition, or gene-editing system comprises:
(i) a polynucleotide comprising from 5′ to 3′: a first homology region, a splice acceptor sequence, a nucleotide sequence encoding a RAG1 polypeptide, and a second homology region, wherein the first homology region is homologous to a region upstream of chr 11: 36569295 and the second homology region is homologous to a region downstream of chr 11: 36569298, and/or a vector comprising said polynucleotide; and
(ii) a guide RNA which comprises or consists of a nucleotide sequence that has at least 90% identity, at least 95% identity or 100% identity to SEQ ID NO: 41 or 53 (preferably SEQ ID NO: 41).

In some embodiments, the kit, composition, or gene-editing system comprises:
(i) a polynucleotide comprising from 5′ to 3′: a first homology region, a splice acceptor sequence, a nucleotide sequence encoding a RAG1 polypeptide, and a second homology region, wherein the first homology region is homologous to a region comprising chr 11: 36569245-36569294 and the second homology region is homologous to a region comprising chr 11: 36569299-36569348, and/or a vector comprising said polynucleotide; and
(ii) a guide RNA which comprises or consists of a nucleotide sequence that has at least 90% identity, at least 95% identity or 100% identity to SEQ ID NO: 41 or 53 (preferably SEQ ID NO: 41).

In some embodiments, the kit, composition, or gene-editing system comprises:
(i) a polynucleotide comprising from 5′ to 3′: a first homology region, a splice acceptor sequence, a nucleotide sequence encoding a RAG1 polypeptide, and a second homology region, wherein the first homology region comprises or consists of a nucleotide sequence that has at least 70% identity, at least 80% identity, at least 90% identity, at least 95% identity, at least 98% identity, or 100% identity to SEQ ID NO: 7 and the second homology region comprises or consists of a nucleotide sequence that has at least 70% identity, at least 80% identity, at least 90% identity, at least 95% identity, at least 98% identity, or 100% identity to SEQ ID NO: 19, and/or a vector comprising said polynucleotide; and
(ii) a guide RNA which comprises or consists of a nucleotide sequence that has at least 90% identity, at least 95% identity or 100% identity to SEQ ID NO: 41 or 53 (preferably SEQ ID NO: 41).

In some embodiments, the kit, composition, or gene-editing system comprises:
(i) a polynucleotide comprising from 5′ to 3′: a first homology region, a splice acceptor sequence, a nucleotide sequence encoding a RAG1 polypeptide, and a second homology region, wherein the first homology region comprises or consists of a nucleotide sequence that has at least 70% identity, at least 80% identity, at least 90% identity, at least 95% identity, at least 98% identity, or 100% identity to SEQ ID NO: 31, or a fragment thereof; and the second homology region comprises or consists of a nucleotide sequence that has at least 70% identity, at least 80% identity, at least 90% identity, at least 95% identity, at least 98% identity, or 100% identity to SEQ ID NO: 32, or a fragment thereof, and/or a vector comprising said polynucleotide; and
(ii) a guide RNA which comprises or consists of a nucleotide sequence that has at least 90% identity, at least 95% identity or 100% identity to SEQ ID NO: 41 or 53 (preferably SEQ ID NO: 41).

The kit, composition, or gene-editing system may further comprise an RNA-guided nuclease. Suitably, the RNA-guided nuclease corresponds to the guide RNA used. For example, if the guide RNA comprises or consists of a nucleotide sequence that has at least 90% identity, at least 95% identity or 100% identity to any one of SEQ ID NOs: 41-52, the RNA-guided nuclease is suitably a Cas9 endonuclease. For example, if the guide RNA comprises or consists of a nucleotide sequence that has at least 90% identity, at least 95% identity or 100% identity to any one of SEQ ID NOs: 53-55, the RNA-guided nuclease is suitably a Cas9 endonuclease.

The RNA-guided nuclease may be in a complex with the guide RNA, i.e. the guide RNA and the RNA-guided nuclease together form a ribonucleoprotein (RNP).

### Cell

In one aspect, the present invention provides a cell which has been edited using the polynucleotide, vector, kit, composition, or gene-editing system of the present invention.

In a related aspect, the present invention provides a cell comprising the polynucleotide, vector and/or genome of the present invention.

Suitably, the cell is an isolated cell. Suitably, the cell is a mammalian cell, for example a human cell.

Suitably, the cell is a hematopoietic stem cell (HSC), a hematopoietic progenitor cell (HPC), or a lymphoid progenitor cell (LPC). In some embodiments, the cell is a HSC or a HPC, optionally the cell is a HSC.

As used herein “hematopoietic stem cells” are stem cells that have no differentiation potential to cells other than hematopoietic cells, “hematopoietic progenitor cells” are progenitor cells that have no differentiation potential to cells other than hematopoietic cells, and “lymphoid progenitor cells” are progenitor cells that have no differentiation potential to cells other than lymphocytes.

The cell can be obtained from any source. The cell may be autologous or allogeneic. The cell may be obtained or obtainable from any biological sample, such as peripheral blood or cord blood. Peripheral blood may be treated with mobilising agent, i.e. may be mobilised peripheral blood. The cell may be a universal cell.

The cell may be isolated or isolatable using commercially available antibodies that bind to cell surface antigens, e.g. CD34, using methods known to those of skill in the art. For example, the antibodies may be conjugated to magnetic beads and immunological procedures utilized to recover the desired cell type. Suitably, the cell is identified by the presence or absence of one or more antigenic markers. Suitable antigenic markers include CD34, CD133, CD90, CD45, CD4, CD19, CD13, CD3, CD56, CD14, CD61/41, CD135, CD45RA, CD33, CD66b, CD38, CD45, CD10, CD11c, CD19, CD7, and CD71.

Suitably, the cell is identified by the presence of the antigenic marker CD34 (CD34+), i.e. the cell is a CD34+ cell. For example, the cell may be a cord blood CD34+ cell or a (mobilised) peripheral blood CD34+ cell. The cell may be a CD34+ HSC, a CD34+ HPC, or a CD34+ LPC, optionally the cell is a CD34+ HSC.

In some embodiments, the cell is identified by the presence of CD34 and the presence or absence or one or more further antigenic markers. The further antigenic markers may be selected from one or more of CD133, CD90, CD3, CD56, CD14, CD61/41, CD135, CD45RA, CD33, CD66b, CD38, CD45, CD10, CD11c, CD19, CD7, and CD71. For example, the cell may be a CD34+CD133+CD90+ cell, a CD34+CD133+CD90- cell, or a CD34+CD133-CD90-cell.

Suitably, the cell is a NALM6 cell, a K562 cell, or other human cell (e.g. a Molt4 cell, a U937 cell, etc.). Suitably, the cell is a T cell.

### Population of Cells

In one aspect, the present invention provides a population or cells comprising the cell of the present invention. Suitably, at least 1%, at least 2%, at least 5%, at least 10%, or at least 20% of the cells in the population of cells are cells of the present invention. Suitably, the population of cells comprises at least 10×10⁵, at least 50×10⁵, or at least 100×10⁵ cells of the present invention.

In a related aspect, the present invention provides a population of cells which have been edited using the polynucleotide, vector, kit, composition, or gene-editing system of the present invention. Suitably, at least 1%, at least 2%, at least 5%, at least 10%, or at least 20% of the cells in the population of cells are cells which have been edited using the polynucleotide, vector, kit, composition, or gene-editing system of the present invention. Suitably, the population of cells comprises at least 10×10⁵, at least 50×10⁵, or at least 100×10⁵ cells which have been edited using the polynucleotide, vector, kit, composition, or gene-editing system of the present invention.

In a related aspect, the present invention provides a population of cells comprising the polynucleotide, vector and/or genome of the present invention. Suitably, at least 1%, at least 2%, at least 5%, at least 10%, or at least 20% of the cells in the population of cells are cells comprising the polynucleotide, vector and/or genome of the present invention. Suitably, the population of cells comprises at least 10×10⁵, at least 50×10⁵, or at least 100×10⁵ cells comprising the polynucleotide, vector and/or genome of the present invention.

Suitably, the population of cells are mammalian cells, for example human cells. The population of cells may be autologous or allogeneic. Suitably, the population of cells are obtained or obtainable from (mobilised) peripheral blood or cord blood. The population of cells may be universal cells.

Suitably, at least 50%, at least 60%, at least 70%, or at least 80% of the population of cells are HSCs, HPCs, and/or LPCs. Suitably, at least 50%, at least 60%, at least 70%, or at least 80% of the population of cells are CD34+ cells.

In some embodiments, at least 1%, at least 2%, at least 5%, at least 10%, or at least 20% of the population of cells are CD34+ cells comprising the polynucleotide, vector and/or genome of the present invention. For example, in some embodiments at least 20% of the population of cells are CD34+ cells comprising the genome of the present invention.

In some embodiments, the population of cells comprises at least 10×10⁵, at least 50×10⁵, or at least 100×10⁵ CD34+ cells comprising the polynucleotide, vector and/or genome of the present invention. For example, in some embodiments the population of cells comprises at least 100×10⁵ CD34+ cells comprising the genome of the present invention.

### Method of Gene Editing

In one aspect, the present invention provides a method of gene editing a cell or a population of cells using polynucleotides, vectors, guide RNAs, kits, compositions and/or gene-editing system of the present invention. The present invention also provide a population of gene-edited cells obtained or obtainable by said methods.

In another aspect the present invention provides use of a polynucleotide, vector, guide RNA, kit, composition, and/or gene-editing system of the present invention for gene editing a cell or a population of cells.

Suitably, the method of gene editing a cell or a population of cells comprises:
(a) providing a cell or a population of cells; and
(b) using a kit, composition, and/or gene-editing system described herein to obtain a gene-edited cell or a population of gene-edited cells.

For example, the method of gene editing a cell or a population of cells comprises:
(a) providing a cell or a population of cells; and
(b) delivering an RNA-guided nuclease, a guide RNA, and/or a polynucleotide or vector of the present invention to the cell or population of cells to obtain a gene-edited cell or a population of gene-edited cells.

The gene-edited cell or population of gene-edited cells may be as defined herein. The present invention also provides a gene-edited cell or population of gene-edited cells obtained or obtainable by said method.

### Step (a) Providing a Cell or a Population of Cells

The population of cells may be obtained or obtainable from any suitable source. Suitably, the population of cells are obtained or obtainable from (mobilised) peripheral blood or cord blood. The population of cells may be obtained or obtainable from a subject, e.g. a subject to be treated. Suitably, the population of cells may be isolated and/or enriched from a biological sample by any method known in the art, for example by FACS and/or magnetic bead sorting.

Suitably, the population of cells are mammalian cells, for example human cells. The population of cells may be, for example, autologous or allogeneic. The population of cells may be, for example, universal cells.

Suitably, the population of cells comprises about 1 × 10⁵ cells per well to about 10 × 10⁵ cells per well, e.g. about 2 × 10⁵ cells per well, or about 5 × 10⁵ cells per well.

The population of cells may comprise HSCs, HPCs, and/or LPCs. Suitably, at least 50%, at least 60%, at least 70%, or at least 80% of the population of cells are HSCs, HPCs, and/or LPCs. In some embodiments, the population of cells consists essentially of HSCs, HPCs, and/or LPCs, or consists of HSCs, HPCs, and/or LPCs.

The population of cells may comprise CD34+ cells, e.g. CD34+ HSCs, HPCs, and/or LPCs. Suitably, at least 50%, at least 60%, at least 70%, or at least 80% of the population of cells are CD34+ cells, e.g. CD34+ HSCs, HPCs, and/or LPCs. In some embodiments, the population of cells consists essentially of CD34+ cells, e.g. CD34+ HSCs, HPCs, and/or LPCs, or consists of CD34+ cells, e.g. CD34+ HSCs, HPCs, and/or LPCs.

The population of cells may comprise CD34+CD133+CD90+ cells, CD34+CD133+CD90-cells, and/or CD34+CD133-CD90-. Suitably, at least 50%, at least 60%, at least 70%, or at least 80% of the population of cells are CD34+CD133+CD90+ cells, CD34+CD133+CD90-cells, and/or CD34+CD133-CD90- cells. In some embodiments, the population of cells consists essentially of CD34+CD133+CD90+ cells, CD34+CD133+CD90- cells, and/or CD34+CD133-CD90- cells, or consists of CD34+CD133+CD90+ cells, CD34+CD133+CD90-cells, and/or CD34+CD133-CD90- cells.

The cell or population of cells may be cultured prior to step (b). The pre-culturing step may comprise a pre-activation step and/or a pre-expansion step, optionally the pre-culturing step is a pre-activation step.

As used herein, a “pre-culturing step” refers to a culturing step which occurs prior to genetic modification of the cells. As used herein, a “pre-activating step” refers to an activation step or stimulation step which occurs prior to genetic modification of the cells. As used herein, a “pre-expansion step” refers to an expansion step which occurs prior to genetic modification of the cells.

Suitably, the method may comprise:
(a1) providing a population of cells;
(a2) pre-culturing (e.g. pre-activating and/or pre-expanding) the population of cells to obtain a pre-cultured (e.g. pre-activated and/or pre-expanded) population of cells;
(b) delivering an RNA-guided nuclease, a guide RNA, and/or a polynucleotide or vector of the present invention to the pre-cultured (e.g. pre-activated and/or pre-expanded) population of cells to obtain a population of gene-edited cells.

The pre-culturing step (e.g. pre-activation step and/or pre-expansion step) may be carried out using any suitable conditions.

During the pre-culturing step (e.g. pre-activation step and/or pre-expansion step) the population of cells may be seeded at a concentration of about 1 × 10⁵ cells/ml to about 10 × 10⁵ cells/ml, e.g. about 2 × 10⁵ cells/ml, or about 5 × 10⁵ cells/ml.

Suitably, the pre-culturing step (e.g. pre-activation step and/or pre-expansion step) is at least 1 day, at least 2 days, or at least 3 days. Suitably, the population of cells are pre-cultured (e.g. pre-activated and/or pre-expanded) for about 3 days. Suitably, the population of cells are pre-cultured in a 5% CO₂ humidified atmosphere at 37° C.

Any suitable culture medium may be used. For example, commercially available medium such as StemSpan medium may be used, which contains bovine serum albumin, insulin, transferrin, and supplements in Iscove’s MDM. The culture medium may be supplemented with one or more antibiotic (e.g. penicillin, streptomycin).

The pre-culturing step (e.g. pre-activation step and/or pre-expansion step) may be carried out in the presence in of one or more cytokines and/or growth factors. As used herein, a “cytokine” is any cell signalling substance and includes chemokines, interferons, interleukins, lymphokines, and tumour necrosis factors. As used herein, a “growth factor” is any substance capable of stimulating cell proliferation, wound healing, or cellular differentiation. The terms “cytokine” and “growth factor” may overlap.

The pre-culturing step (e.g. pre-activation step and/or pre-expansion step) may be carried out in the presence of one or more early-acting cytokine, one or more transduction enhancer, and/or one or more expansion enhancer.

### Early-Acting Cytokines

As used herein, an “early-acting cytokine” is a cytokine which stimulates HSCs, HPCS, and/or LPCs or CD34+ cells. Early-acting cytokines include thrombopoietin (TPO), stem cell factor (SCF), Flt3-ligand (FLT3-L), interleukin (IL)-3, and IL-6. In some embodiments, the pre-culturing step (e.g. pre-activation step and/or pre-expansion step) is carried out in the presence of at least one early-acting cytokine. Any suitable concentration of early-acting cytokine may be used. For example, 1-1000 ng/ml, or 10-1000 ng/ml, or 10-500 ng/ml.

In some embodiments, the pre-culturing step (e.g. pre-activation step and/or pre-expansion step) is carried out in the presence of SCF. The concentration of SCF may be about 10-1000 ng/ml, about 50-500 ng/ml, or about 100-300 ng/ml.

In some embodiments, the pre-culturing step (e.g. pre-activation step and/or pre-expansion step) is carried out in the presence of FLT3-L. The concentration of FLT3-L may be about 10-1000 ng/ml, about 50-500 ng/ml, or about 100-300 ng/ml.

In some embodiments, the pre-culturing step (e.g. pre-activation step and/or pre-expansion step) is carried out in the presence of TPO. The concentration of TPO may be about 5-500 ng/ml, about 10-200 ng/ml, or about 20-100 ng/ml.

In some embodiments, the pre-culturing step (e.g. pre-activation step and/or pre-expansion step) is carried out in the presence of IL-3. The concentration of IL-3 may be about 10-200 ng/ml, about 20-100 ng/ml, or about 60 ng/ml.

In some embodiments, the pre-culturing step (e.g. pre-activation step and/or pre-expansion step) is carried out in the presence of IL-6. The concentration of IL-6 may be about 5-100 ng/ml, about 10-50 ng/ml, or about 20 ng/ml.

In some embodiments, the pre-culturing step (e.g. pre-activation step and/or pre-expansion step) is carried out in the presence of SCF (e.g. in a concentration of about 100 ng/ml), FLT3-L (e.g. in a concentration of about 100 ng/ml), TPO (e.g. in a concentration of about 20 ng/ml) and IL-6 (e.g. in a concentration of about 20 ng/ml), in particular when the population of cells are cord-blood CD34+ cells.

In some embodiments, the pre-culturing step (e.g. pre-activation step and/or pre-expansion step) is carried out in the presence of SCF (e.g. in a concentration of about 300 ng/ml), FLT3-L (e.g. in a concentration of about 300 ng/ml), TPO (e.g. in a concentration of about 100 ng/ml) and IL-3 (e.g. in a concentration of about 60 ng/ml), in particular when the population of cells are (mobilised) peripheral blood CD34+ cells.

### Transduction Enhancers

As used herein, a “transduction enhancer” is a substance that is capable of improving viral transduction of HSCs, HPCS, and/or LPCs or CD34+ cells. Suitable transduction enhancers include LentiBOOST, prostaglandin E2 (PGE2), protamine sulfate (PS), Vectofusin-1, ViraDuctin, RetroNectin, staurosporine (Stauro), 7-hydroxy-stauro, human serum albumin, polyvinyl alcohol, and cyclosporin H (CsH). In some embodiments, the pre-culturing step (e.g. pre-activation step and/or pre-expansion step) is carried out in the presence of at least one transduction enhancer. Any suitable concentration of transduction enhancer may be used, for example as described in Schott, J.W., et al., 2019. Molecular Therapy-Methods & Clinical Development, 14, pp.134-147 or Yang, H., et al., 2020. Molecular Therapy-Nucleic Acids, 20, pp. 451-458.

In some embodiments, the pre-culturing step (e.g. pre-activation step and/or pre-expansion step) is carried out in the presence of PGE2. Suitably, the PGE2 is 16,16-dimethyl prostaglandin E2 (dmPGE2). The concentration of PGE2 may be about 1-100 µM, about 5-20 µM, or about 10 µM.

In some embodiments, the pre-culturing step (e.g. pre-activation step and/or pre-expansion step) is carried out in the presence of CsH. The concentration of CsH may be about 1-50 µM, 5-50 µM, about 10-50 µM, or about 10 µM.

### Expansion Enhancers

As used herein, an “expansion enhancer” is a substance that is capable of improving expansion of HSCs, HPCS, and/or LPCs or CD34+ cells. Suitable expansion enhancers include UM171, UM729, StemRegenin1 (SR1), diethylaminobenzaldehyde (DEAB), LG1506, BIO (GSK3β inhibitor), NR-101, trichostatin A (TSA), garcinol (GAR), valproic acid (VPA), copper chelator, tetraethylenepentamine, and nicotinamide. In some embodiments, the pre-culturing step (e.g. pre-activation step and/or pre-expansion step) is carried out in the presence of at least one expansion enhancer. Any suitable concentration of expansion enhancer may be used, for example as described in Huang, X., et al., 2019. F1000Research, 8, 1833.

In some embodiments, the pre-culturing step (e.g. pre-activation step and/or pre-expansion step) is carried out in the presence of UM171 or UM729. The concentration of UM171 may be about 10-200 nM, about 20-100 nM, or about 50 nM.

In some embodiments, the pre-culturing step (e.g. pre-activation step and/or pre-expansion step) is carried out in the presence of SR1. The concentration of SR1 may be about 0.1-10 µM, about 0.5-5 µM, or about 1 µM.

In some embodiments, the pre-culturing step (e.g. pre-activation step and/or pre-expansion step) is carried out in the presence of UM171 (e.g. in a concentration of about 50 nM) or UM729 and SR1 (e.g. in a concentration of about 1 µM).

In some embodiments, the pre-culturing step (e.g. pre-activation step and/or pre-expansion step) is carried out in the presence of SCF (e.g. in a concentration of about 100 ng/ml), FLT3-L (e.g. in a concentration of about 100 ng/ml), TPO (e.g. in a concentration of about 20 ng/ml), IL-6 (e.g. in a concentration of about 20 ng/ml), PGE2 (e.g. in a concentration of about 10 µM), UM171 (e.g. in a concentration of about 50 nM), and SR1 (e.g. in a concentration of about 1 µM), in particular when the population of cells are cord-blood CD34+ cells.

In some embodiments, the pre-culturing step (e.g. pre-activation step and/or pre-expansion step) is carried out in the presence of SCF (e.g. in a concentration of about 300 ng/ml), FLT3-L (e.g. in a concentration of about 300 ng/ml), TPO (e.g. in a concentration of about 100 ng/ml), IL-3 (e.g. in a concentration of about 60 ng/ml), PGE2 (e.g. in a concentration of about 10 µM), UM171 (e.g. in a concentration of about 50 nM), and SR1 (e.g. in a concentration of about 1 µM), in particular when the population of cells are (mobilised) peripheral blood CD34+ cells.

### Step (b) Obtaining a Gene-Edited Cell or a Population of Gene-Edited Cells

A kit, composition, and/or gene-editing system comprising an RNA-guided nuclease, a guide RNA, and/or a polynucleotide or vector of the present invention may, for example, be used to obtain the gene-edited cell or a population of gene-edited cells.

The RNA-guided nuclease, guide RNA, and/or polynucleotide or vector may be any suitable combination described herein. The guide RNA may correspond to the same DSB site targeted by the homology arms. The RNA-guided nuclease may correspond to the guide RNA used. For example:
(i) the RNA-guided nuclease may be a Cas9 endonuclease;
(ii) the guide RNA may be a guide RNA comprising or consisting of a nucleotide sequence that has at least 90% identity or at least 95% identity to any of SEQ ID NOs: 41-52 or 53-55, optionally wherein the guide RNA comprises or consists of a nucleotide sequence that has at least 90% identity or at least 95% identity to SEQ ID NO: 41 or 53 (preferably SEQ ID NO: 41); and
(iii) the polynucleotide may be a polynucleotide comprising from 5′ to 3′: a first homology region, a splice acceptor sequence, a nucleotide sequence encoding a RAG1 polypeptide, and a second homology region, wherein the first homology region comprises or consists of a nucleotide sequence that has at least 70% identity, at least 80% identity, at least 90% identity, at least 95% identity, at least 98% identity, or 100% identity to any of SEQ ID NOs: 7-18 and/or the second homology region comprises or consists of a nucleotide sequence that has at least 70% identity, at least 80% identity, at least 90% identity, at least 95% identity, at least 98% identity, or 100% identity to any of SEQ ID NOs: 19-30; or the vector may be a vector comprising said polynucleotide.

In some embodiments:
(i) the RNA-guided nuclease may be a Cas9 endonuclease;
(ii) the guide RNA comprises or consists of a nucleotide sequence that has at least 90% identity, at least 95% identity or 100% identity to SEQ ID NO: 41 or 53 (preferably SEQ ID NO: 41); and
(iii) the polynucleotide comprises from 5′ to 3′: a first homology region, a splice acceptor sequence, a nucleotide sequence encoding a RAG1 polypeptide, and a second homology region, wherein the first homology region comprises or consists of a nucleotide sequence that has at least 70% identity, at least 80% identity, at least 90% identity, at least 95% identity, at least 98% identity, or 100% identity to SEQ ID NO: 31, or a fragment thereof; and the second homology region comprises or consists of a nucleotide sequence that has at least 70% identity, at least 80% identity, at least 90% identity, at least 95% identity, at least 98% identity, or 100% identity to SEQ ID NO: 32, or a fragment thereof; or the vector comprises said polynucleotide.

### Delivery of a RNA-Guided Nuclease, Guide RNA, And/or Polynucleotide or Vector

The RNA-guided nuclease, guide RNA, and/or polynucleotide or vector may be delivered to the cell by any suitable technique. For example, the RNA-guided nuclease may be delivered directly using electroporation, microinjection, bead loading or the like, or indirectly via transfection and/or transduction. The guide RNA, and/or polynucleotide or vector may be introduced by transfection and/or transduction.

As used herein “transfection” is a process using a non-viral vector to deliver a polypeptide and/or polynucleotide to a target cell. Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) and combinations thereof.

As used herein “transduction” is a process using a viral vector to deliver a polynucleotide to a target cell. Typical transduction methods include infection with recombinant viral vectors, such as adeno-associated viral, retroviral, lentiviral, adenoviral, baculoviral and herpes simplex viral vectors.

The RNA-guided nuclease and the guide RNA may be delivered by any suitable method, for instance any method described in Wilbie, D., et al., 2019. Accounts of chemical research, 52(6), pp.1555-1564. Suitably, the RNA-guided nuclease and the guide RNA are delivered together preassembled as in the form of a RNP complex. The RNP complex may be delivered by electroporation.

Any suitable dose of the RNA-guided nuclease and/or the guide RNA may be used. For example, the guide RNA may be delivered at a dose of about 10-100 pmol/well, optionally about 50 pmol/well. For example, the RNP may be delivered at a dose of about 1-10 µM, optionally 1-2.5 µM.

The RNA-guided nuclease and/or the guide RNA may be delivered prior to the vector and/or simultaneously with the polynucleotide or vector of the invention. Suitably, the RNA-guided nuclease and/or the guide RNA are delivered prior to the polynucleotide or vector. For example, the RNA-guided nuclease and/or the guide RNA may be delivered about 1-100 minutes, about 5-30, or about 15 minutes, prior to the polynucleotide or vector.

The polynucleotide or vector of the invention may be delivered by any suitable method. For example, when the polynucleotide may be in a viral vector or the vector may be a viral vector and delivered by transduction.

Any suitable dose of the polynucleotide or vector may be used. For example, the vector may be delivered at a MOI of about 10⁴ to 10⁵ vg/cell, optionally about 10⁴ vg/cell.

### Delivery of a P53 Inhibitor And/or HDR Enhancer

The method may further comprise a step of delivering a p53 inhibitor and/or HDR enhancer. The p53 inhibitor and/or HDR enhancer may be delivered simultaneously. The p53 inhibitor and/or HDR enhancer may be delivered simultaneously with or after the RNA-guided nuclease and/or the guide RNA.

As used herein, a “p53 inhibitor” is a substance which inhibits activation of the p53 pathway. The p53 pathway plays a role in regulation or progression through the cell cycle, apoptosis, and genomic stability by means of several mechanisms including: activation of DNA repair proteins, arrest of the cell cycle; and initiation of apoptosis. Inhibition of this p53 response by delivery during editing has been shown to increase hematopoietic repopulation by treated cells (Schiroli, G. et al. 2019. Cell Stem Cell 24, 551-565). Suitably, the p53 inhibitors is a dominant-negative p53 mutant protein, e.g. GSE56.

GSE56 may have the amino acid sequence:

CPGRDRRTEEENFRKKEEHCPELPPGSAKRALPTSTSSSPQQKKKPLDGE
YFTLKIRGRERFEMFRELNEALELKDARAAEESGDSRAHSSYPK

(SEQ ID NO: 67)

In one embodiment, the p53 dominant negative peptide is a variant of GSE56 comprising 1, 2, 3, 4, 5, 6, 7, 8, 9 or 10 amino acid substitutions, additions or deletions, while retaining the activity of GSE56, for example in reducing or preventing p53 signalling.

In one embodiment, the p53 dominant negative peptide comprises an amino acid sequence having at least 70%, 75%, 80%, 85%, 90%, 95%, 96%, 97%, 98% or 99% identity to SEQ ID NO: 67.

As used herein, an “HDR enhancer” is a substance that is capable of improving HDR efficiency in HSCs, HPCS, and/or LPCs or CD34+ cells. HDR is constrained in long-term-repopulating HSCs. Any suitable HDR enhancer may be used, for example as described in Ferrari, S., et al., 2020. Nature Biotechnology, pp.1-11. Suitably, the HDR enhancer is the adenovirus 5 E4orf6/7 protein. Adenovirus 5 E4orf6/7 proteins may be as disclosed in WO 2020/002380 (incorporated herein by reference).

The p53 inhibitor and the HDR enhancer may be delivered by any suitable method. The p53 inhibitor and/or the HDR enhancer may be transiently expressed, for example the p53 inhibitor and/or the HDR enhancer may delivered via mRNA. The p53 inhibitor and the HDR enhancer may be delivered by separate mRNAs or on a single mRNA encoding a fusion protein, optionally with a self-cleaving peptide (e.g. P2A). Any suitable dose of the p53 inhibitor and/or the HDR enhancer may be used, for example mRNA be delivered at a concentration of about 10-1000 µg/ml, about 50-500 µg/ml, or about 150 µg/ml.

In some embodiments, step (b) comprises:
(b1) delivering a RNA-guided nuclease and a guide RNA of the invention, optionally preassembled in the form of a RNP complex by electroporation;
(b2) optionally, delivering a p53 inhibitor and/or a HDR enhancer; and
(b3) delivering a polynucleotide or vector of the invention by transduction to provide a gene-edited population of cells.

### Culturing the Gene-Edited Cell or Population of Gene-Edited Cells

The method may further comprise a step of culturing the population of gene-edited cells. This may be an expansion step, i.e. the method may further comprises a step of expanding the population of gene-edited cells.

The culturing step (e.g. expansion step) may be carried out using any suitable conditions.

During the culturing step (e.g. expansion step) the population of cells may be seeded at a concentration of about 1 × 10⁵ cells/ml to about 10 × 10⁵ cells/ml, e.g. about 2 × 10⁵ cells/ml, or about 5 × 10⁵ cells/ml. Suitably, the culturing step (e.g. expansion step) is for at least one day, or one to five days. For example, the culturing step (e.g. expansion step) may be for about one day. Suitably, the population of cells are cultured in a 5% CO₂ humidified atmosphere at 37° C.

Any suitable culture medium may be used. For example, commercially available medium such as StemSpan medium may be used, which contains bovine serum albumin, insulin, transferrin, and supplements in Iscove’s MDM. The culture medium may be supplemented with one or more antibiotic (e.g. penicillin, streptomycin). The culturing step (e.g. expansion step) may be carried out in the presence in of one or more cytokines and/or growth factors.

In some embodiments, step (b) comprises:
(b1) delivering a RNA-guided nuclease and a guide RNA of the invention, optionally preassembled in the form of a RNP complex by electroporation;
(b2) optionally, delivering a p53 inhibitor and/or a HDR enhancer;
(b3) delivering a polynucleotide or vector of the invention by transduction to provide a gene-edited population of cells; and
(b4) culturing (e.g. expanding) the gene-edited population of cells.

### Methods of Treatment

In one aspect the present invention provides a method of treating a subject using polynucleotides, vectors, guide RNAs, kits, compositions, gene-editing systems, cells and/or populations of cells of the present invention. Suitably, the method of treating a subject may comprise administering a cell or population of cells of the present the invention.

In a related aspect the present invention provides a polynucleotide, vector, guide RNA, kit, composition, gene-editing system, cell and/or populations of cells of the present invention for use as a medicament. Suitably, the cell or population of cells of the present the invention may be used as a medicament.

In a related aspect, the present invention provides use of a polynucleotide, vector, guide RNA, kit, composition, gene-editing system, cell and/or populations of cells of the present invention for the manufacture of a medicament. Suitably, the cell or population of cells of the present the invention may be used for the manufacture of a medicament.

Suitably, a method of treating a subject may comprise:
(a) providing a cell or a population of cells;
(b) using a kit, composition, and/or gene-editing system described herein to obtain a gene-edited cell or a population of gene-edited cells; and
(c) administering the population of gene-edited cells to the subject.

For example, a method of treating a subject may comprise:
(a) providing a cell or a population of cells;
(b) delivering an RNA-guided nuclease, a guide RNA, and/or a polynucleotide or vector of the present invention to the cell or population of cells to obtain a gene-edited cell or a population of gene-edited cells; and
(c) administering the population of gene-edited cells to the subject.

Steps (a) and (b) may be identical to the steps described in the section above.

Suitably, the cell of population of cells may be isolated and/or enriched from the subject to be treated, e.g. the population of cells may be an autologous population of CD34+ cells. Suitably, the population of cells are isolated from (mobilised) peripheral blood or cord blood of the subject to be treated and subsequently enriched (e.g. by FACS and/or magnetic bead sorting).

The subject may be immunocompromised and/or the disease to be treated may be an immunodeficiency, i.e. the medicament may be for treating an immunodeficiency. As used herein, an “immunodeficiency” is a disease in which the immune system’s ability to fight infectious disease and cancer is compromised or entirely absent. A subject who has an immunodeficiency is said to be “immunocompromised”. An immunocompromised person may be particularly vulnerable to opportunistic infections, in addition to normal infections that could affect everyone.

### RAG Deficient-Immunodeficiency

The subject may have RAG deficiency, e.g. a RAG1 deficiency. A RAG1 deficiency may be due to a loss-of-function mutation in the RAG1 gene, optionally a loss-of-function mutation in the RAG1 exon 2.

The immunodeficiency may be a RAG deficient-immunodeficiency. As used herein, a “RAG deficient-immunodeficiency” is an immunodeficiency characterised by loss of RAG1/RAG2 activity. A RAG deficient-immunodeficiency may, for example be caused by a mutation in RAG genes.

Suitably, the RAG deficient-immunodeficiency may be a RAG1 deficiency. A RAG1 deficiency may be due to a loss-of-function mutation in the RAG1 gene, optionally a loss-of-function mutation in the RAG1 exon 2.

Mutations of the RAG genes in humans are associated with distinct clinical phenotypes, which are characterized by variable association of infections and autoimmunity. In some cases, environmental factors have been shown to contribute to such phenotypic heterogeneity. In humans, RAG1 deficiency can cause a broad spectrum of phenotypes, including T- B- SCID, Omenn syndrome (OS), atypical SCID (AS) and combined immunodeficiency with granuloma/autoimmunity (CID-G/Al). (Notarangelo, L.D., et al., 2016. Nature Reviews Immunology, 16(4), pp.234-246 and Delmonte, O.M., et al., 2018. Journal of clinical immunology, 38(6), pp.646-655).

In some embodiments, the RAG deficient-immunodeficiency is T- B- SCID, Omenn syndrome, atypical SCID, or CID-G/Al.

Severe combined immunodeficiency (SCID) comprises a heterogeneous group of disorders that are characterized by profound abnormalities in the development and function of T cells (and also B cells in some forms of SCID), and are associated with early-onset severe infections. This condition is inevitably fatal early in life, unless immune reconstitution is achieved, usually with HSCT. Following the introduction of newborn screening for SCID in the United States, it has become possible to establish that RAG mutations account for 19% of all cases of SCID and SCID-related conditions, and are a prominent cause of atypical SCID and Omenn syndrome in particular. (Notarangelo, L.D., et al., 2016. Nature Reviews Immunology, 16(4), pp.234-246).

In 1996, RAG mutations were identified as the main cause of T-B- SCID with normal cellular radiosensitivity. A distinct phenotype characterizes Omenn syndrome, which was first described in 1965. These patients manifest early-onset generalized erythroderma, lymphadenopathy, hepatosplenomegaly, eosinophilia and severe hypogammaglobulinaemia with increased IgE levels, which are associated with the presence of autologous, oligoclonal and activated T cells that infiltrate multiple organs. In some patients with hypomorphic RAG mutations, a residual presence of autologous T cells was demonstrated without clinical manifestations of Omenn syndrome. This condition is referred to as ‘atypical’ or ‘leaky’ SCID. A distinct SCID phenotype involving the oligoclonal expansion of autologous γδ T cells (referred to here as γδ T+ SCID) has been reported in infants with RAG deficiency and disseminated cytomegalovirus (CMV) infection. (Notarangelo, L.D., et al., 2016. Nature Reviews Immunology, 16(4), pp.234-246).

Whereas SCID, atypical SCID and Omenn syndrome are inevitably fatal early in life if untreated, several forms of RAG deficiency with a milder clinical course and delayed presentation have been reported in recent years. In particular, the occurrence of CID-G/Al was reported in three unrelated girls with RAG mutations who manifested granulomas in the skin, mucous membranes and internal organs, and had severe complications after viral infections, including B cell lymphoma. Following this description, several other cases of CID-G/Al with various autoimmune manifestations (such as cytopaenias, vitiligo, psoriasis, myasthenia gravis and Guillain-Barré syndrome) have been reported. (Notarangelo, L.D., et al., 2016. Nature Reviews Immunology, 16(4), pp.234-246).

Additional phenotypes that are associated with RAG deficiency include idiopathic CD4+ T cell lymphopaenia, common variable immunodeficiency, IgA deficiency, selective deficiency of polysaccharide-specific antibody responses, hyper-lgM syndrome and sterile chronic multifocal osteomyelitis. (Notarangelo, L.D., et al., 2016. Nature Reviews Immunology, 16(4), pp.234-246).

The skilled person will understand that they can combine all features of the invention disclosed herein without departing from the scope of the invention as disclosed.

Preferred features and embodiments of the invention will now be described by way of nonlimiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E.F. and Maniatis, T. (1989) Molecular Cloning: A Laboratory Manual, 2nd Edition, Cold Spring Harbor Laboratory Press; Ausubel, F.M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J.M. and McGee, J.O’D. (1990) In Situ Hybridization: Principles and Practice, Oxford University Press; Gait, M.J. (1984) Oligonucleotide Synthesis: A Practical Approach, IRL Press; and Lilley, D.M. and Dahlberg, J.E. (1992) Methods in Enzymology: DNA Structures Part A: Synthesis and Physical Analysis of DNA, Academic Press. Each of these general texts is herein incorporated by reference.

### EXAMPLES

### EXAMPLE 1 - Editing the RAG1 Gene

### Results

We have developed a platform to correct CD34⁺ hematopoietic stem cells by exploiting a gene targeting approach.

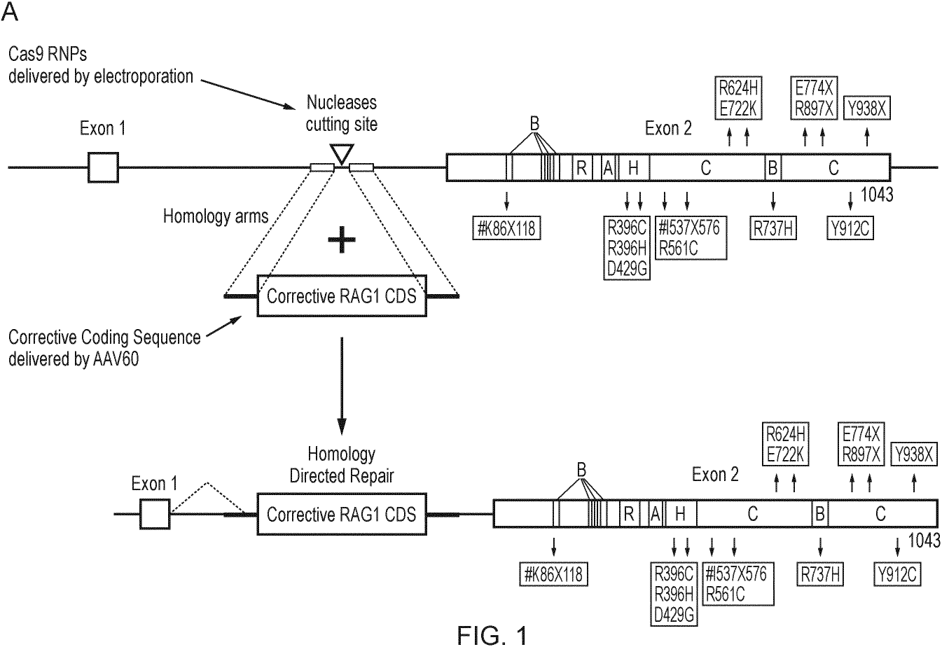

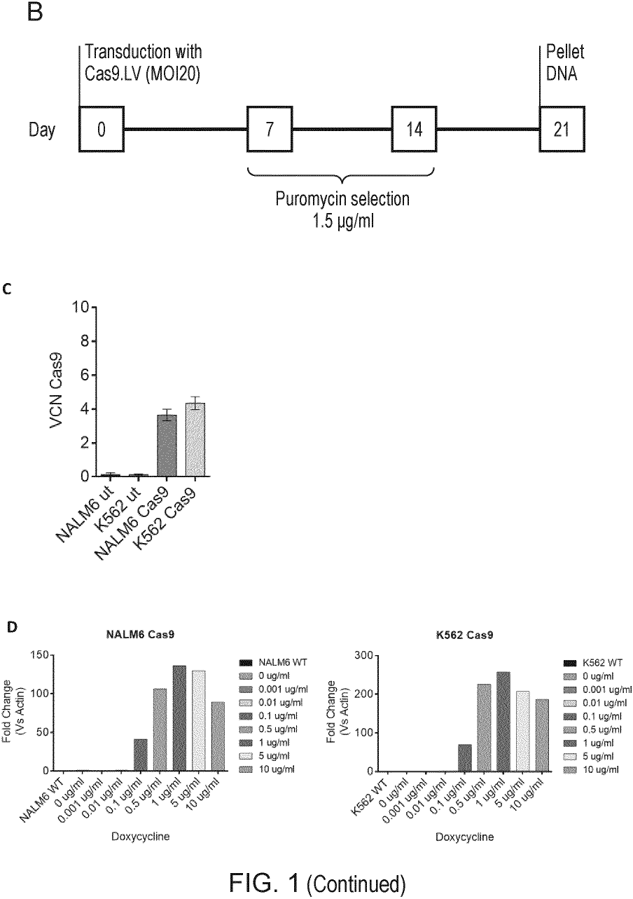

In the approach described herein, we deliver by nucleofection a Cas9 ribonucleoprotein (RNP) that introduces a DNA double strand break (DSB) in the first intron of RAG1 gene. Following the DNA DSB, the corrective donor DNA, delivered by AAV6 vector, is integrated by homology directed repair (HDR), thanks to the presence of two sequences, flanking the corrective donor, that are homologous to the Cas9 cutting site. An alternative splicing acceptor (SA) upstream of the corrective DNA allows the endogenous promoter of RAG1 to control the expression of the transgene (FIG. 1 panel A). Of note, RAG1 exon 2 contains the whole coding sequence, thus integrating a corrective RAG1 coding sequence upstream of exon 2 may be therapeutic for any RAG1 mutation with clinical relevance.

### Generation of NALM6 and K562 Cas9 Cell Lines

First, to test our panel of Cas9 guide RNAs we generated two cell lines with inducible Cas9 expression. NALM6 and K562 cell lines were transduced with a lentiviral vector carrying the Cas9 cassette under the control of a TET-inducible promoter and a cassette that confers resistance to puromycin. After transduction with MOI 20 the two cell lines were kept in culture with puromycin 1.5 µg/ml for one week to select the transduced cells (FIG. 1 panel B). After puromycin selection, a VCN 3.65 and a VCN 4.35 were verified by LTR specific ddPCR in NALM6 Cas9 and K562 Cas9 cell line respectively (FIG. 1 panel C). Efficient Cas9 expression was also verified by RT-qPCR after two days of induction with scaling doses of doxycycline (FIG. 1 panel D). The highest Cas9 expression was found at the dose of 1 µg/ml of doxycyclin in both the cell lines.

### RAG1 Guide Selection

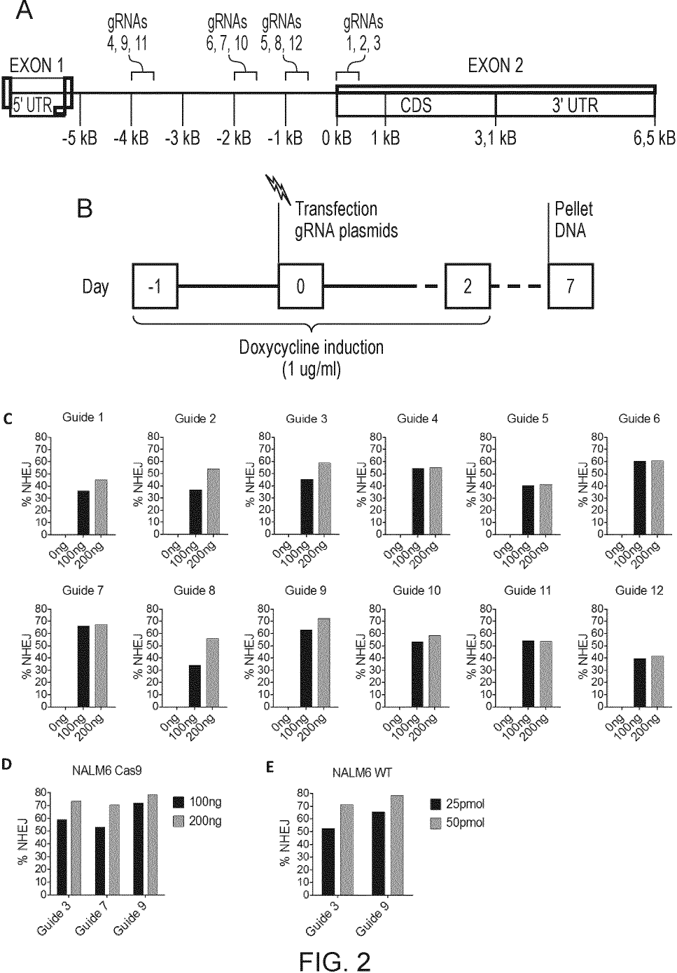

A panel of nine guides was first identified to target three non-repeated loci of RAG1 intron 1. In addition, three guides (gRNA 1,2,3) targeting the first 200 bp of RAG1 exon 2 were designed with the final aim to integrate the corrective RAG1 coding sequence in frame with the endogenous ATG. This strategy would exploit the endogenous splice acceptor thus preserving any putative endogenous splicing regulations (FIG. 2 A).

Guides were electroporated as plasmid DNAs in K562 Cas9 and NALM6 Cas9 cell lines considering two different doses (100 ng/well and 200 ng/well.) Cas9 expression was induced the day before the electroporation and for the two following days by adding doxycycline (1 µg/ml) to the medium. Genomic DNA was extracted at day 7 and cutting frequency was evaluated measuring the percentage of NHEJ-mediated indel mutations by T7 nuclease assay (scheme shown in FIG. 2 B).

The majority of the tested guides had good cutting frequency showing similar results in both cell lines. In particular, Guide 9 was the best performing guide targeting the intron with a cutting frequency up to 72.7% in K562 Cas9 and 78.5% in NALM6 Cas9. Similar cutting frequencies were also achieved by Guide 7, that showed a cutting frequency up to 67.5% in K562 Cas9 and 70.5% in NALM6 Cas9 cell lines. Guide 3 was the best performing guide targeting the exon with a cutting frequency up to 58.9% in K562 Cas9 (FIG. 2 C) and 73.5% in NALM6 Cas9 (FIG. 2 D). Of note, despite the higher expression of Cas9 expression in K562 Cas9 than in NALM6 Cas9 cell line, no difference in the overall cutting efficiency was observed. Cutting frequency was also tested in NALM6 WT using in vitro preassemble RNP of guide 9 and guide 3 at the dose of 25 or 50 pmol/well (FIG. 2 E). Both guides retained a good activity, guide 3 reached up to 71.5% cutting frequency and guide 9 up to 78.5% at the higher dose of RNP.

### Donor DNA Optimization

Guide 9 was further tested in NALM6 Cas9 and K562 Cas9 cell lines to verify the correct integration of the PGK_GFP reporter cassette flanked by two homology arms.

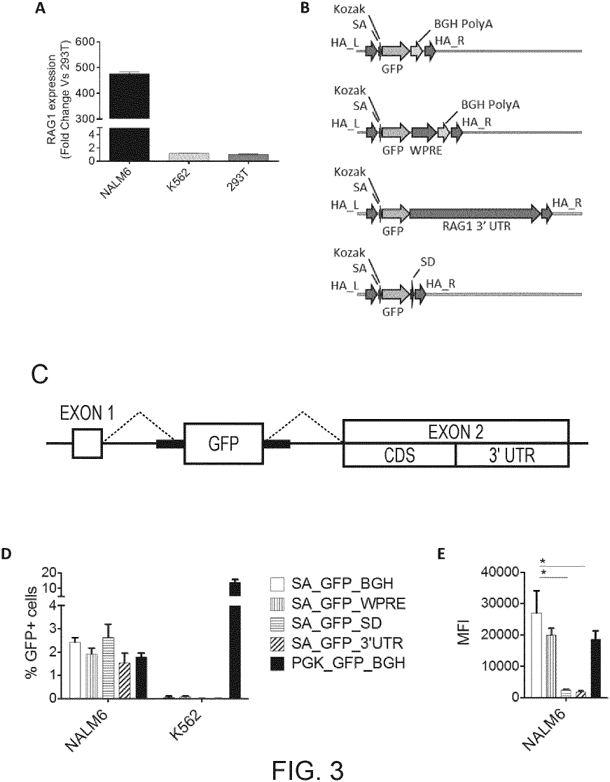

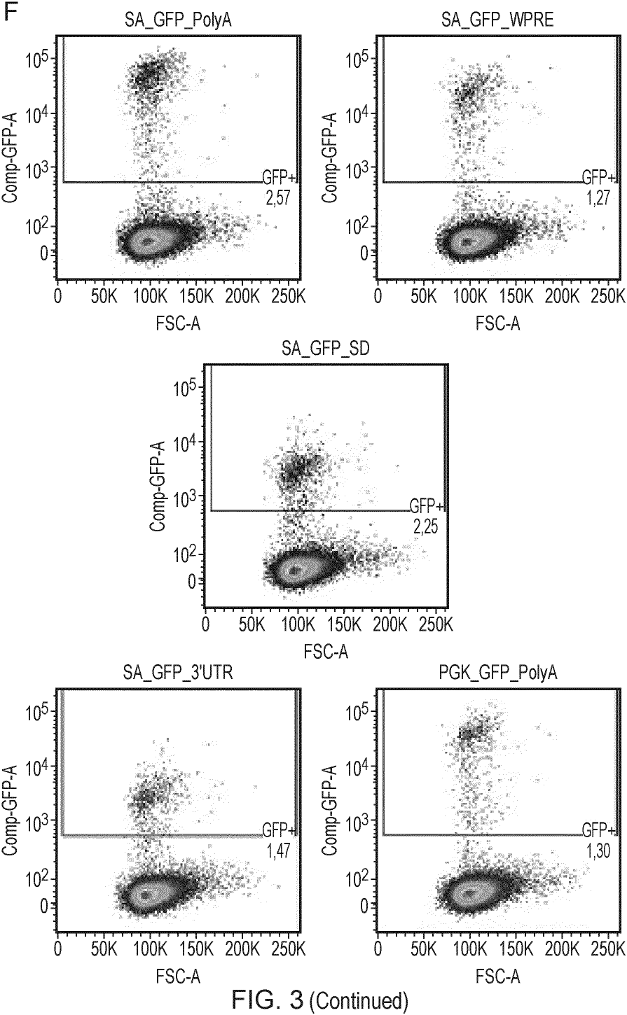

We also assessed the ability of the endogenous RAG1 promoter to induce the expression of the GFP in the absence of the PGK promoter using a donor plasmid containing splice acceptor (SA) SA_GFP cassette. RAG1 expression occurs only during lymphocytes differentiation at DN2 T and pro-B cell stages. To assess whether the endogenous promoter of RAG1 was able to induce the expression of the GFP cassette, we exploited NALM6 cell line, a Pre-B cell line that constitutively expresses RAG1 (FIG. 3 A). As mentioned, RAG1 genomic region is composed of two exons and the whole coding sequence, which is 3.1 Kb, is encoded by the second exon, followed by a long 3′UTR region of 3.3 Kb. Our correction strategy plans to deliver an AAV6 vector containing the entire coding sequence targeting the intronic region upstream of exon 2. The 3′UTR region (>3 Kb) downstream of the RAG1 coding sequence was not inserted because of the limited size hosted by the AAV6 vector.

To assess whether the 3′UTR of RAG1 is necessary for the efficient expression of our corrective donor, we generated four different SA_GFP donor DNAs (FIG. 3 B):
i. construct carrying the bovine growth hormone (BGH) PolyA downstream the SA_GFP (SA_GFP_BGH);
ii. construct carrying the Woodchuck hepatitis virus post-transcriptional regulatory element (WPRE) downstream the SA_GFP and upstream the BGH PolyA (SA_GFP_WPRE). WPRE has been reported to generally enhance transgene expression;
iii. construct with the same endogenous RAG1 3′UTR following the SA_GFP cassette (SA_GFP_3′UTR);
iv. construct containing a splice donor downstream the SA_GFP cassette (SA_GFP_SD) to obtain a fusion transcript including the corrected sequence and endogenous RAG1 followed by the 3′ UTR sequence (FIG. 3 C).

NALM6 Cas 9 and K562 Cas9 cell lines, previously stimulated with doxycycline to induce Cas9, were transfected with guide 9 plasmid DNA (100 ng/well) and of various linearized DNA donors (1600 ng/well). Stable integration of the donor DNA was verified by flow cytometry as GFP expression.

The PGK_GFP positive control was stably integrated in both cell lines. In particular, ten days after transfection, 14% K562 Cas9 and 1.8% of NALM6 Cas9 were GFP positive (FIG. 3 D ). Of note NALM6 cell line is particularly tricky to edit and we expected a lower efficiency as compared K562. Similar frequencies of GFP+ cells were observed in NALM6 Cas9 transfected with the different SA_GFP donors, while almost no GFP⁺ cells were detectable in the K562 cell lines transfected with the SA_GFP donors. This observation confirms that the endogenous RAG1 promoter efficiently induces the expression of the SA_GFP cassette in the NALM6 Cas9 cell line. Of note, the absence of GFP⁺ cells in K562 Cas9 cell line, which lacks RAG1 expression, further confirms that the GFP expression observed in NALM6 is specifically dependent on RAG1 promoter activity.

The effect of constructs carrying different 3′UTR was evaluated in NALM6 Cas9 cell line by fluorescence intensity (MFI) of GFP⁺ events at flow cytometry. The analysis suggested that the endogenous RAG1 3′UTR negatively affects the expression of the transgene. GFP MFI obtained upon transfection with SA_GFP_SD and SA_GFP_3′UTR constructs was significantly lower than MFI obtained by SA_GFP_BGH (FIGS. 3 E, F). No improvement was noticed using SA_GFP_ WPRE. Based on these data reporting GFP expression level, we decided to clone our vectors with the BGH_PolyA.

### Off-Target Analysis

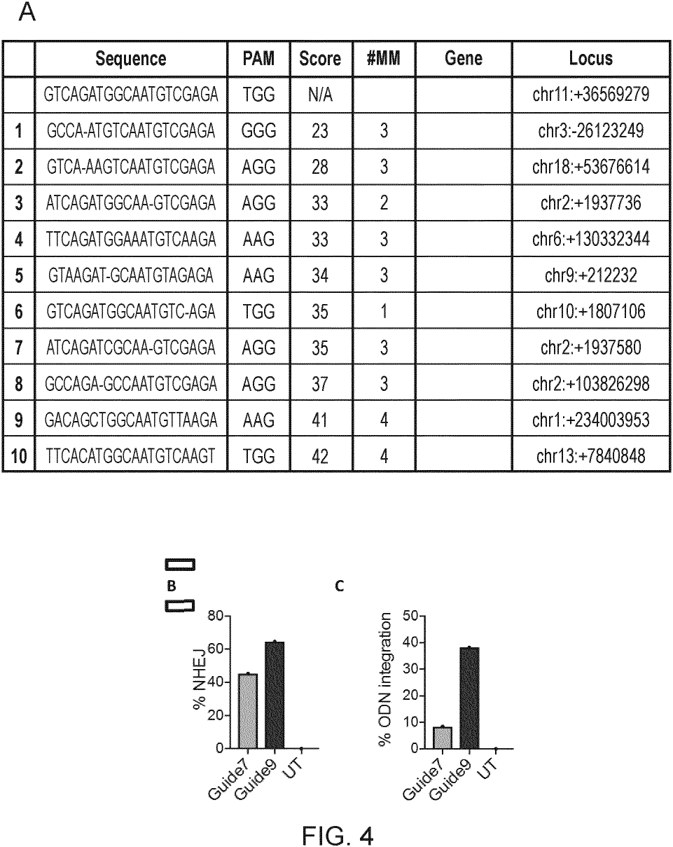

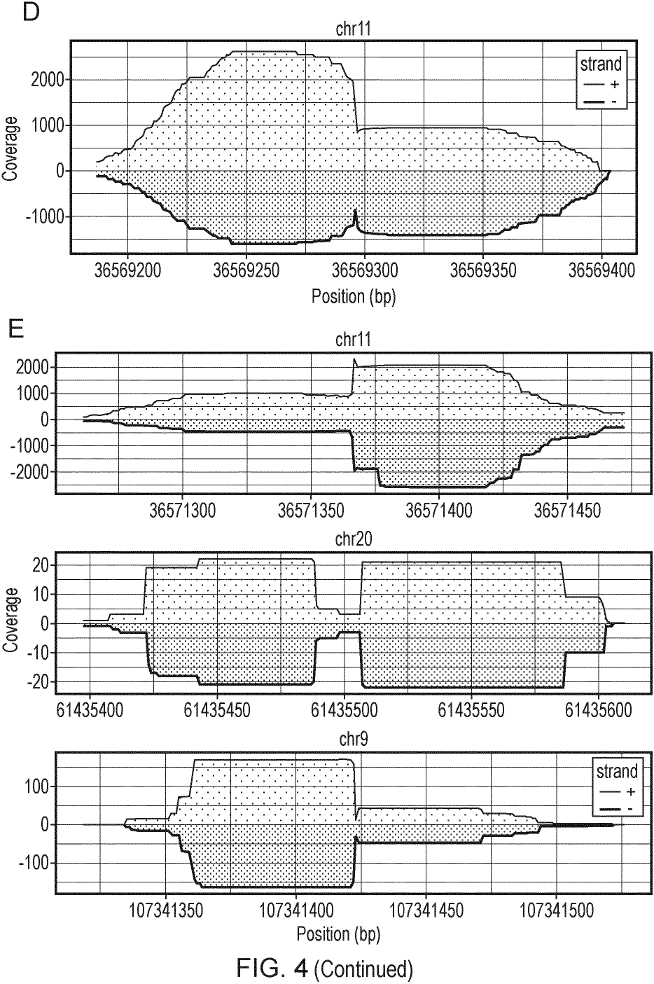

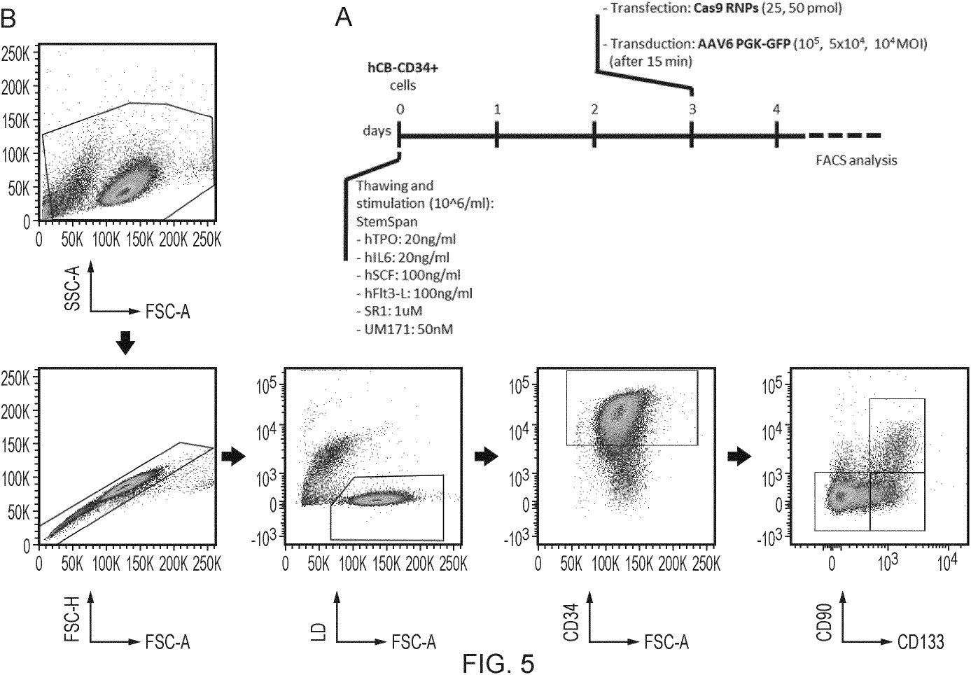

Preliminary in silico analysis demonstrated a promising off-target profile of guide 9 and showed that most likely off-targets fall in intronic regions thus suggesting a low risk of off-target related gene disruption events (FIG. 4 A). A deeper characterization of the off-target profile of guide 7 and 9 was pursued by an unbiased off-target detection assay (GUIDE-seq, Tsai SQ, et al. Nat Biotechnol. 2015;33(2):187-97). The analysis was performed using 50 pmol of High Fidelity Cas9 Nuclease V3 on K562 cells resulting in 45.3% and 64.6% cutting frequency by guide 7 and 9, respectively (FIG. 4 B). We achieved low (8.4%) ODN integration for guide 7, but good frequency of integration for the guide 9 (38.2%) allowing the analysis of off-target in the samples (FIG. 4 C). According to the analysis performed using the R Bioconductor package GUIDE-seq (Zhu LJ, et al. BMC Genomics. 2017;18(1)) using default parameters, no off-target site was identified for both guides. To deepen the investigation also to very weak potential off-targets, a second analysis with relaxed constraints was performed, and two off-target sites were found only for guide 7. These off-target sites fall into intronic or intergenic regions, with a number of mismatches >9 and at low frequency, indicating the low risk profile of guide 7. It is worth noting that no off-target sites were identified for Guide 9.

### Optimization of the Gene Editing Protocol on Human Cord blood-CD34+ Cells

The editing procedure was then optimized in human CD34⁺ cells from cord blood (hCB-CD34). To this end, hCB-CD34 cells were thawed at day 0 and prestimulated for three days seeding 1×10⁶ cells/ml in StemSpan enriched with cytokines (hTPO 20 ng/ml, hlL6 20 ng/ml, hSCF 100 ng/ml, hFlt3-L 100 ng/ml, SR1 1 uM, UM171 50 nM).

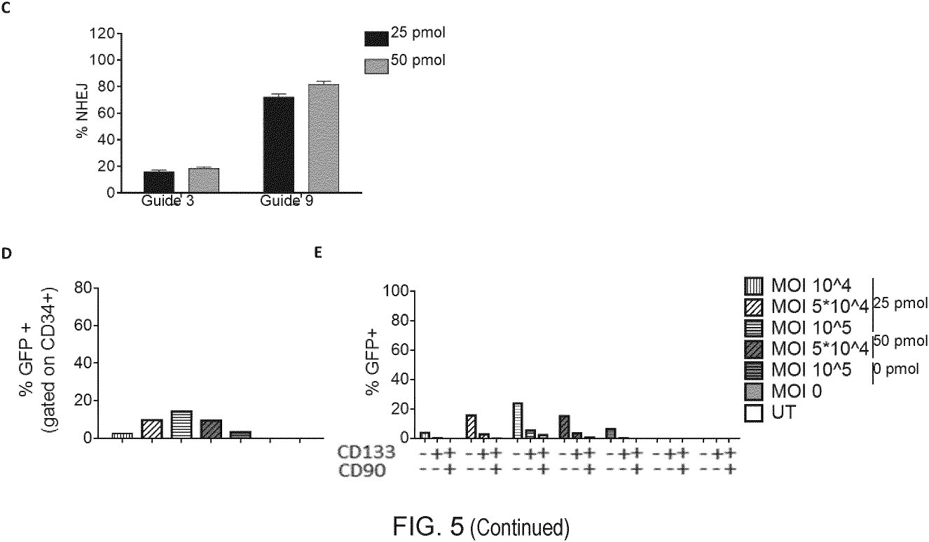

At day 3, guides 3 and 9 were delivered by electroporation as in vitro preassembled RNPs and two doses were considered 25 and 50 pmol/well. To enhance cellular stability, chemical modification consisting in 2′-O-methyl 3′phosphorothioate were added at the last three terminal nucleotides at 5′ and 3′ ends of the guide RNAs. After 15′, AAV6 vectors were added to the medium using three (10⁴, 5×10⁴, 10⁵) MOI doses (FIG. 5 A). To easily track edited cells using flow cytometry approach, two AAV6 donors (one for each guide) were used, carrying the PGK_GFP_BGH cassette flanked by two arms homologous to each of the two cutting sites. The toxicity of the procedure was assessed 24 hours after the treatment, by staining the cells with 7AAD and Annexin V and measuring the fraction of necrotic and apoptotic cells by flow cytometry. Four days after electroporation, we performed multiparametric flow cytometry analysis to evaluate the composition of various cellular subpopulations composing the bulk treated cell culture and measure the percentage of GFP+ cells within these subpopulations. For this analysis, we took advantage of surface markers that allow identifying the primitive (CD34⁺CD133⁺CD90⁺), early (CD34⁺CD133⁺CD90⁻) and more committed (CD34⁺CD133⁻ CD90⁻) progenitors (FIG. 5 B). Moreover, genomic DNA was extracted to determine the activity of the nucleases by T7 nuclease assay.

Guide 9 retained an activity comparable to that verified in NALM6 and K562 cell lines, 73.9% cutting frequency was observed with 25 pmol/well and 80.1% with 50 pmol/well. Guide 3 displayed a lower activity in hCB-CD34 with a cutting frequency of 16.9% and 19.3% with 25 and 50 pmol/well respectively (FIG. 5 C). In line with the latter observation, targeted integration with guide 3 was less efficient and at the dose of 25pmol/well, with the highest MOI (10⁵), levels of integration were 18.3% in the bulk CD34⁺ and 1.25% in the most primitive subpopulation (FIGS. 5 D, E).

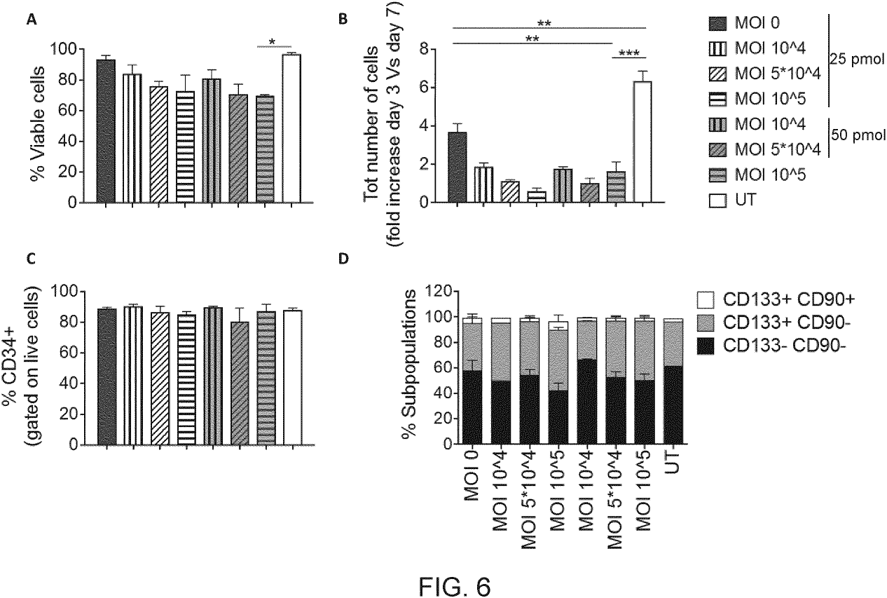

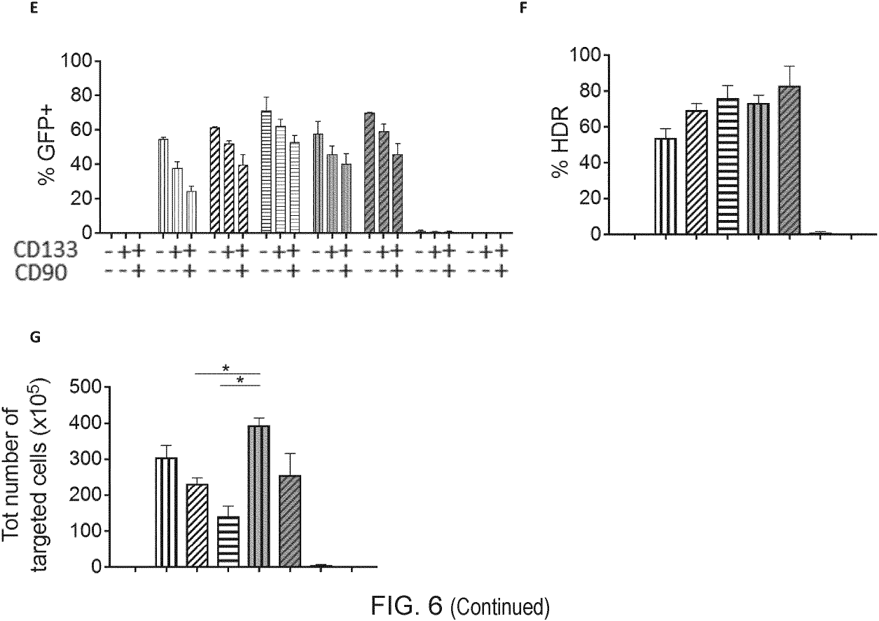

Guide 9 promoted a highly efficient targeted integration of the PGK_GFP cassette. Apoptosis analysis showed a low toxicity associated with the editing procedure, and viability (7AAD⁻ AnnexinV⁻ cells) was above 70% the day after the editing for all the conditions tested (FIG. 6 A). The analysis also suggested that AAV6 transduction had a stronger impact on cell viability than Cas9 transfection. In line with this observation, AAV6 transduction with MOI 10⁵ impaired cell growth more than the transfection with 25 pmol Cas9, suggesting that cell fitness may be affected (FIG. 6 B). High frequency of CD34⁺ cells (87.5%) in edited conditions was comparable to the untreated control (FIG. 6 C). No major differences were observed in distribution of the three CD34⁺ subpopulations among different conditions (FIG. 6 D).

Analysis of integration frequency showed that that the most primitive subpopulation (CD34⁺CD133⁺CD90⁺) was the less permissive fraction. The highest editing frequency in this subpopulation was obtained using 25 pmol of Cas9 and the MOI 10⁵ (52.8%). At lower MOI, the higher Cas9 dose (50 pmol) enhanced the editing efficiency particularly in the most primitive subpopulation, indeed, with a MOI 10⁴, editing frequency was 24.6% and 40.5% with 25 and 50 pmol of Cas9 respectively (FIG. 6 E). To confirm at molecular level, the integration observed by flow cytometry, genomic DNA was analysed by a ddPCR assay using a set of primers specific for the on-target integration. The percentages of GFP measured by flow cytometry and the percentages of HDR obtained with the ddPCR were comparable, thus corroborating that most integrations were on-target (FIG. 6 F).

Overall, these data suggest that using this platform, we were able to obtain efficient targeting even in the most primitive CD34⁺ subpopulation. The editing protocol does not affect the phenotype of the cells (both in terms of total CD34⁺ cells and in terms of subpopulation distribution). In particular, we identified a guide RNA promoting high frequency of targeted integration and set up editing conditions (50 pmol/well Cas9 and MOI 10⁴ Vg/cell) that allow the best compromise between toxicity and targeting frequency (FIG. 6 G).

### In Vivo Transplantation of Gene Edited hC8-CD34⁺ Cells

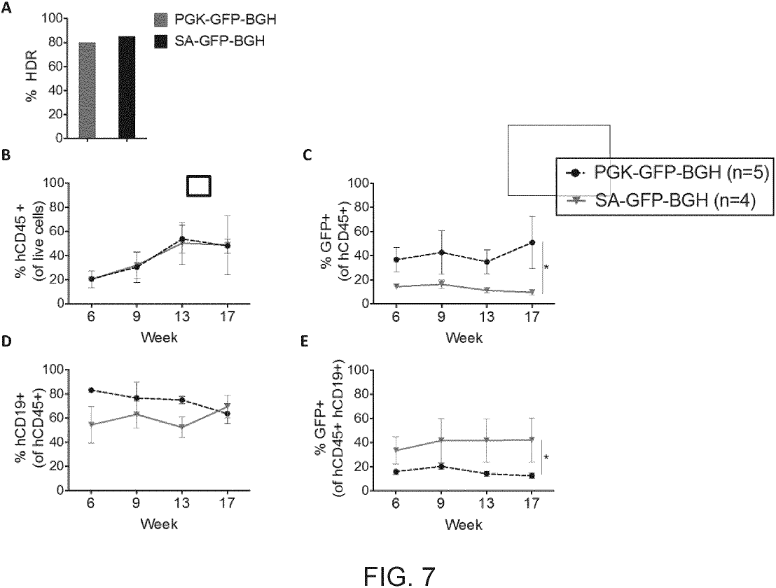

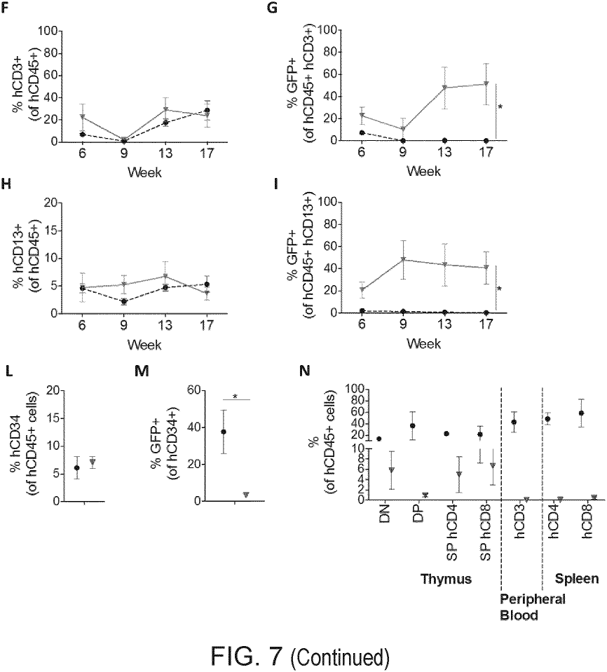

In order to assess if our procedure allows targeted integration in HSCs while preserving their long-term repopulating activity, edited CD34⁺ cells were transplanted into sublethally irradiated NOD-scid IL2Rg^{null} mice (NSG) mice. Following the same protocol used in the previous experiment, after 3 days of stimulation, hCB-CD34⁺ cells were electroporated with 50 pmol/well of guide 9 RNP and 15 minutes later transduced with AAV6 at MOI 10⁴ Vg/cell. In this experiment two distinct AAV6 vectors were used. The first AAV6 vector carrying the PGK_GFP_BGH was used as a positive control to easily follow engraftment of edited cells. The second donor carrying a SA_GFP_BGH was used to assess the in vivo expression of GFP gene under the control of RAG1 endogenous promoter. The day following the editing procedure, treated hCB-CD34⁺ 350,000 cells/mouse were injected in 4-5 NSG mice per group, 6 hours after sublethal total body irradiation (120 rad). In order to assess the levels of gene targeting efficiency after the treatment, few cells were maintained in culture for 4 more days. Using both the AAV6 vectors we measured ~80% of targeted integration by ddPCR (FIG. 7 A ), thus recapitulating the results obtained in the previous experiments. Flow cytometric analysis of the peripheral blood obtained from transplanted mice was performed 6, 9, 13 weeks after transplantation and at sacrifice at 17 weeks. Analysis of frequency of hCD45⁺ cells on total live cells in peripheral blood confirmed that treated cells were present at normal levels (up to -56%), suggesting long-term engraftment, and with a similar kinetics in the two groups (FIG. 7 B). With regard to peripheral blood composition, mice showed no major skew in the subpopulation composition and a normal presence of B, T and myeloid cells in both the groups confirming that the editing procedure does not affect multi-lineage differentiation (FIGS. 7 D, F, H).

In the group of mice receiving cells treated with PGK_GFP_BGH vector, edited hCD45⁺ GFP⁺ cells were maintained over time at high percentage (-40-50%), thus suggesting that the treatment was tolerated from the most primitive cells and confirming their long-term survival in vivo (FIG. 7 C). Similar levels of edited hCD45⁺ GFP⁺ cells were found among B cells, T cells and myeloid cells in peripheral blood, confirming that edited cells maintained multilineage differentiation capacity (FIGS. 7 E, G, I). In mice transplanted with SA_GFP_BGH treated cells, despite the efficient targeting frequency observed in vitro, we observed a reduced frequency of GFP+ cells in peripheral blood (FIG. 7 C). Myeloid and circulating T cells were GFP negative, as expected, because these two cell populations do not express RAG1 (FIGS. 7 G, I). Conversely, relevant percentage (-18%) of GFP⁺ cells was observed among circulating B cells (FIG. 7 E) likely due to their immature phenotype as the majority of B cells expressed CD24 and CD38.

At sacrifice, analysis of the bone marrow confirmed the engraftment of treated CD34⁺ stem cells. Moreover, in the PGK_GFP_BGH group, a high frequency of GFP⁺ targeted cells (~38%) was observed among the CD34⁺ cells further suggesting efficient engraftment of long-term repopulating stem cells (FIGS. 7 L and M). Although the thymus in NSG mice is atrophic and dysfunctional, we analyzed GFP expression during thymopoiesis according to CD4 and CD8 expression (FIG. 7 N). With the PGK_GFP_BGH cassette GFP expression was uniform among the developmental stages and no differences were observed between immature thymocytes and mature circulating T cells. Conversely, using SA_GFP_BGH cassette as donor, GFP expression was found in developing thymocytes, while almost no GFP expression was detected in peripheral blood and splenic T cells (FIG. 7 N).

Taken together these observations suggest that we have established an efficient protocol for the editing of long-term repopulating stem cells without affecting their engraftment and multilineage differentiation capacity. Our data further suggest an in vivo controlled expression pattern of the transgene, in the absence of exogenous promoters, highlighting that the expression is lymphoid specific and limited to immature lymphocytes.

### Test Corrective Donor on hMPB-CD34+ Cells

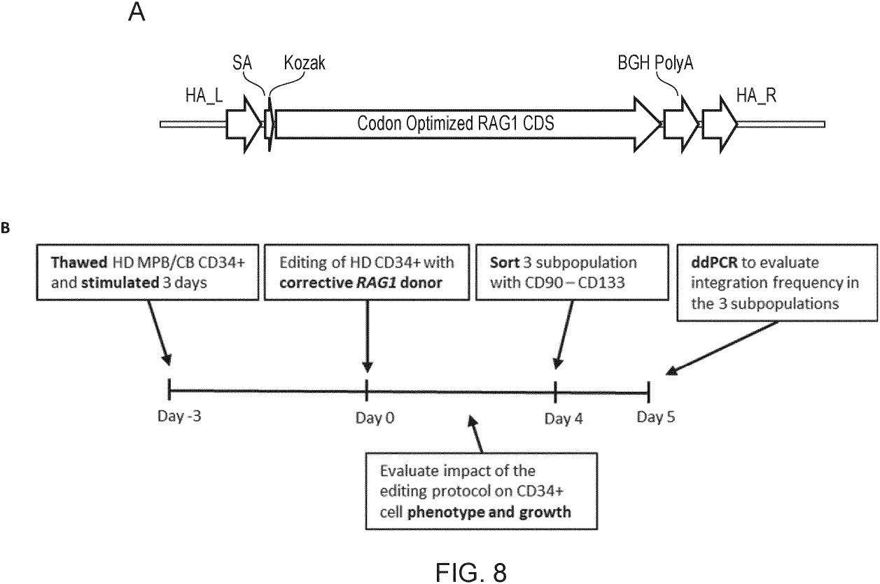

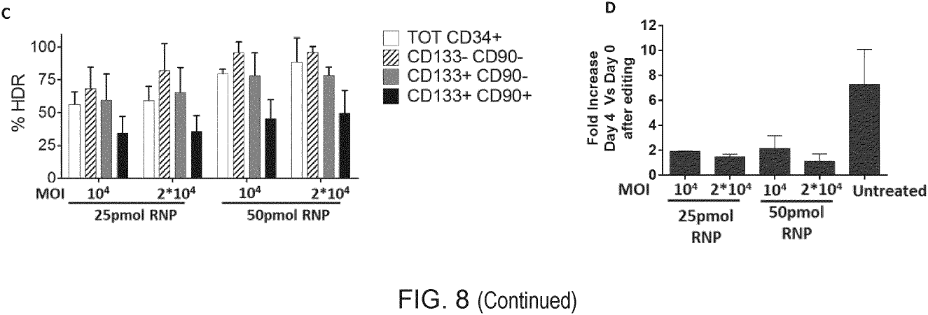

Next we designed and tested the corrective AAV6 vector carrying RAG1 coding sequence. In particular, the corrective donor included the two homology arms at the 3′ and 5′ extremities, a splice acceptor followed by the Kozak sequence, the RAG1 coding sequence and the BGH PolyA for a total length of 4.1 Kb (FIG. 8 A). RAG1 coding sequence was codon optimized replacing more “rare” codons with more frequent ones without changing the amino acid sequence, thus enhancing protein translation. We tested the new donor DNA on hCD34⁺ cells obtained from mobilized peripheral blood (MPB) to verify whether the dimension of the donor DNA could affect the efficiency of the integration and/or the toxicity profile.

MPB-CD34⁺ cells from normal donors (commercially purchased by AllCells California, US) were thawed and prestimulated for three days. We adjusted the editing protocol as follows: Stem cell factor (SCF) 300 ng/ml, Flt3 ligand (Flt-3L) 300 ng/ml, Thrombopoietin (TPO) 100 ng/ml, Interleukin 3 (IL-3) 60 ng/ml, StemRegenin1 (SR1, 1 uM) and 16,16-dimethyl prostaglandin E2 (dmPGE2, 10 uM), UM171 35 nM.

Cas9 was electroporated as in vitro preassembled RNP at two doses (25 pmol/well and 50 pmol/well). Since our previous observation suggested that high AAV6 vector MOI could impair cell fitness, we considered two low MOI (10⁴ and 2*10⁴).

Impact of the editing procedure was evaluated considering cell growth and cell phenotype by flow cytometry. Since the corrective donor does not include any reporter gene, we assessed the integration by molecular assays. Four days after editing, cells were sorted based on the expression of CD34, CD133, and CD90 to identify and analyze primitive, early and committed progenitor subpopulations. Genomic DNA from sorted subpopulations was extracted, and targeted integration of the corrective donor was verified by ddPCR assay, using a set of primers specific for the on-target integration and for the codon optimized donor sequence (FIG. 8 B). In accordance with previous observations, the editing protocol did not affect cell phenotype based on the expression of CD133 and CD90 (data not shown) and high on-target integration frequency was observed in all CD34 subpopulation. In particular, in the most primitive subpopulation a targeting frequency of 45.3% was observed using 50 pmol/well Cas9 and 10⁴ MOI of AAV6 vector (FIG. 8 C) also showing lower impact on cell growth as compared to the higher MOI (FIG. 8 D). No differences were noticed between hCD34⁺ cells from MPB or CB both in terms of efficiency and toxicity.

### In Vivo Transplantation of Edited hMPB-CD34+ Cells From HD and Patient

To assess whether our gene editing procedure may affect engraftment capability, edited hMPB-CD34⁺ cells were transplanted into sub-lethally irradiated NSG mice. Following the same protocol used in the previous experiment, after 3 days of stimulation, hMPB-CD34⁺ cells were electroporated with 50pmol/well of guide 9 RNP and 15 minutes later transduced with corrective AAV6 at MOI 10⁴ Vg/cell. To dampen the previously reported editing-induced p53 response, which decreases hematopoietic reconstitution by edited HSPCs, we added to the electroporation mixture an mRNA encoding for the dominant-negative p53 inhibitor GSE56 (Schiroli G, et al. Cell Stem Cell. 2019;24(4):551-565.e8).

To evaluate in vivo gene correction, we had access to hMPB-CD34⁺ cells obtained from a patient (NIHPID0021) carrying hypomorphic mutations in RAG1 gene. Of note NIHPID0021 is an adult patient with CID-G/Al due to missense RAG1 mutations (C1228T; G1520A) allowing residual development of B and T cells. The patient presented B cells 23/uL, T cells 665/uL (8% naïve), normal NK counts. Of note, the very low B cell counts in the periphery was also due to the treatment with anti-CD20 mAb to control severe autoimmune manifestations.

RAG1 patients received G-CSF/Plerixafor, and CD34⁺ cells were collected by the NIH clinical facility and their purity was verified by flow cytometry (>97% CD34⁺).

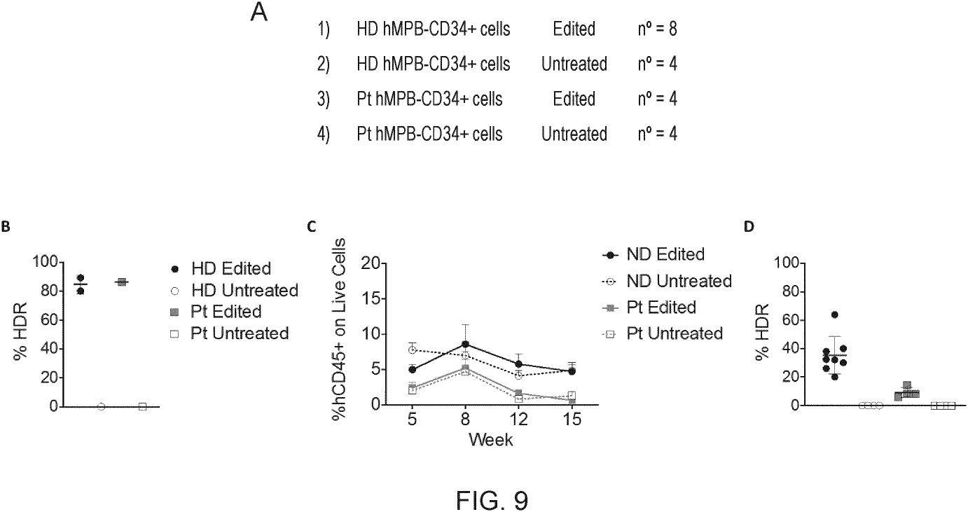

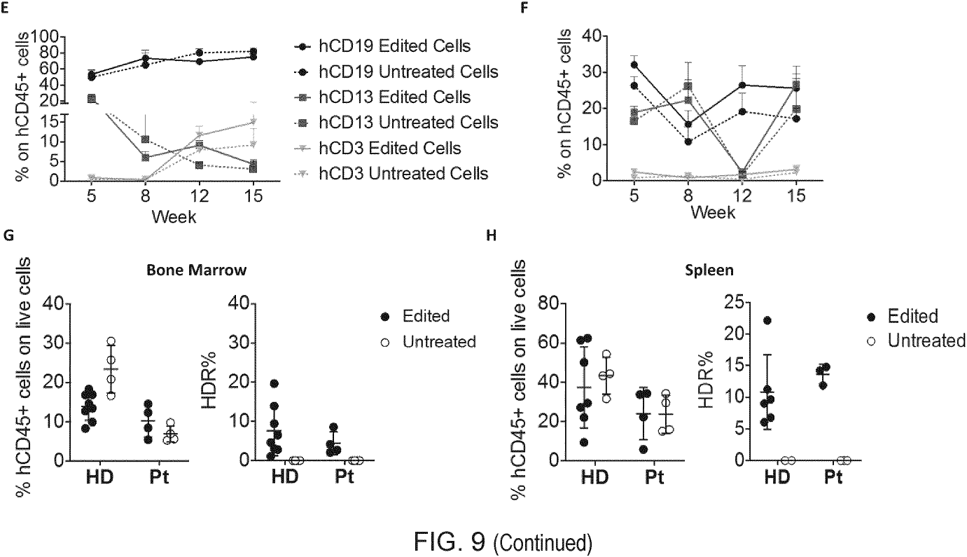

hMPB-CD34⁺ cells from two independent healthy donors (commercially purchased) were used in parallel. The day following the editing, 1×10⁶ of treated or untreated cells were injected in sublethally irradiated mice (120 rad) (FIG. 9 A). In order to assess the levels of gene targeting efficiency after the treatment, few cells were maintained in culture for four more days. ddPCR showed a targeted frequency of 86% in patient cells, while 89% and 80% were observed in the two healthy donor batches respectively, thus recapitulating the results obtained in the previous experiment (FIG. 9 B).

Flow cytometric analysis on the peripheral blood was performed 5, 8, 12 weeks after transplantation, and mice were sacrificed at 15 weeks.

The analysis of the peripheral blood showed that engraftment of hMPB-CD34⁺ was significantly lower than hCB-CD34⁺. Frequency of hCD45⁺ cells from HDs assessed in the blood was between 4.4% and 8.7% in all time points, and engraftment of the two batches was superimposable. Conversely, in CID/AG NIHPID002 patient the frequency of hCD45⁺ cells in PB was generally lower (between 2.1 and 5.2% at the first two time points) and decreased at later time points suggesting exhaustion of the engraftment. Of note, in both cases (CID/AG Patient and HD cells) no differences between treated and untreated cells were noticed in terms of frequency of hCD45⁺ cells in PB, confirming that engraftment capability was not affected by the editing protocol (FIG. 9 C).

Molecular analysis performed by ddPCR assay revealed a targeting frequency of 35.3% in human cells obtained from peripheral blood of mice receiving gene edited MPB-CD34⁺ HD cells, thus recapitulating previous observations obtained with the reporter gene and further confirming that targeting procedure does not affect the engraftment (FIG. 9 D). Lower targeting frequency (9.3%) was obtained in the PB 8 weeks after transplant with gene edited MPB patient CD34⁺ cells (FIG. 9 D).

With regard to peripheral blood composition, NSG mice transplanted with treated HD cells showed no major skewing in the subpopulation composition and a comparable frequency of B, T and myeloid cells was observed in mice receiving treated or untreated cells, confirming that multilineage differentiation was not impaired (FIG. 9 E). Untreated patient cells showed a partial skew in B- and T- cell compartment, when compared to the HD, in line with the immune phenotype of patients carrying hypomorphic mutations (Delmonte OM, et al. Blood. 2020;135(9):610-9). At the last time point, mice receiving untreated patient cells, B-cell frequency was 17.2% (HD untreated = 81.9%) and T-cell frequency was 2.3% (HD untreated = 9.2%) with a high myeloid cell frequency that was 19.9% (HD untreated = 3.0%). These observations confirm that despite defects in B- and T-cell development, some circulating Band T-lymphocytes can be detected. No significant differences were noticed between mice receiving untreated or treated patient cells in terms of peripheral blood immune composition, even though we observed a slight increase in B-cell frequency in treated patient cells that was maintained over time (FIG. 9 F).

Mice were sacrificed 17 weeks after the transplant to analyze the engraftment of edited cells in bone marrow, thymus and spleen. In the bone marrow and spleen, frequencies of human CD45+ cells were higher than those retrieved mice peripheral blood (FIGS. 8G, H left panels and 8C) . NSG mice transplanted with edited MPB CD34 cells from HD showed 13.9% of hCD45⁺ in the bone marrow, whilst 23.4% in untreated group (FIG. 9 G, left panel). Similar engraftment levels were achieved in mice receiving edited RAG1 patient cells (10.2%), but lower proportion of hCD45⁺ cells was found in mice receiving untreated RAG1 patient cells (6.9%) (FIG. 9 G, left panel). hCD45⁺ cells engraftment was even higher in the spleen for both edited and untreated cells of HD and patient. In mice receiving HD cells, the frequency of hCD45⁺ cells was 37.4% and 43.3% in mice with edited or untreated cells, respectively (FIG. 9 H, left panel), indicating the absence of differences between edited and not edited cells. Similarly, the frequency of hCD45⁺ cells was 24% and 23.7% in mice with edited or untreated cells derived from the RAG1-patient, respectively (FIG. 9 H, left panel).

HDR targeting efficiency assessed by ddPCR on DNA samples extracted from bone marrow and spleen showed a range from 1.1% to 19.6% in edited cells from the bone marrow, while 2.1% to 8.5% in the case of patient cells (FIG. 9 G, right panel). The spleen showed the highest targeting frequency, with a range between 6.1% and 22.2% for mice with edited HD cells, and between 11.9% and 14.8% for mice with edited patient cells (FIG. 9 H, right panel).

Overall, these findings confirmed the feasibility of gene editing approach to target the human RAG1 locus in HSCs derived from HD and patient with RAG1 mutation. The GE procedure did not affect the engraftment capability and the multilineage differentiation of HSCs.

### Discussion

Classical gene-addition based gene therapy strategies rely upon the use of integrating vectors. The introduction of new generation vectors, whose improved design confers a safer integration profile, alleviated but did not abolish the risk of insertional mutagenesis caused by vector semi-random integration into the genome (Doi K, Takeuchi Y. Vol. 65, Uirusu. 2015. p. 27-36). Furthermore, the use of ubiquitous promoters dramatically hampers the physiological expression of therapeutic transgene whose expression is cell specific or tightly controlled during cell cycle.

RAG1 molecule mediates the site-specific DNA double stranded breaks necessary for initiating V(D)J recombination (Oettinger MA, et al. Science. 1990;248(4962):1517-23). DNA double strand breaks are per se dangerous lesions that can result in pathological genome rearrangements or chromosomal translocations. An important mechanism that ensures the fidelity of V(D)J recombination resides in the fine control of RAG1 expression that is restricted to specific target cells at specific developmental stages. RAG1 expression regulation is also indispensable for the selection of functional, non-self-reactive lymphocyte through complex mechanisms of “allelic exclusion” or BCR and TCR receptor editing (Ten Boekel E, et al. Immunity. 1998;8(2):199-207).

In the past, several attempts to correct RAG1 deficiency by retrovirus or lentivirus-mediated gene transfer have led to variable T and B cell reconstitution with development of inflammatory infiltrates and autoimmunity when suboptimal immune reconstitution is achieved (Pike-Overzet K, et al. Leukemia. 2011;25(9):1471-83; Pike-Overzet K, et al. Vol. 134, Journal of Allergy and Clinical Immunology. 2014. p. 242-3; Lagresle-Peyrou C, et al. Blood. 2006;107(1):63-72; and van Til NP, et al. J Allergy Clin Immunol. 2014;133(4):1116-23). In parallel, use of exogenous and ubiquitous promoters may lead to genotoxicity (Zhang Y, et al. Advances in Immunology. 2010. p. 93-133; and Papaemmanuil E, et al. Nat Genet. 2014;46(2):116-25).

The development of a gene editing platform represents a strategy to overcome several issues raised by conventional gene addition protocol. We have been focusing on HSC-based genome editing strategy to correct the broad spectrum of RAG1 deficiencies. To this end, we designed a strategy targeting the first RAG1 intron thus replacing the RAG1 coding sequence entirely contained in the exon 2. Our strategy has the advantage to cure most of disease-causing RAG1 mutations, while conserving the expression of the gene driven by its own promoter. To this purpose, we identified the best combination of nuclease reagents and corrective cDNA donors in NALM6 and K562 cell lines. Cas9 was electroporated as in vitro preassembled RNP in order to ensure a robust and short-term persistence in cells as prolonged persistence of Cas9 protein in primary cells could lead to off-target cleavage, potentially affecting cell homeostasis and functionality (Kim S, et al. Genome Res. 2014;24(6):1012-9). Delivering Cas9 as preassembled RNP is well tolerated and partially protect the gRNA from intracellular degradation thus improving stability and activity of the nuclease (Hendel A, et al. Nat Biotechnol. 2015;33(9):985-9). To further improve Cas9 activity profile, chemically modified gRNAs were used to enhance the stability, together with High Fidelity Cas9 variant in order to reduce off-target related toxicity (Vakulskas CA, et al. Nat Med. 2018;24(8):1216-24).

Prediction analysis of gRNA activity using Cas9 expressing cell line revealed reliable results for the guide targeting the intron (guide 9).

Next, we turned to hCB-CD34⁺ cells. HSPC were prestimulated to favour the transit through S/G2 phases when HDR preferably occurs (Genovese P, et al. Nature. 2014;510(7504):235-40; and Kass EM, Jasin M. Vol. 584, FEBS Letters. 2010. p. 3703-8) resulting in a moderate cell expansion while preserving original stemness phenotype considering expression of CD34, CD133 and CD90 markers.

Using guide 9 (50 pmol/well), Cas9 RNP and AAV6 vector (MOI 10⁴) carrying the PGK_GFP reporter cassette, we obtained good levels of targeting frequency (40.5%) in CD34⁺CD133⁺CD90⁺ the most primitive cell subpopulation. Molecular analysis assessed by ddPCR analysis showed that the majority of the integration was on target. Notably, during Cas9 and AAV6 dosage optimization, we noticed that high MOI of AAV6 had a strong impact on cell fitness. In vivo experiments further confirmed in vitro data. Transplantation of treated hCB-CD34⁺ cells in sublethally irradiated NSG mice showed long-term engraftment both in the bone marrow and peripheral blood, confirming multi-lineage differentiation capacity and long-term engraftment of targeted cells. We also tested SA_GFP cassette in which GFP expression is controlled by RAG1 endogenous promoter. In vivo data in NSG mice indicated a controlled lymphoid specific expression pattern of the transgene, that was restricted to immature lymphocytes in which RAG1 is physiologically expressed. To assess the impact of the endogenous RAG1 3′UTR in the donor DNA, we tested different donor constructs carrying GFP reporter gene. Analysis of donor AAV6 carrying endogenous RAG1-3′UTR indicates a reduction of GFP expression as compared to the level obtained using a donor with BGH_PolyA. These data associated with the lack of clinically relevant mutations in the RAG1 3′UTR so far reported in literature, suggest that this region could be dispensable in the design of the corrective donor. Finally, SA_GFP_ WPRE did not show advantage in GFP expression suggesting that WPRE-mediated expression enhancement could be promoter and cell line dependent. Based on this evidence, BGH PolyA sequence that allows the highest transgene expression level was cloned in the donor DNA. Furthermore, to further enhance protein translation, human RAG1 coding sequence was codon optimized replacing more “rare” codons with more frequent ones without changing the final amino acid sequence.

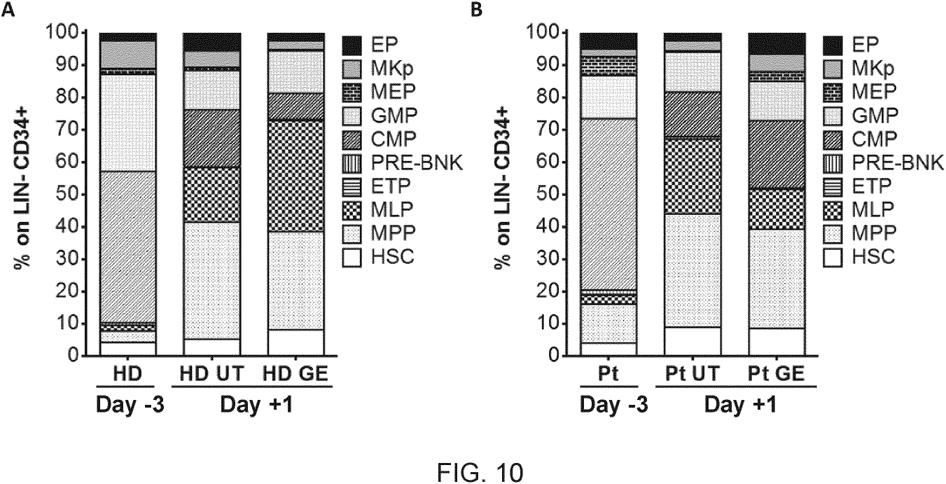

The newly designed donor AAV6 vector (including a SA sequence followed by the Kozak sequence, the RAG1 codon optimized followed by BGH_PolyA) was tested also in hMPB-CD34⁺ cells. We observed the same efficiency obtained with the previous donors, confirming that our protocol is reproducible using several donors and several HSPC sources. Moreover, the multiparametric analysis of HSPC composition in untreated and edited HD cells showed a redistribution of HSPC subtypes in cultured cells as compared to cells analyzed before the expansion phase (FIG. 10 A). In untreated and edited cells, we observed an expansion of hematopoietic stem cells (HSC), multipotent progenitors (MPP) and multilymphoid progenitors (MLP) at the expense of common myeloid progenitors (CMP), indicating that editing protocol preserves stemness composition (FIG. 10 A).

Notably, ddPCR analysis showed more than 80% HDR in total CD34⁺ cells and 45% of targeting frequency was observed in the most primitive (CD133⁺ CD90⁺) subpopulation subset. In vivo experiments in NSG mice transplanted with treated hMPB-CD34⁺ cells showed good level of engraftment and multilineage differentiation capability as those treated with unedited cells.

We had access to hMPB-CD34⁺ cells from a CID-G/Al RAG1 patient carrying hypomorphic mutations and presenting with a combined immunodeficiency associated to severe inflammation and autoimmune signs. We confirmed that the editing procedure did not affect the HSPC composition in RAG1-deficient cells (FIG. 10 B). Even in this case, we achieved 86% of targeting frequency as shown by ddPCR analysis. The in vivo transplant of treated and untreated cells showed lower engraftment of edited cells in peripheral blood of NSG mice with patient cells as compared to HD donor cells. In contrast, comparable engraftment was observed in bone marrow and spleen between HD and patient-treated mice, suggesting that gene edited patient-derived CD34⁺ cells preserve the engraftment and multi-lineage differentiation capability in vivo comparable analysis of central and peripheral lymphoid organs. Severe inflammatory conditions occurring in CID patients and/or effects of drug administration (anti-CD20 monoclonal antibody or high doses of corticosteroid) may influence the CD34⁺ cells fitness.

Overall, we have established an efficient and promising genome editing platform for the correction of RAG1 deficiency.

### Materials and Methods

### Lentiviral Vector Production and Titration

LVs were produced by transient transfection of 293T cells. 24 hours before transfection 9×10⁶ cells were plated in a 15 cm dish, 2 hours before transfection Iscove’s Modified Dulbecco’s (IMDM) medium was changed. The required transfer vector (34 µg) was mixed with 9 µg of VSV-G envelope encoding plasmid, 12.5 µg pMDLg/pRRE, 6.25 µg of REV plasmid and 15 µg of pADVANTAGE per 15 cm dish. This mixture was added to 293T cells by calcium phosphate precipitation. After 12-14 hours the medium was replaced with fresh complete IMDM supplemented with 1 mM of sodium butyrate. Collection and filtration of the supernatant took place 30 hours after this medium change. Following collection, the LV was concentrated 500 times by ultracentrifugation (2 hr, 20.000 rpm, 20°). A serial dilution was made of a known amount of 293T cells infected by the LV. After 3 days genomic DNA (gDNA) of the different dilutions was isolated with the DNeasy® Blood and Tissue Kit. Vector copy number (VCN) of the LV was measured by ddPCR. Titer was calculated by using the following formula: Titer = VCN x dilution factor x number of infected 293T cells. p24 HIV protein by ELISA assay (Abcam 218268) in order to estimate the amount of vector particles and calculate the relative infectivity of the vector preparation.

### Cas9 Inducible Cell Lines

NALM6 Cas9 cell line was generated by transducing NALM6 cells with a lentiviral vector expressing Cas9 protein under the control of a TET-inducible promoter and with a vector that constitutively expresses the TET transactivator (Clackson T. Vol. 7, Gene Therapy. 2000. p. 120-5). When doxycycline is administered to the culture media, the TET transactivator can bind the promoter of the Cas9 and induce its expression in the cells. K562 Cas9 cell line was generated with the same vector. Doxycycline was administered 24 h before electroporation of the nuclease. Cell lines were maintained in RPMI 1640 medium supplemented with 10% FBS, glutamine and penicillin/streptomycin antibiotics (complete medium).

### gRNA and RNP Assembly

Cas9 protein and custom RNA guides were purchased from Integrated DNA Technologies (IDT) and assembled following the manufacturer protocol. To enhance cellular stability, chemically modified guide RNAs were used. Briefly crRNA and trRNA were annealed heating them at 95° C. for 5 minutes and letting them slowly cool down at RT for 10 minutes. Cas9 protein was then incubated for 15 minutes at room temperature with the annealed guide RNA fragments, to assemble the ribonucleoprotein (RNP).

Guide sequences are shown in the table below:

| | |
|---|---|
| Guide 1 | TTTTCCGGATCGATGTGA |
| Guide 2 | GACATCTCTGCCGCATCTG |
| Guide 3 | GTGGGTGCTGAATTTCATC |
| Guide 4 | GATTGTGGGCCAAGTAACG |
| Guide 5 | GAAAGTCACTGTTGGTCGA |
| Guide 6 | CAATTTTGAGGTGTTCGTT |
| Guide 7 | GGGTTGAGTTCAACCTAAG |
| Guide 8 | TTAGCCTCATTGTACTAGC |
| Guide 9 | TCAGATGGCAATGTCGAGA |
| Guide 10 | GCAATTTTGAGGTGTTCGT |
| Guide 11 | ACCAGCCTCGGGATCTCAA |
| Guide 12 | TCAAATCAGTCGGGTTTCC |
| Guide RAG1KO | CCTTCTCAGCATTCCGA |
| Guide RAG1KO | AACATCTTCTGTCGCTGACT |

When used directly as RNA, the following guide sequences for guides 3, 7, 9 and RAG1KO may be used:

| | |
|---|---|
| Guide 3 | TGTGGGTGCTGAATTTCATC |
| Guide 7 | GGGGTTGAGTTCAACCTAAG |
| Guide 9 | GTCAGATGGCAATGTCGAGA |
| Guide RAG1KO | GTACCTTCTCAGCATTCCGA |

### Mismatch Selective Endonuclease Assay

A T7 endonuclease (T7E1) assay was used to measure indels induced by NHEJ. Briefly, gDNA of gene edited cells was extracted and amplified by PCR with primers flanking the Cas9 RNP target site. The PCR product was denatured, slowly re- annealed and digested with T7 endonuclease (New England BioLabs) for 1 h, 37°. T7 nuclease only cut DNA at sites where there is a mismatch between the DNA strands, thus between re-annealed wild type and mutant alleles. Fragments were separated on LabChip GXII Touch High Resolution DNA Chip (PerkinElmer®) and analysed by the provided software. The ratio of the uncleaved parental fragment versus cleaved fragments was calculated and it gives a good estimation of NHEJ efficiency of the artificial nuclease. Calculation of % NHEJ: (sum cleaved fragment)/(sum cleaved fragments + parental fragment) x 100. Primer used for NHEJ assay:

| | |
|---|---|
| Guides 1, 2, 3 FW | CCATAAACACTGTCAGAAGAGG |
| Guides 1, 2, 3 RV | GTGTTGCAGATGTCACAGG |
| Guides 4, 9, 11 FW | GAAGTGGTTCATGCAAGAGG |
| Guides 4, 9, 11 RV | GGATGAACATGGAGAAAGCAG |
| Guides 6, 7, 10 FW | GGGGAGAAATGTGTAGGGAAG |
| Guides 6, 7, 10 RV | CTCAAAAACAAAGAAATGGGCG |
| Guides 5, 8, 12 FW | ATAGGTGGATGGGATGATGG |
| Guides 5, 8, 12 RV | CCTCTTCTGACAGTGTTTATGG |
| Guides RAG1KO FW | GGAAAATGAATGCCAGGCAG |
| Guides RAG1KO RV | AGGTCATCATGCTGTACAAATG |
| Guides RAG1KO FW | TCCATGCTTCCCTACTGAC |
| Guides RAG1KO RV | CTCCCATTCCATCACAAGAC |

### Off-Target Analysis

In silico prediction of off-target profile was performed with COSMID (CRISPR Off-target Sites with Mismatches, Insertions, and Deletions) (Cradick TJ, et al. Mol Ther - Nucleic Acids. 2014;3(12):e214) to search genomes for potential CRISPR off-target sites. For GUIDE-Seq analysis K562 cells were electroporated with 50 pmol of High Fidelity Cas9 Nuclease V3 guide7 or guide 9 (as RNP) and dsODN to tag the breaks via an end-joining process consistent with NHEJ. dsODN integration sites in genomic DNA were precisely mapped at the nucleotide level using unbiased amplification and next-generation sequencing (Tsai SQ, et al. Nat Biotechnol. 2015;33(2):187-97). Library construction and GUIDE-Seq sequencing were performed by Creative Biogen Biotechnology (NY, USA) using Unique Molecular Identifier (UMI) for tracking PCR duplicates. Quality checking and trimming were performed on the sequencing reads, using FastQC and Trim_galore, respectively. High quality reads were aligned against the human reference genome (GRCh38), using Bowtie2 (Langmead B, Salzberg SL. Nat Methods. 2012;9(4):357-9) in the “very-sensitive-local” mode, in order to achieve optimal alignments. GUIDE-Seq data analysis was performed employing the R/Bioconductor package GUIDE-seq (Zhu LJ, et al. BMC Genomics. 2017;18(1)), and using UMI to deduplicate reads.

### Donor Constructs

The cloning of plasmids was performed using basic molecular biology techniques. In short, plasmids were digested using restriction enzymes (New England BioLabs) and correct fragments were separated and purified by agarose gel electrophoresis. Fragments were inserted into a dephosphorylated linearized backbone with either Quick Ligase or T4 Ligase after purification with QIAquick PCR Purification Kit (QIAGEN). After ligation, TOP10 chemically competent E. Coli bacteria were transformed and plated on plates containing antibiotics. Plasmid DNA was extracted and purified with Wizard Plus SV Minipreps DNA Purification System (Promega) and EndoFree Plasmid Maxi Kit (QIAGEN). Colonies were screened with control digestions and sequenced. Sequences of vector inserts with main features are reported below:

### AAV6 Vector Carrying SA_GFP_BGHPolyA, Guide 9:

INSERT

tgagcacacagttattacttggaaattgtgtacagactaagttgaagatg
ttaggagggaagattgtgggccaagtaacggggtgtatgtgtgtgggtat
agggtgggcagctgggatggaaatggggggctgctgctgctgctgcaccc
tggcctcctgaactaatgatatcactcaccagaaactactgttcctgcac
tgtccaagccaccccaaactagtttgtcaaaatgaatctgtgctgtgtgg
agggaggcacgcctgtagctctgatgtcagatggcaatgtgaattcctga
cctcttctcttcctcccacaggccgccaccatggtgagcaagggcgagga
gctgttcaccggggtggtgcccatcctggtcgagctggacggcgacgtaa
acggccacaagttcagcgtgtccggcgagggcgagggcgatgccacctac
ggcaagctgaccctgaagttcatctgcaccaccggcaagctgcccgtgcc
ctggcccaccctcgtgaccaccctgacctacggcgtgcagtgcttcagtc
gctaccccgaccacatgaagcagcacgacttcttcaagtccgccatgccc
gaaggctacgtccaggagcgcaccatcttcttcaaggacgacggcaacta
caagacccgcgccgaggtgaagttcgagggcgacaccctggtgaaccgca
tcgagctgaagggcatcgacttcaaggaggacggcaacatcctggggcac
aagctggagtacaactacaacagccacaacgtctatatcatggccgacaa
gcagaagaacggcatcaaggtgaacttcaagatccgccacaacatcgagg
acggcagcgtgcagctcgccgaccactaccagcagaacacccccatcggc
gacggccccgtgctgctgcctgacaaccactacctgagcacccagtccgc
cctgagcaaagaccccaacgagaagcgcgatcacatggtcctgctggagt
tcgtgaccgccgccgggatcactctcggcatggacgagctgtacaagtaa
actgtgccttctagttgccagccatctgttgtttgcccctcccccgtgcc
ttccttgaccctggaaggtgccactcccactgccctttcctaataaaatg
aggaaattgcatcgcattgtctgagtaggtgtcattctattctggggggt
ggggtggggcaggacagcaagggggaggattgggaagacaatagcaggca
tgctggggatgcggtgggctctatggggatccggaacaggtgtgataatg
agagatcttgcgttccaacgagaaactaatgtttctagaatggcagtggc
cggtggggacagggctgagccagcaccaaccactcagcctttgagatccc
gaggctggtctactgctgagaccttttgttagaagagaggagatcaagca
tttgcaaggtttctgagtgtcaaaatatgaatccaagataactctttcac
aatcctaacttcatgctgtctacaggtccatattttagcctgctttctcc
atgttcatccgaaaagaaagaaaagctaagggtggtggtcatatttgaaa
ttagccagatcttaagtttttctgggggaaatttagaagaaaatatggaa
aagtgactatgagcaca

HA Left

tgagcacacagttattacttggaaattgtgtacagactaagttgaagatg
ttaggagggaagattgtgggccaagtaacggggtgtatgtgtgtgggtat
agggtgggcagctgggatggaaatggggggctgctgctgctgctgcaccc
tggcctcctgaactaatgatatcactcaccagaaactactgttcctgcac
tgtccaagccaccccaaactagtttgtcaaaatgaatctgtgctgtgtgg
agggaggcacgcctgtagctctgatgtcagatggcaatgt

Splice Acceptor

ctgacctcttctcttcctcccacag

KOZAK

gccgccaccatg

GFP

atggtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagtcgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcctgacaaccac
tacctgagcacccagtccgccctgagcaaagaccccaacgagaagcgcga
tcacatggtcctgctggagttcgtgaccgccgccgggatcactctcggca
tggacgagctgtacaagtaa

PolyA

actgtgccttctagttgccagccatctgttgtttgcccctcccccgtgcc
ttccttgaccctggaaggtgccactcccactgccctttcctaataaaatg
aggaaattgcatcgcattgtctgagtaggtgtcattctattctggggggt
ggggtggggcaggacagcaagggggaggattgggaagacaatagcaggca
tgctggggatgcggtgggctctatgg

HA Right

atggcagtggccggtggggacagggctgagccagcaccaaccactcagcc
tttgagatcccgaggctggtctactgctgagaccttttgttagaagagag
gagatcaagcatttgcaaggtttctgagtgtcaaaatatgaatccaagat
aactctttcacaatcctaacttcatgctgtctacaggtccatattttagc
ctgctttctccatgttcatccgaaaagaaagaaaagctaagggtggtggt
catatttgaaattagccagatcttaagtttttctgggggaaatttagaag
aaaatatggaaaagtgactatgagcaca

### AAV6 Vector Carrying SA_GFP_WPRE_BGHPolyA, Guide 9:

INSERT

tgagcacacagttattacttggaaattgtgtacagactaagttgaagatg
ttaggagggaagattgtgggccaagtaacggggtgtatgtgtgtgggtat
agggtgggcagctgggatggaaatggggggctgctgctgctgctgcaccc
tggcctcctgaactaatgatatcactcaccagaaactactgttcctgcac
tgtccaagccaccccaaactagtttgtcaaaatgaatctgtgctgtgtgg
agggaggcacgcctgtagctctgatgtcagatggcaatgtgaattcctga
cctcttctcttcctcccacaggccgccaccatggtgagcaagggcgagga
gctgttcaccggggtggtgcccatcctggtcgagctggacggcgacgtaa
acggccacaagttcagcgtgtccggcgagggcgagggcgatgccacctac
ggcaagctgaccctgaagttcatctgcaccaccggcaagctgcccgtgcc
ctggcccaccctcgtgaccaccctgacctacggcgtgcagtgcttcagtc
gctaccccgaccacatgaagcagcacgacttcttcaagtccgccatgccc
gaaggctacgtccaggagcgcaccatcttcttcaaggacgacggcaacta
caagacccgcgccgaggtgaagttcgagggcgacaccctggtgaaccgca
tcgagctgaagggcatcgacttcaaggaggacggcaacatcctggggcac
aagctggagtacaactacaacagccacaacgtctatatcatggccgacaa
gcagaagaacggcatcaaggtgaacttcaagatccgccacaacatcgagg
acggcagcgtgcagctcgccgaccactaccagcagaacacccccatcggc
gacggccccgtgctgctgcctgacaaccactacctgagcacccagtccgc
cctgagcaaagaccccaacgagaagcgcgatcacatggtcctgctggagt
tcgtgaccgccgccgggatcactctcggcatggacgagctgtacaagtaa
actagtactcgacaatcaacctctggattacaaaatttgtgaaagattga
ctggtattcttaactatgttgctccttttacgctatgtggatacgctgct
ttaatgcctttgtatcatgctattgcttcccgtatggctttcattttctc
ctccttgtataaatcctggttgctgtctctttatgaggagttgtggcccg
ttgtcaggcaacgtggcgtggtgtgcactgtgtttgctgacgcaaccccc
actggttggggcattgccaccacctgtcagctcctttccgggactttcgc
tttccccctccctattgccacggcggaactcatcgccgcctgccttgccc
gctgctggacaggggctcggctgttgggcactgacaattccgtggtgttg
tcggggaaatcatcgtcctttccttggctgctcgcctgtgttgccacctg
gattctgcgcgggacgtccttctgctacgtcccttcggccctcaatccag
cggaccttccttcccgcggcctgctgccggctctgcggcctcttccgcgt
cttcgccttcgccctcagacgagtcggatctccctttgggccgcctcccc
gcctggaatggatcctaaactgtgccttctagttgccagccatctgttgt
ttgcccctcccccgtgccttccttgaccctggaaggtgccactcccactg
ccctttcctaataaaatgaggaaattgcatcgcattgtctgagtaggtgt
cattctattctggggggtggggtggggcaggacagcaagggggaggattg
ggaagacaatagcaggcatgctggggatgcggtgggctctatggtctaga
atggcagtggccggtggggacagggctgagccagcaccaaccactcagcc
tttgagatcccgaggctggtctactgctgagaccttttgttagaagagag
gagatcaagcatttgcaaggtttctgagtgtcaaaatatgaatccaagat
aactctttcacaatcctaacttcatgctgtctacaggtccatattttagc
ctgctttctccatgttcatccgaaaagaaagaaaagctaagggtggtggt
catatttgaaattagccagatcttaagtttttctgggggaaatttagaag
aaaatatggaaaagtgactatgagcaca

HA Left

tgagcacacagttattacttggaaattgtgtacagactaagttgaagatg
ttaggagggaagattgtgggccaagtaacggggtgtatgtgtgtgggtat
agggtgggcagctgggatggaaatggggggctgctgctgctgctgcaccc
tggcctcctgaactaatgatatcactcaccagaaactactgttcctgcac
tgtccaagccaccccaaactagtttgtcaaaatgaatctgtgctgtgtgg
agggaggcacgcctgtagctctgatgtcagatggcaatgt

Splice Acceptor

ctgacctcttctcttcctcccacag

KOZAK

gccgccaccatg

GFP

Atggtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagtcgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcctgacaaccac
tacctgagcacccagtccgccctgagcaaagaccccaacgagaagcgcga
tcacatggtcctgctggagttcgtgaccgccgccgggatcactctcggca
tggacgagctgtacaagtaa

WPRE

aatcaacctctggattacaaaatttgtgaaagattgactggtattcttaa
ctatgttgctccttttacgctatgtggatacgctgctttaatgcctttgt
atcatgctattgcttcccgtatggctttcattttctcctccttgtataaa
tcctggttgctgtctctttatgaggagttgtggcccgttgtcaggcaacg
tggcgtggtgtgcactgtgtttgctgacgcaacccccactggttggggca
ttgccaccacctgtcagctcctttccgggactttcgctttccccctccct
attgccacggcggaactcatcgccgcctgccttgcccgctgctggacagg
ggctcggctgttgggcactgacaattccgtggtgttgtcggggaaatcat
cgtcctttccttggctgctcgcctgtgttgccacctggattctgcgcggg
acgtccttctgctacgtcccttcggccctcaatccagcggaccttccttc
ccgcggcctgctgccggctctgcggcctcttccgcgtcttcgccttcgcc
ctcagacgagtcggatctccctttgggccgcctccccgcctg

PolyA

actgtgccttctagttgccagccatctgttgtttgcccctcccccgtgcc
ttccttgaccctggaaggtgccactcccactgccctttcctaataaaatg
aggaaattgcatcgcattgtctgagtaggtgtcattctattctggggggt
ggggtggggcaggacagcaagggggaggattgggaagacaatagcaggca
tgctggggatgcggtgggctctatgg

HA Right

atggcagtggccggtggggacagggctgagccagcaccaaccactcagcc
tttgagatcccgaggctggtctactgctgagaccttttgttagaagagag
gagatcaagcatttgcaaggtttctgagtgtcaaaatatgaatccaagat
aactctttcacaatcctaacttcatgctgtctacaggtccatattttagc
ctgctttctccatgttcatccgaaaagaaagaaaagctaagggtggtggt
catatttgaaattagccagatcttaagtttttctgggggaaatttagaag
aaaatatggaaaagtgactatgagcaca

### AAV6 Vector Carrying SA_GFP_SD, Guide 9:

INSERT

tgagcacacagttattacttggaaattgtgtacagactaagttgaagatg
ttaggagggaagattgtgggccaagtaacggggtgtatgtgtgtgggtat
agggtgggcagctgggatggaaatggggggctgctgctgctgctgcaccc
tggcctcctgaactaatgatatcactcaccagaaactactgttcctgcac
tgtccaagccaccccaaactagtttgtcaaaatgaatctgtgctgtgtgg
agggaggcacgcctgtagctctgatgtcagatggcaatgtgaattcctga
cctcttctcttcctcccacaggccgccaccatggtgagcaagggcgagga
gctgttcaccggggtggtgcccatcctggtcgagctggacggcgacgtaa
acggccacaagttcagcgtgtccggcgagggcgagggcgatgccacctac
ggcaagctgaccctgaagttcatctgcaccaccggcaagctgcccgtgcc
ctggcccaccctcgtgaccaccctgacctacggcgtgcagtgcttcagtc
gctaccccgaccacatgaagcagcacgacttcttcaagtccgccatgccc
gaaggctacgtccaggagcgcaccatcttcttcaaggacgacggcaacta
caagacccgcgccgaggtgaagttcgagggcgacaccctggtgaaccgca
tcgagctgaagggcatcgacttcaaggaggacggcaacatcctggggcac
aagctggagtacaactacaacagccacaacgtctatatcatggccgacaa
gcagaagaacggcatcaaggtgaacttcaagatccgccacaacatcgagg
acggcagcgtgcagctcgccgaccactaccagcagaacacccccatcggc
gacggccccgtgctgctgcctgacaaccactacctgagcacccagtccgc
cctgagcaaagaccccaacgagaagcgcgatcacatggtcctgctggagt
tcgtgaccgccgccgggatcactctcggcatggacgagctgtacaagtaa
actggatccaggtaagttctagaatggcagtggccggtggggacagggct
gagccagcaccaaccactcagcctttgagatcccgaggctggtctactgc
tgagaccttttgttagaagagaggagatcaagcatttgcaaggtttctga
gtgtcaaaatatgaatccaagataactctttcacaatcctaacttcatgc
tgtctacaggtccatattttagcctgctttctccatgttcatccgaaaag
aaagaaaagctaagggtggtggtcatatttgaaattagccagatcttaag
tttttctgggggaaatttagaagaaaatatggaaaagtgactatgagcac
a

HA Left

tgagcacacagttattacttggaaattgtgtacagactaagttgaagatg
ttaggagggaagattgtgggccaagtaacggggtgtatgtgtgtgggtat
agggtgggcagctgggatggaaatggggggctgctgctgctgctgcaccc
tggcctcctgaactaatgatatcactcaccagaaactactgttcctgcac
tgtccaagccaccccaaactagtttgtcaaaatgaatctgtgctgtgtgg
agggaggcacgcctgtagctctgatgtcagatggcaatgt

Splice Acceptor

ctgacctcttctcttcctcccacag

KOZAZ

gccgccaccatg

GFP

atggtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagtcgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcctgacaaccac
tacctgagcacccagtccgccctgagcaaagaccccaacgagaagcgcga
tcacatggtcctgctggagttcgtgaccgccgccgggatcactctcggca
tggacgagctgtacaagtaa

Splice Donor

aggtaagt

HA Right

atggcagtggccggtggggacagggctgagccagcaccaaccactcagcc
tttgagatcccgaggctggtctactgctgagaccttttgttagaagagag
gagatcaagcatttgcaaggtttctgagtgtcaaaatatgaatccaagat
aactctttcacaatcctaacttcatgctgtctacaggtccatattttagc
ctgctttctccatgttcatccgaaaagaaagaaaagctaagggtggtggt
catatttgaaattagccagatcttaagtttttctgggggaaatttagaag
aaaatatggaaaagtgactatgagcaca

### AAV6 Vector Carrying PGK_GFP_BGHPolyA, Guide 9:

INSERT

tgagcacacagttattacttggaaattgtgtacagactaagttgaagatg
ttaggagggaagattgtgggccaagtaacggggtgtatgtgtgtgggtat
agggtgggcagctgggatggaaatggggggctgctgctgctgctgcaccc
tggcctcctgaactaatgatatcactcaccagaaactactgttcctgcac
tgtccaagccaccccaaactagtttgtcaaaatgaatctgtgctgtgtgg
agggaggcacgcctgtagctctgatgtcagatggcaatgtgaattcccat
ggccacggggttggggttgcgccttttccaaggcagccctgggtttgcgc
agggacgcggctgctctgggcgtggttccgggaaacgcagcggcgccgac
cctgggtctcgcacattcttcacgtccgttcgcagcgtcacccggatctt
cgccgctacccttgtgggccccccggcgacgcttcctgctccgcccctaa
gtcgggaaggttccttgcggttcgcggcgtgccggacgtgacaaacggaa
gccgcacgtctcactagtaccctcgcagacggacagcgccagggagcaat
ggcagcgcgccgaccgcgatgggctgtggccaatagcggctgctcagcag
ggcgcgccgagagcagcggccgggaaggggcggtgcgggaggcggggtgt
ggggcggtagtgtgggccctgttcctgcccgcgcggtgttccgcattctg
caagcctccggagcgcacgtcggcagtcggctccctcgttgaccgaatca
ccgacctctctccccagggccgccaccatggtgagcaagggcgaggagct
gttcaccggggtggtgcccatcctggtcgagctggacggcgacgtaaacg
gccacaagttcagcgtgtccggcgagggcgagggcgatgccacctacggc
aagctgaccctgaagttcatctgcaccaccggcaagctgcccgtgccctg
gcccaccctcgtgaccaccctgacctacggcgtgcagtgcttcagtcgct
accccgaccacatgaagcagcacgacttcttcaagtccgccatgcccgaa
ggctacgtccaggagcgcaccatcttcttcaaggacgacggcaactacaa
gacccgcgccgaggtgaagttcgagggcgacaccctggtgaaccgcatcg
agctgaagggcatcgacttcaaggaggacggcaacatcctggggcacaag
ctggagtacaactacaacagccacaacgtctatatcatggccgacaagca
gaagaacggcatcaaggtgaacttcaagatccgccacaacatcgaggacg
gcagcgtgcagctcgccgaccactaccagcagaacacccccatcggcgac
ggccccgtgctgctgcctgacaaccactacctgagcacccagtccgccct
gagcaaagaccccaacgagaagcgcgatcacatggtcctgctggagttcg
tgaccgccgccgggatcactctcggcatggacgagctgtacaagtaaact
gtgccttctagttgccagccatctgttgtttgcccctcccccgtgccttc
cttgaccctggaaggtgccactcccactgccctttcctaataaaatgagg
aaattgcatcgcattgtctgagtaggtgtcattctattctggggggtggg
gtggggcaggacagcaagggggaggattgggaagacaatagcaggcatgc
tggggatgcggtgggctctatggggatccggaacaggtgtgataatgaga
gatcttgcgttccaacgagaaactaatgtttctagaatggcagtggccgg
tggggacagggctgagccagcaccaaccactcagcctttgagatcccgag
gctggtctactgctgagaccttttgttagaagagaggagatcaagcattt
gcaaggtttctgagtgtcaaaatatgaatccaagataactctttcacaat
cctaacttcatgctgtctacaggtccatattttagcctgctttctccatg
ttcatccgaaaagaaagaaaagctaagggtggtggtcatatttgaaatta
gccagatcttaagtttttctgggggaaatttagaagaaaatatggaaaag
tgactatgagcaca

HA Left

tgagcacacagttattacttggaaattgtgtacagactaagttgaagatg
ttaggagggaagattgtgggccaagtaacggggtgtatgtgtgtgggtat
agggtgggcagctgggatggaaatggggggctgctgctgctgctgcaccc
tggcctcctgaactaatgatatcactcaccagaaactactgttcctgcac
tgtccaagccaccccaaactagtttgtcaaaatgaatctgtgctgtgtgg
agggaggcacgcctgtagctctgatgtcagatggcaatgt

PGK promoter

ccacggggttggggttgcgccttttccaaggcagccctgggtttgcgcag
ggacgcggctgctctgggcgtggttccgggaaacgcagcggcgccgaccc
tgggtctcgcacattcttcacgtccgttcgcagcgtcacccggatcttcg
ccgctacccttgtgggccccccggcgacgcttcctgctccgcccctaagt
cgggaaggttccttgcggttcgcggcgtgccggacgtgacaaacggaagc
cgcacgtctcactagtaccctcgcagacggacagcgccagggagcaatgg
cagcgcgccgaccgcgatgggctgtggccaatagcggctgctcagcaggg
cgcgccgagagcagcggccgggaaggggcggtgcgggaggcggggtgtgg
ggcggtagtgtgggccctgttcctgcccgcgcggtgttccgcattctgca
agcctccggagcgcacgtcggcagtcggctccctcgttgaccgaatcacc
gacctctctccccagg

KOZAK

ccatg

GFP

atggtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagtcgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcctgacaaccac
tacctgagcacccagtccgccctgagcaaagaccccaacgagaagcgcga
tcacatggtcctgctggagttcgtgaccgccgccgggatcactctcggca
tggacgagctgtacaagtaa

PolyA

actgtgccttctagttgccagccatctgttgtttgcccctcccccgtgcc
ttccttgaccctggaaggtgccactcccactgccctttcctaataaaatg
aggaaattgcatcgcattgtctgagtaggtgtcattctattctggggggt
ggggtggggcaggacagcaagggggaggattgggaagacaatagcaggca
tgctggggatgcggtgggctctatgg

HA Right

atggcagtggccggtggggacagggctgagccagcaccaaccactcagcc
tttgagatcccgaggctggtctactgctgagaccttttgttagaagagag
gagatcaagcatttgcaaggtttctgagtgtcaaaatatgaatccaagat
aactctttcacaatcctaacttcatgctgtctacaggtccatattttagc
ctgctttctccatgttcatccgaaaagaaagaaaagctaagggtggtggt
catatttgaaattagccagatcttaagtttttctgggggaaatttagaag
aaaatatggaaaagtgactatgagcaca

### AAV6 Vector Carrying SA_GFP_3′UTR-RAG1, Guide 9:

INSERT

tgagcacacagttattacttggaaattgtgtacagactaagttgaagatg
ttaggagggaagattgtgggccaagtaacggggtgtatgtgtgtgggtat
agggtgggcagctgggatggaaatggggggctgctgctgctgctgcaccc
tggcctcctgaactaatgatatcactcaccagaaactactgttcctgcac
tgtccaagccaccccaaactagtttgtcaaaatgaatctgtgctgtgtgg
agggaggcacgcctgtagctctgatgtcagatggcaatgtgaattcctga
cctcttctcttcctcccacaggccgccaccatggtgagcaagggcgagga
gctgttcaccggggtggtgcccatcctggtcgagctggacggcgacgtaa
acggccacaagttcagcgtgtccggcgagggcgagggcgatgccacctac
ggcaagctgaccctgaagttcatctgcaccaccggcaagctgcccgtgcc
ctggcccaccctcgtgaccaccctgacctacggcgtgcagtgcttcagtc
gctaccccgaccacatgaagcagcacgacttcttcaagtccgccatgccc
gaaggctacgtccaggagcgcaccatcttcttcaaggacgacggcaacta
caagacccgcgccgaggtgaagttcgagggcgacaccctggtgaaccgca
tcgagctgaagggcatcgacttcaaggaggacggcaacatcctggggcac
aagctggagtacaactacaacagccacaacgtctatatcatggccgacaa
gcagaagaacggcatcaaggtgaacttcaagatccgccacaacatcgagg
acggcagcgtgcagctcgccgaccactaccagcagaacacccccatcggc
gacggccccgtgctgctgcctgacaaccactacctgagcacccagtccgc
cctgagcaaagaccccaacgagaagcgcgatcacatggtcctgctggagt
tcgtgaccgccgccgggatcactctcggcatggacgagctgtacaagtaa
actggatccgtagggcaaccacttatgagttggtttttgcaattgagttt
ccctctgggttgcattgagggcttctcctagcaccctttactgctgtgta
tggggcttcaccatccaagaggtggtaggttggagtaagatgctacagat
gctctcaagtcaggaatagaaactgatgagctgattgcttgaggctttta
gtgagttccgaaaagcaacaggaaaaatcagttatctgaaagctcagtaa
ctcagaacaggagtaactgcaggggaccagagatgagcaaagatctgtgt
gtgttggggagctgtcatgtaaatcaaagccaaggttgtcaaagaacagc
cagtgaggccaggaaagaaattggtcttgtggttttcatttttttccccc
ttgattgattatattttgtattgagatatgataagtgccttctatttcat
ttttgaataattcttcatttttataattttacatatcttggcttgctata
taagattcaaaagagctttttaaatttttctaataatatcttacatttgt
acagcatgatgacctttacaaagtgctctcaatgcatttacccattcgtt
atataaatatgttacatcaggacaactttgagaaaatcagtcctttttta
tgtttaaattatgtatctattgtaaccttcagagtttaggaggtcatctg
ctgtcatggatttttcaataatgaatttagaatacacctgttagctacag
ttagttattaaatcttctgataatatatgtttacttagctatcagaagcc
aagtatgattctttatttttactttttcatttcaagaaatttagagtttc
caaatttagagcttctgcatacagtcttaaagccacagaggcttgtaaaa
atataggttagcttgatgtctaaaaatatatttcatgtcttactgaaaca
ttttgccagactttctccaaatgaaacctgaatcaatttttctaaatcta
ggtttcatagagtcctctcctctgcaatgtgttattctttctataatgat
cagtttactttcagtggattcagaattgtgtagcaggataaccttgtatt
tttccatccgctaagtttagatggagtccaaacgcagtacagcagaagag
ttaacatttacacagtgctttttaccactgtggaatgttttcacactcat
ttttccttacaacaattctgaggagtaggtgttgttattatctccatttg
atgggggtttaaatgatttgctcaaagtcatttaggggtaataaatactt
ggcttggaaatttaacacagtccttttgtctccaaagcccttcttctttc
caccacaaattaatcactatgtttataaggtagtatcagaatttttttag
gattcacaactaatcactatagcacatgaccttgggattacatttttatg
gggcaggggtaagcaagtttttaaatcatttgtgtgctctggctcttttg
atagaagaaagcaacacaaaagctccaaagggccccctaaccctcttgtg
gctccagttatttggaaactatgatctgcatccttaggaatctgggattt
gccagttgctggcaatgtagagcaggcatggaattttatatgctagtgag
tcataatgatatgttagtgttaattagttttttcttcctttgattttatt
ggccataattgctactcttcatacacagtatatcaaagagcttgataatt
tagttgtcaaaagtgcatcggcgacattatctttaattgtatgtatttgg
tgcttcttcagggattgaactcagtatctttcattaaaaaacacagcagt
tttccttgctttttatatgcagaatatcaaagtcatttctaatttagttg
tcaaaaacatatacatattttaacattagtttttttgaaaactcttggtt
ttgtttttttggaaatgagtgggccactaagccacactttcccttcatcc
tgcttaatccttccagcatgtctctgcactaataaacagctaaattcaca
taatcatcctatttactgaagcatggtcatgctggtttatagatttttta
cccatttctactctttttctctattggtggcactgtaaatactttccagt
attaaattatccttttctaacactgtaggaactattttgaatgcatgtga
ctaagagcatgatttatagcacaacctttccaataatcccttaatcagat
cacattttgataaaccctgggaacatctggctgcaggaatttcaatatgt
agaaacgctgcctatggttttttgcccttactgttgagactgcaatatcc
tagaccctagttttatactagagttttatttttagcaatgcctattgcaa
gtgcaattatatactccagggaaattcaccacactgaatcgagcatttgt
gtgtgtatgtgtgaagtatatactgggacttcagaagtgcaatgtatttt
tctcctgtgaaacctgaatctacaagttttcctgccaagccactcaggtg
cattgcagggaccagtgataatggctgatgaaaattgatgattggtcagt
gaggtcaaaaggagccttgggattaataaacatgcactgagaagcaagag
gaggagaaaaagatgtctttttcttccaggtgaactggaatttagttttg
cctcagatttttttcccacaagatacagaagaagataaagatttttttgg
ttgagagtgtgggtcttgcattacatcaaacagagttcaaattccacaca
gataagaggcaggatatataagcgccagtggtagttgggaggaataaacc
attatttggatgcaggtggtttttgattgcaaatatgtgtgtgtcttcag
tgattgtatgacagatgatgtattcttttgatgttaaaagattttaagta
agagtagatacattgtacccattttacattttcttattttaactacagta
atctacataaatatacctcagaaatcatttttggtgattattttttgttt
tgtagaattgcacttcagtttattttcttacaaataaccttacattttgt
ttaatggcttccaagagccttttttttttttgtatttcagagaaaattca
ggtaccaggatgcaatggatttatttgattcaggggacctgtgtttccat
gtcaaatgttttcaaataaaatgaaatatgagtttcaatactttttatat
tttaatatttccattcattaatattatggttattgtcagcaattttatgt
ttgaatatttgaaataaaagtttaagatttgaaaatggtatgtattataa
tttctattcaaatattaataataatattgagtgcagcatttctagaatgg
cagtggccggtggggacagggctgagccagcaccaaccactcagcctttg
agatcccgaggctggtctactgctgagaccttttgttagaagagaggaga
tcaagcatttgcaaggtttctgagtgtcaaaatatgaatccaagataact
ctttcacaatcctaacttcatgctgtctacaggtccatattttagcctgc
tttctccatgttcatccgaaaagaaagaaaagctaagggtggtggtcata
tttgaaattagccagatcttaagtttttctgggggaaatttagaagaaaa
tatggaaaagtgactatgagcaca

HA Left

tgagcacacagttattacttggaaattgtgtacagactaagttgaagatg
ttaggagggaagattgtgggccaagtaacggggtgtatgtgtgtgggtat
agggtgggcagctgggatggaaatggggggctgctgctgctgctgcaccc
tggcctcctgaactaatgatatcactcaccagaaactactgttcctgcac
tgtccaagccaccccaaactagtttgtcaaaatgaatctgtgctgtgtgg
agggaggcacgcctgtagctctgatgtcagatggcaatgt

Splice Acceptor

ctgacctcttctcttcctcccacag

KOZAK

gccgccaccatg

GFP

atggtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagtcgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcctgacaaccac
tacctgagcacccagtccgccctgagcaaagaccccaacgagaagcgcga
tcacatggtcctgctggagttcgtgaccgccgccgggatcactctcggca
tggacgagctgtacaagtaa

3′UTR

gtagggcaaccacttatgagttggtttttgcaattgagtttccctctggg
ttgcattgagggcttctcctagcaccctttactgctgtgtatggggcttc
accatccaagaggtggtaggttggagtaagatgctacagatgctctcaag
tcaggaatagaaactgatgagctgattgcttgaggcttttagtgagttcc
gaaaagcaacaggaaaaatcagttatctgaaagctcagtaactcagaaca
ggagtaactgcaggggaccagagatgagcaaagatctgtgtgtgttgggg
agctgtcatgtaaatcaaagccaaggttgtcaaagaacagccagtgaggc
caggaaagaaattggtcttgtggttttcatttttttcccccttgattgat
tatattttgtattgagatatgataagtgccttctatttcatttttgaata
attcttcatttttataattttacatatcttggcttgctatataagattca
aaagagctttttaaatttttctaataatatcttacatttgtacagcatga
tgacctttacaaagtgctctcaatgcatttacccattcgttatataaata
tgttacatcaggacaactttgagaaaatcagtccttttttatgtttaaat
tatgtatctattgtaaccttcagagtttaggaggtcatctgctgtcatgg
atttttcaataatgaatttagaatacacctgttagctacagttagttatt
aaatcttctgataatatatgtttacttagctatcagaagccaagtatgat
tctttatttttactttttcatttcaagaaatttagagtttccaaatttag
agcttctgcatacagtcttaaagccacagaggcttgtaaaaatataggtt
agcttgatgtctaaaaatatatttcatgtcttactgaaacattttgccag
actttctccaaatgaaacctgaatcaatttttctaaatctaggtttcata
gagtcctctcctctgcaatgtgttattctttctataatgatcagtttact
ttcagtggattcagaattgtgtagcaggataaccttgtatttttccatcc
gctaagtttagatggagtccaaacgcagtacagcagaagagttaacattt
acacagtgctttttaccactgtggaatgttttcacactcatttttcctta
caacaattctgaggagtaggtgttgttattatctccatttgatgggggtt
taaatgatttgctcaaagtcatttaggggtaataaatacttggcttggaa
atttaacacagtccttttgtctccaaagcccttcttctttccaccacaaa
ttaatcactatgtttataaggtagtatcagaatttttttaggattcacaa
ctaatcactatagcacatgaccttgggattacatttttatggggcagggg
taagcaagtttttaaatcatttgtgtgctctggctcttttgatagaagaa
agcaacacaaaagctccaaagggccccctaaccctcttgtggctccagtt
atttggaaactatgatctgcatccttaggaatctgggatttgccagttgc
tggcaatgtagagcaggcatggaattttatatgctagtgagtcataatga
tatgttagtgttaattagttttttcttcctttgattttattggccataat
tgctactcttcatacacagtatatcaaagagcttgataatttagttgtca
aaagtgcatcggcgacattatctttaattgtatgtatttggtgcttcttc
agggattgaactcagtatctttcattaaaaaacacagcagttttccttgc
tttttatatgcagaatatcaaagtcatttctaatttagttgtcaaaaaca
tatacatattttaacattagtttttttgaaaactcttggttttgtttttt
tggaaatgagtgggccactaagccacactttcccttcatcctgcttaatc
cttccagcatgtctctgcactaataaacagctaaattcacataatcatcc
tatttactgaagcatggtcatgctggtttatagattttttacccatttct
actctttttctctattggtggcactgtaaatactttccagtattaaatta
tccttttctaacactgtaggaactattttgaatgcatgtgactaagagca
tgatttatagcacaacctttccaataatcccttaatcagatcacattttg
ataaaccctgggaacatctggctgcaggaatttcaatatgtagaaacgct
gcctatggttttttgcccttactgttgagactgcaatatcctagacccta
gttttatactagagttttatttttagcaatgcctattgcaagtgcaatta
tatactccagggaaattcaccacactgaatcgagcatttgtgtgtgtatg
tgtgaagtatatactgggacttcagaagtgcaatgtatttttctcctgtg
aaacctgaatctacaagttttcctgccaagccactcaggtgcattgcagg
gaccagtgataatggctgatgaaaattgatgattggtcagtgaggtcaaa
aggagccttgggattaataaacatgcactgagaagcaagaggaggagaaa
aagatgtctttttcttccaggtgaactggaatttagttttgcctcagatt
tttttcccacaagatacagaagaagataaagatttttttggttgagagtg
tgggtcttgcattacatcaaacagagttcaaattccacacagataagagg
caggatatataagcgccagtggtagttgggaggaataaaccattatttgg
atgcaggtggtttttgattgcaaatatgtgtgtgtcttcagtgattgtat
gacagatgatgtattcttttgatgttaaaagattttaagtaagagtagat
acattgtacccattttacattttcttattttaactacagtaatctacata
aatatacctcagaaatcatttttggtgattattttttgttttgtagaatt
gcacttcagtttattttcttacaaataaccttacattttgtttaatggct
tccaagagccttttttttttttgtatttcagagaaaattcaggtaccagg
atgcaatggatttatttgattcaggggacctgtgtttccatgtcaaatgt
tttcaaataaaatgaaatatgagtttcaatactttttatattttaatatt
tccattcattaatattatggttattgtcagcaattttatgtttgaatatt
tgaaataaaagtttaagatttgaaaatggtatgtattataatttctattc
aaatattaataataatattgagtgcagcatt

HA Right

atggcagtggccggtggggacagggctgagccagcaccaaccactcagcc
tttgagatcccgaggctggtctactgctgagaccttttgttagaagagag
gagatcaagcatttgcaaggtttctgagtgtcaaaatatgaatccaagat
aactctttcacaatcctaacttcatgctgtctacaggtccatattttagc
ctgctttctccatgttcatccgaaaagaaagaaaagctaagggtggtggt
catatttgaaattagccagatcttaagtttttctgggggaaatttagaag
aaaatatggaaaagtgactatgagcaca

### AAV6 Vector Carrying SA_RAG1-CDS_BGHPolyA, Guide 9:

INSERT

tgagcacacagttattacttggaaattgtgtacagactaagttgaagatg
ttaggagggaagattgtgggccaagtaacggggtgtatgtgtgtgggtat
agggtgggcagctgggatggaaatggggggctgctgctgctgctgcaccc
tggcctcctgaactaatgatatcactcaccagaaactactgttcctgcac
tgtccaagccaccccaaactagtttgtcaaaatgaatctgtgctgtgtgg
agggaggcacgcctgtagctctgatgtcagatggcaatgtgaattcctga
cctcttctcttcctcccacaggccgccaccatggccgcctccttcccacc
tacccttggattgtcctccgcccctgacgaaattcaacatccccacatca
aattctcggagtggaagttcaagctctttcgcgtgcgctcgttcgaaaag
acccccgaggaagcccaaaaggagaagaaagactcattcgaaggaaaacc
cagcctcgaacagtccccggccgtcctggacaaggccgacgggcagaagc
ctgtgccgacccagccgctgctgaaagcgcacccgaaattctccaagaag
tttcacgataacgagaaggcccggggaaaggccatccaccaagcaaacct
tagacacctgtgccgcatctgtgggaactcattcagagccgacgaacata
accggagataccctgtgcatggccctgtcgacggaaagaccctggggctc
ctgagaaagaaggagaagagggcgacatcctggccggacctgatcgcaaa
ggtgttcagaatcgacgtgaaggcagatgtggacagcatccacccaaccg
agttctgccacaactgctggagcattatgcaccggaagttcagctcagcg
ccctgtgaagtgtacttcccgcgcaacgtgactatggagtggcatccaca
cactccgtcctgcgacatctgtaacactgctcggcgcggactcaagagga
agtccctgcagccgaatctgcagctgagcaagaagcttaagaccgtgctg
gaccaggctcggcaggcccgccagcacaagcgacgcgcccaggcccggat
ctcatctaaggatgtgatgaagaagatcgccaattgcagcaaaatccacc
tgtctaccaagctgctggcggtggacttcccggagcacttcgtgaagtcc
atcagctgtcagatctgcgagcatattctcgccgaccccgtggagactaa
ttgcaagcacgtgttctgccgcgtgtgcatcctgcgctgcctgaaggtca
tgggctcctattgcccttcctgccggtacccctgtttccctactgatctg
gagtccccggtcaagtccttcttgtccgtgctgaactccctgatggtcaa
atgtcccgcaaaggagtgcaatgaggaagtgtccctggaaaagtacaacc
accacatcagcagccacaaggagtccaaagaaatctttgtgcacattaac
aagggcggtcggccccggcagcatctgctctcgctgactcgccgggccca
gaagcacaggctccgggagctgaagctgcaagtcaaggccttcgccgaca
aggaagagggaggagatgtgaagtccgtgtgcatgaccctgtttttgctg
gcgctgcgggctcggaacgaacacagacaagctgatgaactggaggccat
catgcagggcaaaggatcgggactccagccggctgtgtgtctcgccatcc
gcgtcaacacattcctctcatgctcccaataccacaagatgtacaggact
gtgaaggccatcaccggacggcagatctttcagccactccacgcccttcg
gaacgcagaaaaggtcttgctgccgggataccatcatttcgaatggcagc
cgcccttgaaaaacgtgtcctcgtccaccgacgtgggcattattgatggg
ctgagcggcctgtcctcctctgtggatgactaccctgtggataccatcgc
caaacggttcagatacgattccgcgctggtgtcggccctgatggacatgg
aggaggacatcctggagggaatgagatcacaagatctggacgactacctc
aacgggcccttcacggtggtggtcaaggaatcgtgcgatggaatgggcga
cgtgtcggagaagcacggttccggacctgtggtgccggaaaaggccgtgc
gcttctccttcaccatcatgaagatcaccattgcgcatagctcccagaac
gtcaaagtgttcgaagaggccaagccgaactcagagctctgctgcaagcc
gctgtgcctgatgttggcggacgagagcgatcacgaaaccctgaccgcca
ttctgtcgcctctgatcgcggagagggaggccatgaagtcctccgaactg
atgctggagctgggcggtattttgcggacttttaagttcatcttccgggg
aaccggttatgacgaaaagctcgtgcgcgaagtggagggcctggaagcct
caggctccgtctacatctgcactctctgcgacgccacccggctggaggcg
tcacagaatcttgtgttccactcgatcactaggtcccacgcggagaacct
ggaacgctatgaggtctggcgctctaacccataccacgaatccgtggaag
aacttcgggacagagtgaagggagtgtcagcaaagcctttcattgaaacc
gtgcctagcatcgacgccctccattgcgacatcggcaacgccgccgagtt
ctacaagatcttccagcttgagatcggggaagtgtacaagaacccgaacg
cctccaaggaagaaagaaagcggtggcaggctacccttgacaaacacctc
cgcaagaagatgaacctgaagcccattatgcggatgaacggaaacttcgc
taggaagctgatgactaaggaaacggtcgacgcggtctgtgaactgatcc
ccagcgaagaacgacatgaagcgctgcgcgaactcatggacctgtacctg
aagatgaagcctgtctggcggagctcgtgccctgccaaggagtgcccgga
gtcgctgtgtcagtacagctttaacagccaaaggttcgcagagctgctgt
cgaccaagttcaagtacagatacgaaggaaagattaccaactacttccac
aagactctcgctcacgtgcccgagattatcgaacgcgatggttccatcgg
ggcctgggcctccgagggcaacgagtcgggcaacaagttgttccgccggt
ttagaaagatgaacgcccgccagtccaagtgctacgaaatggaagatgtg
ctgaagcatcactggctgtatacctccaagtacctccagaagttcatgaa
cgcacataacgccctcaagacctccgggttcaccatgaacccccaggcct
ccctcggtgaccctctgggaattgaagatagcttggagagccaggactcg
atggaattctagctgtgccttctagttgccagccatctgttgtttgcccc
tcccccgtgccttccttgaccctggaaggtgccactcccactgtcctttc
ctaataaaatgaggaaattgcatcgcattgtctgagtaggtgtcattcta
ttctggggggtggggtggggcaggacagcaagggggaggattgggaagac
aatagcaggcatgctggggatgcggtgggctctatggtctagaatggcag
tggccggtggggacagggctgagccagcaccaaccactcagcctttgaga
tcccgaggctggtctactgctgagaccttttgttagaagagaggagatca
agcatttgcaaggtttctgagtgtcaaaatatgaatccaagataactctt
tcacaatcctaacttcatgctgtctacaggtccatattttagcctgcttt
ctccatgttcatccgaaaagaaagaaaagctaagggtggtggtcatattt
gaaattagccagatcttaagtttttctgggggaaatttagaagaaaatat
ggaaaagtgactatgagcaca

HA Left

tgagcacacagttattacttggaaattgtgtacagactaagttgaagatg
ttaggagggaagattgtgggccaagtaacggggtgtatgtgtgtgggtat
agggtgggcagctgggatggaaatggggggctgctgctgctgctgcaccc
tggcctcctgaactaatgatatcactcaccagaaactactgttcctgcac
tgtccaagccaccccaaactagtttgtcaaaatgaatctgtgctgtgtgg
agggaggcacgcctgtagctctgatgtcagatggcaatgt

SA

ctgacctcttctcttcctcccacag

KOZAK

gccgccaccatg

RAG1-CDS

ctgacctcttctcttcctcccacaggtacctcagccagcatggccgcctc
cttcccacctacccttggattgtcctccgcccctgacgaaattcaacatc
cccacatcaaattctcggagtggaagttcaagctctttcgcgtgcgctcg
ttcgaaaagacccccgaggaagcccaaaaggagaagaaagactcattcga
aggaaaacccagcctcgaacagtccccggccgtcctggacaaggccgacg
ggcagaagcctgtgccgacccagccgctgctgaaagcgcacccgaaattc
tccaagaagtttcacgataacgagaaggcccggggaaaggccatccacca
agcaaaccttagacacctgtgccgcatctgtgggaactcattcagagccg
acgaacataaccggagataccctgtgcatggccctgtcgacggaaagacc
ctggggctcctgagaaagaaggagaagagggcgacatcctggccggacct
gatcgcaaaggtgttcagaatcgacgtgaaggcagatgtggacagcatcc
acccaaccgagttctgccacaactgctggagcattatgcaccggaagttc
agctcagcgccctgtgaagtgtacttcccgcgcaacgtgactatggagtg
gcatccacacactccgtcctgcgacatctgtaacactgctcggcgcggac
tcaagaggaagtccctgcagccgaatctgcagctgagcaagaagcttaag
accgtgctggaccaggctcggcaggcccgccagcacaagcgacgcgccca
ggcccggatctcatctaaggatgtgatgaagaagatcgccaattgcagca
aaatccacctgtctaccaagctgctggcggtggacttcccggagcacttc
gtgaagtccatcagctgtcagatctgcgagcatattctcgccgaccccgt
ggagactaattgcaagcacgtgttctgccgcgtgtgcatcctgcgctgcc
tgaaggtcatgggctcctattgcccttcctgccggtacccctgtttccct
actgatctggagtccccggtcaagtccttcttgtccgtgctgaactccct
gatggtcaaatgtcccgcaaaggagtgcaatgaggaagtgtccctggaaa
agtacaaccaccacatcagcagccacaaggagtccaaagaaatctttgtg
cacattaacaagggcggtcggccccggcagcatctgctctcgctgactcg
ccgggcccagaagcacaggctccgggagctgaagctgcaagtcaaggcct
tcgccgacaaggaagagggaggagatgtgaagtccgtgtgcatgaccctg
tttttgctggcgctgcgggctcggaacgaacacagacaagctgatgaact
ggaggccatcatgcagggcaaaggatcgggactccagccggctgtgtgtc
tcgccatccgcgtcaacacattcctctcatgctcccaataccacaagatg
tacaggactgtgaaggccatcaccggacggcagatctttcagccactcca
cgcccttcggaacgcagaaaaggtcttgctgccgggataccatcatttcg
aatggcagccgcccttgaaaaacgtgtcctcgtccaccgacgtgggcatt
attgatgggctgagcggcctgtcctcctctgtggatgactaccctgtgga
taccatcgccaaacggttcagatacgattccgcgctggtgtcggccctga
tggacatggaggaggacatcctggagggaatgagatcacaagatctggac
gactacctcaacgggcccttcacggtggtggtcaaggaatcgtgcgatgg
aatgggcgacgtgtcggagaagcacggttccggacctgtggtgccggaaa
aggccgtgcgcttctccttcaccatcatgaagatcaccattgcgcatagc
tcccagaacgtcaaagtgttcgaagaggccaagccgaactcagagctctg
ctgcaagccgctgtgcctgatgttggcggacgagagcgatcacgaaaccc
tgaccgccattctgtcgcctctgatcgcggagagggaggccatgaagtcc
tccgaactgatgctggagctgggcggtattttgcggacttttaagttcat
cttccggggaaccggttatgacgaaaagctcgtgcgcgaagtggagggcc
tggaagcctcaggctccgtctacatctgcactctctgcgacgccacccgg
ctggaggcgtcacagaatcttgtgttccactcgatcactaggtcccacgc
ggagaacctggaacgctatgaggtctggcgctctaacccataccacgaat
ccgtggaagaacttcgggacagagtgaagggagtgtcagcaaagcctttc
attgaaaccgtgcctagcatcgacgccctccattgcgacatcggcaacgc
cgccgagttctacaagatcttccagcttgagatcggggaagtgtacaaga
acccgaacgcctccaaggaagaaagaaagcggtggcaggctacccttgac
aaacacctccgcaagaagatgaacctgaagcccattatgcggatgaacgg
aaacttcgctaggaagctgatgactaaggaaacggtcgacgcggtctgtg
aactgatccccagcgaagaacgacatgaagcgctgcgcgaactcatggac
ctgtacctgaagatgaagcctgtctggcggagctcgtgccctgccaagga
gtgcccggagtcgctgtgtcagtacagctttaacagccaaaggttcgcag
agctgctgtcgaccaagttcaagtacagatacgaaggaaagattaccaac
tacttccacaagactctcgctcacgtgcccgagattatcgaacgcgatgg
ttccatcggggcctgggcctccgagggcaacgagtcgggcaacaagttgt
tccgccggtttagaaagatgaacgcccgccagtccaagtgctacgaaatg
gaagatgtgctgaagcatcactggctgtatacctccaagtacctccagaa
gttcatgaacgcacataacgccctcaagacctccgggttcaccatgaacc
cccaggcctccctcggtgaccctctgggaattgaagatagcttggagagc
caggactcgatggaattctagctgtgccttctagttgccagccatctgtt
gtttgcccctcccccgtgccttccttgaccctggaaggtgccactcccac
tgtcctttcctaataaaatgaggaaattgcatcgcattgtctgagtaggt
gtcattctattctggggggtggggtggggcaggacagcaagggggaggat
tgggaagacaatagcaggcatgctggggatgcggtgggctctatggatcc
ggaacaggtgtgataatgagagatcttgcgttccaacgagaaactaatgt
t

PolyA

gctgtgccttctagttgccagccatctgttgtttgcccctcccccgtgcc
ttccttgaccctggaaggtgccactcccactgtcctttcctaataaaatg
aggaaattgcatcgcattgtctgagtaggtgtcattctattctggggggt
ggggtggggcaggacagcaagggggaggattgggaagacaatagcaggca
tgctggggatgcggtgggctctatgg

HA Right

atggcagtggccggtggggacagggctgagccagcaccaaccactcagcc
tttgagatcccgaggctggtctactgctgagaccttttgttagaagagag
gagatcaagcatttgcaaggtttctgagtgtcaaaatatgaatccaagat
aactctttcacaatcctaacttcatgctgtctacaggtccatattttagc
ctgctttctccatgttcatccgaaaagaaagaaaagctaagggtggtggt
catatttgaaattagccagatcttaagtttttctgggggaaatttagaag
aaaatatggaaaagtgactatgagcaca

### AAV6 Vector Carrying PGK_GFP_BGHPolyA, Guide 3:

INSERT

gcaaagatgaatcaaagattctgtccttaaagaccttaaggtttttgtgg
aaggaaataaaactttacatgtatatatttaagcacttatatgtgtgtaa
caggtataagtaaccataaacactgtcagaagaggaaataactctatgat
cagcacctaacatgatatattaaggtagaagatttaatacatatcttttg
gaatacatgaataaataattgaatgtatttatttttattatttataagat
acatcagtgggatattgatattggtcttaatatgacttgttttcattgtt
ctcaggtacctcagccagcatggcagcctctttcccacccaccttgggac
tcagttctgccccagataccggtatggccacggggttggggttgcgcctt
ttccaaggcagccctgggtttgcgcagggacgcggctgctctgggcgtgg
ttccgggaaacgcagcggcgccgaccctgggtctcgcacattcttcacgt
ccgttcgcagcgtcacccggatcttcgccgctacccttgtgggccccccg
gcgacgcttcctgctccgcccctaagtcgggaaggttccttgcggttcgc
ggcgtgccggacgtgacaaacggaagccgcacgtctcactagtaccctcg
cagacggacagcgccagggagcaatggcagcgcgccgaccgcgatgggct
gtggccaatagcggctgctcagcagggcgcgccgagagcagcggccggga
aggggcggtgcgggaggcggggtgtggggcggtagtgtgggccctgttcc
tgcccgcgcggtgttccgcattctgcaagcctccggagcgcacgtcggca
gtcggctccctcgttgaccgaatcaccgacctctctccccagggccgcca
ccatggtgagcaagggcgaggagctgttcaccggggtggtgcccatcctg
gtcgagctggacggcgacgtaaacggccacaagttcagcgtgtccggcga
gggcgagggcgatgccacctacggcaagctgaccctgaagttcatctgca
ccaccggcaagctgcccgtgccctggcccaccctcgtgaccaccctgacc
tacggcgtgcagtgcttcagtcgctaccccgaccacatgaagcagcacga
cttcttcaagtccgccatgcccgaaggctacgtccaggagcgcaccatct
tcttcaaggacgacggcaactacaagacccgcgccgaggtgaagttcgag
ggcgacaccctggtgaaccgcatcgagctgaagggcatcgacttcaagga
ggacggcaacatcctggggcacaagctggagtacaactacaacagccaca
acgtctatatcatggccgacaagcagaagaacggcatcaaggtgaacttc
aagatccgccacaacatcgaggacggcagcgtgcagctcgccgaccacta
ccagcagaacacccccatcggcgacggccccgtgctgctgcctgacaacc
actacctgagcacccagtccgccctgagcaaagaccccaacgagaagcgc
gatcacatggtcctgctggagttcgtgaccgccgccgggatcactctcgg
catggacgagctgtacaagtaaactgtgccttctagttgccagccatctg
ttgtttgcccctcccccgtgccttccttgaccctggaaggtgccactccc
actgccctttcctaataaaatgaggaaattgcatcgcattgtctgagtag
gtgtcattctattctggggggtggggtggggcaggacagcaagggggagg
attgggaagacaatagcaggcatgctggggatgcggtgggctctatgggg
atccggaacaggtgtgataatgagagatcttgcgttccaacgagaaacta
atgtttctagaaattcagcacccacatattaaattttcagaatggaaatt
taagctgttccgggtgagatcctttgaaaagacacctgaagaagctcaaa
aggaaaagaaggattcctttgaggggaaaccctctctggagcaatctcca
gcagtcctggacaaggctgatggtcagaagccagtcccaactcagccatt
gttaaaagcccaccctaagttttcaaagaaatttcacgacaacgagaaag
caagaggcaaagcgatccatcaagccaaccttcgacatctctgccgcatc
tgtgggaattcttttagagctgatgagcacaacaggagatatccagtcca
tggtcctgtggatggtaaaaccctaggccttttacgaaagaaggaaaaga
gagc

HA Left

gcaaagatgaatcaaagattctgtccttaaagaccttaaggtttttgtgg
aaggaaataaaactttacatgtatatatttaagcacttatatgtgtgtaa
caggtataagtaaccataaacactgtcagaagaggaaataactctatgat
cagcacctaacatgatatattaaggtagaagatttaatacatatcttttg
gaatacatgaataaataattgaatgtatttatttttattatttataagat
acatcagtgggatattgatattggtcttaatatgacttgttttcattgtt
ctcaggtacctcagccagcatggcagcctctttcccacccaccttgggac
tcagttctgccccagat

PGK promoter

ccacggggttggggttgcgccttttccaaggcagccctgggtttgcgcag
ggacgcggctgctctgggcgtggttccgggaaacgcagcggcgccgaccc
tgggtctcgcacattcttcacgtccgttcgcagcgtcacccggatcttcg
ccgctacccttgtgggccccccggcgacgcttcctgctccgcccctaagt
cgggaaggttccttgcggttcgcggcgtgccggacgtgacaaacggaagc
cgcacgtctcactagtaccctcgcagacggacagcgccagggagcaatgg
cagcgcgccgaccgcgatgggctgtggccaatagcggctgctcagcaggg
cgcgccgagagcagcggccgggaaggggcggtgcgggaggcggggtgtgg
ggcggtagtgtgggccctgttcctgcccgcgcggtgttccgcattctgca
agcctccggagcgcacgtcggcagtcggctccctcgttgaccgaatcacc
gacctctctccccagg

KOZAK

ccatg

GFP

atggtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagtcgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcctgacaaccac
tacctgagcacccagtccgccctgagcaaagaccccaacgagaagcgcga
tcacatggtcctgctggagttcgtgaccgccgccgggatcactctcggca
tggacgagctgtacaagtaa

PolyA

actgtgccttctagttgccagccatctgttgtttgcccctcccccgtgcc
ttccttgaccctggaaggtgccactcccactgccctttcctaataaaatg
aggaaattgcatcgcattgtctgagtaggtgtcattctattctggggggt
ggggtggggcaggacagcaagggggaggattgggaagacaatagcaggca
tgctggggatgcggtgggctctatgg

HA Right

aattcagcacccacatattaaattttcagaatggaaatttaagctgttcc
gggtgagatcctttgaaaagacacctgaagaagctcaaaaggaaaagaag
gattcctttgaggggaaaccctctctggagcaatctccagcagtcctgga
caaggctgatggtcagaagccagtcccaactcagccattgttaaaagccc
accctaagttttcaaagaaatttcacgacaacgagaaagcaagaggcaaa
gcgatccatcaagccaaccttcgacatctctgccgcatctgtgggaattc
ttttagagctgatgagcacaacaggagatatccagtccatggtcctgtgg
atggtaaaaccctaggccttttacgaaagaaggaaaagagagc

### Lentiviral Vector Carrying TET_Cas9_PGK_Puro

INSERT

atggactataaggaccacgacggagactacaaggatcatgatattgatta
caaagacgatgacgataagatggccccaaagaagaagcggaaggtcggta
tccacggagtcccagcagccgacaagaagtacagcatcggcctggacatc
ggcaccaactctgtgggctgggccgtgatcaccgacgagtacaaggtgcc
cagcaagaaattcaaggtgctgggcaacaccgaccggcacagcatcaaga
agaacctgatcggagccctgctgttcgacagcggcgaaacagccgaggcc
acccggctgaagagaaccgccagaagaagatacaccagacggaagaaccg
gatctgctatctgcaagagatcttcagcaacgagatggccaaggtggacg
acagcttcttccacagactggaagagtccttcctggtggaagaggataag
aagcacgagcggcaccccatcttcggcaacatcgtggacgaggtggccta
ccacgagaagtaccccaccatctaccacctgagaaagaaactggtggaca
gcaccgacaaggccgacctgcggctgatctatctggccctggcccacatg
atcaagttccggggccacttcctgatcgagggcgacctgaaccccgacaa
cagcgacgtggacaagctgttcatccagctggtgcagacctacaaccagc
tgttcgaggaaaaccccatcaacgccagcggcgtggacgccaaggccatc
ctgtctgccagactgagcaagagcagacggctggaaaatctgatcgccca
gctgcccggcgagaagaagaatggcctgttcggaaacctgattgccctga
gcctgggcctgacccccaacttcaagagcaacttcgacctggccgaggat
gccaaactgcagctgagcaaggacacctacgacgacgacctggacaacct
gctggcccagatcggcgaccagtacgccgacctgtttctggccgccaaga
acctgtccgacgccatcctgctgagcgacatcctgagagtgaacaccgag
atcaccaaggcccccctgagcgcctctatgatcaagagatacgacgagca
ccaccaggacctgaccctgctgaaagctctcgtgcggcagcagctgcctg
agaagtacaaagagattttcttcgaccagagcaagaacggctacgccggc
tacattgacggcggagccagccaggaagagttctacaagttcatcaagcc
catcctggaaaagatggacggcaccgaggaactgctcgtgaagctgaaca
gagaggacctgctgcggaagcagcggaccttcgacaacggcagcatcccc
caccagatccacctgggagagctgcacgccattctgcggcggcaggaaga
tttttacccattcctgaaggacaaccgggaaaagatcgagaagatcctga
ccttccgcatcccctactacgtgggccctctggccaggggaaacagcaga
ttcgcctggatgaccagaaagagcgaggaaaccatcaccccctggaactt
cgaggaagtggtggacaagggcgcttccgcccagagcttcatcgagcgga
tgaccaacttcgataagaacctgcccaacgagaaggtgctgcccaagcac
agcctgctgtacgagtacttcaccgtgtataacgagctgaccaaagtgaa
atacgtgaccgagggaatgagaaagcccgccttcctgagcggcgagcaga
aaaaggccatcgtggacctgctgttcaagaccaaccggaaagtgaccgtg
aagcagctgaaagaggactacttcaagaaaatcgagtgcttcgactccgt
ggaaatctccggcgtggaagatcggttcaacgcctccctgggcacatacc
acgatctgctgaaaattatcaaggacaaggacttcctggacaatgaggaa
aacgaggacattctggaagatatcgtgctgaccctgacactgtttgagga
cagagagatgatcgaggaacggctgaaaacctatgcccacctgttcgacg
acaaagtgatgaagcagctgaagcggcggagatacaccggctggggcagg
ctgagccggaagctgatcaacggcatccgggacaagcagtccggcaagac
aatcctggatttcctgaagtccgacggcttcgccaacagaaacttcatgc
agctgatccacgacgacagcctgacctttaaagaggacatccagaaagcc
caggtgtccggccagggcgatagcctgcacgagcacattgccaatctggc
cggcagccccgccattaagaagggcatcctgcagacagtgaaggtggtgg
acgagctcgtgaaagtgatgggccggcacaagcccgagaacatcgtgatc
gaaatggccagagagaaccagaccacccagaagggacagaagaacagccg
cgagagaatgaagcggatcgaagagggcatcaaagagctgggcagccaga
tcctgaaagaacaccccgtggaaaacacccagctgcagaacgagaagctg
tacctgtactacctgcagaatgggcgggatatgtacgtggaccaggaact
ggacatcaaccggctgtccgactacgatgtggaccatatcgtgcctcaga
gctttctgaaggacgactccatcgacaacaaggtgctgaccagaagcgac
aagaaccggggcaagagcgacaacgtgccctccgaagaggtcgtgaagaa
gatgaagaactactggcggcagctgctgaacgccaagctgattacccaga
gaaagttcgacaatctgaccaaggccgagagaggcggcctgagcgaactg
gataaggccggcttcatcaagagacagctggtggaaacccggcagatcac
aaagcacgtggcacagatcctggactcccggatgaacactaagtacgacg
agaatgacaagctgatccgggaagtgaaagtgatcaccctgaagtccaag
ctggtgtccgatttccggaaggatttccagttttacaaagtgcgcgagat
caacaactaccaccacgcccacgacgcctacctgaacgccgtcgtgggaa
ccgccctgatcaaaaagtaccctaagctggaaagcgagttcgtgtacggc
gactacaaggtgtacgacgtgcggaagatgatcgccaagagcgagcagga
aatcggcaaggctaccgccaagtacttcttctacagcaacatcatgaact
ttttcaagaccgagattaccctggccaacggcgagatccggaagcggcct
ctgatcgagacaaacggcgaaaccggggagatcgtgtgggataagggccg
ggattttgccaccgtgcggaaagtgctgagcatgccccaagtgaatatcg
tgaaaaagaccgaggtgcagacaggcggcttcagcaaagagtctatcctg
cccaagaggaacagcgataagctgatcgccagaaagaaggactgggaccc
taagaagtacggcggcttcgacagccccaccgtggcctattctgtgctgg
tggtggccaaagtggaaaagggcaagtccaagaaactgaagagtgtgaaa
gagctgctggggatcaccatcatggaaagaagcagcttcgagaagaatcc
catcgactttctggaagccaagggctacaaagaagtgaaaaaggacctga
tcatcaagctgcctaagtactccctgttcgagctggaaaacggccggaag
agaatgctggcctctgccggcgaactgcagaagggaaacgaactggccct
gccctccaaatatgtgaacttcctgtacctggccagccactatgagaagc
tgaagggctcccccgaggataatgagcagaaacagctgtttgtggaacag
cacaagcactacctggacgagatcatcgagcagatcagcgagttctccaa
gagagtgatcctggccgacgctaatctggacaaagtgctgtccgcctaca
acaagcaccgggataagcccatcagagagcaggccgagaatatcatccac
ctgtttaccctgaccaatctgggagcccctgccgccttcaagtactttga
caccaccatcgaccggaagaggtacaccagcaccaaagaggtgctggacg
ccaccctgatccaccagagcatcaccggcctgtacgagacacggatcgac
ctgtctcagctgggaggcgacaaaaggccggcggccacgaaaaaggccgg
ccaggcaaaaaagaaaaagtaaggatccggggttggggttgcgccttttc
caaggcagccctgggtttgcgcagggacgcggctgctctgggcgtggttc
cgggaaacgcagcggcgccgaccctgggtctcgcacattcttcacgtccg
ttcgcagcgtcacccggatcttcgccgctacccttgtgggccccccggcg
acgcttcctgctccgcccctaagtcgggaaggttccttgcggttcgcggc
gtgccggacgtgacaaacggaagccgcacgtctcactagtaccctcgcag
acggacagcgccagggagcaatggcagcgcgccgaccgcgatgggctgtg
gccaatagcggctgctcagcagggcgcgccgagagcagcggccgggaagg
ggcggtgcgggaggcggggtgtggggcggtagtgtgggccctgttcctgc
ccgcgcggtgttccgcattctgcaagcctccggagcgcacgtcggcagtc
ggctccctcgttgaccgaatcaccgacctctctccccagcaattcaccat
gaccgagtacaagcccacggtgcgcctcgccacccgcgacgacgtcccca
gggccgtacgcaccctcgccgccgcgttcgccgactaccccgccacgcgc
cacaccgtcgatccggaccgccacatcgagcgggtcaccgagctgcaaga
actcttcctcacgcgcgtcgggctcgacatcggcaaggtgtgggtcgcgg
acgacggcgccgcggtggcggtctggaccacgccggagagcgtcgaagcg
ggggcggtgttcgccgagatcggcccgcgcatggccgagttgagcggttc
ccggctggccgcgcagcaacagatggaaggcctcctggcgccgcaccggc
ccaaggagcccgcgtggttcctggccaccgtcggcgtctcgcccgaccac
cagggcaagggtctgggcagcgccgtcgtgctccccggagtggaggcggc
cgagcgcgccggggtgcccgccttcctggagacctccgcgccccgcaacc
tccccttctacgagcggctcggcttcaccgtcaccgccgacgtcgaggtg
cccgaaggaccgcgcacctggtgcatgacccgcaagcccggtgccgaagg
tagaggttctctcctcacttgtggtgatgttgaagaaaaccctggtccaa
tgtctagactggacaagagcaaagtcataaacggagctctggaattactc
aatggtgtcggtatcgaaggcctgacgacaaggaaactcgctcaaaagct
gggagttgagcagcctaccctgtactggcacgtgaagaacaagcgggccc
tgctcgatgccctgccaatcgagatgctggacaggcatcatacccacttc
tgccccctggaaggcgagtcatggcaagactttctgcggaacaacgccaa
gtcataccgctgtgctctcctctcacatcgcgacggggctaaagtgcatc
tcggcacccgcccaacagagaaacagtacgaaaccctggaaaatcagctc
gcgttcctgtgtcagcaaggcttctccctggagaacgcactgtacgctct
gtccgccgtgggccactttacactgggctgcgtattggaggaacaggagc
atcaagtagcaaaagaggaaagagagacacctaccaccgattctatgccc
ccacttctgagacaagcaattgagctgttcgaccggcagggagccgaacc
tgccttccttttcggcctggaactaatcatatgtggcctggagaaacagc
taaagtgcgaaagcggcgggccgaccgacgcccttgacgattttgactta
gacatgctcccagccgatgcccttgacgactttgaccttgatatgctgcc
tgctgacgctcttgacgattttgaccttgacatgctccccgggtaaccga
caatcaacctctggattacaaaatttgtgaaagattgactggtattctta
actatgttgctccttttacgctatgtggatacgctgctttaatgcctttg
tatcatgctattgcttcccgtatggctttcattttctcctccttgtataa
atcctggttgctgtctctttatgaggagttgtggcccgttgtcaggcaac
gtggcgtggtgtgcactgtgtttgctgacgcaacccccactggttggggc
attgccaccacctgtcagctcctttccgggactttcgctttccccctccc
tattgccacggcggaactcatcgccgcctgccttgcccgctgctggacag
gggctcggctgttgggcactgacaattccgtggtgttgtcggggaaatca
tcgtcctttccttggctgctcgcctgtgttgccacctggattctgcgcgg
gacgtccttctgctacgtcccttcggccctcaatccagcggaccttcctt
cccgcggcctgctgccggctctgcggcctcttccgcgtcttcgccttcgc
cctcagacgagtcggatctccctttgggccgcctccccgcctg

Cas9

atggactataaggaccacgacggagactacaaggatcatgatattgatta
caaagacgatgacgataagatggccccaaagaagaagcggaaggtcggta
tccacggagtcccagcagccgacaagaagtacagcatcggcctggacatc
ggcaccaactctgtgggctgggccgtgatcaccgacgagtacaaggtgcc
cagcaagaaattcaaggtgctgggcaacaccgaccggcacagcatcaaga
agaacctgatcggagccctgctgttcgacagcggcgaaacagccgaggcc
acccggctgaagagaaccgccagaagaagatacaccagacggaagaaccg
gatctgctatctgcaagagatcttcagcaacgagatggccaaggtggacg
acagcttcttccacagactggaagagtccttcctggtggaagaggataag
aagcacgagcggcaccccatcttcggcaacatcgtggacgaggtggccta
ccacgagaagtaccccaccatctaccacctgagaaagaaactggtggaca
gcaccgacaaggccgacctgcggctgatctatctggccctggcccacatg
atcaagttccggggccacttcctgatcgagggcgacctgaaccccgacaa
cagcgacgtggacaagctgttcatccagctggtgcagacctacaaccagc
tgttcgaggaaaaccccatcaacgccagcggcgtggacgccaaggccatc
ctgtctgccagactgagcaagagcagacggctggaaaatctgatcgccca
gctgcccggcgagaagaagaatggcctgttcggaaacctgattgccctga
gcctgggcctgacccccaacttcaagagcaacttcgacctggccgaggat
gccaaactgcagctgagcaaggacacctacgacgacgacctggacaacct
gctggcccagatcggcgaccagtacgccgacctgtttctggccgccaaga
acctgtccgacgccatcctgctgagcgacatcctgagagtgaacaccgag
atcaccaaggcccccctgagcgcctctatgatcaagagatacgacgagca
ccaccaggacctgaccctgctgaaagctctcgtgcggcagcagctgcctg
agaagtacaaagagattttcttcgaccagagcaagaacggctacgccggc
tacattgacggcggagccagccaggaagagttctacaagttcatcaagcc
catcctggaaaagatggacggcaccgaggaactgctcgtgaagctgaaca
gagaggacctgctgcggaagcagcggaccttcgacaacggcagcatcccc
caccagatccacctgggagagctgcacgccattctgcggcggcaggaaga
tttttacccattcctgaaggacaaccgggaaaagatcgagaagatcctga
ccttccgcatcccctactacgtgggccctctggccaggggaaacagcaga
ttcgcctggatgaccagaaagagcgaggaaaccatcaccccctggaactt
cgaggaagtggtggacaagggcgcttccgcccagagcttcatcgagcgga
tgaccaacttcgataagaacctgcccaacgagaaggtgctgcccaagcac
agcctgctgtacgagtacttcaccgtgtataacgagctgaccaaagtgaa
atacgtgaccgagggaatgagaaagcccgccttcctgagcggcgagcaga
aaaaggccatcgtggacctgctgttcaagaccaaccggaaagtgaccgtg
aagcagctgaaagaggactacttcaagaaaatcgagtgcttcgactccgt
ggaaatctccggcgtggaagatcggttcaacgcctccctgggcacatacc
acgatctgctgaaaattatcaaggacaaggacttcctggacaatgaggaa
aacgaggacattctggaagatatcgtgctgaccctgacactgtttgagga
cagagagatgatcgaggaacggctgaaaacctatgcccacctgttcgacg
acaaagtgatgaagcagctgaagcggcggagatacaccggctggggcagg
ctgagccggaagctgatcaacggcatccgggacaagcagtccggcaagac
aatcctggatttcctgaagtccgacggcttcgccaacagaaacttcatgc
agctgatccacgacgacagcctgacctttaaagaggacatccagaaagcc
caggtgtccggccagggcgatagcctgcacgagcacattgccaatctggc
cggcagccccgccattaagaagggcatcctgcagacagtgaaggtggtgg
acgagctcgtgaaagtgatgggccggcacaagcccgagaacatcgtgatc
gaaatggccagagagaaccagaccacccagaagggacagaagaacagccg
cgagagaatgaagcggatcgaagagggcatcaaagagctgggcagccaga
tcctgaaagaacaccccgtggaaaacacccagctgcagaacgagaagctg
tacctgtactacctgcagaatgggcgggatatgtacgtggaccaggaact
ggacatcaaccggctgtccgactacgatgtggaccatatcgtgcctcaga
gctttctgaaggacgactccatcgacaacaaggtgctgaccagaagcgac
aagaaccggggcaagagcgacaacgtgccctccgaagaggtcgtgaagaa
gatgaagaactactggcggcagctgctgaacgccaagctgattacccaga
gaaagttcgacaatctgaccaaggccgagagaggcggcctgagcgaactg
gataaggccggcttcatcaagagacagctggtggaaacccggcagatcac
aaagcacgtggcacagatcctggactcccggatgaacactaagtacgacg
agaatgacaagctgatccgggaagtgaaagtgatcaccctgaagtccaag
ctggtgtccgatttccggaaggatttccagttttacaaagtgcgcgagat
caacaactaccaccacgcccacgacgcctacctgaacgccgtcgtgggaa
ccgccctgatcaaaaagtaccctaagctggaaagcgagttcgtgtacggc
gactacaaggtgtacgacgtgcggaagatgatcgccaagagcgagcagga
aatcggcaaggctaccgccaagtacttcttctacagcaacatcatgaact
ttttcaagaccgagattaccctggccaacggcgagatccggaagcggcct
ctgatcgagacaaacggcgaaaccggggagatcgtgtgggataagggccg
ggattttgccaccgtgcggaaagtgctgagcatgccccaagtgaatatcg
tgaaaaagaccgaggtgcagacaggcggcttcagcaaagagtctatcctg
cccaagaggaacagcgataagctgatcgccagaaagaaggactgggaccc
taagaagtacggcggcttcgacagccccaccgtggcctattctgtgctgg
tggtggccaaagtggaaaagggcaagtccaagaaactgaagagtgtgaaa
gagctgctggggatcaccatcatggaaagaagcagcttcgagaagaatcc
catcgactttctggaagccaagggctacaaagaagtgaaaaaggacctga
tcatcaagctgcctaagtactccctgttcgagctggaaaacggccggaag
agaatgctggcctctgccggcgaactgcagaagggaaacgaactggccct
gccctccaaatatgtgaacttcctgtacctggccagccactatgagaagc
tgaagggctcccccgaggataatgagcagaaacagctgtttgtggaacag
cacaagcactacctggacgagatcatcgagcagatcagcgagttctccaa
gagagtgatcctggccgacgctaatctggacaaagtgctgtccgcctaca
acaagcaccgggataagcccatcagagagcaggccgagaatatcatccac
ctgtttaccctgaccaatctgggagcccctgccgccttcaagtactttga
caccaccatcgaccggaagaggtacaccagcaccaaagaggtgctggacg
ccaccctgatccaccagagcatcaccggcctgtacgagacacggatcgac
ctgtctcagctgggaggcgacaaaaggccggcggccacgaaaaaggccgg
ccaggcaaaaaagaaaaag

PGK promoter

cggggttggggttgcgccttttccaaggcagccctgggtttgcgcaggga
cgcggctgctctgggcgtggttccgggaaacgcagcggcgccgaccctgg
gtctcgcacattcttcacgtccgttcgcagcgtcacccggatcttcgccg
ctacccttgtgggccccccggcgacgcttcctgctccgcccctaagtcgg
gaaggttccttgcggttcgcggcgtgccggacgtgacaaacggaagccgc
acgtctcactagtaccctcgcagacggacagcgccagggagcaatggcag
cgcgccgaccgcgatgggctgtggccaatagcggctgctcagcagggcgc
gccgagagcagcggccgggaaggggcggtgcgggaggcggggtgtggggc
ggtagtgtgggccctgttcctgcccgcgcggtgttccgcattctgcaagc
ctccggagcgcacgtcggcagtcggctccctcgttgaccgaatcaccgac
ctctctccccag

Puromycin

atgaccgagtacaagcccacggtgcgcctcgccacccgcgacgacgtccc
cagggccgtacgcaccctcgccgccgcgttcgccgactaccccgccacgc
gccacaccgtcgatccggaccgccacatcgagcgggtcaccgagctgcaa
gaactcttcctcacgcgcgtcgggctcgacatcggcaaggtgtgggtcgc
ggacgacggcgccgcggtggcggtctggaccacgccggagagcgtcgaag
cgggggcggtgttcgccgagatcggcccgcgcatggccgagttgagcggt
tcccggctggccgcgcagcaacagatggaaggcctcctggcgccgcaccg
gcccaaggagcccgcgtggttcctggccaccgtcggcgtctcgcccgacc
accagggcaagggtctgggcagcgccgtcgtgctccccggagtggaggcg
gccgagcgcgccggggtgcccgccttcctggagacctccgcgccccgcaa
cctccccttctacgagcggctcggcttcaccgtcaccgccgacgtcgagg
tgcccgaaggaccgcgcacctggtgcatgacccgcaag

rTTA

atgtctagactggacaagagcaaagtcataaacggagctctggaattact
caatggtgtcggtatcgaaggcctgacgacaaggaaactcgctcaaaagc
tgggagttgagcagcctaccctgtactggcacgtgaagaacaagcgggcc
ctgctcgatgccctgccaatcgagatgctggacaggcatcatacccactt
ctgccccctggaaggcgagtcatggcaagactttctgcggaacaacgcca
agtcataccgctgtgctctcctctcacatcgcgacggggctaaagtgcat
ctcggcacccgcccaacagagaaacagtacgaaaccctggaaaatcagct
cgcgttcctgtgtcagcaaggcttctccctggagaacgcactgtacgctc
tgtccgccgtgggccactttacactgggctgcgtattggaggaacaggag
catcaagtagcaaaagaggaaagagagacacctaccaccgattctatgcc
cccacttctgagacaagcaattgagctgttcgaccggcagggagccgaac
ctgccttccttttcggcctggaactaatcatatgtggcctggagaaacag
ctaaagtgcgaaagcggcgggccgaccgacgcccttgacgattttgactt
agacatgctcccagccgatgcccttgacgactttgaccttgatatgctgc
ctgctgacgctcttgacgattttgaccttgacatgctccccgggtaa

WPRE

aatcaacctctggattacaaaatttgtgaaagattgactggtattcttaa
ctatgttgctccttttacgctatgtggatacgctgctttaatgcctttgt
atcatgctattgcttcccgtatggctttcattttctcctccttgtataaa
tcctggttgctgtctctttatgaggagttgtggcccgttgtcaggcaacg
tggcgtggtgtgcactgtgtttgctgacgcaacccccactggttggggca
ttgccaccacctgtcagctcctttccgggactttcgctttccccctccct
attgccacggcggaactcatcgccgcctgccttgcccgctgctggacagg
ggctcggctgttgggcactgacaattccgtggtgttgtcggggaaatcat
cgtcctttccttggctgctcgcctgtgttgccacctggattctgcgcggg
acgtccttctgctacgtcccttcggccctcaatccagcggaccttccttc
ccgcggcctgctgccggctctgcggcctcttccgcgtcttcgccttcgcc
ctcagacgagtcggatctccctttgggccgcctccccgcctg

### Flow Cytometry Analysis (FACS) and Cell Sorting

The analysis was performed to assess integration of the GFP cassette in different cell types and cell populations. Unstained and single-stained cells or compensation beads were used as negative and positive controls. For apoptosis/necrosis detection cells were stained with 7-Aminoactinomycin D (7-AAD, BD Pharming) and Pacific Blue (PB) Annexin V (Biolegend). HSCs were stained with phycoerythrin cyanine 7 (PECy7) CD34 (Clone: AC136, Miltenyi Biotec), phycoerythrin (PE) CD133 (Miltenyi Biotec) allophycocyanin (APC) CD90 (BD Biosciences). Cell sorting on CD133/CD90 edited cells was performed using MoFlo XDP Cell Sorter (Beckman Coulter).

For mice analysis single-cell suspensions were obtained from bone marrow, spleen, thymus and peripheral blood and stained with the following anti-human antibodies: CD45 (clone REA757), CD3(clone REA613) (Miltenyi biotech), CD19 (clone SJ25C1), CD13 (clone WM15) (BD Biosciences). Human and murine Fc blocking was performed before each staining using human F-Block and murine CD16/CD32 from BD Pharmingen. Live/Dead Fixable Yellow (Thermo Fisher Scientific, Waltham, MA) was added to the antibody mix to exclude dead cells. Samples were acquired on a FACSCanto II (BD) and analyzed with FlowJo software (TreeStar, Ashland, Ore).

Analysis of HSPC composition of MPB-CD34+ cells was performed according to the protocol described in Basso-Ricci L, et al. Cytom Part A. 2017; 91(10):952-65. Briefly, 1.5×10⁵ cells were labeled with fluorescent antibodies against CD3, CD56, CD14, CD61/41, CD135, CD34, CD45RA (Biolegend) and CD33, CD66b, CD38, CD45, CD90, CD10, CD11c, CD19, CD7, and CD71 (BD Biosciences). All samples were acquired through BD LSR-Fortessa (BD Bioscience) cytofluorimeter after Rainbow beads (Spherotech) calibration and raw data were collected through DIVA software (BD Biosciences). The data were subsequently analyzed with FlowJo software Version 9.3.2 (TreeStar) and the graphical output was automatically generated through Prism 6.0c (GraphPad software).

### AAV6 Production and Titration

AAV vectors were produced by transient triple transfection of HEK293 cells by calcium phosphate. The following day, the medium was changed with serum-free DMEM and cells were harvested 72 hours after transfection. Cells were lysed by three rounds of freeze-thaw to release the viral particles and the lysate was incubated with DNAsel and RNAse I to eliminate nucleic acids. AAV vector was then purified by two sequential rounds of Cesium Cloride (CsCI2) gradient. For each viral preparation, physical titres (genome copies/mL) were determined by PCR quantification using TaqMan.

### AAV6 Gene-Editing Protocol in Cell Lines

2×10⁵ / 5×10⁵ cells per well were electroporated (Lonza, SF Cell line 4D Nucleofector X Kit, program FF120 for K562 or program DC100 for NALM6) with either plasmids or RNPs. Fifteen minutes after electroporation, cells were infected with AAV6 at different MOl: 10⁴; 5×10⁴; 10⁵ Vector Genome/cell, Vg/cell.

### CD34+ Cells

Human cord blood CD34+ cells (CB CD34+ cells) were obtained from Lonza (PoieticsTM cat# 2C 101). CB CD34+ cells/ml were stimulated in StemSpan medium supplemented with penicillin/streptomycin antibiotics and early-acting cytokines: Stem cell factor (SCF) 100 ng/ml, Flt3 ligand (Flts-L) 100 ng/ml, Thrombopoietin (TPO) 20 ng/ml, Interleukin 6 (IL- 6) 20 ng/ml, StemRegenin1 (SR1) (1 uM) and 16,16-dimethyl prostaglandin E2 (dmPGE2) (10 uM), UM171 50 nM. Patient mobilized peripheral blood CD34+ cells (CB CD34+ cells) were kindly provided by Dr. Luigi Notarangelo (Laboratory of Clinical Immunology and Microbiology, Division of Intramural Research, National Institute of Allergy and Infectious Diseases, National Institutes of Health, Bethesda, MD, United States). MPB CD34+ cells/ml were stimulated in StemSpan medium supplemented with penicillin/streptomycin antibiotics and early-acting cytokines: Stem cell factor (SCF) 300 ng/ml, Flt3 ligand (Flts-L) 300 ng/ml, Thrombopoietin (TPO) 100 ng/ml, Interleukin 3 (IL- 3) 60 ng/ml, StemRegenin1 (SR1) (1 uM) and 16,16-dimethyl prostaglandin E2 (dmPGE2) (10 uM), UM171 50 nM.

### AAV6 Gene-Editing Protocol in CD34+ Cells

After 3 days of expansion 2×10⁵ CD34+ cells per condition were electroporated (Lonza, P3 Primary Cell 4DNucleofectorX Kit, CD34+ program) with RNPs, GSE56 mRNA (p53 inhibitor) was added at a dose of 150 µg/ml when cells were aimed at being transplanted. 15 minutes after electroporation, CD34+ cells were infected with AAV6 at different MOl: 10⁴; 5×10⁴; 10⁵ Vg/cell.

### Digital PCR

Digital PCR (ddPCR) was performed to assess targeted integration. In short, gDNA was quantified using Nanodrop, and diluted in H₂O to reach 5-10 ng per reaction (1-2 ng/ul). It is possible to increase the gDNA quantity per reaction but it is important to remain below the saturation limit of the system. ddPCR master mix was prepared by adding 11 ul ddPCR Supermix for Probes (no dUTP; BioRad), 1.1 ul primer mix Primer forward + Primer reverse (final concentration 0.9 uM) + Probe (final concentration 0.25 uM), 1.1 ul normalizer primer mix, 4.9 ul H₂O per reaction. Finally, 17 ul of ddPCR master mix and 5 ul of diluted gDNA were added to each well (we included UT and H₂O as negative controls, and mono- or bi allelic clone as positive control to validate the system). Droplets were prepared on the BioRad AutoDG Automated Droplet Generator and the droplet plate was sealed with foil using BioRad PX1 PCR Plate Sealer. The sealed plate was placed into BioRad T100 Thermal Cycler and we ran the appropriate PCR program. The run was read in BioRad QX200 Droplet Reader.

Calculation copies per genome: concentration (copies/µl) gene of interest / concentration (copies/µl) normalizer gene x 2 Calculation percentage of HDR: copies per genome x 100.

Optimized PCR program (40 cycles):
95° C. x 10 min
40 × 94° x 30 sec
55° x 1 min
72° x 2 min
98° x 10 min
4° hold

Primers and Probes used for ddPCR assay are the following:

| | |
|---|---|
| PGK_GFP cassette FW | CAAGAGGTTGTCTGAAGGAAG |
| PGK_GFP cassette RV | GACGTGAAGAATGTGCGAG |
| PGK_GFP cassette PROBE FAM | CTGCTGCACCCTGGCCTCCTGAACTAA |
| Corrective CDS FW | GTGGAACAGGTGTGATAATGAG |
| Corrective CDS RV | GGAGGACAATCCAAGGGTAG |
| Corrective CDS PROBE FAM | TGCTGCTGCACCCTGGCCTCCTGAA |

### Mice and Transplantation Protocol

NOD-scid IL2Rgnull mice (NSG; Charles River) were purchased from Charles River Laboratories Inc. (Calco, Italy) and were maintained in specific pathogen-free (SPF) conditions. Mice were transplanted at 8-10 weeks approximately 6 hours after sublethal total body irradiation (120 rad), via intravenous injection of treated HSCPs in phosphate-buffered saline. Gentamicin sulfate (Italfarmaco, Milan, Italy) was administered in drinking water (8 mg/mL) for the first 2 weeks after transplantation to prevent infections. Mice were followed until the sacrifice and then euthanized for ex vivo analyses.

### Statistical Analysis

When normality assumptions were not met, non-parametric statistical tests were performed. Kruskal-Wallis test with multiple comparison post-test was performed when comparing more groups. When normality assumptions were met, two-way analysis of variance (ANOVA) was used. For repeated measures over time, two-way ANOVA with Bonferroni’s multiple comparison post-test was utilized. Values are expressed as Mean ± SD.

### Example 2

### Results and Discussion

### Corrective Donor Screening

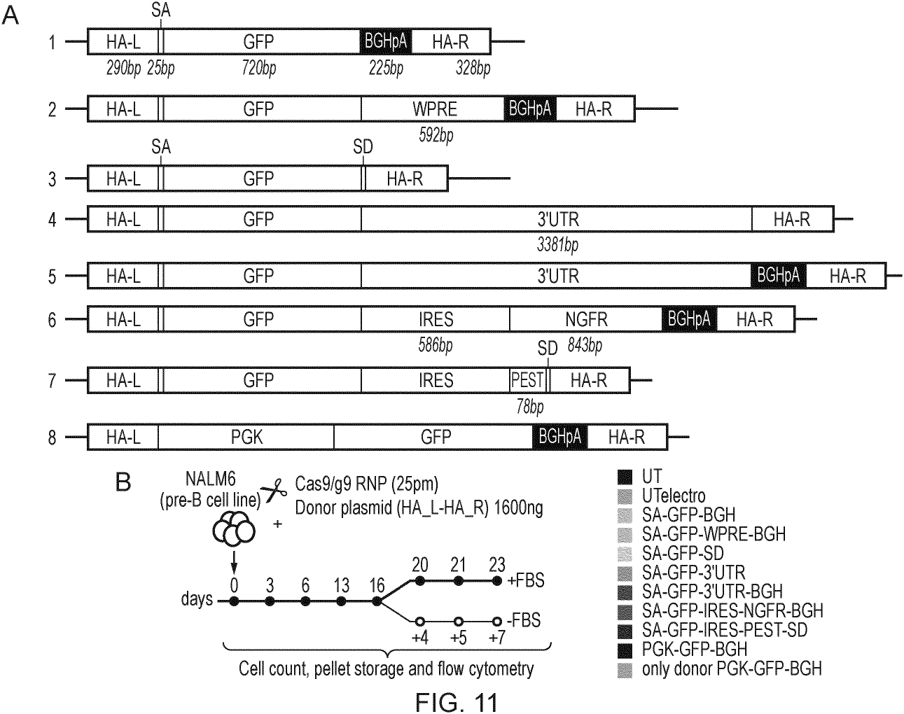

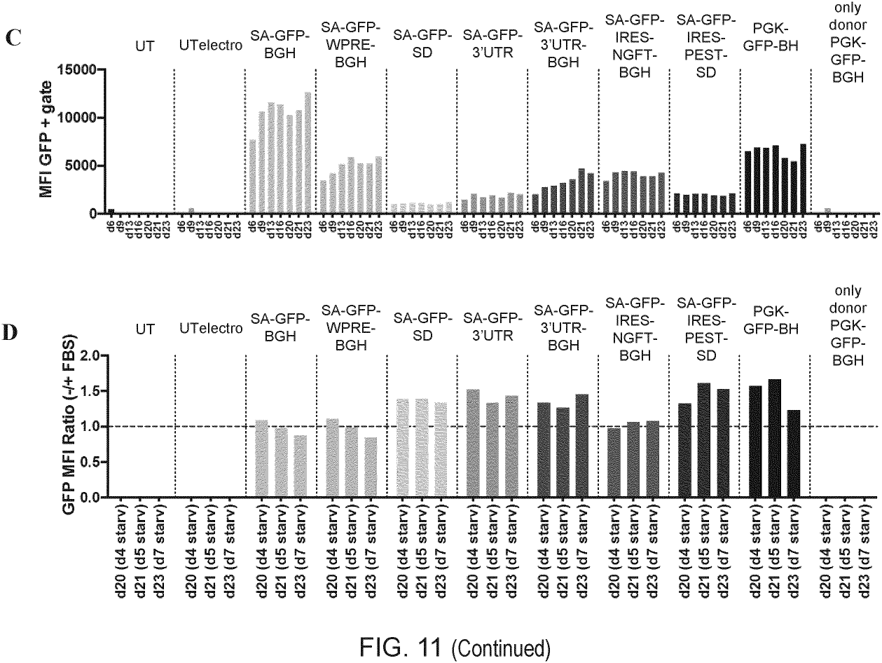

To further explore the role of the 3′UTR and the selection strategy, further corrective donor sequences numbered 5-8 below were designed and compared with the sequences numbered 1-4 below (FIG. 11A):
1. Construct carrying the bovine growth hormone (BGH) PolyA downstream of the SA_GFP (SA_GFP_BGH);
2. Construct carrying the Woodchuck hepatitis virus post-transcriptional regulatory element (WPRE) downstream of the SA_GFP and upstream of the BGH PolyA (SA_GFP_WPRE);
3. Construct containing a splice donor downstream of the SA_GFP cassette (SA_GFP_SD) to obtain a fusion transcript including the corrected sequence and endogenous RAG1 followed by the 3′ UTR sequence;
4. Construct with the same endogenous RAG13′UTR following the SA_GFP cassette (SA_GFP_3′UTR);
5. Construct with the SA_GFP cassette followed by the endogenous RAG1 3′UTR and BGH PolyA (SA_GFP_3′UTR_BGH);
6. Construct with the SA_GFP cassette followed by the internal ribosome entry site sequence (IRES), a clinically compatible selector (C❏terminal truncated low❏ affinity NGFR receptor, hereafter named NGFR) and the BGH PolyA sequences (SA_GFP_IRES_NGFR_BGH) - this strategy might allow the enrichment of edited cells by the NGFR selector and the improvement of GFP expression through the IRES and mRNA stabilization;
7. Construct with the SA_GFP cassette followed by the IRES, a peptide sequence rich in proline (P), glutamic acid (E), serine (S), and threonine (T) (PEST) and the splice donor sequence (SA_GFP_IRES_PEST_SD) - this construct will result in a fusion transcript including the corrected sequence and the endogenous RAG1 followed by the 3′ UTR sequence (it is expected that the endogenous RAG1 protein will be destabilized by the PEST signal peptide via proteasome degradation;
8. Construct with GFP expression driven by a PGK promoter as an internal positive control (PGK_GFP-BGH).

To screen the donors described above, NALM6 cells were transfected with guide 9 and Cas9 as an RNP (25 pmol) and donors as linearized DNA fragments (1600 ng), and then kept in culture with RPMI and 10% FBS. To synchronize cell cycles at G0/G1 phase when the RAG1 gene is mainly expressed, cells were serum starved 16 days after the transfection (FIG. 11B).

We evaluated GFP expression as the percentage of GFP+ cells and GFP mean fluorescence intensity (MFI) by flow cytometry over time. The proportion of GFP+ cells was low in all conditions as expected because NALM6 are poorly permissive to the editing. We confirmed data described in FIG. 3 showing that cells edited with SA_GFP_SD and SA_GFP_3′UTR constructs have a lower MFI than that obtained by SA_GFP_BGH (FIG. 11C). Moreover, SA_GFP_IRES_NGFR_BGH and SA_GFP_IRES_PEST_SD did not ameliorate GFP expression compared to other constructs (FIG. 11C).

We analyzed the GFP expression 4, 5 and 7 days after serum starvation to evaluate the modulation of transgene expression when regulated by the RAG1 promoter. We found that all the donors carrying the 3′UTR or using the endogenous 3′UTR (by SD sequence) resulted in a modulation of GFP expression upon starvation (FIG. 11D).

### Editing Enhancer Effects on HDR Efficiency of RAG1 Locus and T Cell Differentiation Potential

To further understand the efficacy of the gene editing approach to correct RAG1 defects, we exploited a novel organoid platform, referred to as artificial thymic organoid (ATO) based on the aggregation of DLL4 expressing stromal cell line (MS5-hDLL4) with CD34⁺ cells isolated from bone marrow or mobilized peripheral blood. The ATO platform (Seet et al. (2017) Nat Methods) is a suitable tool to study the first steps of human T cell differentiation. We adopted this platform to assess the impact of the gene editing procedure on T cell differentiation and to evaluate the extent to which precise correction allows the overcoming of a T cell differentiation block.

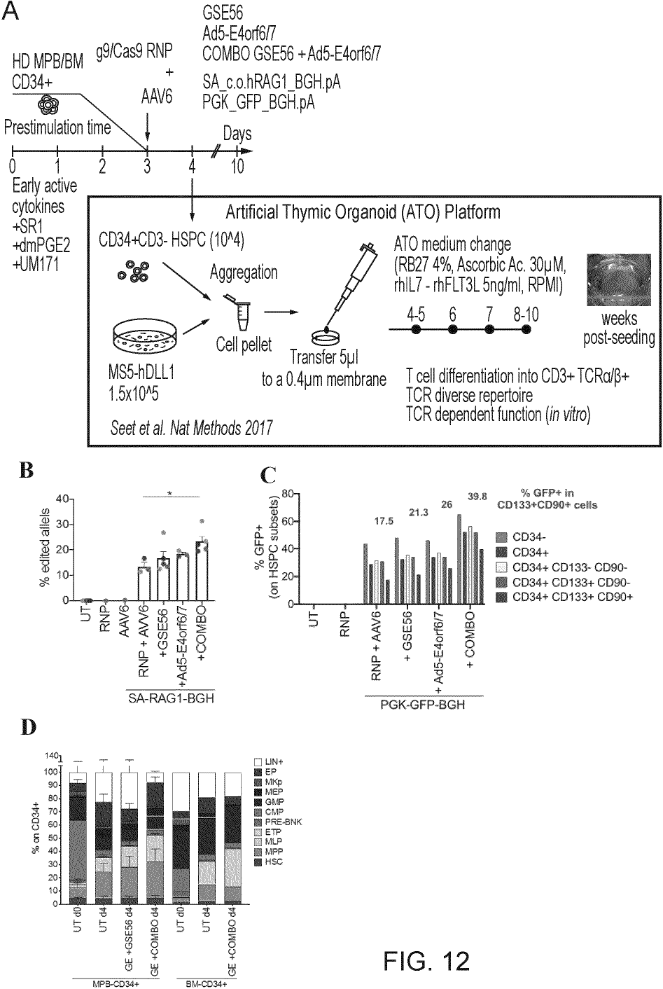

To this end, we set up and optimized the ATO system using CD34+ cells obtained from healthy donor (HD) mobilized peripheral blood (MPB) or bone marrow (BM). One day after editing, CD34+ cells were aggregated with MS5-hDLL4 cells and kept in culture for 4 to 7 weeks to assess the T cell differentiation potential and the editing efficiency (FIG. 12). ATOs generated with gene edited CD34+ cells showed lower cell viability as compared to ATO containing untreated CD34+ cells.

To overcome the high toxicity likely caused by the exacerbated p53 response and at the same time to enhance HDR efficiency, we tested the effect of gene editing enhancer compounds: to this end we exploited the messenger RNA for the dominant negative p53 GSE56 with or without Ad5-E4orf6/7, or Ad5-E4orf6/7 alone during the editing procedure. Ad5-E4orf6/7 is an adenoviral protein known as a helper in Ad-AAV co-infection, which interacts with several components involved in survival and cell cycle.

We electroporated CD34+ cells in the presence of gene editing enhancers: GSE56 or Ad5-E4orf6/7 alone or the combination of GSE56 and Ad5-E4orf6/7 (COMBO). Cells were then transduced with AAV6 vectors: the corrective donor vector carrying the codon optimized RAG1 downstream of the splice acceptor (SA) and followed by the BGH polyA (SA_coRAG1_BGH polyA) or the AAV6 vector carrying the PGK_GFP_BGHpolyA to track edited cells in HPSC cell subsets (FIG. 12A). Seven days after gene editing, HDR efficiency was assessed by ddPCR for CD34+ cells edited with SA-coRAG1-BGHpolyA, while by flow cytometry for CD34+ cells edited with PGK_GFP_BGHpolyA. In the presence of the corrective donor, molecular analysis revealed a significant increase of the frequency of edited alleles in the gene editing condition performed in the presence of GSE56+Ad5-E4orf6/7 (COMBO) (FIG. 12B). Remarkably, CD34+ cells undergoing gene editing with AAV6 PGK_GFP_BGHpolyA revealed a frequency of 40% of GFP positive cells within the most primitive HSPC subset (CD133+ CD90+) (FIG. 12C).

Moreover, we performed multiparametric analysis of MPB or BM HSPC compositions before (day 0) and after gene editing (day 4) (FIG. 12D). We confirmed previous data (FIG. 10) showing a redistribution of HSPC subpopulations mainly due to the expansion protocol. In untreated and edited CD34+ cells at day 4, we observed a relative expansion of hematopoietic stem cells (HSC), multipotent progenitors (MPP) and multilymphoid progenitors (MLP) at the expense of common myeloid progenitors (CMP), indicating that gene editing protocols using GSE56+Ad5-E4orf6/7 (COMBO) preserve stemness in the composition (FIG. 12D).

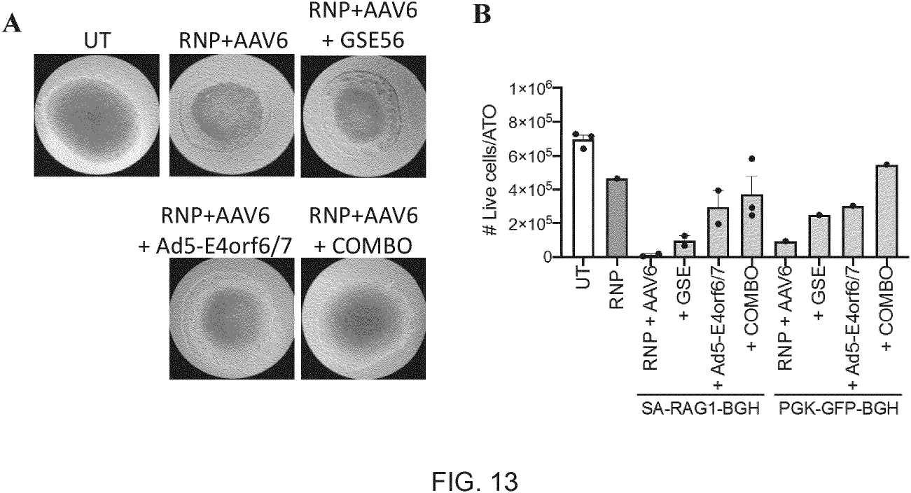

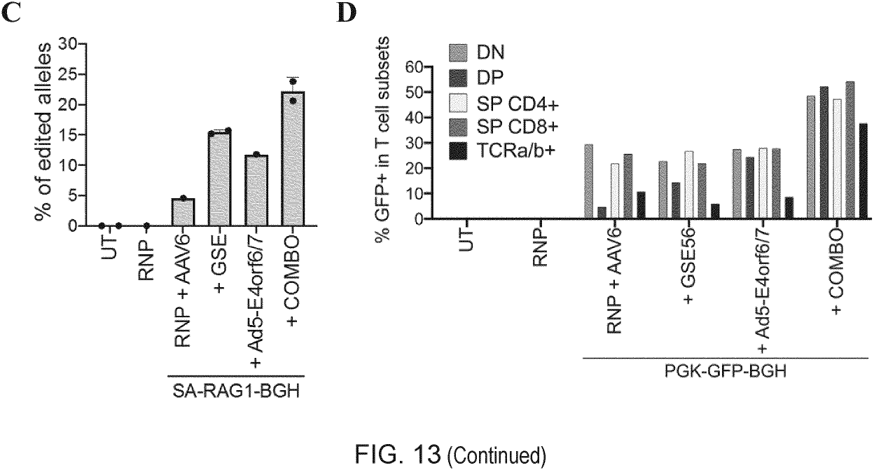

After 24 hours from gene editing (at day 4), CD34+ cells were washed, counted and seeded in the presence of MS5-hDLL4 to form thymic organoids to follow T cell differentiation for 4-7 weeks. Starting from the fourth week after the seeding, ATOs were dissociated, and bulk cells edited with the corrective donor were analyzed for HDR efficiency by molecular analysis (ddPCR), while cells edited with pGK_GFP_BGHpolyA AAV6 vector were analyzed by flow cytometry to detect the frequency of GFP+ cells in different T cell subsets. Evaluation of ATOs, showed an improvement of organoid morphology in the presence of the combined action of GSE56+E4orf6/7 (FIG. 13A). This finding was confirmed by the increased number of cells harvested from ATOs seeded with CD34+ edited with Ad5-E4orf6/7 and reaching the highest values with the COMBO treatment (FIG. 13B).

The molecular analysis of HDR frequency in T cells differentiated from CD34+ edited with SA_coRAG1_BGHpolyA further confirmed the synergistic effect of GSE56+Ad5-E4orf6/7 revealing the higher proportion of edited alleles in the COMBO condition as compared to others (FIG. 13C). Flow cytometric analysis of double negative (DN), double positive (DP), single positive (SP) T cells obtained from the ATO seeded with CD34+ cells edited and transduced with the AAV6 PGK_GFP_BGHpolyA showed the highest frequency of GFP+ cells in the COMBO condition (FIG. 13D). The synergistic effect of GSE56+Ad5-E4orf6/7 was more evident in TCRα/β+ cell subset, a relevant subpopulation absent in RAG1-deficient patients.

Overall, these data indicate that the use of gene editing enhancers dramatically enhance HDR editing efficiency in CD34+ cells while preserving their ability to differentiate towards T cell lineage.

### Material and Methods

### Donor Constructs

The cloning of plasmids was performed using general molecular biology techniques. Briefly, plasmids were digested using restriction enzymes (New England BioLabs) and correct fragments were separated and purified by agarose gel electrophoresis. Fragments were inserted into a dephosphorylated linearized backbone with either Quick Ligase or T4 Ligase after purification with QIAquick PCR Purification Kit (QIAGEN). After ligation, TOP10 chemically competent E. Coli bacteria were transformed and plated on plates containing antibiotics. Plasmid DNA was extracted and purified with Wizard Plus SV Minipreps DNA Purification System (Promega) and EndoFree Plasmid Maxi Kit (QIAGEN). Colonies were screened with control digestions and sequenced. Sequences of the further inserts are shown below:

### AAV6 Vector Carrying SA_GFP_3′UTR_BGH, Guide 9:

INSERT

tgagcacacagttattacttggaaattgtgtacagactaagttgaagatg
ttaggagggaagattgtgggccaagtaacggggtgtatgtgtgtgggtat
agggtgggcagctgggatggaaatggggggctgctgctgctgctgcaccc
tggcctcctgaactaatgatatcactcaccagaaactactgttcctgcac
tgtccaagccaccccaaactagtttgtcaaaatgaatctgtgctgtgtgg
agggaggcacgcctgtagctctgatgtcagatggcaatgtgaattcctga
cctcttctcttcctcccacaggccgccaccatggtgagcaagggcgagga
gctgttcaccggggtggtgcccatcctggtcgagctggacggcgacgtaa
acggccacaagttcagcgtgtccggcgagggcgagggcgatgccacctac
ggcaagctgaccctgaagttcatctgcaccaccggcaagctgcccgtgcc
ctggcccaccctcgtgaccaccctgacctacggcgtgcagtgcttcagtc
gctaccccgaccacatgaagcagcacgacttcttcaagtccgccatgccc
gaaggctacgtccaggagcgcaccatcttcttcaaggacgacggcaacta
caagacccgcgccgaggtgaagttcgagggcgacaccctggtgaaccgca
tcgagctgaagggcatcgacttcaaggaggacggcaacatcctggggcac
aagctggagtacaactacaacagccacaacgtctatatcatggccgacaa
gcagaagaacggcatcaaggtgaacttcaagatccgccacaacatcgagg
acggcagcgtgcagctcgccgaccactaccagcagaacacccccatcggc
gacggccccgtgctgctgcctgacaaccactacctgagcacccagtccgc
cctgagcaaagaccccaacgagaagcgcgatcacatggtcctgctggagt
tcgtgaccgccgccgggatcactctcggcatggacgagctgtacaagtaa
actggatccgtagggcaaccacttatgagttggtttttgcaattgagttt
ccctctgggttgcattgagggcttctcctagcaccctttactgctgtgta
tggggcttcaccatccaagaggtggtaggttggagtaagatgctacagat
gctctcaagtcaggaatagaaactgatgagctgattgcttgaggctttta
gtgagttccgaaaagcaacaggaaaaatcagttatctgaaagctcagtaa
ctcagaacaggagtaactgcaggggaccagagatgagcaaagatctgtgt
gtgttggggagctgtcatgtaaatcaaagccaaggttgtcaaagaacagc
cagtgaggccaggaaagaaattggtcttgtggttttcatttttttccccc
ttgattgattatattttgtattgagatatgataagtgccttctatttcat
ttttgaataattcttcatttttataattttacatatcttggcttgctata
taagattcaaaagagctttttaaatttttctaataatatcttacatttgt
acagcatgatgacctttacaaagtgctctcaatgcatttacccattcgtt
atataaatatgttacatcaggacaactttgagaaaatcagtcctttttta
tgtttaaattatgtatctattgtaaccttcagagtttaggaggtcatctg
ctgtcatggatttttcaataatgaatttagaatacacctgttagctacag
ttagttattaaatcttctgataatatatgtttacttagctatcagaagcc
aagtatgattctttatttttactttttcatttcaagaaatttagagtttc
caaatttagagcttctgcatacagtcttaaagccacagaggcttgtaaaa
atataggttagcttgatgtctaaaaatatatttcatgtcttactgaaaca
ttttgccagactttctccaaatgaaacctgaatcaatttttctaaatcta
ggtttcatagagtcctctcctctgcaatgtgttattctttctataatgat
cagtttactttcagtggattcagaattgtgtagcaggataaccttgtatt
tttccatccgctaagtttagatggagtccaaacgcagtacagcagaagag
ttaacatttacacagtgctttttaccactgtggaatgttttcacactcat
ttttccttacaacaattctgaggagtaggtgttgttattatctccatttg
atgggggtttaaatgatttgctcaaagtcatttaggggtaataaatactt
ggcttggaaatttaacacagtccttttgtctccaaagcccttcttctttc
caccacaaattaatcactatgtttataaggtagtatcagaatttttttag
gattcacaactaatcactatagcacatgaccttgggattacatttttatg
gggcaggggtaagcaagtttttaaatcatttgtgtgctctggctcttttg
atagaagaaagcaacacaaaagctccaaagggccccctaaccctcttgtg
gctccagttatttggaaactatgatctgcatccttaggaatctgggattt
gccagttgctggcaatgtagagcaggcatggaattttatatgctagtgag
tcataatgatatgttagtgttaattagttttttcttcctttgattttatt
ggccataattgctactcttcatacacagtatatcaaagagcttgataatt
tagttgtcaaaagtgcatcggcgacattatctttaattgtatgtatttgg
tgcttcttcagggattgaactcagtatctttcattaaaaaacacagcagt
tttccttgctttttatatgcagaatatcaaagtcatttctaatttagttg
tcaaaaacatatacatattttaacattagtttttttgaaaactcttggtt
ttgtttttttggaaatgagtgggccactaagccacactttcccttcatcc
tgcttaatccttccagcatgtctctgcactaataaacagctaaattcaca
taatcatcctatttactgaagcatggtcatgctggtttatagatttttta
cccatttctactctttttctctattggtggcactgtaaatactttccagt
attaaattatccttttctaacactgtaggaactattttgaatgcatgtga
ctaagagcatgatttatagcacaacctttccaataatcccttaatcagat
cacattttgataaaccctgggaacatctggctgcaggaatttcaatatgt
agaaacgctgcctatggttttttgcccttactgttgagactgcaatatcc
tagaccctagttttatactagagttttatttttagcaatgcctattgcaa
gtgcaattatatactccagggaaattcaccacactgaatcgagcatttgt
gtgtgtatgtgtgaagtatatactgggacttcagaagtgcaatgtatttt
tctcctgtgaaacctgaatctacaagttttcctgccaagccactcaggtg
cattgcagggaccagtgataatggctgatgaaaattgatgattggtcagt
gaggtcaaaaggagccttgggattaataaacatgcactgagaagcaagag
gaggagaaaaagatgtctttttcttccaggtgaactggaatttagttttg
cctcagatttttttcccacaagatacagaagaagataaagatttttttgg
ttgagagtgtgggtcttgcattacatcaaacagagttcaaattccacaca
gataagaggcaggatatataagcgccagtggtagttgggaggaataaacc
attatttggatgcaggtggtttttgattgcaaatatgtgtgtgtcttcag
tgattgtatgacagatgatgtattcttttgatgttaaaagattttaagta
agagtagatacattgtacccattttacattttcttattttaactacagta
atctacataaatatacctcagaaatcatttttggtgattattttttgttt
tgtagaattgcacttcagtttattttcttacaaataaccttacattttgt
ttaatggcttccaagagccttttttttttttgtatttcagagaaaattca
ggtaccaggatgcaatggatttatttgattcaggggacctgtgtttccat
gtcaaatgttttcaaataaaatgaaatatgagtttcaatactttttatat
tttaatatttccattcattaatattatggttattgtcagcaattttatgt
ttgaatatttgaaataaaagtttaagatttgaaaatggtatgtattataa
tttctattcaaatattaataataatattgagtgcagcatttctaggatcc
taaactgtgccttctagttgccagccatctgttgtttgcccctcccccgt
gccttccttgaccctggaaggtgccactcccactgccctttcctaataaa
atgaggaaattgcatcgcattgtctgagtaggtgtcattctattctgggg
ggtggggtggggcaggacagcaagggggaggattgggaagacaatagcag
gcatgctggggatgcggtgggctctatggtctagatggcagtggccggtg
gggacagggctgagccagcaccaaccactcagcctttgagatcccgaggc
tggtctactgctgagaccttttgttagaagagaggagatcaagcatttgc
aaggtttctgagtgtcaaaatatgaatccaagataactctttcacaatcc
taacttcatgctgtctacaggtccatattttagcctgctttctccatgtt
catccgaaaagaaagaaaagctaagggtggtggtcatatttgaaattagc
cagatcttaagtttttctgggggaaatttagaagaaaatatggaaaagtg
actatgagcaca

HA Left

tgagcacacagttattacttggaaattgtgtacagactaagttgaagatg
ttaggagggaagattgtgggccaagtaacggggtgtatgtgtgtgggtat
agggtgggcagctgggatggaaatggggggctgctgctgctgctgcaccc
tggcctcctgaactaatgatatcactcaccagaaactactgttcctgcac
tgtccaagccaccccaaactagtttgtcaaaatgaatctgtgctgtgtgg
agggaggcacgcctgtagctctgatgtcagatggcaatgt

Splice Acceptor

ctgacctcttctcttcctcccacag

KOZAK

gccgccaccatg

GFP

atggtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagtcgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcctgacaaccac
tacctgagcacccagtccgccctgagcaaagaccccaacgagaagcgcga
tcacatggtcctgctggagttcgtgaccgccgccgggatcactctcggca
tggacgagctgtacaagtaa

3′UTR

gtagggcaaccacttatgagttggtttttgcaattgagtttccctctggg
ttgcattgagggcttctcctagcaccctttactgctgtgtatggggcttc
accatccaagaggtggtaggttggagtaagatgctacagatgctctcaag
tcaggaatagaaactgatgagctgattgcttgaggcttttagtgagttcc
gaaaagcaacaggaaaaatcagttatctgaaagctcagtaactcagaaca
ggagtaactgcaggggaccagagatgagcaaagatctgtgtgtgttgggg
agctgtcatgtaaatcaaagccaaggttgtcaaagaacagccagtgaggc
caggaaagaaattggtcttgtggttttcatttttttcccccttgattgat
tatattttgtattgagatatgataagtgccttctatttcatttttgaata
attcttcatttttataattttacatatcttggcttgctatataagattca
aaagagctttttaaatttttctaataatatcttacatttgtacagcatga
tgacctttacaaagtgctctcaatgcatttacccattcgttatataaata
tgttacatcaggacaactttgagaaaatcagtccttttttatgtttaaat
tatgtatctattgtaaccttcagagtttaggaggtcatctgctgtcatgg
atttttcaataatgaatttagaatacacctgttagctacagttagttatt
aaatcttctgataatatatgtttacttagctatcagaagccaagtatgat
tctttatttttactttttcatttcaagaaatttagagtttccaaatttag
agcttctgcatacagtcttaaagccacagaggcttgtaaaaatataggtt
agcttgatgtctaaaaatatatttcatgtcttactgaaacattttgccag
actttctccaaatgaaacctgaatcaatttttctaaatctaggtttcata
gagtcctctcctctgcaatgtgttattctttctataatgatcagtttact
ttcagtggattcagaattgtgtagcaggataaccttgtatttttccatcc
gctaagtttagatggagtccaaacgcagtacagcagaagagttaacattt
acacagtgctttttaccactgtggaatgttttcacactcatttttcctta
caacaattctgaggagtaggtgttgttattatctccatttgatgggggtt
taaatgatttgctcaaagtcatttaggggtaataaatacttggcttggaa
atttaacacagtccttttgtctccaaagcccttcttctttccaccacaaa
ttaatcactatgtttataaggtagtatcagaatttttttaggattcacaa
ctaatcactatagcacatgaccttgggattacatttttatggggcagggg
taagcaagtttttaaatcatttgtgtgctctggctcttttgatagaagaa
agcaacacaaaagctccaaagggccccctaaccctcttgtggctccagtt
atttggaaactatgatctgcatccttaggaatctgggatttgccagttgc
tggcaatgtagagcaggcatggaattttatatgctagtgagtcataatga
tatgttagtgttaattagttttttcttcctttgattttattggccataat
tgctactcttcatacacagtatatcaaagagcttgataatttagttgtca
aaagtgcatcggcgacattatctttaattgtatgtatttggtgcttcttc
agggattgaactcagtatctttcattaaaaaacacagcagttttccttgc
tttttatatgcagaatatcaaagtcatttctaatttagttgtcaaaaaca
tatacatattttaacattagtttttttgaaaactcttggttttgtttttt
tggaaatgagtgggccactaagccacactttcccttcatcctgcttaatc
cttccagcatgtctctgcactaataaacagctaaattcacataatcatcc
tatttactgaagcatggtcatgctggtttatagattttttacccatttct
actctttttctctattggtggcactgtaaatactttccagtattaaatta
tccttttctaacactgtaggaactattttgaatgcatgtgactaagagca
tgatttatagcacaacctttccaataatcccttaatcagatcacattttg
ataaaccctgggaacatctggctgcaggaatttcaatatgtagaaacgct
gcctatggttttttgcccttactgttgagactgcaatatcctagacccta
gttttatactagagttttatttttagcaatgcctattgcaagtgcaatta
tatactccagggaaattcaccacactgaatcgagcatttgtgtgtgtatg
tgtgaagtatatactgggacttcagaagtgcaatgtatttttctcctgtg
aaacctgaatctacaagttttcctgccaagccactcaggtgcattgcagg
gaccagtgataatggctgatgaaaattgatgattggtcagtgaggtcaaa
aggagccttgggattaataaacatgcactgagaagcaagaggaggagaaa
aagatgtctttttcttccaggtgaactggaatttagttttgcctcagatt
tttttcccacaagatacagaagaagataaagatttttttggttgagagtg
tgggtcttgcattacatcaaacagagttcaaattccacacagataagagg
caggatatataagcgccagtggtagttgggaggaataaaccattatttgg
atgcaggtggtttttgattgcaaatatgtgtgtgtcttcagtgattgtat
gacagatgatgtattcttttgatgttaaaagattttaagtaagagtagat
acattgtacccattttacattttcttattttaactacagtaatctacata
aatatacctcagaaatcatttttggtgattattttttgttttgtagaatt
gcacttcagtttattttcttacaaataaccttacattttgtttaatggct
tccaagagccttttttttttttgtatttcagagaaaattcaggtaccagg
atgcaatggatttatttgattcaggggacctgtgtttccatgtcaaatgt
tttcaaataaaatgaaatatgagtttcaatactttttatattttaatatt
tccattcattaatattatggttattgtcagcaattttatgtttgaatatt
tgaaataaaagtttaagatttgaaaatggtatgtattataatttctattc
aaatattaataataatattgagtgcagcatt

BGH

actgtgccttctagttgccagccatctgttgtttgcccctcccccgtgcc
ttccttgaccctggaaggtgccactcccactgccctttcctaataaaatg
aggaaattgcatcgcattgtctgagtaggtgtcattctattctggggggt
ggggtggggcaggacagcaagggggaggattgggaagacaatagcaggca
tgctggggatgcggtgggctctatgg

HA Right

atggcagtggccggtggggacagggctgagccagcaccaaccactcagcc
tttgagatcccgaggctggtctactgctgagaccttttgttagaagagag
gagatcaagcatttgcaaggtttctgagtgtcaaaatatgaatccaagat
aactctttcacaatcctaacttcatgctgtctacaggtccatattttagc
ctgctttctccatgttcatccgaaaagaaagaaaagctaagggtggtggt
catatttgaaattagccagatcttaagtttttctgggggaaatttagaag
aaaatatggaaaagtgactatgagcaca

### AAV6 Vector Carrying SA_GFP_IRES_NGFR_BGH-RAG1, Guide 9:

INSERT

tgagcacacagttattacttggaaattgtgtacagactaagttgaagatg
ttaggagggaagattgtgggccaagtaacggggtgtatgtgtgtgggtat
agggtgggcagctgggatggaaatggggggctgctgctgctgctgcaccc
tggcctcctgaactaatgatatcactcaccagaaactactgttcctgcac
tgtccaagccaccccaaactagtttgtcaaaatgaatctgtgctgtgtgg
agggaggcacgcctgtagctctgatgtcagatggcaatgtgaattcctga
cctcttctcttcctcccacaggccgccaccatggtgagcaagggcgagga
gctgttcaccggggtggtgcccatcctggtcgagctggacggcgacgtaa
acggccacaagttcagcgtgtccggcgagggcgagggcgatgccacctac
ggcaagctgaccctgaagttcatctgcaccaccggcaagctgcccgtgcc
ctggcccaccctcgtgaccaccctgacctacggcgtgcagtgcttcagtc
gctaccccgaccacatgaagcagcacgacttcttcaagtccgccatgccc
gaaggctacgtccaggagcgcaccatcttcttcaaggacgacggcaacta
caagacccgcgccgaggtgaagttcgagggcgacaccctggtgaaccgca
tcgagctgaagggcatcgacttcaaggaggacggcaacatcctggggcac
aagctggagtacaactacaacagccacaacgtctatatcatggccgacaa
gcagaagaacggcatcaaggtgaacttcaagatccgccacaacatcgagg
acggcagcgtgcagctcgccgaccactaccagcagaacacccccatcggc
gacggccccgtgctgctgcctgacaaccactacctgagcacccagtccgc
cctgagcaaagaccccaacgagaagcgcgatcacatggtcctgctggagt
tcgtgaccgccgccgggatcactctcggcatggacgagctgtacaagtaa
actggatccgaattaactcgaggaattccgcccctctccctccccccccc
ctaacgttactggccgaagccgcttggaataaggccggtgtgcgtttgtc
tatatgttattttccaccatattgccgtcttttggcaatgtgagggcccg
gaaacctggccctgtcttcttgacgagcattctaggggtctttcccctct
cgccaaaggaatgcaaggtctgttgaatgtcgtgaaggaagcagttcctc
tggaagcttcttgaagacaaacaacgtctgtagcgaccctttgcaggcag
cggaaccccccacctggcgacaggtgcctctgcggccaaaagccaacgtg
tataagatacacctgcaaaggcggcacaaccccagtgccacgttgtgagt
tggatagttgtggaaagagtcaaatggctctcctcaagcgtattcaacaa
ggggctgaaggatgcccagaaggtaccccattgtatgggatctgatctgg
ggcctcggtgcacatgctttacatgtgtttagtcgaggttaaaaaacgtc
taggccccccgaaccacggggacgtggttttcctttgaaaaacacgatga
taatatggccacaaccatgggagctggtgctaccggcagagctatggatg
gacctagactgctgctcctgctgctgctcggagtttctcttggcggagcc
aaagaggcctgtcctaccggcctgtatacacactctggcgagtgctgcaa
ggcctgcaatcttggagaaggcgtggcacagccttgcggcgctaatcaga
cagtgtgcgagccttgcctggacagcgtgacctttagcgacgtggtgtct
gccaccgagccatgcaagccttgtaccgagtgtgtgggcctgcagagcat
gtctgccccttgtgtggaagccgacgatgccgtgtgtagatgcgcctacg
gctactaccaggacgagacaacaggcagatgcgaggcctgtagagtgtgt
gaagccggctctggactggtgttcagctgccaagacaagcagaacaccgt
gtgcgaggaatgccccgatggcacctatagcgacgaggccaaccatgtag
atccctgcctgccttgtactgtgtgcgaagataccgagcggcagctgcgc
gagtgtacaagatgggctgatgccgagtgcgaagagatccccggcagatg
gatcaccagaagcacacctccagagggcagcgatagcacagccccttcta
cacaagagcccgaggctcctcctgagcaggatctgattgcctctacagtg
gccggcgtggtcacaacagtgatgggatcttctcagcccgtggtcaccag
aggcaccaccgacaatctgatccccgtgtactgtagcatcctggccgccg
tggttgtgggactcgtggcctatatcgccttcaagcggtggaaccggggc
atcctgtaatgatctagcaacccgctgatcagcctcgactgtgccttcta
gttgccagccatctgttgtttgcccctcccccgtgccttccttgaccctg
gaaggtgccactcccactgtcctttcctaataaaatgaggaaattgcatc
gcattgtctgagtaggtgtcattctattctggggggtggggtggggcagg
acagcaagggggaggattgggaagacaatagcaggcatgctggggatgcg
gtgggctctatggtctagaatggcagtggccggtggggacagggctgagc
cagcaccaaccactcagcctttgagatcccgaggctggtctactgctgag
accttttgttagaagagaggagatcaagcatttgcaaggtttctgagtgt
caaaatatgaatccaagataactctttcacaatcctaacttcatgctgtc
tacaggtccatattttagcctgctttctccatgttcatccgaaaagaaag
aaaagctaagggtggtggtcatatttgaaattagccagatcttaagtttt
tctgggggaaatttagaagaaaatatggaaaagtgactatgagcaca

HA Left

tgagcacacagttattacttggaaattgtgtacagactaagttgaagatg
ttaggagggaagattgtgggccaagtaacggggtgtatgtgtgtgggtat
agggtgggcagctgggatggaaatggggggctgctgctgctgctgcaccc
tggcctcctgaactaatgatatcactcaccagaaactactgttcctgcac
tgtccaagccaccccaaactagtttgtcaaaatgaatctgtgctgtgtgg
agggaggcacgcctgtagctctgatgtcagatggcaatgt

Splice Acceptor

ctgacctcttctcttcctcccacag

KOZAK

gccgccaccatg

GFP

atggtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagtcgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcctgacaaccac
tacctgagcacccagtccgccctgagcaaagaccccaacgagaagcgcga
tcacatggtcctgctggagttcgtgaccgccgccgggatcactctcggca
tggacgagctgtacaagtaa

IRES

gaattaactcgaggaattccgCccctctccctcccccccccctaacgtta
ctggccgaagccgcttggaataaggccggtgtgcgtttgtctatatgtta
ttttccaccatattgccgtcttttggcaatgtgagggcccggaaacctgg
ccctgtcttcttgacgagcattctaggggtctttcccctctcgccaaagg
aatgcaaggtctgttgaatgtcgtgaaggaagcagttcctctggaagctt
cttgaagacaaacaacgtctgtagcgaccctttgcaggcagcggaacccc
ccacctggcgacaggtgcctctgcggccaaaagccaacgtgtataagata
cacctgcaaaggcggcacaaccccagtgccacgttgtgagttggatagtt
gtggaaagagtcaaatggctctcctcaagcgtattcaacaaggggctgaa
ggatgcccagaaggtaccccattgtatgggatctgatctggggcctcggt
gcacatgctttacatgtgtttagtcgaggttaaaaaacgtctaggccccc
cgaaccacggggacgtggttttcctttgaaaaacacgatgataatatggc
cacaacc

NGFR

atgggagctggtgctaccggcagagctatggatggacctagactgctgct
cctgctgctgctcggagtttctcttggcggagccaaagaggcctgtccta
ccggcctgtatacacactctggcgagtgctgcaaggcctgcaatcttgga
gaaggcgtggcacagccttgcggcgctaatcagacagtgtgcgagccttg
cctggacagcgtgacctttagcgacgtggtgtctgccaccgagccatgca
agccttgtaccgagtgtgtgggcctgcagagcatgtctgccccttgtgtg
gaagccgacgatgccgtgtgtagatgcgcctacggctactaccaggacga
gacaacaggcagatgcgaggcctgtagagtgtgtgaagccggctctggac
tggtgttcagctgccaagacaagcagaacaccgtgtgcgaggaatgcccc
gatggcacctatagcgacgaggccaaccatgtagatccctgcctgccttg
tactgtgtgcgaagataccgagcggcagctgcgcgagtgtacaagatggg
ctgatgccgagtgcgaagagatccccggcagatggatcaccagaagcaca
cctccagagggcagcgatagcacagccccttctacacaagagcccgaggc
tcctcctgagcaggatctgattgcctctacagtggccggcgtggtcacaa
cagtgatgggatcttctcagcccgtggtcaccagaggcaccaccgacaat
ctgatccccgtgtactgtagcatcctggccgccgtggttgtgggactcgt
ggcctatatcgccttcaagcggtggaaccggggcatcctgtaa

BGH

ctgtgccttctagttgccagccatctgttgtttgcccctcccccgtgcct
tccttgaccctggaaggtgccactcccactgtcctttcctaataaaatga
ggaaattgcatcgcattgtctgagtaggtgtcattctattctggggggtg
gggtggggcaggacagcaagggggaggattgggaagacaatagcaggcat
gctggggatgcggtgggctctatgg

HA Right

atggcagtggccggtggggacagggctgagccagcaccaaccactcagcc
tttgagatcccgaggctggtctactgctgagaccttttgttagaagagag
gagatcaagcatttgcaaggtttctgagtgtcaaaatatgaatccaagat
aactctttcacaatcctaacttcatgctgtctacaggtccatattttagc
ctgctttctccatgttcatccgaaaagaaagaaaagctaagggtggtggt
catatttgaaattagccagatcttaagtttttctgggggaaatttagaag
aaaatatggaaaagtgactatgagcaca

### AAV6 Vector Carrying SA_GFP_IRES_PEST_SD-RAG1, Guide 9:

INSERT

tgagcacacagttattacttggaaattgtgtacagactaagttgaagatg
ttaggagggaagattgtgggccaagtaacggggtgtatgtgtgtgggtat
agggtgggcagctgggatggaaatggggggctgctgctgctgctgcaccc
tggcctcctgaactaatgatatcactcaccagaaactactgttcctgcac
tgtccaagccaccccaaactagtttgtcaaaatgaatctgtgctgtgtgg
agggaggcacgcctgtagctctgatgtcagatggcaatgtgaattcctga
cctcttctcttcctcccacaggccgccaccatggtgagcaagggcgagga
gctgttcaccggggtggtgcccatcctggtcgagctggacggcgacgtaa
acggccacaagttcagcgtgtccggcgagggcgagggcgatgccacctac
ggcaagctgaccctgaagttcatctgcaccaccggcaagctgcccgtgcc
ctggcccaccctcgtgaccaccctgacctacggcgtgcagtgcttcagtc
gctaccccgaccacatgaagcagcacgacttcttcaagtccgccatgccc
gaaggctacgtccaggagcgcaccatcttcttcaaggacgacggcaacta
caagacccgcgccgaggtgaagttcgagggcgacaccctggtgaaccgca
tcgagctgaagggcatcgacttcaaggaggacggcaacatcctggggcac
aagctggagtacaactacaacagccacaacgtctatatcatggccgacaa
gcagaagaacggcatcaaggtgaacttcaagatccgccacaacatcgagg
acggcagcgtgcagctcgccgaccactaccagcagaacacccccatcggc
gacggccccgtgctgctgcctgacaaccactacctgagcacccagtccgc
cctgagcaaagaccccaacgagaagcgcgatcacatggtcctgctggagt
tcgtgaccgccgccgggatcactctcggcatggacgagctgtacaagtaa
actggatccgaattaactcgaggaattccgcccctctccctccccccccc
ctaacgttactggccgaagccgcttggaataaggccggtgtgcgtttgtc
tatatgttattttccaccatattgccgtcttttggcaatgtgagggcccg
gaaacctggccctgtcttcttgacgagcattctaggggtctttcccctct
cgccaaaggaatgcaaggtctgttgaatgtcgtgaaggaagcagttcctc
tggaagcttcttgaagacaaacaacgtctgtagcgaccctttgcaggcag
cggaaccccccacctggcgacaggtgcctctgcggccaaaagccaacgtg
tataagatacacctgcaaaggcggcacaaccccagtgccacgttgtgagt
tggatagttgtggaaagagtcaaatggctctcctcaagcgtattcaacaa
ggggctgaaggatgcccagaaggtaccccattgtatgggatctgatctgg
ggcctcggtgcacatgctttacatgtgtttagtcgaggttaaaaaacgtc
taggccccccgaaccacggggacgtggttttcctttgaaaaacacgatga
taatatggccacaaccatgaggaccgaggcccccgagggcaccgagagcg
agatggagacccccagcgccatcaacggcaaccccagctggcacccggat
ccaggtaagttctagaatggcagtggccggtggggacagggctgagccag
caccaaccactcagcctttgagatcccgaggctggtctactgctgagacc
ttttgttagaagagaggagatcaagcatttgcaaggtttctgagtgtcaa
aatatgaatccaagataactctttcacaatcctaacttcatgctgtctac
aggtccatattttagcctgctttctccatgttcatccgaaaagaaagaaa
agctaagggtggtggtcatatttgaaattagccagatcttaagtttttct
gggggaaatttagaagaaaatatggaaaagtgactatgagcaca

HA Left

tgagcacacagttattacttggaaattgtgtacagactaagttgaagatg
ttaggagggaagattgtgggccaagtaacggggtgtatgtgtgtgggtat
agggtgggcagctgggatggaaatggggggctgctgctgctgctgcaccc
tggcctcctgaactaatgatatcactcaccagaaactactgttcctgcac
tgtccaagccaccccaaactagtttgtcaaaatgaatctgtgctgtgtgg
agggaggcacgcctgtagctctgatgtcagatggcaatgt

Splice Acceptor

ctgacctcttctcttcctcccacag

KOZAK

gccgccaccatg

GFP

atggtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagtcgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcctgacaaccac
tacctgagcacccagtccgccctgagcaaagaccccaacgagaagcgcga
tcacatggtcctgctggagttcgtgaccgccgccgggatcactctcggca
tggacgagctgtacaagtaa

IRES

gaattaactcgaggaattccgCccctctccctcccccccccctaacgtta
ctggccgaagccgcttggaataaggccggtgtgcgtttgtctatatgtta
ttttccaccatattgccgtcttttggcaatgtgagggcccggaaacctgg
ccctgtcttcttgacgagcattctaggggtctttcccctctcgccaaagg
aatgcaaggtctgttgaatgtcgtgaaggaagcagttcctctggaagctt
cttgaagacaaacaacgtctgtagcgaccctttgcaggcagcggaacccc
ccacctggcgacaggtgcctctgcggccaaaagccaacgtgtataagata
cacctgcaaaggcggcacaaccccagtgccacgttgtgagttggatagtt
gtggaaagagtcaaatggctctcctcaagcgtattcaacaaggggctgaa
ggatgcccagaaggtaccccattgtatgggatctgatctggggcctcggt
gcacatgctttacatgtgtttagtcgaggttaaaaaacgtctaggccccc
cgaaccacggggacgtggttttcctttgaaaaacacgatgataatatggc
cacaacc

PEST

atgaggaccgaggcccccgagggcaccgagagcgagatggagacccccag
cgccatcaacggcaaccccagctggcac

Splice Donor

aggtaagt

HA Right

atggcagtggccggtggggacagggctgagccagcaccaaccactcagcc
tttgagatcccgaggctggtctactgctgagaccttttgttagaagagag
gagatcaagcatttgcaaggtttctgagtgtcaaaatatgaatccaagat
aactctttcacaatcctaacttcatgctgtctacaggtccatattttagc
ctgctttctccatgttcatccgaaaagaaagaaaagctaagggtggtggt
catatttgaaattagccagatcttaagtttttctgggggaaatttagaag
aaaatatggaaaagtgactatgagcaca

### AAV6 Gene-Editing Protocol in Cell Lines

5×10⁵ cells per well were electroporated (Lonza, SF Cell line 4D Nucleofector X Kit, program FF120 for K562 or program DS100 for NALM6) with either plasmids or RNPs. Donor DNA was delivered by electroporation as fragment plasmid spanning the region between the left and right homology arms at a dose of 1600 ng.

### CD34+ Cells

Human MPB or BM CD34+ cells were obtained from Lonza and stimulated in StemSpan medium supplemented with penicillin/streptomycin antibiotics and early-acting cytokines: Stem cell factor (SCF) 300 ng/ml, Flt3 ligand (Flt3-L) 300 ng/ml, Thrombopoietin (TPO) 100 ng/ml, StemRegenin1 (SR1) (1 µM) and 16,16-dimethyl prostaglandin E2 (dmPGE2) (10 µM), UM171 35 nM.

### AAV6 Gene-Editing Protocol in CD34+ Cells

After 3 days of expansion, 2-5×10⁵ CD34+ cells per condition were electroporated (Lonza, P3 Primary Cell 4DNucleofector X Kit, CD34+ program) with RNPs, GSE56 mRNA (3 ug/test), Ad5-E4orf6/7 (1.5 ug/test) or GSE56+Ad5-E4orf6/7 as fusion protein with P2A self cleaving peptide (5 ug/test). 15 minutes after electroporation, CD34+ cells were infected with AAV6 at 10⁴ Vg/cell and kept in culture with StemSpan medium supplemented with penicillin/streptomycin antibiotics and early-acting cytokines: Stem cell factor (SCF) 300 ng/ml, Flt3 ligand (Flt3-L) 300 ng/ml, Thrombopoietin (TPO) 100 ng/ml, StemRegenin1 (SR1) (1 µM) and UM171 35 nM.

### Flow Cytometry Analysis (FACS) and Sorting

For the analysis of GFP expression, unstained and single-stained cells or compensation beads were used as negative and positive controls. For apoptosis/necrosis detection, cells were stained with 7-Aminoactinomycin D (7-AAD, BD Pharming). CD34+ cells were stained with phycoerythrin cyanine 7 (PECy7) CD34 (Clone: AC136, Miltenyi Biotec), phycoerythrin (PE) CD133 (Miltenyi Biotec) allophycocyanin (APC) CD90 (BD Biosciences). Cell sorting on CD133/CD90 edited cells was performed using MoFlo XDP Cell Sorter (Beckman Coulter).

Analysis of HSPC composition of MPB/BM-CD34+ cells was performed according to the protocol in (Basso-Ricci et al. (2017) Cytom Part A. 91: 952-65). Briefly, 1.5×10⁵ cells were labeled with fluorescent antibodies against CD3, CD56, CD14, CD61/41, CD135, CD34, CD45RA (Biolegend) and CD33, CD66b, CD38, CD45, CD90, CD10, CD11c, CD19, CD7, and CD71 (BD Biosciences). All samples were acquired through BD LSR-Fortessa (BD Bioscience) cytofluorimeter after Rainbow bead (Spherotech) calibration and raw data were collected through DIVA software (BD Biosciences).

T cell differentiation was analyzed after cell harvesting from ATOs by flow cytometry using the following mAb: TCRab APC (cl. IP26, eBioscience), CD4 Alexa Fluor 700 (cl. OKT4, eBioscience), CD19 PerCP-Cy5.5 (cl. HIB19, Biolegend), CD56 FITC (cl. MEM-188, Biolegend), CD8a PE/Dazzle (cl. RPA-T8, Biolegend), CD45 V500 (cl. HI30, BD Biosciences), CD3 BV421 (cl. UCHT1, BD Biosciences), CD8b PE (cl. 2ST8.5H7, BD Biosciences) LIVE/DEAD™ Fixable Yellow Dead Cell Stain Kit (Invitrogen). All samples were acquired through BD Cantoll (BD Bioscience) cytofluorimeter after Rainbow bead (Spherotech) calibration and raw data were collected through DIVA software (BD Biosciences).

The data were subsequently analyzed with FlowJo software Version 9.3.2 (TreeStar) and the graphical output was automatically generated through Prism 6.0c (GraphPad software).

### Clonogenic Assay

CFU-C assay was performed 24 h after editing procedure by plating 600 cells in methylcellulose-based medium (MethoCult H4434, StemCell Technologies) supplemented with 100 IU/ml penicillin and 100 µg/ml streptomycin. Three technical replicates were performed for each condition. Two weeks after plating, colonies were counted and identified according to morphological criteria.

### ATO Culture System

ATOs were generated as described in Seet et al (Seet et al. (2017) Nat Methods). Briefly, one day after the editing procedure 5000-10000 CD34⁺ from BM or MPB samples (commercially available, Lonza) were combined with 150000 MS5-hDLL4 cells per ATO. We normalized the number of “true” live CD34+ cells according to the flow cytometry analysis excluding dead and CD34- cells. Each ATO (5 µI) was then plated in a 0.4 µM Millicell Transwell insert, placed on a well of a 6-well plate containing 1 ml complete RB27 medium supplemented with rhlL-7 (5 ng/ml), rhFlt3-L (5 ng/ml) and 30 µM I-ascorbic acid 2-phosphate sesquimagnesium salt hydrate. Each insert contained a maximum of two ATOs. Medium was changed every 3-4 days. From weeks 4 to 9, ATOs were collected by adding MACS buffer (PBS with 7.5% BSA and 0.5 M EDTA) to each well and pipetting to dissociate the ATOs. Cells were then resuspended in FACS Buffer (PBS 2% FBS), counted and stained with the following antibodies: CD14 PE, CD45 PerCP-Cy5.5, CD1a APC, CD7 Alexa Fluor 700, CD5 PE-Cy7, CD34 VioBlue, CD56 FITC, CD8a APC, TCRab PerCP-Cy5.5, CD3 APC, CD4 PeVio770, CD8b PE. Yellow live dead was used to exclude dead cells. Samples were analyzed using FlowJo software version 10.5.2 (FlowJo, LLC, Ashland, OR).

### Digital PCR

Digital PCR (ddPCR) was performed to assess targeted integration. Briefly, gDNA was quantified using Nanodrop, and diluted in H₂O to reach 5-10 ng per reaction (1-2 ng/ul). It is possible to increase the gDNA quantity per reaction but it is important to remain below the saturation limit of the system. ddPCR master mix was prepared by adding 11 ul ddPCR Supermix for Probes (no dUTP; BioRad), 1.1 ul primer mix Primer forward + Primer reverse (final concentration 0.9 uM) + Probe (final concentration 0.25 uM), 1.1 ul normalizer primer mix, 4.9 ul H₂O per reaction. Finally, 17 ul of ddPCR master mix and 5 ul of diluted gDNA were added to each well (we included UT and H₂O as negative controls, and mono- or bi allelic clone as positive control to validate the system). Droplets were prepared on the BioRad AutoDG Automated Droplet Generator and the droplet plate was sealed with foil using BioRad PX1 PCR Plate Sealer. The sealed plate was placed into BioRad T100 Thermal Cycler and we ran the appropriate PCR program. The run was read in BioRad QX200 Droplet Reader.

Calculation copies per genome: concentration (copies/µl) gene of interest / concentration (copies/µl) normalizer gene x 2 Calculation percentage of HDR: copies per genome x 100.

Optimized PCR program (40 cycles):
95° C. × 10 min
40 × 94° x 30 sec
55° × 1 min
72° × 2 min
98° × 10 min
4° hold

Primers and Probes used for the ddPCR assay are the following:
PGK_GFP cassette FW CAAGAGGTTGTCTGAAGGAAG
PGK_GFP cassette RV GACGTGAAGAATGTGCGAG
PGK_GFP cassette PROBE FAM CTGCTGCACCCTGGCCTCCTGAACTAA
Corrective CDS FW GTGGAACAGGTGTGATAATGAG
Corrective CDS RV GGAGGACAATCCAAGGGTAG
Corrective CDS PROBE FAM TGCTGCTGCACCCTGGCCTCCTGAA

### RT-qPCR

For gene expression analyses, total RNA was extracted using RNeasy Plus Micro Kit (QIAGEN), according to the manufacturer’s instructions and DNase treatment was performed using RNase-free DNase Set (QIAGEN). cDNA was synthetized with the High Capacity cDNA Reverse Transcription kit (Applied Biosystem). cDNA was then used for qPCR in a Viia7 Real-time PCR thermal cycler using Power Syber Green PCR Master Mix (Applied Biosystems). Data were analyzed with Viia7 Real-Time PCR software (Applied Biosystem). Relative expression of each target gene was represented as fold changes (2^{-ΔCt}) relative to the beta-actin normalizer.

### Example 3

### Results and Discussion

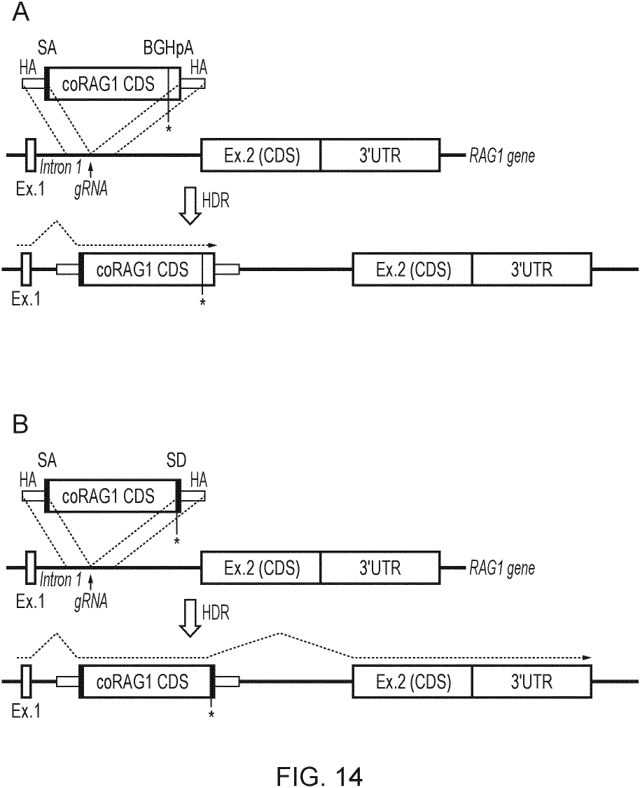

Two further donor constructs were designed and generated:
i) a SA_coRAG1 CDS_BGHpA donor carrying the bovine growth hormone (BGH) PolyA downstream the SA_ coRAG1CDS allowing the transcription termination of the corrective RAG1 CDS (FIG. 14A);
ii) a SA_coRAG1 CDS_SD containing a splice donor (SD) sequence to obtain a fusion transcript including the corrected codon optimized sequence and endogenous RAG1 followed by the 3′ UTR sequence (FIG. 14B).

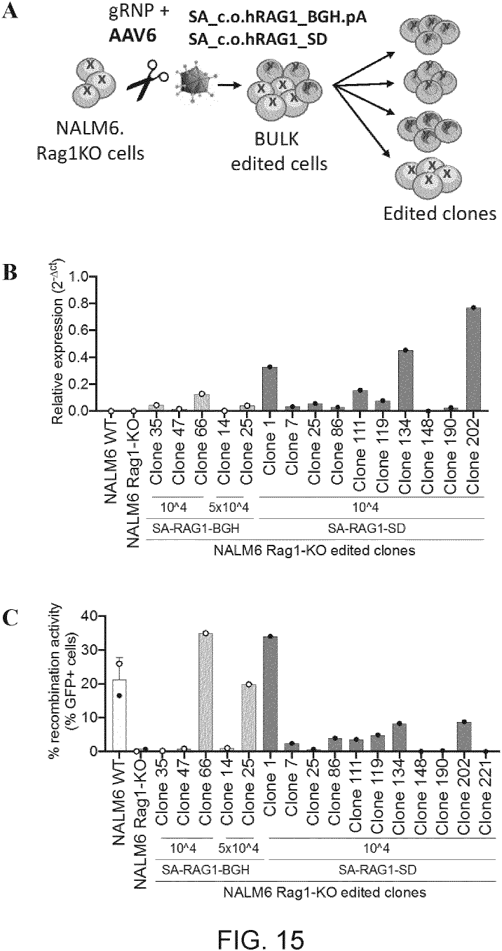

To test the two corrective donors, NALM6.Rag1KO cells were transfected with guide 9 and Cas9 as RNP (50pmol) and transduced with SA_coRAG1 CDS_BGHpA or SA_coRAG1 CDS_SD AAV6 donor at two doses (10⁴ and 5×10⁴) (FIG. 15A). As expected, we obtained low proportion of edited alleles in bulk edited NALM6.Rag1KO cells due to the low permissiveness of NALM6 cells to HDR-mediated editing. To evaluate gene editing efficiency in terms of RAG1 expression and recombination activity, edited bulk NALM6.Rag1KO cells were subcloned to isolate various single colonies carrying mono- or bi-allelic editing (FIG. 15A). We screened 429 clones by ddPCR and we identified 5 mono-allelic clones edited by SA_coRAG1 CDS_BGHpA and 11 mono-allelic clones edited by SA_coRAG1 CDS_SD.

To compare the correction efficiency of the two donors into the selected edited clones, we analyzed the RAG1 CDS expression by RT-qPCR and the recombination activity assessed by the transduction of cells with a LV carrying an inverted GFP cassette which is recombined in presence of a functional RAG1 protein (Liang HE, et al. Immunity. 2002;17:639-651; Bredemeyer AL, et al. Nature. 2006;442(7101):466-470; De Ravin SS, et al. Blood. 2010;116:1263-1271; Lee YN, et al., J Allergy Clin Immunol. 2014;133(4):1099-10).

We observed the increase of RAG1 CDS expression (FIG. 15B) and recombination activity (FIG. 15C) in the majority of clones edited by SA_coRAG1 CDS_BGHpA or SA_coRAG1 CDS_SD AAV6 donor.

To compare the two donors in terms of impact on hematopoietic stem and progenitor cells (HSPC), we edited HSPC derived from the mobilized peripheral blood of HD with guide 9 and Cas9 as RNP (50pmol) in presence of the combination of editing enhancers (GSE56 and Ad5-E4orf6/7) followed by the transduction with SA_coRAG1 CDS_BGHpA or SA_coRAG1 CDS_SD AAV6 donor at three different doses.

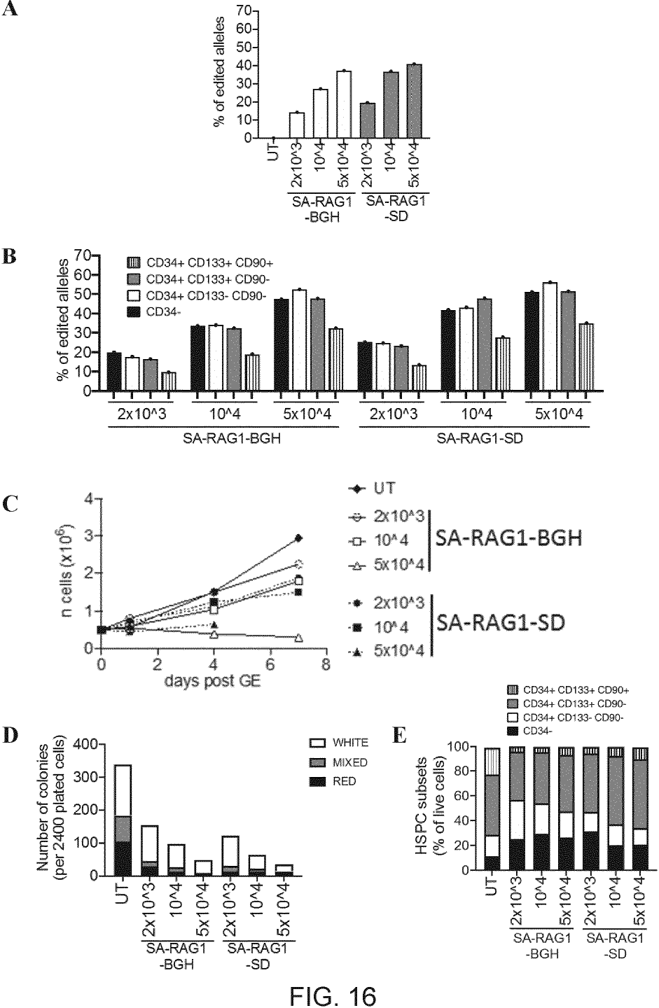

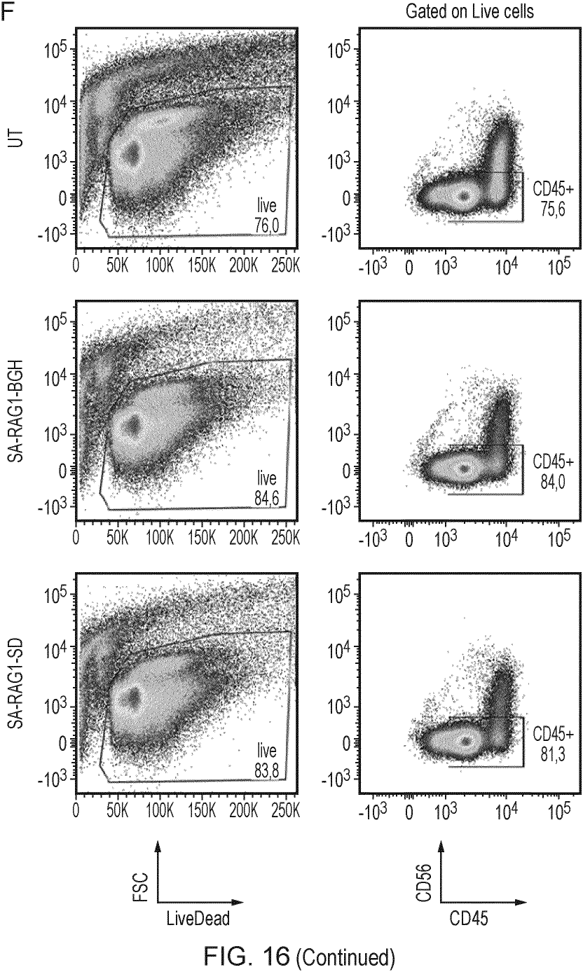

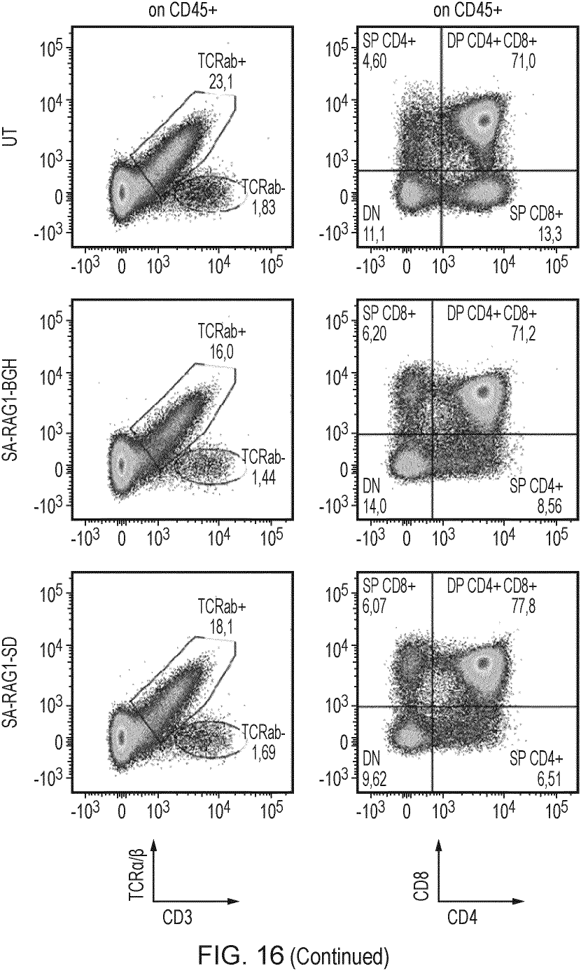

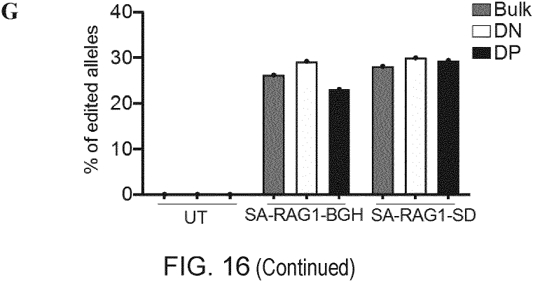

We observed comparable editing efficiencies between HSPC edited by SA_coRAG1 CDS_BGHpA or SA_coRAG1 CDS_SD AAV6 donor, increasing according to the dose (FIG. 16A) as also confirmed by the analysis of editing efficiency in sorted HSPC (FIG. 16B). Beside the known impact of gene editing on cell growth (FIG. 16C) and clonogenic potential (FIG. 16D) as compared to untreated cells, HSPC edited by SA_coRAG1 CDS_BGHpA or SA_coRAG1 CDS_SD AAV6 donor showed similar i) kinetics of growing (FIG. 16C), ii) generation of erythroid and myeloid colonies (FIG. 16D), and iii) cell subset composition with preservation of the most primitive CD34+ CD133+ CD90+ cells (FIG. 16E).

To further compare the two AAV6 donor constructs, we exploited the artificial thymic organoid (ATO) platform to differentiate edited HSPC towards the T cell lineage by applying the protocol previously described (FIG. 12). Hematopoietic stem and progenitor cells edited by the two donors similarly differentiated in early and late T cell subsets (FIG. 16F) with comparable levels of editing efficiency in sorted double negative CD4- CD8- cells and double positive CD4+ CD8+ cells (FIG. 16G).

Overall, these data indicate that both corrective donors are able to obtain efficient targeting while preserving the most primitive CD34+ CD133+ CD90+ cells subpopulation.

All publications mentioned in the above specification are herein incorporated by reference. Various modifications and variations of the disclosed polynucleotides, vectors, RNAs, methods, cells, kits, compositions, systems and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. An isolated polynucleotide comprising from 5′ to 3′: a first homology region, a splice acceptor sequence, a nucleotide sequence encoding a RAG1 polypeptide, and a second homology region.

2. The isolated polynucleotide according to claim 1, wherein:
(i) the first homology region is homologous to a first region of the RAG1 intron 1 and the second homology region is homologous to a second region of the RAG1 intron 1; or
(ii) the first homology region is homologous to a first region of the RAG1 intron 1 or the RAG1 exon 2 and the second homology region is homologous to a second region of the RAG1 exon 2.

3. The isolated polynucleotide according to claim 1 or claim 2, wherein the first homology region is homologous to a first region of the RAG1 intron 1 and the second homology region is homologous to a second region of the RAG1 intron 1.

4. The isolated polynucleotide according to any preceding claim, wherein:
(i) the first homology region is homologous to a region upstream of chr 11: 36569295 and the second homology region is homologous to a region downstream of chr 11: 36569298;
(ii) the first homology region is homologous to a region upstream of chr 11: 36573790 and the second homology region is homologous to a region downstream of chr 11: 36573793;
(iii) the first homology region is homologous to a region upstream of chr 11: 36573641 and the second homology region is homologous to a region downstream of chr 11: 36573644;
(iv) the first homology region is homologous to a region upstream of chr 11: 36573351 and the second homology region is homologous to a region downstream of chr 11: 36573354;
(v) the first homology region is homologous to a region upstream of chr 11: 36569080 and the second homology region is homologous to a region downstream of chr 11: 36569083;
(vi) the first homology region is homologous to a region upstream of chr 11: 36572472 and the second homology region is homologous to a region downstream of chr 11: 36572475;
(vii) the first homology region is homologous to a region upstream of chr 11: 36571458 and the second homology region is homologous to a region downstream of chr 11: 36571461;
(viii) the first homology region is homologous to a region upstream of chr 11: 36571366 and the second homology region is homologous to a region downstream of chr 11: 36571369;
(ix) the first homology region is homologous to a region upstream of chr 11: 36572859 and the second homology region is homologous to a region downstream of chr 11: 36572862;
(x) the first homology region is homologous to a region upstream of chr 11: 36571457 and the second homology region is homologous to a region downstream of chr 11: 36571460;
(xi) the first homology region is homologous to a region upstream of chr 11: 36569351 and the second homology region is homologous to a region downstream of chr 11: 36569354; or
(xii) the first homology region is homologous to a region upstream of chr 11: 36572375 and the second homology region is homologous to a region downstream of chr 11: 36572378.

5. The isolated polynucleotide according to any preceding claim, wherein:
(i) the first homology region is homologous to a region upstream of chr 11: 36569295 and the second homology region is homologous to a region downstream of chr 11: 36569298;
(ii) the first homology region is homologous to a region upstream of chr 11: 36573351 and the second homology region is homologous to a region downstream of chr 11: 36573354; or
(iii) the first homology region is homologous to a region upstream of chr 11: 36571366 and the second homology region is homologous to a region downstream of chr 11: 36571369;
preferably wherein the first homology region is homologous to a region upstream of chr 11: 36569295 and the second homology region is homologous to a region downstream of chr 11: 36569298.

6. The isolated polynucleotide according to any preceding claim, wherein the first homology region is homologous to a region comprising chr 11: 36569245-chr 11: 36569294 and/or the second homology region is homologous to a region comprising chr 11: 36569299-chr 11: 36569348.

7. The isolated polynucleotide according to any preceding claim, wherein the 3′ terminal sequence of the first homology region comprises or consists of a nucleotide sequence that has at least 70% identity to SEQ ID NO: 7 and/or the 5′ terminal sequence of the second homology region comprises or consists of a nucleotide sequence that has at least 70% identity to SEQ ID NO: 19.

8. The isolated polynucleotide according to any preceding claim, wherein the first homology region comprises or consists of a nucleotide sequence that has at least 70% identity to SEQ ID NO: 31, or a fragment thereof and/or the second homology region comprises or consists of a nucleotide sequence that has at least 70% identity to SEQ ID NO: 32, or a fragment thereof.

9. The isolated polynucleotide according to any preceding claim, wherein the first and second homology regions are each 50-1000 bp in length, 100-500 bp in length, or 200-400 bp in length.

10. The isolated polynucleotide according to any preceding claim, wherein the nucleotide sequence encoding a RAG1 polypeptide comprises or consists of a nucleotide sequence encoding an amino acid sequence that has at least 70% identity to SEQ ID NO: 4 or SEQ ID NO: 5.

11. The isolated polynucleotide according to any preceding claim, wherein the nucleotide sequence encoding a RAG1 polypeptide comprises or consists of a nucleotide sequence that has at least 70% identity to SEQ ID NO: 6.

12. The isolated polynucleotide according to any preceding claim, wherein the splice acceptor site comprises or consists of a nucleotide sequence that has at least 70% identity to SEQ ID NO: 33.

13. The isolated polynucleotide according to any preceding claim, wherein the nucleotide sequence encoding a RAG1 polypeptide is operably linked to a polyadenylation sequence, optionally wherein the polyadenylation sequence is a bGH polyadenylation sequence.

14. The isolated polynucleotide according to any preceding claim, wherein the nucleotide sequence encoding a RAG1 polypeptide is operably linked to a polyadenylation sequence comprising or consisting of a nucleotide sequence that has at least 70% identity to SEQ ID NO: 35.

15. The isolated polynucleotide according to any preceding claim, wherein the nucleotide sequence encoding a RAG1 polypeptide is operably linked a Kozak sequence, optionally wherein the Kozak sequence comprises or consists of a nucleotide sequence that has at least 70% identity to SEQ ID NO: 36.

16. The isolated polynucleotide according to any preceding claim, wherein the polynucleotide comprises or consists of a nucleotide sequence that has at least 70% identity to SEQ ID NO: 39.

17. A vector comprising the polynucleotide according to any preceding claim.

18. The vector according to claim 17, wherein the vector is a viral vector, optionally an adeno-associated viral (AAV) vector such as an AAV6 vector.

19. A guide RNA comprising or consisting of a nucleotide sequence that has at least 90% identity to any of SEQ ID NOs: 41-52 or 53-55, optionally wherein the guide RNA comprises or consists of a nucleotide sequence that has at least 90% identity to SEQ ID NO: 41 or 53 (preferably SEQ ID NO: 41).

20. The guide RNA according to claim 19, wherein from one to five of the terminal nucleotides at 5′ end and/or 3′ end of the guide RNA are chemically modified to enhance stability, optionally wherein three terminal nucleotides at 5′ end and/or 3′ end if the guide RNA are chemically modified to enhance stability, optionally wherein the chemical modification is modification with 2′-O-methyl 3′phosphorothioate.

21. A kit, a composition, or a gene-editing system, comprising the polynucleotide according to any one of claims 1 to 16 or the vector according to any one of claims 17 or 18.

22. The kit, composition, gene-editing system according to claim 21, wherein the kit, composition, or gene-editing system further comprises a guide RNA according to claim 19 or claim 20.

23. The kit, composition, or gene-editing system, according to claim 21 or claim 22, wherein the kit, composition, or gene-editing system, further comprises a RNA-guided nuclease, optionally wherein the RNA-guided nuclease is a Cas9 endonuclease.

24. Use of the isolated polynucleotide according to any one of claims 1 to 16, the vector according to any one of claims 17 or 18, the guide RNA according to any one of claims 19 or 20, or the kit, composition, or gene-editing system according to any one of claims 21 to 23, for gene editing a cell or a population of cells.

25. An isolated genome comprising the polynucleotide according to any one of claims 1 to 16.

26. An isolated cell comprising the polynucleotide according to any one of claims 1 to 16 or the genome according to claim 25.

27. The isolated cell according to claim 26, wherein the cell is a hematopoietic stem cell (HSC), a hematopoietic progenitor cell (HPC), or a lymphoid progenitor cell (LPC).

28. The isolated cell according to claim 26 or claim 27, wherein the cell is a CD34+ cell.

29. A population of cells comprising one or more isolated cells according to any one of claims 26 to 28.

30. The population of cells according to claim 29, wherein at least 50% of the population of cells are CD34+ cells.

31. The population of cells according to claim 29 or claim 30, wherein at least 20% of the population of cells are CD34+ cells comprising the genome according to claim 25.

32. A method of gene editing a population of cells comprising:
(a) providing a population of cells; and
(b) delivering an RNA-guided nuclease, a guide RNA according to claim 19 or claim 20, and a vector according to claim 17 or claim 18, to the population of cells to obtain a population of gene-edited cells.

33. A method of treating a RAG-deficient immunodeficiency in a subject comprising:
(a) providing a population of cells;
(b) delivering an RNA-guided nuclease, a guide RNA according to claim 19 or claim 20, and a vector according to claim 17 or claim 18, to the population of cells to obtain a population of gene-edited cells.
(c) administering the population of gene-edited cells to the subject.

34. The method according to claim 32 or claim 33, wherein the population of cells comprises or consists of HSCs, HPCs, and/or LPCs and/or wherein the population of cells comprises or consists of CD34+ cells.

35. The method according to any one of claims 32 to 34, wherein the population of cells is pre-activated, optionally wherein the population of cells is cultured with one or more cytokines selected from: one or more early acting cytokines such as TPO, IL-6, IL-3, SCF, FLT3-L; one or more transduction enhancers such as PGE2; and one or more expansion enhancers such as UM171, UM729, SR1.

36. The method according to any one of claims 32 to 35, wherein the RNA-guided nuclease and/or guide RNA is delivered prior to the vector and/or simultaneously with the vector.

37. The method according to any one of claims 32 to 36, wherein the RNA-guided nuclease is Cas9, optionally wherein the Cas9 and the guide RNA are delivered preassembled as Cas9 RNPs.

38. The method according to any one of claims 32 to 37, wherein the method further comprises delivering a p53 inhibitor and/or a HDR enhancer, optionally wherein the p53 inhibitor and/or a HDR enhancer is delivered simultaneously with the RNA-guided nuclease and/or guide RNA.

39. The method according to any one of claims 32 to 38, wherein the population of gene-edited cells is defined according to any one of claims 29 to 31.

40. A population of gene-edited cells obtainable by the method according to any one of claims 32 to 39.

41. A method of treating a RAG-deficient immunodeficiency comprising administering the isolated cell according to any one of claims 26 to 28, the population of cells according to any one of claims 29 to 31, or the population of gene-edited cells according to claim 40, to a subject in need thereof.

42. The isolated cell according to any one of claims 26 to 28, the population of cells according to any one of claims 29 to 31, or the population of gene-edited cells according to claim 40, for use in treating a RAG-deficient immunodeficiency in a subject.

43. The method according to claim 41, or the isolated cell, population of cells, or population of gene-edited cells for use according to claim 42, wherein the RAG-deficient immunodeficiency is T- B- severe combined immunodeficiency (SCID), Omenn syndrome, atypical SCID or combined immunodeficiency with granuloma/autoimmunity (CID-G/AI).

44. The method according to claim 41 or claim 43, or the isolated cell, population of cells, or population of gene-edited cells for use according to claim 42 or claim 43, wherein the subject has a RAG1 deficiency.

45. The method according to any one of claims 41, 43, or 44, or the isolated cell, population of cells, or population of gene-edited cells for use according to any one of claims 42 to 44, wherein the subject has a mutation in the RAG1 gene, optionally in RAG1 exon 2.
